SEQUENCE LISTING (6 sequences)

SEQ ID NO: 1 - DNA, 25 bp, Artificial Sequence
  Left HA-rev primer
tcaccatttggacgagcaggctgga

SEQ ID NO: 2 - DNA, 35 bp, Artificial Sequence
  Left HA-for primer
gacggccagtgaattcttccccagcctagttcacg

SEQ ID NO: 3 - DNA, 35 bp, Artificial Sequence
  Right HA-rev primer
cgactctagaggatcccagtggcagagcccatttc

SEQ ID NO: 4 - DNA, 24 bp, Artificial Sequence
  Right HA-for primer
tccccgcggccaaggccgagatgc

SEQ ID NO: 5 - DNA, 28 bp, Artificial Sequence
  Reporter-for primer
tcgtccaaatggtgagcaagggcgagga

SEQ ID NO: 6 - DNA, 28 bp, Artificial Sequence
  Reporter-rev primer
ccttggccgcggggaggcggcccaaagg

(19) United States
(12) Patent Application Publication
(10) Pub. No.: US 2020/0223837 A1
(43) Pub. Date: Jul. 16, 2020

(54) BENZOFURAN AMIDES AND HETEROAROMATIC ANALOGUES THEREOF FOR USE IN THERAPY

(71) Applicant: European Molecular Biology Laboratory, Heidelberg (DE)

(72) Inventors: David William Will, Heidelberg (DE); George Reid, Heidelberg (DE); Iryna Charapitsa, Heidelberg (DE); Joe David Lewis, Dielheim (DE)

(21) Appl. No.: 16/621,580

(22) PCT Filed: Jun. 14, 2018
(86) PCT No.: PCT/EP2018/065814

§ 371 Date: Dec. 11, 2019

(30) Foreign Application Priority Data
Jun. 14, 2017 (EP) 17175892.3

(51) Int. Cl. C07D 413/12; A61P 35/00

(52) U.S. Cl. CPC C07D 413/12; A61P 35/00

(57) ABSTRACT

The present invention relates to a pharmaceutical composition comprising a compound of the formula I as described below or a tautomer or a pharmaceutically acceptable salt thereof; to the compound of the formula I as described below or a tautomer or a pharmaceutically acceptable salt thereof for use as a medicament, especially for use in the treatment or prevention of a disease or disorder selected from the group consisting of an inflammatory disease, a hyperproliferative disease or disorder, a hypoxia-related pathology and a disease characterized by excessive vascularization, and to certain novel compounds of the formula I as described below or a tautomer or a pharmaceutically acceptable salt thereof. Formula (I) wherein X¹ is CR¹ or N; X² is CR² or N; X³ is CR³ or N; X⁴ is CR⁴ or N; with the proviso that at most two of X¹, X², X³ and X⁴ are N; L¹, L² are a bond or a bivalent radical such as C₁-C₆-alkylene or C₃-C₈-cycloalkylene; A is 3-, 4-, 5-, 6-, 7- or 8-membered saturated, partially unsaturated or maximally unsaturated carbocyclic ring which may carry one or more substituents R⁹; or L²-A forms a group C₁-C₆-alkylene-OR¹³, C₁-C₆-alkylene-SR¹⁴ or C₁-C₆-alkylene-NR¹⁵R¹⁶; and R¹, R², R³, R⁴, R⁵, R⁶, R⁹, R¹³, R¹⁴, R¹⁵ and R¹⁶ are as defined in the claims and the description.

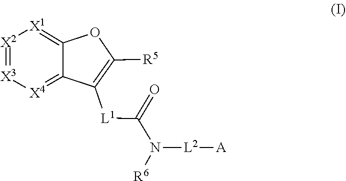

### CROSS-REFERENCE TO RELATED APPLICATION(S)

This patent application claims the benefit of priority of EP Application No. 17175892.3, filed Jun. 14, 2017.

### SEQUENCE LISTING

The instant application contains a Sequence Listing which has been submitted in ASCII format via EFS-Web and is hereby incorporated by reference in its entirety. Said ASCII copy, created on Feb. 21, 2020, is named 05710_037US1_SL.txt and is 1,403 bytes in size.

### FIELD OF THE INVENTION

The present invention relates to a pharmaceutical composition containing benzofuran amides or heteroaromatic analogues thereof, to these compounds for use in therapy, especially for use in the treatment or prevention of a disease or disorder selected from the group consisting of an inflammatory disease, a hyperproliferative disease or disorder, a hypoxia-related pathology and a disease characterized by excessive vascularization, and to certain novel benzofuran amides or heteroaromatic analogues thereof.

### BACKGROUND OF THE INVENTION

Despite the recent extraordinary progress seen in cancer therapy using molecularly targeted drugs, cancer remains a major cause of death worldwide. The major barrier to successful treatment and prevention of cancer lies in the fact that many cancers are resistant or refractory to current chemotherapeutic and/or immunotherapy intervention, and many individuals suffer recurrence or death, even after aggressive therapy. Therefore, there is an ongoing need for expanding the treatment options for cancer patients, including the provision of new drugs.

Reductive characterization of tumors has uncovered a set of phenotypic states necessary for malignancy. These phenotypic states consist of distinct traits that are necessary and sufficient for malignancy. One of the earliest and most consistent traits of malignancy is the acquisition of a distinct metabolic programme, where cells limit their generation of energy largely to glycolytic fermentation, even when oxygen is available. This phenotype, known as aerobic glycolysis or the Warburg effect, was first reported by the Nobel laureate Otto Warburg in the 1930s' (O. Warburg et al., Berlin-Dahlem. London: Constable & Co. Ltd. (1930); O. Warburg, Science, 1956, 123, 309-314; O. Warburg, Science, 1956, 124, 269-270) and differentiates proliferating cells from quiescent cells. Substrates for this aerobic glycolysis are glucose or amino acids, in particular glutamine or asparagine.

The PI3K-Akt-mTOR (phosphotidyl inositol 3 kinase, Akt Serine/Threonine Kinase and Mechanistic Target Of Rapamycin) cascade is a major signaling pathway that induces aerobic glycolysis and is associated with the development of the majority of cancers. The Akt signaling pathway is, thus, a major target for the development of cancer therapeutics (J. S. Brown et al., Pharmacol Ther., 2017, 172, 101-115).

The egr1 gene is an immediate early gene whose activity is controlled by expression. Its expression product, EGR1, is a transcription factor belonging to the family of Cys₂-His₂ zinc finger proteins. EGR1 is known to have a significant role in cancer (Baron et al., Cancer Gene Therapy, 2006, 13, 115-124). EGR1 integrates signals from many different pathways (I. Gudernova et al., Elife. 6:e21536 (2017)). EGR1 can act as tumor suppressor gene in fibrosarcoma, glioblastoma and in lung and breast cancer (C. Liu et al., J Biol Chem, 1999, 274(7), 4400-4411; C. Liu et al., J Biol Chem, 2000, 275(27), 20315-20323; M. M. Shareef et al., Cancer Res, 2007, 67(24), 11811-11820; R. P. Huang et al., Int J Cancer, 1997, 72(1), 102-109). EGR1 suppresses tumourogenesis by transactivating expression of TGFβ1, PTEN, fibronectin and p53 and by cooperating with Sp1, Jun-B and p21 (C. Liu et al., J Biol Chem, 1999, 274(7), 4400-4411; C. Liu et al., Cancer Gene Ther, 1998, 5(1), 3-28; V. Baron et al., Cancer Gene Ther, 2006, 13(2), 115-124). Therefore, compounds causing up-regulation of EGR1 expression at low dosage are considered to be useful in therapy of cancer and other proliferative diseases.

HSF1 (heat shock factor 1) is a transcription factor that is the master regulator of the expression of heat shock transcripts. C. Dai et al., Cell. 130:1005-18 (2007) found that HSF1 knock-out mice are resistant to chemically induced carcinogenesis and concluded that HSF1 is a central player in cancer. Moreover, HSF1 facilitates oncogenesis promoted by mutant p53. A large body of work has verified the importance of HSF1 in tumorigenesis and in cancer progression (see e.g. L. Whitesell et al., Expert Opin. Ther. Targets 2009, 13, 469-478; C. L. Moore, et al., ACS Chem. Biol. 2016, 11, 200-210, E. de Billy, et al., Oncotarget 2012, 3, 741 743). HSF1 supports the most aggressive forms of breast, lung and colon cancer, with HSF1-driven transcriptional programmes strongly associated with metastasis and death in a wide range of cancer (Mendillo et al., Cell 150: 549 (2012)). Finally, Kaplan Meier analysis demonstrates that patients whose tumors express high levels of HSF1 have a much poorer prognosis than patients expressing less HSF1, in multiple tumor types (B. Gyorffy et al. PLos One 8:e82241 (2013). C. Dai et al., Cell. 130:1005-18 (2007) further found that fibroblasts from HSF1 knockout mice have a lower requirement for glucose. Additionally, rohinitib, a rocaglamide that, amongst other activities (M. Li-Weber, Int J Cancer, 2015, 137(8), 1791-1799), prevents HSF1 binding to target enhancer elements, reduces glucose uptake of tumour cells (S. Santagata et al., Science, 2013, 341(6143):1238303). In conclusion, HSF1 has a sentinel, permissive role in licensing aerobic glycolysis by modulating glucose and neutral amino acid metabolism. Consequently, compromising HSF1 activity is an attractive target for new, effective and safe cancer treatment.

Pirin is a non-haem, iron containing protein that acts as a redox sensor in cells. It is ubiquitously expressed and is frequently expressed at higher levels in tumor cells than in surrounding normal tissue. For example, pirin has been linked to metastasis in myeloma (S. Licciulli et al., Am J Pathol, 2011, 178(5), 2397-2406; I. Miyazaki et al., Nat Chem Biol, 2010, 6(9), 667-673), is upregulated in the spleen and kidney of superoxide dismutase deficient mice (K. Brzoska et al., Redox Rep, 2011, 16(3), 129-133) and in the lungs of chronic smokers (B. D. Gelbman et al., Respir Res, 2007, 8:10). Pirin undergoes a conformational switch upon oxidation of the bound iron from Fe²⁺ to Fe³⁺. Oxidized pirin promotes the interaction of target promoters with the transcription factor NF-kB, a critical mediator of intracellular signaling that has been linked to cellular responses to proinflammatory signals and which controls the expression of a large array of genes involved in immune and stress responses (Lui et al., Proc. Natl. Acad. Sci. USA, 110:9722-7 (2013)).

M. D. Cheeseman et al., J Med Chem. 60:180-201 (2017) recently found that pirin is a key regulator of HSF1 and that small molecule ligands to pirin efficiently inhibit HSF1-mediated stress pathway. The authors could confirm in a human ovarian carcinoma xenograft model that their pirin ligand showed 70% tumor growth inhibition.

It is apparent from the foregoing that small molecule ligands to pirin will likely be useful in therapy of cancer and other proliferative diseases and also for therapy of inflammatory diseases, hypoxia-related pathologies and diseases characterized by excessive vascularization.

It is an object of the present invention to provide new therapeutic agents which allow for an efficient treatment of different proliferative and inflammatory diseases or disorders, hypoxia-related pathologies and/or diseases characterized by excessive vascularization. The compounds should be efficient ligands to pirin at low dosage, should cause up-regulation of EGR1 expression at low EC50 values, and/or downregulate the HSF1 expression. Expediently, the compounds should also show good bioavailability and/or metabolic stability and/or low blockade of the hERG channel.

It was now found that the compounds of formula (I) as described herein are efficient ligands to pirin that efficiently cause up-regulation of EGR1 expression at low EC50 values. It was also found that these compounds downregulate the HSF1 expression, the master regulator of the heat shock response and a powerful driver of oncogenesis, and block PI3K-Akt-mTOR signalling. Collectively, these changes provoke profound down-regulation of the transcription and expression of multiple solute transporters and glycolytic enzymes. Moreover, it could be confirmed by using in vivo and in vitro models that the compounds of formula (I) inhibit tumor growth. The compounds of formula (I) show good bioavalabilty and metabolic stability.

### SUMMARY OF THE INVENTION

The present invention relates to a pharmaceutical composition comprising a compound of the formula I as described below or a tautomer or a pharmaceutically acceptable salt thereof; to the compound of the formula I as described below or a tautomer or a pharmaceutically acceptable salt thereof for use as a medicament, especially for use in the treatment or prevention of a disease or disorder selected from the group consisting of an inflammatory disease, a hyperproliferative disease or disorder, a hypoxia-related pathology and a disease characterized by excessive vascularization, and to certain novel compounds of the formula I as described below or a tautomer or a pharmaceutically acceptable salt thereof.

Thus, in one aspect, the present invention relates to a pharmaceutical composition comprising a compound of the formula I or a tautomer or a pharmaceutically acceptable salt thereof

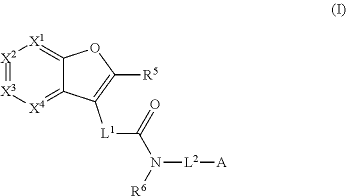

wherein
X¹ is CR¹ or N;
X² is CR² or N;
X³ is CR³ or N;
X⁴ is CR⁴ or N;
with the proviso that at most two of X¹, X², X³ and X⁴ are N;
L¹ is a bond, C₁-C₆-alkylene which may carry one or more substituents R⁷, or C₃-C₈-cycloalkylene which may carry one or more substituents R⁸;
L² is a bond, C₁-C₆-alkylene which may carry one or more substituents R⁷, C₃-C₈-cycloalkylene which may carry one or more substituents R⁸, C₁-C₆-alkylene-O, C₁-C₆-alkylene-S, C₁-C₆-alkylene-NR¹⁵, where the alkylene moiety in the three last-mentioned radicals may carry one or more substituents R⁷; C₃-C₈-cycloalkylene-O, C₃-C₈-cycloalkylene-S or C₃-C₈-cycloalkylene-NR¹⁵, where the cycloalkylene moiety in the three last-mentioned radicals may carry one or more substituents R⁸;
A is 3-, 4-, 5-, 6-, 7- or 8-membered saturated, partially unsaturated or maximally unsaturated carbocyclic ring which may carry one or more substituents R⁹; or a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁰;
or L²-A forms a group C₁-C₆-alkylene-OR¹³, C₁-C₆-alkylene-SR¹⁴ or C₁-C₆-alkylene-NR¹⁵R¹⁶;
R¹, R², R³ and R⁴, independently of each other, are selected from the group consisting of hydrogen, halogen, CN, nitro, SF₅, C₁-C₆-alkyl which may carry one or more substituents R¹¹, C₁-C₆-haloalkyl, C₃-C₈-cycloalkyl which may carry one or more substituents R¹², OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
or R¹ and R², or R² and R³, or R³ and R⁴, together with the carbon atoms they are bound to, form a 3-, 4-, 5-, 6- or 7-membered saturated, partially unsaturated or maximally unsaturated carbocyclic or heterocyclic ring, where the heterocyclic ring contains 1, 2 or 3 heteroatoms or heteroatom-containing groups selected from the group consisting of O, N, S, NO, SO and SO₂ as ring members, where the carbocyclic or heterocyclic ring may carry one or more substituents R¹⁸;
R⁵ is selected from the group consisting of hydrogen, C₁-C₆-alkyl, C₁-C₆-haloalkyl, aryl, aryl-C₁-C₃-alkyl, where the aryl moiety in the two last-mentioned radicals may carry one or more substituents R¹⁸; hetaryl and hetaryl-C₁-C₃-alkyl, where hetaryl is a 5- or 6-membered heteroaromatic ring containing 1, 2, 3, or 4 heteroatoms selected from the group consisting of O, S and N as ring members, where the heteroaromatic ring may carry one or more substituents R¹⁸;
R⁶ is selected from the group consisting of hydrogen, C₁-C₆-alkyl which may carry one or more substituents R¹¹, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-cycloalkyl-C₁-C₄-alkyl, where cycloalkyl in the two last-mentioned radicals may carry one or more substituents R¹²; C₁-C₆-alkoxy, C₁-C₆-haloalkoxy, aryl, aryl-C₁-C₃-alkyl, where the aryl moiety in the two last-mentioned radicals may carry one or more substituents R¹⁸; heterocyclyl and heterocyclyl-C₁-C₃-alkyl, where heterocyclyl is a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
R⁷ and R⁸, independently of each other and independently of each occurrence, are selected from the group consisting of F, CN, nitro, SF₅, C₁-C₆-alkyl which may carry one or more substituents R¹¹, C₁-C₆-haloalkyl (preferably fluorinated C₁-C₆-alkyl), C₃-C₈-cycloalkyl which may carry one or more substituents R¹², OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸; or two radicals R⁷ bound on the same carbon atom of the alkylene group, or two radicals R⁸ bound on the same carbon atom of the cycloalkylene group form together a group ═O or ═S;
each R⁹ is independently selected from the group consisting of halogen, CN, nitro, SF₅, C₁-C₆-alkyl which may carry one or more substituents R¹¹, C₁-C₆-haloalkyl, C₃-C₈-cycloalkyl which may carry one or more substituents R¹², OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
or two radicals R⁹ bound on adjacent ring atoms, together with the ring atoms they are bound to, may form a saturated, partially unsaturated or maximally unsaturated 3-, 4-, 5- or 6-membered carbocyclic ring which may be substituted by one or more radicals selected from the group consisting of halogen, CN, nitro, SF₅, C₁-C₆-alkyl which may carry one or more substituents R¹¹, C₁-C₆-haloalkyl, C₃-C₈-cycloalkyl which may carry one or more substituents R¹², OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
or two radicals R⁹ bound on non-adjacent ring atoms may form a bridge —CH₂— or —(CH₂)₂—;
each R¹⁰ is independently selected from the group consisting of halogen, CN, nitro, SF₅, C₁-C₆-alkyl which may carry one or more substituents R¹¹, C₁-C₆-haloalkyl, C₃-C₈-cycloalkyl which may carry one or more substituents R¹², OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
or two radicals R¹⁰ bound on adjacent ring atoms, together with the ring atoms they are bound to, may form a saturated, partially unsaturated or maximally unsaturated 3-, 4-, 5- or 6-membered carbocyclic or heterocyclic ring, where the heterocyclic ring contains 1, 2, 3 or 4 heteroatoms or heteroatom-containing groups selected from the group consisting of O, N, S, NO, SO and SO₂ as ring members, where the carbocyclic or heterocyclic ring may be substituted by one or more radicals selected from the group consisting of halogen, CN, nitro, SF₅, C₁-C₆-alkyl which may carry one or more substituents R¹¹, C₁-C₆-haloalkyl, C₃-C₈-cycloalkyl which may carry one or more substituents R¹², OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
each R¹¹ is independently selected from the group consisting of CN, nitro, SF₅, C₃-C₈-cycloalkyl which may carry one or more substituents R¹², OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
each R¹² is independently selected from the group consisting of halogen, CN, nitro, SF₅, C₁-C₆-alkyl, C₁-C₆-haloalkyl, C₃-C₈-cycloalkyl, C₃-C₈-halocycloalkyl, OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
each R¹³ is independently selected from the group consisting of hydrogen, C₁-C₆-alkyl which may carry one or more substituents R¹⁹, C₁-C₆-haloalkyl, C₃-C₈-cycloalkyl which may carry one or more substituents R²⁰, S(O)ₘR¹⁴, C(O)R¹⁷, C(O)OR²¹, C(O)NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
each R¹⁴ is independently selected from the group consisting of hydrogen, C₁-C₆-alkyl which may carry one or more substituents R¹⁹, C₁-C₆-haloalkyl, C₃-C₈-cycloalkyl which may carry one or more substituents R²⁰, OR²¹, NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
R¹⁵ and R¹⁶, independently of each other and independently of each occurrence, are selected from the group consisting of hydrogen, C₁-C₆-alkyl which may carry one or more substituents R¹⁹, C₁-C₆-haloalkyl, C₃-C₈-cycloalkyl which may carry one or more substituents R²⁰, OR²¹, S(O)ₘR²², C(O)R¹⁷, C(O)OR²¹, C(O)NR²³R²⁴, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
or R¹⁵ and R¹⁶, together with the nitrogen atom they are bound to, form a saturated, partially unsaturated or maximally unsaturated 3-, 4-, 5- or 6-membered heterocyclic ring, where the heterocyclic ring may additionally contain 1 or 2 further heteroatoms or heteroatom-containing groups selected from the group consisting of O, N, S, NO, SO and SO₂ as ring members, where the heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo;
each R¹⁷ is independently selected from the group consisting of hydrogen, C₁-C₆-alkyl which may carry one or more substituents R¹⁹, C₁-C₆-haloalkyl, C₃-C₈-cycloalkyl which may carry one or more substituents R²⁰, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
each R¹⁸ is independently selected from the group consisting of halogen, CN, nitro, OH, SH, SF₅, C₁-C₆-alkyl which may carry one or more substituents selected from the group consisting of CN, OH, C₁-C₆-alkoxy, C₁-C₆-haloalkoxy, SH, C₁-C₆-alkylthio, C₁-C₆-haloalkylthio, C₁-C₆-alkylsulfonyl, C₁-C₆-haloalkylsulfonyl, NR²³R²⁴ and phenyl; C₁-C₆-haloalkyl, C₃-C₈-cycloalkyl which may carry one or more substituents selected from the group consisting of halogen, CN, OH, C₁-C₆-alkyl, C₁-C₆-haloalkyl, C₁-C₆-alkoxy, C₁-C₆-haloalkoxy, SH, C₁-C₆-alkylthio, C₁-C₆-haloalkylthio, C₁-C₆-alkylsulfonyl, C₁-C₆-haloalkylsulfonyl and phenyl; C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, carboxyl, C₁-C₆-alkylcarbonyl, C₁-C₆-haloalkylcarbonyl, C₁-C₆-alkoxycarbonyl, C₁-C₆-haloalkoxycarbonyl, aryl and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where aryl or the heterocyclic ring may carry one or more substituents selected from the group consisting of halogen, CN, OH, C₁-C₆-alkyl, C₁-C₆-haloalkyl, C₁-C₆-alkoxy and C₁-C₆-haloalkoxy;
or two radicals R¹⁸ bound on adjacent ring atoms, together with the ring atoms they are bound to, may form a saturated, partially unsaturated or maximally unsaturated 3-, 4-, 5- or 6-membered carbocyclic or heterocyclic ring, where the heterocyclic ring contains 1, 2, 3 or 4 heteroatoms or heteroatom-containing groups selected from the group consisting of O, N, S, NO, SO and SO₂ as ring members, where the carbocyclic or heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo;
each R¹⁹ is independently selected from the group consisting of CN, OH, C₃-C₈-cycloalkyl, C₃-C₈-halocycloalkyl, C₁-C₆-alkoxy, C₁-C₆-haloalkoxy, SH, C₁-C₆-alkylthio, C₁-C₆-haloalkylthio, C₁-C₆-alkylsulfonyl, C₁-C₆-haloalkylsulfonyl, NR²³R²⁴, aryl and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where aryl or the heterocyclic ring may carry one or more substituents R¹⁸, where R¹⁸ is in particular selected from the group consisting of halogen, CN, OH, C₁-C₆-alkyl, C₁-C₆-haloalkyl, C₁-C₆-alkoxy and C₁-C₆-haloalkoxy;
each R²⁰ is independently selected from the group consisting of halogen, CN, OH, C₁-C₆-alkyl, C₁-C₆-haloalkyl, C₁-C₆-alkoxy, C₁-C₆-haloalkoxy, SH, C₁-C₆-alkylthio, C₁-C₆-haloalkylthio, C₁-C₆-alkylsulfonyl, C₁-C₆-haloalkylsulfonyl and phenyl;
R²¹ and R²², independently of each other and independently of each occurrence, are selected from the group consisting of hydrogen, C₁-C₆-alkyl which may carry one or more substituents R¹⁹, C₁-C₆-haloalkyl, C₃-C₈-cycloalkyl, C₃-C₈-halocycloalkyl, aryl and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where aryl or the heterocyclic ring may carry one or more substituents selected from the group consisting of halogen, CN, OH, C₁-C₆-alkyl, C₁-C₆-haloalkyl, C₁-C₆-alkoxy and C₁-C₆-haloalkoxy;
R²³ and R²⁴, independently of each other and independently of each occurrence, are selected from the group consisting of hydrogen, C₁-C₆-alkyl, C₁-C₆-haloalkyl, C₃-C₈-cycloalkyl, C₃-C₈-halocycloalkyl, C₁-C₆-alkylcarbonyl, C₁-C₆-haloalkylcarbonyl, C₁-C₆-alkoxycarbonyl, C₁-C₆-haloalkoxycarbonyl, C₁-C₆-alkylsulfonyl, C₁-C₆-haloalkylsulfonyl, aryl and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where aryl or the heterocyclic ring may carry one or more substituents selected from the group consisting of halogen, CN, OH, C₁-C₆-alkyl, C₁-C₆-haloalkyl, C₁-C₆-alkoxy and C₁-C₆-haloalkoxy;
m is 1 or 2; and
n is 0, 1 or 2;
and
at least one pharmaceutically acceptable carrier and/or auxiliary substance.

In another aspect, the invention relates to a compound of formula I or a tautomer or a pharmaceutically acceptable salt thereof for use as a medicament.

In another aspect, the invention relates to a compound of formula I or a tautomer or a pharmaceutically acceptable salt thereof for use in the treatment of conditions, disorders or diseases selected from the group consisting of inflammatory diseases, hyperproliferative diseases or disorders, a hypoxia related pathology and a disease characterized by pathophysiological hypervascularization.

In yet another aspect, the invention relates to the use of a compound of formula I or a tautomer or a pharmaceutically acceptable salt thereof for preparing a medicament for the treatment of conditions, disorders or diseases selected from the group consisting of inflammatory diseases, hyperproliferative diseases or disorders, a hypoxia related pathology and a disease characterized by pathophysiological hypervascularization.

In yet another aspect, the invention relates to a method for treating conditions, disorders or diseases selected from the group consisting of inflammatory diseases, hyperproliferative diseases or disorders, a hypoxia related pathology and a disease characterized by pathophysiological hypervascularization, which method comprises administering to a subject in need thereof a compound of formula I or a tautomer or a pharmaceutically acceptable salt thereof or a pharmaceutical composition containing a compound of formula I or a tautomer or a pharmaceutically acceptable salt thereof.

Finally, the invention relates to certain novel compounds I and to their tautomers and pharmaceutically acceptable salts. These compounds are specified below.

### DETAILED DESCRIPTION OF THE INVENTION

Provided the compounds of the formula I of a given constitution may exist in different spatial arrangements, for example if they possess one or more centers of asymmetry, polysubstituted rings or double bonds, or as different tautomers, the invention also relates to enantiomeric mixtures, in particular racemates, diastereomeric mixtures and tautomeric mixtures, preferably, however, the respective essentially pure enantiomers (enantiomerically pure), diastereomers and tautomers of the compounds of formula (I) and/or of their salts.

One center of asymmetry is for example L¹ if this is methylene substituted by one R⁷ or by two different R⁷, or is C₂-C₆-alkylene with at least one asymmetric C atom, or is C₃-C₈-cycloalkylene with at least one asymmetric C atom. One example for such L¹ being a center of asymmetry is CH(CH₃). Analogously, L² can be a center of asymmetry if this is methylene substituted by one R⁷ or by two different R⁷, or is C₂-C₆-alkylene with at least one asymmetric C atom, or is C₃-C₈-cycloalkylene with at least one asymmetric C atom. Other centers of chirality are for example compounds I in which A is saturated or partially unsaturated carbocyclic or heterocyclic ring containing at least one asymmetric C atom.

Racemates obtained can be resolved into the isomers mechanically or chemically by methods known per se. Diastereomers are preferably formed from the racemic mixture by reaction with an optically active resolving agent. Examples of suitable resolving agents are optically active acids, such as the D and L forms of tartaric acid, diacetyltartaric acid, dibenzoyltartaric acid, mandelic acid, malic acid, lactic acid or the various optically active camphorsulfonic acids, such as D- or L-camphorsulfonic acid. Also advantageous is enantiomer resolution with the aid of a column filled with an optically active resolving agent (for example dinitrobenzoylphenylglycine); an example of a suitable eluent is a hexane/isopropanol/acetonitrile mixture. The diastereomer resolution can also be carried out by standard purification processes, such as, for example, chromatography or fractional crystallization. It is also possible to obtain optically active compounds of formula (I) by the methods described below by using starting materials which are already optically active.

The invention also relates to “pharmaceutically acceptable salts” of the compounds of the formula (I), especially acid addition salts with physiologically tolerated, i.e. pharmaceutically acceptable acids. Examples of suitable physiologically tolerated organic and inorganic acids include, but are not limited to, hydrochloric acid, hydrobromic acid, phosphoric acid, sulfuric acid, C₁-C₄-alkylsulfonic acids, such as methanesulfonic acid, aromatic sulfonic acids, such as benzenesulfonic acid and toluenesulfonic acid, carboxylic acids such as oxalic acid, malic acid, maleic acid, fumaric acid, lactic acid, tartaric acid, adipic acid, mandelic acid, salicylic acid, phenylpropionic acid, nicotinic acid, benzoic acid acetate, alginic acid, ascorbic acid, aspartic acid, tannic acid, butyric acid, camphoric acid, citric acid, clavulanic acid, cyclopentanepropionic acid, gluconic acid, formic acid, acetic acid, propionic acid, pivalic acid, valeric acid, hexoic acid, heptoic acid, oleic acid, palmitic acid, pantothenic acid, pectinic acid, stearic acid, hexylresorcinic acid, hydroxynaphthoic acid, lactobionic acid and mucic acid. Other utilizable acids are described in Fortschritte der Arzneimittelforschung [Advances in drug research], Volume 10, pages 224 ff., Birkhäuser Verlag, Basel and Stuttgart, 1966 and in Berge, S. M., et al., “Pharmaceutical Salts”, Journal of Pharmaceutical Science, 1977, 66, 1-19. Illustrative examples of pharmaceutically acceptable salts include but are not limited to: acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formiate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like. Certain specific compounds of the present invention contain both basic and acidic functionalities that allow the compounds to be converted into either base or acid addition salts. Furthermore, where the compound of the invention carries an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counteranions such as halide, hydroxide, carboxylate, sulfate, phosphate, nitrate, alkyl sulfonate and aryl sulfonate).

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

The invention also relates to N-oxides of the compounds of the formula (I), provided that those compounds contain a basic nitrogen atom, such as the nitrogen atom of a nitrogen containing heterocycle which may be present A, or one of X¹ to X⁴ being N. Examples of nitrogen containing heterocycle, where the nitrogen may be present in the form of an N-oxide, include pyridinyl, pyrimidinyl, pyrazinyl, pyridazinyl, pyrazolyl, imidazolyl, oxazolyl, oxadiazolyl, triazolyl and the like.

The invention moreover relates to tautomers of compounds I as depicted. For instance, amide/imidic acid tautomerism in the depicted C(O)—NH group may be present. Analogously, tautomerism may be present if in ring A a NH ring member is adjacent to C═O or inversely ring A contains a moiety —C(OH)═N—. Also if X¹ is N and X² is C—OH or X² is N and X¹ or X³ is C—OH or X³ is N and X² or X⁴ is C—OH or X⁴ is N and X³ is C—OH, tautomerism may be present. Further, keto/enol tautomerism may be present if A contains a moiety —C(═O)—CH₂— or —C(═O)—CHR⁹— or —C(═O)—CHR¹⁰— or —C(OH)═CH— or —C(OH)═CR⁹— or —C(OH)═CR¹⁰—.

In addition to salt forms, the N-oxides, the salts of the N-oxides and the tautomers, the present invention provides compounds which are in a prodrug form. Prodrugs of the compounds described herein are those compounds that readily undergo chemical changes under physiological conditions to provide a compound of general formula (I). A prodrug is a pharmacologically active or inactive compound that is modified chemically through in vivo physiological action, such as hydrolysis, metabolism and the like, into a compound of this invention following administration of the prodrug to a patient. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme. The suitability and techniques involved in making and using prodrugs are well known by those skilled in the art. For a general discussion of prodrugs involving esters, see Svensson and Tunek, Drug Metabolism Reviews 16.5 (1988), and Bundgaard, Design of Prodrugs, Elsevier (1985). Examples of a masked acidic anion include a variety of esters, such as alkyl (for example, methyl, ethyl), cycloalkyl (for example, cyclohexyl), aralkyl (for example, benzyl, p-methoxybenzyl), and alkylcarbonyloxyalkyl (for example, pivaloyloxymethyl). Amines have been masked as arylcarbonyloxymethyl substituted derivatives which are cleaved by esterases in vivo releasing the free drug and formaldehyde (Bungaard J. Med. Chem. 2503 (1989)). Also, drugs containing an acidic NH group, such as imidazole, imide, indole and the like, have been masked with N-acyloxymethyl groups (Bundgaard Design of Prodrugs, Elsevier (1985)). Hydroxy groups have been masked as esters and ethers. EP 0 039 051 (Sloan and Little, Apr. 11, 1981) discloses Mannich-base hydroxamic acid prodrugs, their preparation and use.

Certain compounds of the present invention can exist in unsolvated forms as well as in solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

The compounds of the present invention may also contain unnatural proportions of atomic isotopes at one or more of the atoms that constitute such compounds. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulfur, fluorine and chlorine such as ²H, ³H, ¹³C, ¹⁴C, ¹⁵N, ¹⁷O, ¹⁸O ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents. All isotopic variations of the compounds and compositions of the present invention, whether radioactive or not, are intended to be encompassed within the scope of the present invention.

If L² is C₁-C₆-alkylene-O, C₁-C₆-alkylene-S, C₁-C₆-alkylene-NR, C₃-C₈-cycloalkylene-O, C₃-C₈-cycloalkylene-S or C₃-C₈-cycloalkylene-NR¹⁵, O, S and NR¹⁵ are bound to the ring A.

The organic moieties mentioned in the above definitions of the variables are—like the term halogen—collective terms for individual listings of the individual group members. The prefix Cₙ-Cₘ indicates in each case the possible number of carbon atoms in the group. If two or more radicals can be selected independently from each other, then the term “independently” means that the radicals may be the same or may be different.

The term “halogen” denotes in each case fluorine, bromine, chlorine or iodine, in particular fluorine, chlorine or bromine. Halogen as a substituent on an aromatic or heteroaromatic group is preferably F or Cl, and on an aliphatic (e.g. on an alkyl, alkenyl, alkynyl, alkylene (derived) group) or cycloaliphatic (e.g. on a cycloalkyl group) group or on a saturated or partially unsaturated heterocyclic ring is F.

The term “alkyl” as used herein and in the alkyl moieties of alkoxy and the like refers to saturated straight-chain or branched hydrocarbon radicals having 1 to 2 (“C₁-C₂-alkyl”), 1 to 3 (“C₁-C₃-alkyl”), 1 to 4 (“C₁-C₄-alkyl”) or 1 to 6 (“C₁-C₆- alkyl”). C₁-C₂-Alkyl is methyl or ethyl. C₁-C₃-Alkyl is additionally propyl and isopropyl. C₁-C₄-Alkyl is additionally butyl, 1-methylpropyl (sec-butyl), 2-methylpropyl (isobutyl) or 1,1-dimethylethyl (tert-butyl). C₁-C₆-Alkyl is additionally also, for example, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl.

The term “haloalkyl” as used herein, which may also be expressed as “alkyl which is partially or fully halogenated”, refers to straight-chain or branched alkyl groups having 1 to 2 (“C₁-C₂-haloalkyl”), 1 to 3 (“C₁-C₃-haloalkyl”), 1 to 4 (“C₁-C₄-haloalkyl”) or 1 to 6 (“C₁-C₆-haloalkyl”) carbon atoms (as mentioned above), where some or all of the hydrogen atoms in these groups are replaced by fluorine atoms. Examples for C₁-C₂-haloalkyl (indeed for fluorinated C₁-C₂-alkyl) are fluoromethyl, difluoromethyl, trifluoromethyl, 1-fluoroethyl, 2-fluoroethyl, 2,2-difluoroethyl, 2,2,2-trifluoroethyl, or pentafluoroethyl. Examples for C₁-C₃-haloalkyl (indeed for fluorinated C₁-C₃-alkyl) are, in addition to those mentioned for C₁-C₂-haloalkyl, 1-fluoropropyl, 2-fluoropropyl, (R)-2-fluoropropyl, (S)-2-fluoropropyl, 3-fluoropropyl, 1,1-difluoropropyl, 2,2-difluoropropyl, 1,2-difluoropropyl, 2,3-difluoropropyl, 3,3-difluoropropyl, 2,2,3-trifluoropropyl, 3,3,3-trifluoropropyl, 2,2,3,3-tetrafluoropropyl, 2,2,3,3,3-pentafluoropropyl, heptafluoropropyl, 1,1,1-trifluoroprop-2-yl, 2-fluoro-1-methylethyl, (R)-2-fluoro-1-methylethyl, (S)-2-fluoro-1-methylethyl, 2,2-difluoro-1-methylethyl, (R)-2,2-difluoro-1-methylethyl, (S)-2,2-difluoro-1-methylethyl, 2,2,2-trifluoro-1-methylethyl, (R)-2,2,2-trifluoro-1-methylethyl, (S)-2,2,2-trifluoro-1-methylethyl, 2-fluoro-1-(fluoromethyl)ethyl, 1-(difluoromethyl)-2,2-difluoroethyl, 1-(trifluoromethyl)-2,2,2-trifluoroethyl, 1-(trifluoromethyl)-1,2,2,2-tetrafluoroethyl and the like. Examples for C₁-C₄-haloalkyl are, in addition to those mentioned for C₁-C₃-haloalkyl, 2-fluorobutyl, (R)-2-fluorobutyl, (S)-2-fluorobutyl, 3-fluorobutyl, (R)-3-fluorobutyl, (S)-3-fluorobutyl, 4-fluorobutyl, 2,2-difluorobutyl, 3,3-difluorobutyl, 4,4-difluorobutyl, 4,4,4-trifluorobutyl, 3,3,4,4-tetrafluorobutyl, 3,4,4,4-tetrafluorobutyl, 2,2,4,4,4-pentafluorobutyl, 3,3,4,4,4-pentafluorobutyl, 2,2,3,4,4,4-hexafluorobutyl, 1-methyl-2,2-3,3-tetrafluoropropyl and the like.

The term “alkenyl” as used herein refers to monounsaturated straight-chain or branched hydrocarbon radicals having 3 or 4 (“C₃-C₄-alkenyl”), 2 to 4 (“C₂-C₄-alkenyl”) or 2 to 6 (“C₂-C₆-alkenyl”) carbon atoms and a double bond in any position. Examples for C₃-C₄-alkenyl are 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl or 2-methyl-2-propenyl. Examples for C₂-C₄-alkenyl are ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl or 2-methyl-2-propenyl. Examples for C₂-C₆-alkenyl are ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl or 1-ethyl-2-methyl-2-propenyl.

The term “haloalkenyl” as used herein, which may also be expressed as “alkenyl which is partially or fully halogenated”, refers to unsaturated straight-chain or branched hydrocarbon radicals having 3 or 4 (“C₃-C₄-haloalkenyl”), 2 to 4 (“C₂-C₄-haloalkenyl”) or 2 to 6 (“C₂-C₆-haloalkenyl”) carbon atoms and a double bond in any position (as mentioned above), where some or all of the hydrogen atoms in these groups are replaced by fluorine atoms, for example fluorovinyl, fluoroallyl and the like.

The term “alkynyl” as used herein refers to straight-chain or branched hydrocarbon groups having 2 or 3 (“C₂-C₃-alkynyl”), 2 to 4 (“C₂-C₄-alkynyl”) or 2 to 6 (“C₂-C₆-alkynyl”) carbon atoms and one triple bond in any position. Examples for C₂-C₃-alkynyl are ethynyl, 1-propynyl or 2-propynyl. Examples for C₂-C₄-alkynyl are ethynyl, 1-propynyl, 2-propynyl, 1-butynyl, 2-butynyl, 3-butynyl or 1-methyl-2-propynyl. Examples for C₂-C₆-alkynyl are ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl or 1-ethyl-1-methyl-2-propynyl.

The term “haloalkynyl” as used herein, which can also be expressed as “alkynyl which is partially or fully halogenated”, refers to unsaturated straight-chain or branched hydrocarbon radicals having 2 or (“C₂-C₃-haloalkynyl”), 2 to 4 (“C₃-C₄-haloalkynyl”) or 2 to 6 (“C₂-C₆-haloalkynyl”) carbon atoms and one triple bond in any position (as mentioned above), where some or all of the hydrogen atoms in these groups are replaced by fluorine atoms.

The term “cycloalkyl” as used herein refers to mono- or bi- or polycyclic saturated hydrocarbon radicals having 3 to 8 (“C₃-C₈-cycloalkyl”), in particular 3 to 6 carbon atoms (“C₃-C₆-cycloalkyl”) or 5 or 6 carbon atoms (“C₅-C₆-cycloalkyl”). Examples of monocyclic radicals having 5 or 6 carbon atoms are cyclopentyl and cyclohexyl. Examples of monocyclic radicals having 3 to 6 carbon atoms comprise cyclopropyl, cyclobutyl, cyclopentyl and cyclohexyl. Examples of monocyclic radicals having 3 to 8 carbon atoms comprise cyclopropyl, cyclobutyl, cyclopentyl, cyclohexyl, cycloheptyl and cyclooctyl. Examples of bicyclic radicals having 7 or 8 carbon atoms comprise bicyclo[2.2.1]heptyl, bicyclo[3.1.1]heptyl, bicyclo[2.2.2]octyl and bicyclo[3.2.1]octyl. Preferably, the term cycloalkyl denotes a monocyclic saturated hydrocarbon radical.

The term “halocycloalkyl” as used herein, which can also be expressed as “cycloalkyl which is partially or fully halogenated”, refers to mono- or bi- or polycyclic saturated hydrocarbon groups having 3 to 8 (“C₃-C₈-halocycloalkyl”) or preferably 3 to 6 (“C₃-C₆-halocycloalkyl”) or 5 or 6 (“C₅-C₆-halocycloalkyl”) carbon ring members (as mentioned above) in which some or all of the hydrogen atoms are replaced by fluorine atoms.

The term “cycloalkyl-C₁-C₄-alkyl” refers to a C₃-C₈-cycloalkyl group (“C₃-C₈-cycloalkyl-C₁-C₄-alkyl”), preferably a C₃-C₆-cycloalkyl group (“C₃-C₆-cycloalkyl-C₁-C₄-alkyl”), more preferably a C₃-C₄-cycloalkyl group (“C₃-C₄-cycloalkyl-C₁-C₄-alkyl”) as defined above (preferably a monocyclic cycloalkyl group) which is bound to the remainder of the molecule via a C₁-C₄-alkyl group, as defined above. Examples for C₃-C₄-cycloalkyl-C₁-C₄-alkyl are cyclopropylmethyl, cyclopropylethyl, cyclopropylpropyl, cyclobutylmethyl, cyclobutylethyl and cyclobutylpropyl, Examples for C₃-C₆-cycloalkyl-C₁-C₄-alkyl are, in addition to those mentioned for C₃-C₄-cycloalkyl-C₁-C₄-alkyl, cyclopentylmethyl, cyclopentylethyl, cyclopentylpropyl, cyclohexylmethyl, cyclohexylethyl and cyclohexylpropyl. Examples for C₃-C₈-cycloalkyl-C₁-C₄-alkyl are, in addition to those mentioned for C₃-C₆-cycloalkyl-C₁-C₄-alkyl, cycloheptylmethyl, cycloheptylethyl, cyclooctylmethyl and the like.

The term “C₃-C₈-halocycloalkyl-C₁-C₄-alkyl” refers to a C₃-C₈-halocycloalkyl group as defined above, i.e. to fluorinated C₃-C₈-cycloalkyl, which is bound to the remainder of the molecule via a C₁-C₄-alkyl group, as defined above.

The term “C₁-C₂-alkoxy” denotes a C₁-C₂-alkyl group, as defined above, attached via an oxygen atom to the remainder of the molecule. The term “C₁-C₃-alkoxy” denotes a C₁-C₃-alkyl group, as defined above, attached via an oxygen atom. The term “C₁-C₄-alkoxy” denotes a C₁-C₄-alkyl group, as defined above, attached via an oxygen atom. The term “C₁-C₆-alkoxy” denotes a C₁-C₆-alkyl group, as defined above, attached via an oxygen atom. C₁-C₂-Alkoxy is methoxy or ethoxy. C₁-C₃-Alkoxy is additionally, for example, n-propoxy or 1-methylethoxy (isopropoxy). C₁-C₄-Alkoxy is additionally, for example, butoxy, 1-methylpropoxy (sec-butoxy), 2-methylpropoxy (isobutoxy) or 1,1-dimethylethoxy (tert-butoxy). C₁-C₆-Alkoxy is additionally, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy.

The term “C₁-C₂-haloalkoxy” denotes a C₁-C₂-haloalkyl group, as defined above, attached via an oxygen atom to the remainder of the molecule. The term “C₁-C₃-haloalkoxy” denotes a C₁-C₃-haloalkyl group, as defined above, attached via an oxygen atom. The term “C₁-C₄-haloalkoxy” denotes a C₁-C₄-haloalkyl group, as defined above, attached via an oxygen atom. The term “C₁-C₆-haloalkoxy” denotes a C₁-C₆-haloalkyl group, as defined above, attached via an oxygen atom. C₁-C₂-Haloalkoxy (indeed fluorinated C₁-C₂-alkoxy) is, for example, OCH₂F, OCHF₂, OCF₃, 2-fluoroethoxy, 2-2,2-difluoroethoxy, 2,2,2-trifluoroethoxy or OC₂F₅. C₁-C₃-Haloalkoxy (indeed fluorinated C₁-C₃-alkoxy) is additionally, for example, 2-fluoropropoxy, 3-fluoropropoxy, 2,2-difluoropropoxy, 2,3-difluoropropoxy, 3,3,3-trifluoropropoxy, OCH₂—C₂F₅, OCF₂—C₂F₅ or 1-(CH₂F)-2-fluoroethoxy. C₁-C₄-Haloalkoxy (indeed fluorinated C₁-C₄-alkoxy) is additionally, for example, 4-fluorobutoxy or nonafluorobutoxy. C₁-C₆-Haloalkoxy (indeed fluorinated C₁-C₆-alkoxy) is additionally, for example, 5-fluoropentoxy, undecafluoropentoxy, 6-fluorohexoxy or dodecafluorohexoxy.

The term “C₁-C₄-alkoxy-C₁-C₄-alkyl” as used herein, refers to a straight-chain or branched alkyl group having 1 to 4 carbon atoms, as defined above, where one hydrogen atom is replaced by a C₁-C₄-alkoxy group, as defined above. The term “C₁-C₆-alkoxy-C₁-C₆-alkyl” as used herein, refers to a straight-chain or branched alkyl group having 1 to 6 carbon atoms, as defined above, where one hydrogen atom is replaced by a C₁-C₆-alkoxy group, as defined above. Examples are methoxymethyl, ethoxymethyl, propoxymethyl, isopropoxymethyl, n-butoxymethyl, sec-butoxymethyl, isobutoxymethyl, tert-butoxymethyl, 1-methoxyethyl, 1-ethoxyethyl, 1-propoxyethyl, 1-isopropoxyethyl, 1-n-butoxyethyl, 1-sec-butoxyethyl, 1-isobutoxyethyl, 1-tert-butoxyethyl, 2-methoxyethyl, 2-ethoxyethyl, 2-propoxyethyl, 2-isopropoxyethyl, 2-n-butoxyethyl, 2-sec-butoxyethyl, 2-isobutoxyethyl, 2-tert-butoxyethyl, 1-methoxypropyl, 1-ethoxypropyl, 1-propoxypropyl, 1-isopropoxypropyl, 1-n-butoxypropyl, 1-sec-butoxypropyl, 1-isobutoxypropyl, 1-tert-butoxypropyl, 2-methoxypropyl, 2-ethoxypropyl, 2-propoxypropyl, 2-isopropoxypropyl, 2-n-butoxypropyl, 2-sec-butoxypropyl, 2-isobutoxypropyl, 2-tert-butoxypropyl, 3-methoxypropyl, 3-ethoxypropyl, 3-propoxypropyl, 3-isopropoxypropyl, 3-n-butoxypropyl, 3-sec-butoxypropyl, 3-isobutoxypropyl, 3-tert-butoxypropyl and the like.

C₁-C₆-Haloalkoxy-C₁-C₆-alkyl is a straight-chain or branched alkyl group having from 1 to 6, especially 1 to 4 carbon atoms (═C₁-C₆-haloalkoxy-C₁-C₄-alkyl), wherein one of the hydrogen atoms is replaced by a C₁-C₆-alkoxy group and wherein at least one, e.g. 1, 2, 3, 4 or all of the remaining hydrogen atoms (either in the alkoxy moiety or in the alkyl moiety or in both) are replaced by fluorine atoms. C₁-C₄-Haloalkoxy-C₁-C₄-alkyl (indeed fluorinated C₁-C₄-alkoxy-C₁-C₄-alkyl) is a straight-chain or branched alkyl group having from 1 to 4 carbon atoms, wherein one of the hydrogen atoms is replaced by a C₁-C₄-alkoxy group and wherein at least one, e.g. 1, 2, 3, 4 or all of the remaining hydrogen atoms (either in the alkoxy moiety or in the alkyl moiety or in both) are replaced by fluorine atoms. Examples are difluoromethoxymethyl (CHF₂OCH₂), trifluoromethoxymethyl, 1-difluoromethoxyethyl, 1-trifluoromethoxyethyl, 2-difluoromethoxyethyl, 2-trifluoromethoxyethyl, difluoro-methoxy-methyl (CH₃OCF₂), 1,1-difluoro-2-methoxyethyl, 2,2-difluoro-2-methoxyethyl and the like.

The term “C₁-C₂-alkylthio” denotes a C₁-C₂-alkyl group, as defined above, attached via a sulfur atom to the remainder of the molecule. The term “C₁-C₃-alkylthio” denotes a C₁-C₃-alkyl group, as defined above, attached via a sulfur atom. The term “C₁-C₄-alkylthio” denotes a C₁-C₄-alkyl group, as defined above, attached via a sulfur atom. The term “C₁-C₆-alkylthio” denotes a C₁-C₆-alkyl group, as defined above, attached via a sulfur atom. C₁-C₂-Alkylthio is methylthio or ethylthio. C₁-C₃-Alkylthio is additionally, for example, n-propylthio or 1-methylethylthio (isopropylthio). C₁-C₄-Alkylthio is additionally, for example, butylthio, 1-methylpropylthio (sec-butylthio), 2-methylpropylthio (isobutylthio) or 1,1-dimethylethylthio (tert-butylthio). C₁-C₆-Alkylthio is additionally, for example, pentylthio, 1-methylbutylthio, 2-methylbutylthio, 3-methylbutylthio, 1,1-dimethylpropylthio, 1,2-dimethylpropylthio, 2,2-dimethylpropylthio, 1-ethylpropylthio, hexylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio or 1-ethyl-2-methylpropylthio.

The term “C₁-C₂-haloalkylthio” denotes a C₁-C₂-haloalkyl group, as defined above, attached via a sulfur atom to the remainder of the molecule. The term “C₁-C₃-haloalkylthio” denotes a C₁-C₃-haloalkyl group, as defined above, attached via a sulfur atom. The term “C₁-C₄-haloalkylthio” denotes a C₁-C₄-haloalkyl group, as defined above, attached via a sulfur atom. The term “C₁-C₆-haloalkylthio” denotes a C₁-C₆-haloalkyl group, as defined above, attached via a sulfur atom. C₁-C₂-Haloalkylthio (indeed fluorinated C₁-C₂-alkylthio) is, for example, SCH₂F, SCHF₂, SCF₃, 2-fluoroethylthio, 2,2-difluoroethylthio, or SC₂F₅. C₁-C₃-Haloalkylthio (indeed fluorinated C₁-C₃-alkylthio) is additionally, for example, 2-fluoropropylthio, 3-fluoropropylthio, 2,2-difluoropropylthio, 2,3-difluoropropylthio, 3,3,3-trifluoropropylthio, SCH₂—C₂F₅, SCF₂—C₂F₅ or 1-(CH₂F)-2-fluoroethylthio., C₁-C₄-Haloalkylthio (indeed fluorinated C₁-C₄-alkylthio) is additionally, for example, 4-fluorobutylthio or nonafluorobutylthio. C₁-C₆-Haloalkylthio (indeed fluorinated C₁-C₆-alkylthio) is additionally, for example, 5-fluoropentylthio, undecafluoropentylthio, 6-fluorohexylthio or dodecafluorohexylthio.

The term “C₁-C₂-alkylsulfonyl” denotes a C₁-C₂-alkyl group, as defined above, attached via a sulfonyl [S(O)₂] group to the remainder of the molecule. The term “C₁-C₃-alkylsulfonyl” denotes a C₁-C₃-alkyl group, as defined above, attached via a sulfonyl [S(O)₂] group. The term “C₁-C₄-alkylsulfonyl” denotes a C₁-C₄-alkyl group, as defined above, attached via a sulfonyl [S(O)₂] group. The term “C₁-C₆-alkylsulfonyl” denotes a C₁-C₆-alkyl group, as defined above, attached via a sulfonyl [S(O)₂] group. C₁-C₂-Alkylsulfonyl is methylsulfonyl or ethylsulfonyl. C₁-C₃-Alkylsulfonyl is additionally, for example, n-propylsulfonyl or 1-methylethylsulfonyl (isopropylsulfonyl). C₁-C₄-Alkylsulfonyl is additionally, for example, butylsulfonyl, 1-methylpropylsulfonyl (sec-butylsulfonyl), 2-methylpropylsulfonyl (isobutylsulfonyl) or 1,1-dimethylethylsulfonyl (tert-butylsulfonyl). C₁-C₆-Alkylsulfonyl is additionally, for example, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl or 1-ethyl-2-methylpropylsulfonyl. C₁-C₈-Alkylsulfonyl is additionally, for example, heptylsulfonyl, octylsulfonyl, 2-ethylhexylsulfonyl and positional isomers thereof. C₁-C₁₀-Alkylsulfonyl is additionally, for example, nonylsulfonyl, decylsulfonyl and positional isomers thereof.

The term “C₁-C₂-haloalkylsulfonyl” denotes a C₁-C₂-haloalkyl group, as defined above, attached via a sulfonyl [S(O)₂] group to the remainder of the molecule. The term “C₁-C₃-haloalkylsulfonyl” denotes a C₁-C₃-haloalkyl group, as defined above, attached via a sulfonyl [S(O)₂] group. The term “C₁-C₄-haloalkylsulfonyl” denotes a C₁-C₄-haloalkyl group, as defined above, attached via a sulfonyl [S(O)₂] group. The term “C₁-C₆-haloalkylsulfonyl” denotes a C₁-C₆-haloalkyl group, as defined above, attached via a sulfonyl [S(O)₂] group. C₁-C₂-Haloalkylsulfonyl (indeed fluorinated C₁-C₂-alkylsulfonyl) is, for example, S(O)₂CH₂F, S(O)₂CHF₂, S(O)₂CF₃, 2-fluoroethylsulfonyl, 2,2-difluoroethylsulfonyl, 2,2,2-trifluoroethylsulfonyl or S(O)₂C₂F₅. C₁-C₃-Haloalkylsulfonyl (indeed fluorinated C₁-C₃-alkylsulfonyl) is additionally, for example, 2-fluoropropylsulfonyl, 3-fluoropropylsulfonyl, 2,2-difluoropropylsulfonyl, 2,3-difluoropropylsulfonyl, 3,3,3-trifluoropropylsulfonyl, S(O)₂CH₂—C₂F₅, S(O)₂CF₂—C₂F₅ or 1-(CH₂F)-2-fluoroethylsulfonyl. C₁-C₄-Haloalkylsulfonyl (indeed fluorinated C₁-C₄-alkylsulfonyl) is additionally, for example, 4-fluorobutylsulfonyl or nonafluorobutylsulfonyl. C₁-C₆-Haloalkylsulfonyl (indeed fluorinated C₁-C₆-alkylsulfonyl) is additionally, for example, 5-fluoropentylsulfonyl, undecafluoropentylsulfonyl, 6-fluorohexylsulfonyl or dodecafluorohexylsulfonyl.

The substituent “oxo” is ═O; i.e. it replaces a CH₂ group by a C(═O) group.

“Carboxyl” is —C(═O)OH group.

The term “alkylcarbonyl” denotes a C₁-C₆-alkyl (“C₁-C₆-alkylcarbonyl”), preferably a C₁-C₄-alkyl (“C₁-C₄-alkylcarbonyl”) group, as defined above, attached to the remainder of the molecule via a carbonyl [C(═O)] group. Examples are acetyl (methylcarbonyl), propionyl (ethylcarbonyl), propylcarbonyl, isopropylcarbonyl, n-butylcarbonyl and the like.

The term “haloalkylcarbonyl” denotes a C₁-C₆-haloalkyl (“C₁-C₆-haloalkylcarbonyl”; indeed fluorinated C₁-C₆-alkylcarbonyl), preferably a C₁-C₄-haloalkyl (“C₁-C₄-haloalkylcarbonyl”; indeed fluorinated C₁-C₄-alkylcarbonyl) group, as defined above, attached to the remainder of the molecule via a carbonyl [C(═O)] group. Examples are trifluoromethylcarbonyl, 2,2,2-trifluoroethylcarbonyl and the like.

The term “alkoxycarbonyl” denotes a C₁-C₆-alkoxy (“C₁-C₆-alkoxycarbonyl”), preferably a C₁-C₄-alkoxy (“C₁-C₄-alkoxycarbonyl”) group, as defined above, attached to the remainder of the molecule via a carbonyl [C(═O)] group. Examples are methoxycarbonyl), ethoxycarbonyl, propoxycarbonyl, isopropoxycarbonyl, n-butoxycarbonyl and the like.

The term “haloalkoxycarbonyl” denotes a C₁-C₆-haloalkoxy (“C₁-C₆-haloalkoxycarbonyl”; indeed fluorinated C₁-C₆-alkoxycarbonyl), preferably a C₁-C₄-haloalkoxy (“C₁-C₄-haloalkoxycarbonyl”; indeed fluorinated C₁-C₄-alkoxycarbonyl) group, as defined above, attached to the remainder of the molecule via a carbonyl [C(═O)] group. Examples are trifluoromethoxycarbonyl, 2,2,2-trifluoroethoxycarbonyl and the like.

The term “3-, 4-, 5-, 6-, 7- or 8-membered saturated, partially unsaturated or maximally unsaturated carbocyclic ring” as used herein denotes monocyclic radicals containing only C atoms as ring members, the monocyclic radicals being saturated, partially unsaturated or maximum unsaturated (including aromatic).

Unsaturated carbocyclic rings contain at least one C═C double bond. Maximally unsaturated rings contain as many conjugated C═C double bonds as allowed by the ring size. Partially unsaturated rings contain less than the maximum number of C═C double bond(s) allowed by the ring size.

A 3-, 4-, 5-, 6-, 7- or 8-membered saturated unsaturated carbocyclic ring is C₃-C₈-cycloalkyl, as defined above.

Examples for 3-, 4-, 5-, 6-, 7- or 8-membered partially unsaturated carbocyclic rings are cyclobut-1-en-1-yl, cyclobut-1-en-3-yl, cyclopent-1-en-1-yl, cyclopent-1-en-3-yl, cyclopent-1-en-4-yl, cyclopenta-1,3-dien-1-yl, cyclopenta-1,3-dien-2-yl, cyclopenta-1,3-dien-5-yl, cyclohex-1-en-1-yl, cyclohex-1-en-3-yl, cyclohex-1-en-4-yl, cyclohexa-1,3-dien-1-yl, cyclohexa-1,3-dien-2-yl, cyclohexa-1,3-dien-5-yl, cyclohexa-1,4-dien-1-yl, cyclohexa-1,4-dien-3-yl, cyclohept-1-en-1-yl, cyclohept-1-en3-yl, cyclohept-1-en-4-yl, cyclohept-1-en-5-yl, cyclohepta-1,3-dien-1-yl, cyclohepta-1,3-dien-2-yl, cyclohepta-1,3-dien-5-yl, cyclohepta-1,3-dien-6-yl, cyclohepta-1,4-dien-1-yl, cyclohepta-1,4-dien-2-yl, cyclohepta-1,4-dien-3-yl, cyclohepta-1,4-dien-6-yl, cyclooct-1-en-1-yl, cyclooct-1-en-3-yl, cyclooct-1-en-4-yl, cyclooct-1-en-5-yl, cycloocta-1,3-dien-1-yl, cycloocta-1,3-dien-2-yl, cycloocta-1,3-dien-5-yl, cycloocta-1,3-dien-6-yl, cycloocta-1,4-dien-1-yl, cycloocta-1,4-dien-2-yl, cycloocta-1,4-dien-3-yl, cycloocta-1,4-dien-6-yl, cycloocta-1,4-dien-7-yl, cycloocta-1,5-dien-1-yl, and cycloocta-1,5-dien-3-yl.

Examples for 3-, 4-, 5-, 6-, 7- or 8-membered maximally unsaturated carbocyclic rings are cycloprop-1-en-1-yl, cycloprop-1-en-3-yl, cyclobutadienyl, cyclopenta-1,3-dien-1-yl, cyclopenta-1,3-dien-2-yl, cyclopenta-1,3-dien-5-yl, phenyl, cyclohepta-1,3,5-trien-1-yl, cyclohepta-1,3,5-trien-2-yl, cyclohepta-1,3,5-trien-3-yl, cyclohepta-1,3,5-trien-7-yl and cyclooctatetraenyl.

Aryl is an aromatic carbocyclic ring containing 6 to 14 carbon atoms. Examples are phenyl, naphthyl, phenanthrenyl and anthracenyl.

The term “aryl-C₁-C₃-alkyl” refers to an aryl group, as defined above, bound to the remainder of the molecule via a C₁-C₃-alkyl group. Examples are benzyl, 1-phenylethyl, 2-phenylethyl (phenethyl), 1-phenylpropyl, 2-phenylpropyl, 3-phenylpropyl, naphth-1-yl-methyl or naphth-2-yl-methyl.

The term “3-, 4-, 5-, 6-, 7- or 8-membered saturated, partially unsaturated or maximally unsaturated heterocyclic ring containing 1, 2, 3 or 4 heteroatoms or heteroatom groups selected from the group consisting of O, N, S, NO, SO and SO₂, as ring members” [wherein “maximum unsaturated” includes also “aromatic” ] as used herein denotes monocyclic radicals, the monocyclic radicals being saturated, partially unsaturated or maximum unsaturated (including aromatic).

Unsaturated rings contain at least one C—C and/or C—N and/or N—N double bond(s). Maximally unsaturated rings contain as many conjugated C—C and/or C—N and/or N—N double bonds as allowed by the ring size. Maximally unsaturated 5- or 6-membered heteromonocyclic rings are generally aromatic. Exceptions are maximally unsaturated 6-membered rings containing O, S, SO and/or SO₂ as ring members, such as pyran and thiopyran, which are not aromatic. Partially unsaturated rings contain less than the maximum number of C—C and/or C—N and/or N—N double bond(s) allowed by the ring size. The heterocyclic ring may be attached to the remainder of the molecule via a carbon ring member or via a nitrogen ring member. As a matter of course, the heterocyclic ring contains at least one carbon ring atom. If the ring contains more than one 0 ring atom, these are not adjacent.

Examples of a 3-, 4-, 5-, 6-, 7- or 8-membered saturated heteromonocyclic ring containing 1, 2, 3 or 4 heteroatoms or heteroatom groups selected from the group consisting of O, N, S, NO, SO and SO₂, as ring members include: Oxiran-2-yl, thiiran-2-yl, aziridin-1-yl, aziridin-2-yl, oxetan-2-yl, oxetan-3-yl, thietan-2-yl, thietan-3-yl, 1-oxothietan-2-yl, 1-oxothietan-3-yl, 1,1-dioxothietan-2-yl, 1,1-dioxothietan-3-yl, azetidin-1-yl, azetidin-2-yl, azetidin-3-yl, tetrahydrofuran-2-yl, tetrahydrofuran-3-yl, tetrahydrothien-2-yl, tetrahydrothien-3-yl, 1-oxotetrahydrothien-2-yl, 1,1-dioxotetrahydrothien-2-yl, 1-oxotetrahydrothien-3-yl, 1,1-dioxotetrahydrothien-3-yl, pyrrolidin-1-yl, pyrrolidin-2-yl, pyrrolidin-3-yl, pyrazolidin-1-yl, pyrazolidin-3-yl, pyrazolidin-4-yl, pyrazolidin-5-yl, imidazolidin-1-yl, imidazolidin-2-yl, imidazolidin-4-yl, oxazolidin-2-yl, oxazolidin-3-yl, oxazolidin-4-yl, oxazolidin-5-yl, isoxazolidin-2-yl, isoxazolidin-3-yl, isoxazolidin-4-yl, isoxazolidin-5-yl, thiazolidin-2-yl, thiazolidin-3-yl, thiazolidin-4-yl, thiazolidin-5-yl, isothiazolidin-2-yl, isothiazolidin-3-yl, isothiazolidin-4-yl, isothiazolidin-5-yl, 1,2,4-oxadiazolidin-2-yl, 1,2,4-oxadiazolidin-3-yl, 1,2,4-oxadiazolidin-4-yl, 1,2,4-oxadiazolidin-5-yl, 1,2,4-thiadiazolidin-2-yl, 1,2,4-thiadiazolidin-3-yl, 1,2,4-thiadiazolidin-4-yl, 1,2,4-thiadiazolidin-5-yl, 1,2,4-triazolidin-1-yl, 1,2,4-triazolidin-3-yl, 1,2,4-triazolidin-4-yl, 1,3,4-oxadiazolidin-2-yl, 1,3,4-oxadiazolidin-3-yl, 1,3,4-thiadiazolidin-2-yl, 1,3,4-thiadiazolidin-3-yl, 1,3,4-triazolidin-1-yl, 1,3,4-triazolidin-2-yl, 1,3,4-triazolidin-3-yl, 1,2,3,4-tetrazolidin-1-yl, 1,2,3,4-tetrazolidin-2-yl, 1,2,3,4-tetrazolidin-5-yl, tetrahydropyran-2-yl, tetrahydropyran-3-yl, tetrahydropyran-4-yl, 1,3-dioxan-2-yl, 1,3-dioxan-4-yl, 1,3-dioxan-5-yl, 1,4-dioxan-2-yl, piperidin-1-yl, piperidin-2-yl, piperidin-3-yl, piperidin-4-yl, hexahydropyridazin-1-yl, hexahydropyridazin-3-yl, hexahydropyridazin-4-yl, hexahydropyrimidin-1-yl, hexahydropyrimidin-2-yl, hexahydropyrimidin-4-yl, hexahydropyrimidin-5-yl, piperazin-1-yl, piperazin-2-yl, 1,3,5-hexahydrotriazin-1-yl, 1,3,5-hexahydrotriazin-2-yl, 1,2,4-hexahydrotriazin-1-yl, 1,2,4-hexahydrotriazin-2-yl, 1,2,4-hexahydrotriazin-3-yl, 1,2,4-hexahydrotriazin-4-yl, 1,2,4-hexahydrotriazin-5-yl, 1,2,4-hexahydrotriazin-6-yl, morpholin-2-yl, morpholin-3-yl, morpholin-4-yl, thiomorpholin-2-yl, thiomorpholin-3-yl, thiomorpholin-4-yl, 1-oxothiomorpholin-2-yl, 1-oxothiomorpholin-3-yl, 1-oxothiomorpholin-4-yl, 1,1-dioxothiomorpholin-2-yl, 1,1-dioxothiomorpholin-3-yl, 1,1-dioxothiomorpholin-4-yl, azepan-1-, -2-, -3- or -4-yl, oxepan-2-, -3-, -4- or -5-yl, hexahydro-1,3-diazepinyl, hexahydro-1,4-diazepinyl, hexahydro-1,3-oxazepinyl, hexahydro-1,4-oxazepinyl, hexahydro-1,3-dioxepinyl, hexahydro-1,4-dioxepinyl, oxocane, thiocane, azocanyl, [1,3]diazocanyl, [1,4]diazocanyl, [1,5]diazocanyl, [1,5]oxazocanyl and the like.

Examples of a 3-, 4-, 5-, 6-, 7- or 8-membered partially unsaturated heteromonocyclic ring containing 1, 2, 3 or 4 heteroatoms or heteroatom groups selected from the group consisting of O, N, S, NO, SO and SO₂, as ring members include: 2,3-dihydrofuran-2-yl, 2,3-dihydrofuran-3-yl, 2,4-dihydrofuran-2-yl, 2,4-dihydrofuran-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4-di- or tetrahydropyridazinyl, 2-di- or tetrahydropyrimidinyl, 4-di- or tetrahydropyrimidinyl, 5-di- or tetrahydropyrimidinyl, di- or tetrahydropyrazinyl, 1,3,5-di- or tetrahydrotriazin-2-yl, 1,2,4-di- or tetrahydrotriazin-3-yl, 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, tetrahydrooxepinyl, such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, tetrahydro-1,3-diazepinyl, tetrahydro-1,4-diazepinyl, tetrahydro-1,3-oxazepinyl, tetrahydro-1,4-oxazepinyl, tetrahydro-1,3-dioxepinyl, tetrahydro-1,4-dioxepinyl, 1,2,3,4,5,6-hexahydroazocine, 2,3,4,5,6,7-hexahydroazocine, 1,2,3,4,5,8-hexahydroazocine, 1,2,3,4,7,8-hexahydroazocine, 1,2,3,4,5,6-hexahydro-[1,5]diazocine,1,2,3,4,7,8-hexahydro[1,5]diazocine and the like.

Examples of a 3-, 4-, 5-, 6-, 7- or 8-membered maximally unsaturated (including aromatic) heteromonocyclic ring containing 1, 2, 3 or 4 heteroatoms or heteroatom groups selected from the group consisting of O, N, S, NO, SO and SO₂, as ring members are 2-furyl, 3-furyl, 2-thienyl, 3-thienyl, 1-pyrrolyl, 2-pyrrolyl, 3-pyrrolyl, 1-pyrazolyl, 3-pyrazolyl, 4-pyrazolyl, 5-pyrazolyl, 1-imidazolyl, 2-imidazolyl, 4-imidazolyl, 5-imidazolyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 3-isothiazolyl, 4-isothiazolyl, 5-isothiazolyl, 1,3,4-triazol-1-yl, 1,3,4-triazol-2-yl, 1,3,4-triazol-3-yl, 1,2,3-triazol-1-yl, 1,2,3-triazol-2-yl, 1,2,3-triazol-4-yl, 1,2,5-oxadiazol-3-yl, 1,2,3-oxadiazol-4-yl, 1,2,3-oxadiazol-5-yl, 1,3,4-oxadiazol-2-yl, 1,2,5-thiadiazol-3-yl, 1,2,3-thiadiazol-4-yl, 1,2,3-thiadiazol-5-yl, 1,3,4-thiadiazol-2-yl, 1,2,3,4-tetrazol-1-yl, 1,2,3,4-tetrazol-2-yl, 1,2,3,4-tetrazol-5-yl, 2-pyridinyl, 3-pyridinyl, 4-pyridinyl, 1-oxopyridin-2-yl, 1-oxopyridin-3-yl, 1-oxopyridin-4-yl, 3-pyridazinyl, 4-pyridazinyl, 2-pyrimidinyl, 4-pyrimidinyl, 5-pyrimidinyl, 2-pyrazinyl, 1,3,5-triazin-2-yl, 1,2,4-triazin-3-yl, 1,2,4-triazin-5-yl, 1,2,3,4-tetrazin-1-yl, 1,2,3,4-tetrazin-2-yl, 1,2,3,4-tetrazin-5-yl, pyran-2-yl, pyran-3-yl, pyran-4-yl, thiopyran-2-yl, thiopryran-3-yl, thiopryran-4-yl, 1-oxothiopryran-2-yl, 1-oxothiopryran-3-yl, 1-oxothiopryran-4-yl, 1,1-dioxothiopryran-2-yl, 1,1-dioxothiopryran-3-yl, 1,1-dioxothiopryran-4-yl, 2H-oxazin-2-yl, 2H-oxazin-3-yl, 2H-oxazin-4-yl, 2H-oxazin-5-yl, 2H-oxazin-6-yl, 4H-oxazin-3-yl, 4H-oxazin-4-yl, 4H-oxazin-5-yl, 4H-oxazin-6-yl, 6H-oxazin-3-yl, 6H-oxazin-4-yl, 7H-oxazin-5-yl, 8H-oxazin-6-yl, 2H-1,3-oxazin-2-yl, 2H-1,3-oxazin-4-yl, 2H-1,3-oxazin-5-yl, 2H-1,3-oxazin-6-yl, 4H-1,3-oxazin-2-yl, 4H-1,3-oxazin-4-yl, 4H-1,3-oxazin-5-yl, 4H-1,3-oxazin-6-yl, 6H-1,3-oxazin-2-yl, 6H-1,3-oxazin-4-yl, 6H-1,3-oxazin-5-yl, 6H-1,3-oxazin-6-yl, 2H-1,4-oxazin-2-yl, 2H-1,4-oxazin-3-yl, 2H-1,4-oxazin-5-yl, 2H-1,4-oxazin-6-yl, 4H-1,4-oxazin-2-yl, 4H-1,4-oxazin-3-yl, 4H-1,4-oxazin-4-yl, 4H-1,4-oxazin-5-yl, 4H-1,4-oxazin-6-yl, 6H-1,4-oxazin-2-yl, 6H-1,4-oxazin-3-yl, 6H-1,4-oxazin-5-yl, 6H-1,4-oxazin-6-yl, 1,4-dioxine-2-yl, 1,4-oxathiin-2-yl, 1H-azepine, 1H-[1,3]-diazepine, 1H-[1,4]-diazepine, [1,3]diazocine, [1,5]diazocine, [1,5]diazocine and the like.

Examples of a 3-, 4-, 5-, 6-, 7- or 8-membered saturated heteromonocyclic ring containing 1 or 2 heteroatoms or heteroatom groups selected from the group consisting of O, N, S, NO, SO and SO₂, as ring members include: Oxiran-2-yl, thiiran-2-yl, aziridin-1-yl, aziridin-2-yl, oxetan-2-yl, oxetan-3-yl, thietan-2-yl, thietan-3-yl, 1-oxothietan-2-yl, 1-oxothietan-3-yl, 1,1-dioxothietan-2-yl, 1,1-dioxothietan-3-yl, azetidin-1-yl, azetidin-2-yl, azetidin-3-yl, tetrahydrofuran-2-yl, tetrahydrofuran-3-yl, tetrahydrothien-2-yl, tetrahydrothien-3-yl, 1-oxotetrahydrothien-2-yl, 1,1-dioxotetrahydrothien-2-yl, 1-oxotetrahydrothien-3-yl, 1,1-dioxotetrahydrothien-3-yl, pyrrolidin-1-yl, pyrrolidin-2-yl, pyrrolidin-3-yl, pyrazolidin-1-yl, pyrazolidin-3-yl, pyrazolidin-4-yl, pyrazolidin-5-yl, imidazolidin-1-yl, imidazolidin-2-yl, imidazolidin-4-yl, oxazolidin-2-yl, oxazolidin-3-yl, oxazolidin-4-yl, oxazolidin-5-yl, isoxazolidin-2-yl, isoxazolidin-3-yl, isoxazolidin-4-yl, isoxazolidin-5-yl, thiazolidin-2-yl, thiazolidin-3-yl, thiazolidin-4-yl, thiazolidin-5-yl, isothiazolidin-2-yl, isothiazolidin-3-yl, isothiazolidin-4-yl, isothiazolidin-5-yl, tetrahydropyran-2-yl, tetrahydropyran-3-yl, tetrahydropyran-4-yl, 1,3-dioxan-2-yl, 1,3-dioxan-4-yl, 1,3-dioxan-5-yl, 1,4-dioxan-2-yl, piperidin-1-yl, piperidin-2-yl, piperidin-3-yl, piperidin-4-yl, hexahydropyridazin-1-yl, hexahydropyridazin-3-yl, hexahydropyridazin-4-yl, hexahydropyrimidin-1-yl, hexahydropyrimidin-2-yl, hexahydropyrimidin-4-yl, hexahydropyrimidin-5-yl, piperazin-1-yl, piperazin-2-yl, morpholin-2-yl, morpholin-3-yl, morpholin-4-yl, thiomorpholin-2-yl, thiomorpholin-3-yl, thiomorpholin-4-yl, 1-oxothiomorpholin-2-yl, 1-oxothiomorpholin-3-yl, 1-oxothiomorpholin-4-yl, 1,1-dioxothiomorpholin-2-yl, 1,1-dioxothiomorpholin-3-yl, 1,1-dioxothiomorpholin-4-yl, azepan-1-, -2-, -3- or -4-yl, oxepan-2-, -3-, -4- or -5-yl, hexahydro-1,3-diazepinyl, hexahydro-1,4-diazepinyl, hexahydro-1,3-oxazepinyl, hexahydro-1,4-oxazepinyl, hexahydro-1,3-dioxepinyl, hexahydro-1,4-dioxepinyl, oxocane, thiocane, azocanyl, [1,3]diazocanyl, [1,4]diazocanyl, [1,5]diazocanyl, [1,5]oxazocanyl and the like.

Examples of a 3-, 4-, 5-, 6-, 7- or 8-membered partially unsaturated heteromonocyclic ring containing 1 or 2 heteroatoms or heteroatom groups selected from the group consisting of O, N, S, NO, SO and SO₂, as ring members include: 2,3-dihydrofuran-2-yl, 2,3-dihydrofuran-3-yl, 2,4-dihydrofuran-2-yl, 2,4-dihydrofuran-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4-di- or tetrahydropyridazinyl, 2-di- or tetrahydropyrimidinyl, 4-di- or tetrahydropyrimidinyl, 5-di- or tetrahydropyrimidinyl, di- or tetrahydropyrazinyl, 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, tetrahydrooxepinyl, such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, tetrahydro-1,3-diazepinyl, tetrahydro-1,4-diazepinyl, tetrahydro-1,3-oxazepinyl, tetrahydro-1,4-oxazepinyl, tetrahydro-1,3-dioxepinyl, tetrahydro-1,4-dioxepinyl, 1,2,3,4,5,6-hexahydroazocine, 2,3,4,5,6,7-hexahydroazocine, 1,2,3,4,5,8-hexahydroazocine, 1,2,3,4,7,8-hexahydroazocine, 1,2,3,4,5,6-hexahydro-[1,5]diazocine,1,2,3,4,7,8-hexahydro-[1,5]diazocine and the like.

Examples of a 3-, 4-, 5-, 6-, 7- or 8-membered maximally unsaturated (including aromatic) heteromonocyclic ring containing 1 or 2 heteroatoms or heteroatom groups selected from the group consisting of O, N, S, NO, SO and SO₂, as ring members are 2-furyl, 3-furyl, 2-thienyl, 3-thienyl, 1-pyrrolyl, 2-pyrrolyl, 3-pyrrolyl, 1-pyrazolyl, 3-pyrazolyl, 4-pyrazolyl, 5-pyrazolyl, 1-imidazolyl, 2-imidazolyl, 4-imidazolyl, 5-imidazolyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 3-isothiazolyl, 4-isothiazolyl, 5-isothiazolyl, 2-pyridinyl, 3-pyridinyl, 4-pyridinyl, 1-oxopyridin-2-yl, 1-oxopyridin-3-yl, 1-oxopyridin-4-yl, 3-pyridazinyl, 4-pyridazinyl, 2-pyrimidinyl, 4-pyrimidinyl, 5-pyrimidinyl, 2-pyrazinyl, pyran-2-yl, pyran-3-yl, pyran-4-yl, thiopyran-2-yl, thiopryran-3-yl, thiopryran-4-yl, 1-oxothiopryran-2-yl, 1-oxothiopryran-3-yl, 1-oxothiopryran-4-yl, 1,1-dioxothiopryran-2-yl, 1,1-dioxothiopryran-3-yl, 1,1-dioxothiopryran-4-yl, 2H-oxazin-2-yl, 2H-oxazin-3-yl, 2H-oxazin-4-yl, 2H-oxazin-5-yl, 2H-oxazin-6-yl, 4H-oxazin-3-yl, 4H-oxazin-4-yl, 4H-oxazin-5-yl, 4H-oxazin-6-yl, 6H-oxazin-3-yl, 6H-oxazin-4-yl, 7H-oxazin-5-yl, 8H-oxazin-6-yl, 2H-1,3-oxazin-2-yl, 2H-1,3-oxazin-4-yl, 2H-1,3-oxazin-5-yl, 2H-1,3-oxazin-6-yl, 4H-1,3-oxazin-2-yl, 4H-1,3-oxazin-4-yl, 4H-1,3-oxazin-5-yl, 4H-1,3-oxazin-6-yl, 6H-1,3-oxazin-2-yl, 6H-1,3-oxazin-4-yl, 6H-1,3-oxazin-5-yl, 6H-1,3-oxazin-6-yl, 2H-1,4-oxazin-2-yl, 2H-1,4-oxazin-3-yl, 2H-1,4-oxazin-5-yl, 2H-1,4-oxazin-6-yl, 4H-1,4-oxazin-2-yl, 4H-1,4-oxazin-3-yl, 4H-1,4-oxazin-4-yl, 4H-1,4-oxazin-5-yl, 4H-1,4-oxazin-6-yl, 6H-1,4-oxazin-2-yl, 6H-1,4-oxazin-3-yl, 6H-1,4-oxazin-5-yl, 6H-1,4-oxazin-6-yl, 1,4-dioxine-2-yl, 1,4-oxathiin-2-yl, 1H-azepine, 1H-[1,3]-diazepine, 1H-[1,4]-diazepine, [1,3]diazocine, [1,5]diazocine, [1,5]diazocine and the like.

Examples of a 5- or 6-membered saturated heteromonocyclic ring containing 1, 2, 3 or 4 heteroatoms or heteroatom groups selected from the group consisting of O, N, S, NO, SO and SO₂, as ring members include: tetrahydrofuran-2-yl, tetrahydrofuran-3-yl, tetrahydrothien-2-yl, tetrahydrothien-3-yl, 1-oxotetrahydrothien-2-yl, 1,1-dioxotetrahydrothien-2-yl, 1-oxotetrahydrothien-3-yl, 1,1-dioxotetrahydrothien-3-yl, pyrrolidin-1-yl, pyrrolidin-2-yl, pyrrolidin-3-yl, pyrazolidin-1-yl, pyrazolidin-3-yl, pyrazolidin-4-yl, pyrazolidin-5-yl, imidazolidin-1-yl, imidazolidin-2-yl, imidazolidin-4-yl, oxazolidin-2-yl, oxazolidin-3-yl, oxazolidin-4-yl, oxazolidin-5-yl, isoxazolidin-2-yl, isoxazolidin-3-yl, isoxazolidin-4-yl, isoxazolidin-5-yl, thiazolidin-2-yl, thiazolidin-3-yl, thiazolidin-4-yl, thiazolidin-5-yl, isothiazolidin-2-yl, isothiazolidin-3-yl, isothiazolidin-4-yl, isothiazolidin-5-yl, 1,2,4-oxadiazolidin-2-yl, 1,2,4-oxadiazolidin-3-yl, 1,2,4-oxadiazolidin-4-yl, 1,2,4-oxadiazolidin-5-yl, 1,2,4-thiadiazolidin-2-yl, 1,2,4-thiadiazolidin-3-yl, 1,2,4-thiadiazolidin-4-yl, 1,2,4-thiadiazolidin-5-yl, 1,2,4-triazolidin-1-yl, 1,2,4-triazolidin-3-yl, 1,2,4-triazolidin-4-yl, 1,3,4-oxadiazolidin-2-yl, 1,3,4-oxadiazolidin-3-yl, 1,3,4-thiadiazolidin-2-yl, 1,3,4-thiadiazolidin-3-yl, 1,3,4-triazolidin-1-yl, 1,3,4-triazolidin-2-yl, 1,3,4-triazolidin-3-yl, 1,2,3,4-tetrazolidin-1-yl, 1,2,3,4-tetrazolidin-2-yl, 1,2,3,4-tetrazolidin-5-yl, tetrahydropyran-2-yl, tetrahydropyran-3-yl, tetrahydropyran-4-yl, 1,3-dioxan-2-yl, 1,3-dioxan-4-yl, 1,3-dioxan-5-yl, 1,4-dioxan-2-yl, piperidin-1-yl, piperidin-2-yl, piperidin-3-yl, piperidin-4-yl, hexahydropyridazin-1-yl, hexahydropyridazin-3-yl, hexahydropyridazin-4-yl, hexahydropyrimidin-1-yl, hexahydropyrimidin-2-yl, hexahydropyrimidin-4-yl, hexahydropyrimidin-5-yl, piperazin-1-yl, piperazin-2-yl, 1,3,5-hexahydrotriazin-1-yl, 1,3,5-hexahydrotriazin-2-yl, 1,2,4-hexahydrotriazin-1-yl, 1,2,4-hexahydrotriazin-2-yl, 1,2,4-hexahydrotriazin-3-yl, 1,2,4-hexahydrotriazin-4-yl, 1,2,4-hexahydrotriazin-5-yl, 1,2,4-hexahydrotriazin-6-yl, morpholin-2-yl, morpholin-3-yl, morpholin-4-yl, thiomorpholin-2-yl, thiomorpholin-3-yl, thiomorpholin-4-yl, 1-oxothiomorpholin-2-yl, 1-oxothiomorpholin-3-yl, 1-oxothiomorpholin-4-yl, 1,1-dioxothiomorpholin-2-yl, 1,1-dioxothiomorpholin-3-yl, 1,1-dioxothiomorpholin-4-yl, and the like.

Examples of a 5- or 6-membered partially unsaturated heteromonocyclic ring containing 1, 2, 3 or 4 heteroatoms or heteroatom groups selected from the group consisting of O, N, S, NO, SO and SO₂, as ring members include: 2,3-dihydrofuran-2-yl, 2,3-dihydrofuran-3-yl, 2,4-dihydrofuran-2-yl, 2,4-dihydrofuran-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4-di- or tetrahydropyridazinyl, 2-di- or tetrahydropyrimidinyl, 4-di- or tetrahydropyrimidinyl, 5-di- or tetrahydropyrimidinyl, di- or tetrahydropyrazinyl, 1,3,5-di- or tetrahydrotriazin-2-yl, 1,2,4-di- or tetrahydrotriazin-3-yl, and the like.

Examples of a 5- or 6-membered maximally unsaturated (including aromatic) heteromonocyclic ring containing 1, 2, 3 or 4 heteroatoms or heteroatom groups selected from the group consisting of O, N, S, NO, SO and SO₂, as ring members are 2-furyl, 3-furyl, 2-thienyl, 3-thienyl, 1-pyrrolyl, 2-pyrrolyl, 3-pyrrolyl, 1-pyrazolyl, 3-pyrazolyl, 4-pyrazolyl, 5-pyrazolyl, 1-imidazolyl, 2-imidazolyl, 4-imidazolyl, 5-imidazolyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 3-isothiazolyl, 4-isothiazolyl, 5-isothiazolyl, 1,3,4-triazol-1-yl, 1,3,4-triazol-2-yl, 1,3,4-triazol-3-yl, 1,2,3-triazol-1-yl, 1,2,3-triazol-2-yl, 1,2,3-triazol-4-yl, 1,2,5-oxadiazol-3-yl, 1,2,3-oxadiazol-4-yl, 1,2,3-oxadiazol-5-yl, 1,3,4-oxadiazol-2-yl, 1,2,5-thiadiazol-3-yl, 1,2,3-thiadiazol-4-yl, 1,2,3-thiadiazol-5-yl, 1,3,4-thiadiazol-2-yl, 1,2,3,4-tetrazol-1-yl, 1,2,3,4-tetrazol-2-yl, 1,2,3,4-tetrazol-5-yl, 2-pyridinyl, 3-pyridinyl, 4-pyridinyl, 1-oxopyridin-2-yl, 1-oxopyridin-3-yl, 1-oxopyridin-4-yl, 3-pyridazinyl, 4-pyridazinyl, 2-pyrimidinyl, 4-pyrimidinyl, 5-pyrimidinyl, 2-pyrazinyl, 1,3,5-triazin-2-yl, 1,2,4-triazin-3-yl, 1,2,4-triazin-5-yl, 1,2,3,4-tetrazin-1-yl, 1,2,3,4-tetrazin-2-yl, 1,2,3,4-tetrazin-5-yl, pyran-2-yl, pyran-3-yl, pyran-4-yl, thiopyran-2-yl, thiopryran-3-yl, thiopryran-4-yl, 1-oxothiopryran-2-yl, 1-oxothiopryran-3-yl, 1-oxothiopryran-4-yl, 1,1-dioxothiopryran-2-yl, 1,1-dioxothiopryran-3-yl, 1,1-dioxothiopryran-4-yl, and the like.

Examples for 5- or 6-membered monocyclic heteroaromatic rings containing 1, 2, 3 or 4 heteroatoms selected from the group consisting of N, O and S as ring members are 2-furyl, 3-furyl, 2-thienyl, 3-thienyl, 1-pyrrolyl, 2-pyrrolyl, 3-pyrrolyl, 1-pyrazolyl, 3-pyrazolyl, 4-pyrazolyl, 5-pyrazolyl, 1-imidazolyl, 2-imidazolyl, 4-imidazolyl, 5-imidazolyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 3-isothiazolyl, 4-isothiazolyl, 5-isothiazolyl, 1,3,4-triazol-1-yl, 1,3,4-triazol-2-yl, 1,3,4-triazol-3-yl, 1,2,3-triazol-1-yl, 1,2,3-triazol-2-yl, 1,2,3-triazol-4-yl, 1,2,5-oxadiazol-3-yl, 1,2,3-oxadiazol-4-yl, 1,2,3-oxadiazol-5-yl, 1,3,4-oxadiazol-2-yl, 1,2,5-thiadiazol-3-yl, 1,2,3-thiadiazol-4-yl, 1,2,3-thiadiazol-5-yl, 1,3,4-thiadiazol-2-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl, 1,2,4-triazin-3-yl, 1,2,4-triazin-5-yl, 1,2,3,4-tetrazin-1-yl, 1,2,3,4-tetrazin-2-yl, 1,2,3,4-tetrazin-5-yl and the like.

Examples for 5- or 6-membered monocyclic heteroaromatic rings containing 1 heteroatom selected from the group consisting of N, O and S as ring member are 2-furyl, 3-furyl, 2-thienyl, 3-thienyl, 1-pyrrolyl, 2-pyrrolyl, 3-pyrrolyl, 2-pyridinyl, 3-pyridinyl and 4-pyridinyl.

Examples for a 5-membered monocyclic heteroaromatic ring containing 1 heteroatom selected from the group consisting of N, O and S as ring member are 2-furyl, 3-furyl, 2-thienyl, 3-thienyl, 1-pyrrolyl, 2-pyrrolyl and 3-pyrrolyl.

“Hetaryl-C₁-C₃-alkyl” refers to a 5- or 6-membered heteroaromatic ring containing 1, 2, 3, or 4 heteroatoms selected from the group consisting of O, S and N as ring members, as defined above, bound to the remainder of the molecule via a C₁-C₃-alkyl group. Examples are 2-furyl-methyl, 3-furyl-methyl, 2-thienyl-methyl, 3-thienyl-methyl, 1-pyrrolyl-methyl, 2-pyrrolyl-methyl, 3-pyrrolyl-methyl, 1-pyrazolyl-methyl, 3-pyrazolyl-methyl, 4-pyrazolyl-methyl, 5-pyrazolyl-methyl, 1-imidazolyl-methyl, 2-imidazolyl-methyl, 4-imidazolyl-methyl, 5-imidazolyl-methyl, 2-oxazolyl-methyl, 4-oxazolyl-methyl, 5-oxazolyl-methyl, 3-isoxazolyl-methyl, 4-isoxazolyl-methyl, 5-isoxazolyl-methyl, 2-thiazolyl-methyl, 4-thiazolyl-methyl, 5-thiazolyl-methyl, 3-isothiazolyl-methyl, 4-isothiazolyl-methyl, 5-isothiazolyl-methyl, 1,3,4-triazol-1-yl-methyl, 1,3,4-triazol-2-yl-methyl, 1,3,4-triazol-3-yl-methyl, 1,2,3-triazol-1-yl-methyl, 1,2,3-triazol-2-yl-methyl, 1,2,3-triazol-4-yl-methyl, 1,2,5-oxadiazol-3-yl-methyl, 1,2,3-oxadiazol-4-yl-methyl, 1,2,3-oxadiazol-5-yl-methyl, 1,3,4-oxadiazol-2-yl-methyl, 1,2,5-thiadiazol-3-yl-methyl, 1,2,3-thiadiazol-4-yl-methyl, 1,2,3-thiadiazol-5-yl-methyl, 1,3,4-thiadiazol-2-yl-methyl, 2-pyridinyl-methyl, 3-pyridinyl-methyl, 4-pyridinyl-methyl, 3-pyridazinyl-methyl, 4-pyridazinyl-methyl, 2-pyrimidinyl-methyl, 4-pyrimidinyl-methyl, 5-pyrimidinyl-methyl, 2-pyrazinyl-methyl, 1,3,5-triazin-2-yl-methyl, 1,2,4-triazin-3-yl-methyl, 1,2,4-triazin-5-yl-methyl, 1,2,3,4-tetrazin-1-yl-methyl, 1,2,3,4-tetrazin-2-yl-methyl, 1,2,3,4-tetrazin-5-yl-methyl and the like.

“Heterocyclyl-C₁-C₃-alkyl” is a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, as defined above, bound to the remainder of the molecule via a C₁-C₃-alkyl group.

“Alkylene” is a linear or branched divalent alkanediyl radical. C₁-C₆-Alkylene is a linear or branched divalent alkyl radical having 1, 2, 3, 4, 5 or 6 carbon atoms. Examples are —CH₂—, —CH₂CH₂—, —CH(CH₃)—, —CH₂CH₂CH₂—, —CH(CH₃)CH₂—, —CH₂CH(CH₃)—, —C(CH₃)₂—, —CH₂CH₂CH₂CH₂—, —CH(CH₃)CH₂CH₂—, —CH₂CH₂CH(CH₃)—, —C(CH₃)₂CH₂—, —CH₂C(CH₃)₂—, —(CH₂)₅—, —(CH₂)₆—, —(CH₂)₇—, —(CH₂)₈—, —(CH₂)₉—, —(CH₂)₁₀— and positional isomers thereof.

“C₃-C₈-Cycloalkylene” stands for a divalent monocyclic, saturated hydrocarbon group having 3 to 8 carbon ring members. Examples are cyclopropane-1,1-diyl, cyclopropane-1,2-diyl, cyclobutane-1,1-diyl, cyclobutane-1,2-diyl, cyclobutane-1,3-diyl, cyclopentane-1,1-diyl, cyclopentane-1,2-diyl, cyclopentane-1,3-diyl, cyclohexane-1,1-diyl, cyclohexane-1,2-diyl, cyclohexane-1,3-diyl, cyclohexane-1,4-diyl, cycloheptane-1,1-diyl, cycloheptane-1,2-diyl, cycloheptane-1,3-diyl, cycloheptane-1,4-diyl, cyclooctane-1,1-diyl, cyclooctane-1,2-diyl, cyclooctane-1,3-diyl, cyclooctane-1,4-diyl, and cyclooctane-1,5-diyl.

The remarks made above and in the following with respect to preferred aspects of the invention, e.g. to preferred meanings of the variables A, X¹, X², X³, X⁴, L¹, L², R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, m and n of compounds I, to preferred compounds I and to preferred embodiments of the methods or the use according to the invention, apply in each case on their own or in particular to combinations thereof.

In one embodiment, X¹ is CR¹, X² is CR², X³ is CR³ and X⁴ is CR⁴. In another embodiment, X¹ is N, X² is CR², X³ is CR³ and X⁴ is CR⁴. In yet another embodiment, X¹ is CR¹, X² is N, X³ is CR³ and X⁴ is CR⁴. In yet another embodiment, X¹ is CR¹, X² is CR², X³ is N and X⁴ is CR⁴. In yet another embodiment, X¹ is CR¹, X² is CR², X³ is CR³ and X⁴ is N. In yet another embodiment, X¹ is N, X² is CR², X³ is N and X⁴ is CR⁴. In yet another embodiment, X¹ is CR¹, X² is N, X³ is CR³ and X⁴ is N.

Preferably,

X¹ is CR¹, X² is CR², X³ is CR³ and X⁴ is CR⁴; or
X¹ is N, X² is CR², X³ is CR³ and X⁴ is CR⁴; or
X¹ is CR¹, X² is N, X³ is CR³ and X⁴ is CR⁴; or
X¹ is CR¹, X² is CR², X³ is N and X⁴ is CR⁴; or
X¹ is CR¹, X² is CR², X³ is CR³ and X⁴ is N.

More preferably,

X¹ is CR¹, X² is CR², X³ is CR³ and X⁴ is CR⁴; or
X¹ is N, X² is CR², X³ is CR³ and X⁴ is CR⁴; or
X¹ is CR¹, X² is CR², X³ is CR³ and X⁴ is N.

Even more preferably,

X¹ is CR¹, X² is CR², X³ is CR³ and X⁴ is CR⁴; or
X¹ is N, X² is CR², X³ is CR³ and X⁴ is CR⁴.

In particular, X¹ is CR¹, X² is CR², X³ is CR³ and X⁴ is CR⁴.

Preferably,
R¹ and R², independently of each other, are selected from the group consisting of hydrogen, halogen, CN, C₁-C₆-alkyl, C₁-C₆-haloalkyl, C₃-C₈-cycloalkyl, C₃-C₈-halocycloalkyl, C₁-C₆-alkoxy, C₁-C₆-haloalkoxy, C₁-C₆-alkylthio, C₁-C₆-haloalkylthio, phenyl which may carry one or more substituents R¹⁸, and a 5- or 6-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸; and
R³ and R⁴, independently of each other, are selected from the group consisting of hydrogen, halogen, CN, C₁-C₆-alkyl, C₁-C₆-haloalkyl, C₁-C₄-alkoxy and C₁-C₄-haloalkoxy;
or R¹ and R², or R² and R³, together with the carbon atoms they are bound to, form a 5- or 6-membered saturated, partially unsaturated or maximally unsaturated carbocyclic or heterocyclic ring, where the heterocyclic ring contains 1, 2 or 3 heteroatoms or heteroatom-containing groups selected from the group consisting of O, N, S, NO, SO and SO, as ring members.

More preferably,
R¹ and R², independently of each other, are selected from the group consisting of hydrogen, halogen, CN, C₁-C₄-alkyl and C₁-C₄-alkoxy; and
R³ and R⁴, independently of each other, are selected from the group consisting of hydrogen, F, C₁-C₄-alkyl and C₁-C₄-alkoxy;
or R¹ and R², or R² and R³ form together a bridging group —CH₂CH₂CH₂—, —CH₂CH₂CH₂CH₂—, or —O—CH₂—O—.

Even more preferably,
R¹ and R², independently of each other, are selected from the group consisting of hydrogen, halogen, CN, C₁-C₄-alkyl, C₁-C₄-alkoxy and C₁-C₄-haloalkoxy;
R³ is selected from the group consisting of hydrogen, C₁-C₄-alkyl and C₁-C₄-alkoxy;
or R² and R³ form together a bridging group —CH₂CH₂CH₂— or —O—CH₂—O—; and
R⁴ is hydrogen or methyl; in particular hydrogen.

In particular,
R¹ and R², independently of each other, are selected from the group consisting of hydrogen, F, Cl, CN, C₁-C₄-alkyl, C₁-C₂-alkoxy and C₁-C₄-haloalkoxy;
R³ is selected from the group consisting of hydrogen and C₁-C₄-alkyl;
or R² and R³ form together a bridging group —CH₂CH₂CH₂— or —O—CH₂—O—; in particular a bridging group —CH₂CH₂CH₂—; and
R⁴ is hydrogen.

Specifically,
R¹ and R², independently of each other, are selected from the group consisting of hydrogen, F, Cl, C₁-C₄-alkyl and C₁-C₂-alkoxy;
R³ is selected from the group consisting of hydrogen and C₁-C₄-alkyl;
or R² and R³ form together a bridging group —CH₂CH₂CH₂—; and
R⁴ is hydrogen.

More specifically,
R¹ and R², independently of each other, are selected from the group consisting of hydrogen, F, Cl, C₁-C₄-alkyl and C₁-C₂-alkoxy;
R³ is selected from the group consisting of hydrogen and C₁-C₄-alkyl; and
R⁴ is hydrogen.

In a particular embodiment of the present invention, R² and R³ have one of the meanings given above, but do not form a bridging group —CH₂CH₂CH₂—.

R⁵ is preferably hydrogen or C₁-C₄ alkyl, and is in particular hydrogen.

R⁶ is preferably selected from the group consisting of hydrogen, C₁-C₄-alkyl, C₃-C₄-alkenyl and phenyl which carries a substituent R¹⁸; where R¹⁸ has one of the above general or, in particular, one of the below preferred meanings. Preferably, in this context R¹⁸ is selected from the group consisting of halogen, C₃-C₆-cycloalkyl, C₁-C₄-alkoxy, C₁-C₄-haloalkoxy, C₁-C₄-alkylthio, C₁-C₄-haloalkylthio, C₁-C₄-alkylsulfonyl, C₁-C₄-haloalkylsulfonyl, and C₁-C₄-alkylcarbonyl; and is specifically C₁-C₄-alkylthio, C₁-C₄-haloalkylthio, or C₁-C₄-alkylcarbonyl.

In one preferred embodiment R⁶ is hydrogen. In another preferred embodiment R⁶ is C₃-C₄-alkenyl or phenyl which carries a substituent R¹⁸; where R¹⁸ has one of the above general or, in particular, one of the above preferred meanings. Preferably, in this context R¹⁸ is selected from the group consisting of halogen, C₃-C₆-cycloalkyl, C₁-C₄-alkoxy, C₁-C₄-haloalkoxy, C₁-C₄-alkylthio, C₁-C₄-haloalkylthio, C₁-C₄-alkylsulfonyl, C₁-C₄-haloalkylsulfonyl, and C₁-C₄-alkylcarbonyl; and is specifically C₁-C₄-alkylthio, C₁-C₄-haloalkylthio, or C₁-C₄-alkylcarbonyl.

In particular, R⁶ is hydrogen.

Preferably, L¹ is C₁-C₆-alkylene which may carry one or more, in particular 1 or 2, substituents R⁷; where R⁷ has one of the above general or, in particular, one of the below preferred meanings. Preferably, however, each R⁷ in this context is independently selected from the group consisting of F, CN, OH, C₁-C₄-alkyl, C₁-C₄-haloalkyl, C₃-C₆-cycloalkyl, C₃-C₆-halocycloalkyl, C₁-C₄-alkoxy, C₁-C₄-haloalkoxy and phenyl which may carry one or more substituents R¹⁸, where R¹⁸ has one of the above general or, in particular, one of the below preferred meanings; or two radicals R⁷ bound on the same carbon atom of the alkylene group, form together a group ═O. Preferably, each R¹⁸ in this context is independently selected from the group consisting of halogen, CN, nitro, OH, SH, C₁-C₆-alkyl which may carry one or more substituents NR²³R²⁴; C₁-C₆-haloalkyl, C₃-C₈-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, carboxyl, C₁-C₆-alkylcarbonyl and C₁-C₆-haloalkylcarbonyl; or two radicals R¹⁸ bound on adjacent ring atoms, together with the ring atoms they are bound to, may form a saturated, partially unsaturated or maximally unsaturated 5- or 6-membered carbocyclic or heterocyclic ring, where the heterocyclic ring contains 1 or 2 heteroatoms or heteroatom-containing groups selected from the group consisting of O, N, S, NO, SO and SO₂ as ring members, where the carbocyclic or heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo. More preferably, each R¹⁸ in this context is independently selected from the group consisting of halogen, CN, C₁-C₄-alky, C₁-C₆-haloalkyl, C₁-C₆-alkoxy and C₁-C₆-haloalkoxy. More preferably, each R⁷ in this context is independently C₁-C₄-alkyl and is specifically methyl.

More preferably, L¹ is CH₂, CH(CH₃) or CH₂CH₂. Specifically, L¹ is CH₂ or CH(CH₃).

Preferably L² is a bond, C₁-C₆-alkylene or C₁-C₆-alkylene-NR¹⁵, where the alkylene moiety in the two last-mentioned radicals may carry one or more substituents R⁷, where R⁷ and R¹⁵ have one of the above general or, in particular, one of the below preferred meanings. Preferably, however, each R⁷ in this context is independently selected from the group consisting of F, CN, OH, C₁-C₄-alkyl, C₁-C₄-haloalkyl, C₃-C₆-cycloalkyl, C₃-C₆-halocycloalkyl, C₁-C₄-alkoxy, C₁-C₄-haloalkoxy and phenyl which may carry one or more substituents R¹⁸; or two radicals R⁷ bound on the same carbon atom of the alkylene group, form together a group ═O. Preferably, each R¹⁸ in this context is independently selected from the group consisting of halogen, CN, nitro, OH, SH, C₁-C₆-alkyl which may carry one or more substituents NR²³R²⁴; C₁-C₆-haloalkyl, C₃-C₈-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, carboxyl, C₁-C₆-alkylcarbonyl and C₁-C₆-haloalkylcarbonyl; or two radicals R¹⁸ bound on adjacent ring atoms, together with the ring atoms they are bound to, may form a saturated, partially unsaturated or maximally unsaturated 5- or 6-membered carbocyclic or heterocyclic ring, where the heterocyclic ring contains 1 or 2 heteroatoms or heteroatom-containing groups selected from the group consisting of O, N, S, NO, SO and SO₂ as ring members, where the carbocyclic or heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo. More preferably, each R¹⁸ in this context is independently selected from the group consisting of halogen, CN, C₁-C₄-alky, C₁-C₆-haloalkyl, C₁-C₆-alkoxy and C₁-C₆-haloalkoxy. More preferably, each R⁷ in this context is independently C₁-C₄-alkyl and is specifically methyl. Also preferably in this context, R¹⁵ is selected from the group consisting of hydrogen, C₁-C₆-alkyl which may carry one or more substituents R¹⁹, C₁-C₆-haloalkyl, C₃-C₆-cycloalkyl, C₃-C₆-halocycloalkyl, C₁-C₆-alkylcarbonyl and C₁-C₆-haloalkylcarbonyl; and is more preferably hydrogen or C₁-C₆-alkyl.

More preferably, L² is a bond, CH₂, CH₂CH₂ or CH₂CH₂NH, and is specifically a bond or CH₂CH₂NH.

A is preferably C₅-C₆-cycloalkyl which may carry one or two substituents R⁹, or is a 5- or 6-membered saturated, partially unsaturated or aromatic heterocyclic ring containing 1 or 2 heteroatoms selected from the group consisting of O, N and S as ring members, where the heterocyclic ring may carry one or more substituents R¹⁰; where R⁹ and R¹⁰ have one of the above general or, in particular, one of the below preferred meanings.

A is more preferably C₅-C₆-cycloalkyl which may carry one or two substituents R⁹, or is a 5-membered saturated or aromatic heterocyclic ring containing 1 or 2 heteroatoms selected from the group consisting of O, N and S as ring members, where the heterocyclic ring may carry one or more substituents R¹⁰; where R⁹ and R¹⁰ have one of the above general or, in particular, one of the below preferred meanings.

Preferably, however,
each R⁹ in this context is independently selected from the group consisting of halogen, C₁-C₆-alkyl which may carry one or more substituents R¹¹, and C₁-C₆-haloalkyl,
or two radicals R⁹ bound on adjacent ring atoms, together with the ring atoms they are bound to, may form a maximally unsaturated 5- or 6-membered carbocyclic ring;
or two radicals R⁹ bound on non-adjacent ring atoms may form a bridge —CH₂—; and
each R¹⁰ in this context is independently selected from the group consisting of CN, C₁-C₆-alkyl which may carry one or more substituents R¹¹, C₁-C₆-haloalkyl, C₁-C₆-alkoxy, C₁-C₆-haloalkoxy, S(O)ₙR¹⁴, C(O)R¹⁷, C(O)OR¹³, C(O)NR¹⁵R¹⁶, aryl which may carry one or more substituents R¹⁸, and a 5- or 6-membered heteroaromatic ring containing 1, 2, 3 or 4 heteroatoms groups selected from the group consisting of O, N and S as ring members, where the heteroaromatic ring may carry one or more substituents R¹⁸;
or two radicals R^{1′} bound on adjacent ring atoms, together with the ring atoms they are bound to, may form a saturated, partially unsaturated or maximally unsaturated 5- or 6-membered carbocyclic or heterocyclic ring, where the heterocyclic ring contains 1, 2, 3 or 4 heteroatoms or heteroatom-containing groups selected from the group consisting of O, N, S, NO, SO and SO₂ as ring members, where the carbocyclic or heterocyclic ring may be substituted by one or more radicals selected from the group consisting of halogen, C₁-C₆-alkyl which may carry one or more substituents R¹¹, C₁-C₆-haloalkyl, C₁-C₆-alkoxy, C₁-C₆-haloalkoxy, C₁-C₆-alkylsulfonyl, C₁-C₆-haloalkylsulfonyl, and phenyl which may carry one or more substituents selected from the group consisting of halogen, C₁-C₆-alkyl, C₁-C₆-haloalkyl, C₁-C₆-alkoxy and C₁-C₆-haloalkoxy; where
   each R¹¹ is independently selected from the group consisting of OH, C₁-C₆-alkoxy, C₁-C₆-haloalkoxy, NR¹⁵R¹⁶, C(O)OR¹³, C(O)NR¹⁵R¹⁶, phenyl which may carry one or more substituents R¹⁸, and a 3-, 4-, 5-, 6-, 7- or 8-membered saturated heterocyclic ring containing 1 or 2 heteroatoms or heteroatom-containing groups selected from the group consisting of O, N, S, NO, SO and SO₂ as ring members, where the heterocyclic ring may carry one or more substituents R¹⁸;
   each R¹³ is independently C₁-C₆-alkyl or C₁-C₆-haloalkyl;
   R¹⁴ is phenyl which may carry one or more substituents R¹⁸;
   R¹⁵ and R¹⁶, independently of each other and independently of each occurrence, are selected from the group consisting of hydrogen, C₁-C₆-alkyl which may carry one or more substituents R¹⁹, C₁-C₆-haloalkyl, C₃-C₆-cycloalkyl, C₃-C₆-halocycloalkyl, C₁-C₆-alkylcarbonyl and C₁-C₆-haloalkylcarbonyl;
   or R¹⁵ and R¹⁶, together with the nitrogen atom they are bound to, form a saturated, partially unsaturated or maximally unsaturated 3-, 4-, 5- or 6-membered heterocyclic ring, where the heterocyclic ring may additionally contain 1 or 2 further heteroatoms or heteroatom-containing groups selected from the group consisting of O, N, S, NO, SO and SO₂ as ring members, where the heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo;
   each R¹⁷ is independently C₁-C₆-alkyl or C₁-C₆-haloalkyl;
   each R¹⁸ is independently selected from the group consisting of halogen, CN, nitro, OH, SH, C₁-C₆-alkyl which may carry one or more substituents NR²³R²⁴; C₁-C₆-haloalkyl, C₃-C₈-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, carboxyl, C₁-C₆-alkylcarbonyl and C₁-C₆-haloalkylcarbonyl;
   or two radicals R¹⁸ bound on adjacent ring atoms, together with the ring atoms they are bound to, may form a saturated, partially unsaturated or maximally unsaturated 5- or 6-membered carbocyclic or heterocyclic ring, where the heterocyclic ring contains 1 or 2 heteroatoms or heteroatom-containing groups selected from the group consisting of O, N, S, NO, SO and SO₂ as ring members, where the carbocyclic or heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo;
   each R¹⁹ is independently selected from the group consisting of CN, OH, C₁-C₆-alkoxy, C₁-C₆-haloalkoxy, SH, C₁-C₆-alkylthio, C₁-C₆-haloalkylthio, C₁-C₆-alkylsulfonyl, C₁-C₆-haloalkylsulfonyl, NR²³R²⁴ and phenyl; and
   R²³ and R²⁴, independently of each other and independently of each occurrence, are selected from the group consisting of hydrogen, C₁-C₆-alkyl, C₁-C₆-haloalkyl, C₃-C₈-cycloalkyl, C₃-C₈-halocycloalkyl, C₁-C₆-alkylcarbonyl, C₁-C₆-haloalkylcarbonyl, C₁-C₆-alkoxycarbonyl, C₁-C₆-haloalkoxycarbonyl, C₁-C₆-alkylsulfonyl, C₁-C₆-haloalkylsulfonyl, aryl and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where aryl or the heterocyclic ring may carry one or more substituents selected from the group consisting of halogen, CN, OH, C₁-C₆-alkyl, C₁-C₆-haloalkyl, C₁-C₆-alkoxy and C₁-C₆-haloalkoxy.

Even more preferably, A is a 5-membered heteroaromatic ring containing one nitrogen atom and one further heteroatom selected from the group consisting of O, N and S as ring members (i.e. A is an oxazole, isoxazole, pyrazole, imidazole, thiazole or isothiazole ring), where the heterocyclic ring may carry one or more substituents R¹⁰; where R¹⁰ has one of the above general or, in particular, one of the above or below preferred meanings.

Preferably, however,
each R¹⁰ in this context is independently selected from the group consisting of CN, C₁-C₄-alkyl which may carry one or more substituents R¹¹, C₁-C₄-haloalkyl, C(O)R¹⁷, C(O)OR¹³, C(O)NR¹⁵R¹⁶, phenyl which may carry one or more substituents R¹⁸, and a 5- or 6-membered heteroaromatic ring containing one heteroatom selected from the group consisting of O, N and S as ring members, where the heteroaromatic ring may carry one or more substituents R¹⁸;
or two radicals R^{1′} bound on adjacent ring atoms form together a bridging group —CH═CH—CH═CH—, —CH₂CH₂CH₂— or —CH₂CH₂CH₂CH₂—, where one of the hydrogen atoms in the bridging group may be substituted by a radical selected from the group consisting of methyl and methoxy; where
   each R¹¹ is independently selected from the group consisting of OH, C₁-C₄-alkoxy, C₁-C₄-haloalkoxy, NR¹⁵R¹⁶ and C(O)NR¹⁵R¹⁶;
   each R¹³ is independently C₁-C₄-alkyl;
   R¹⁵ and R¹⁶, independently of each other and independently of each occurrence, are selected from the group consisting of hydrogen, C₁-C₄-alkyl and C₁-C₄-alkylcarbonyl;
   R¹⁷ is C₁-C₄-alkyl;
   each R¹⁸ is independently selected from the group consisting of halogen, C₁-C₆-alkyl which may carry one substituent NR²³R²⁴; C₃-C₈-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, and C₁-C₆-alkylcarbonyl;
   or two radicals R¹⁸ bound on adjacent ring atoms, together with the ring atoms they are bound to, may form a saturated 5- or 6-membered heterocyclic ring containing 1 or 2 heteroatoms or heteroatom-containing groups selected from the group consisting of O, N, S, NO, SO and SO₂ as ring members, where the heterocyclic ring may be substituted by one or more radicals selected from the group consisting of halogen, C₁-C₄-alkyl, C₁-C₄-haloalkyl, C₁-C₄-alkoxy, C₁-C₄-haloalkoxy and oxo; and
R²³ and R²⁴, independently of each other and independently of each occurrence, are selected from the group consisting of hydrogen and C₁-C₄-alkylcarbonyl.

In particular, A is a 5-membered heteroaromatic ring containing one nitrogen atom and one further heteroatom selected from the group consisting of O, N and S as ring members (i.e. A is an oxazole, isoxazole, pyrazole, imidazole, thiazole or isothiazole ring), where the heterocyclic ring may carry one or more substituents R¹⁰; where R¹⁰ has one of the above general or, in particular, one of the above or below preferred meanings.

Preferably, however,
each R¹⁰ in this context is independently selected from the group consisting of CN, C₁-C₄-alkyl which may carry one or more substituents R¹¹, C₁-C₄-haloalkyl, C(O)R¹⁷, C(O)OR¹³, C(O)NR¹⁵R¹⁶, phenyl which may carry one or two substituents R¹⁸, and a 5- or 6-membered heteroaromatic ring containing one heteroatom selected from the group consisting of O, N and S as ring members, where the heteroaromatic ring may carry one or more substituents R¹⁸;
or two radicals R¹⁰ bound on adjacent ring atoms form together a bridging group —CH═CH—CH═CH— or —CH₂CH₂CH₂—, where one of the hydrogen atoms in the bridging group may be substituted by a radical selected from the group consisting of methyl and methoxy; where
   each R¹¹ is independently selected from the group consisting of OH, C₁-C₄-alkoxy, C₁-C₄-haloalkoxy, NR¹⁵R¹⁶ and C(O)NR¹⁵R¹⁶;
   each R¹³ is independently C₁-C₄-alkyl;
   R¹⁵ and R¹⁶, independently of each other, are selected from the group consisting of hydrogen, C₁-C₄-alkyl and C₁-C₄-alkylcarbonyl;
   R¹⁷ is C₁-C₄-alkyl;
   each R¹⁸ is independently selected from the group consisting of halogen, C₁-C₆-alkyl which may carry one substituent NR²³R²⁴; C₃-C₆-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, and C₁-C₆-alkylcarbonyl;
   or two radicals R¹⁸ bound on adjacent ring atoms, together with the ring atoms they are bound to, may form a saturated 5- or 6-membered heterocyclic ring containing one nitrogen ring atom or one or two oxygen atoms as ring members, where the heterocyclic ring may be substituted by an oxo group; and
   R²³ and R²⁴, independently of each other and independently of each occurrence, are selected from the group consisting of hydrogen and C₁-C₄-alkylcarbonyl.

In one specific embodiment of the invention, A is selected from the group consisting of oxazolyl, thiazolyl and imidazolyl, in particular from oxazol-2-yl, thiazol-2-yl and imidazol-2-yl, where oxazolyl, thiazolyl, imidazolyl and in particular oxazol-2-yl, thiazol-2-yl and imidazol-2-yl may carry one or more substituents R^{1′}, where R¹⁰ has one of the above general or, in particular, one of the above or below preferred meanings.

Preferably, however,
each R¹⁰ in this context is independently selected from the group consisting of CN, C₁-C₄-alkyl which may carry one or more substituents R¹¹, C₁-C₄-haloalkyl, C(O)R¹⁷, C(O)OR¹³, C(O)NR¹⁵R¹⁶, phenyl which may carry one or two substituents R¹⁸, and a 5- or 6-membered heteroaromatic ring containing one heteroatom selected from the group consisting of O, N and S as ring members, where the heteroaromatic ring may carry one or more substituents R¹⁸;
or two radicals R¹⁰ bound on adjacent ring atoms form together a bridging group —CH═CH—CH═CH— or —CH₂CH₂CH₂—, where one of the hydrogen atoms in the bridging group may be substituted by a radical selected from the group consisting of methyl and methoxy; where
   each R¹¹ is independently selected from the group consisting of OH, C₁-C₄-alkoxy, C₁-C₄-haloalkoxy, NR¹⁵R¹⁶ and C(O)NR¹⁵R¹⁶;
   each R¹³ is independently C₁-C₄-alkyl;
   R¹⁵ and R¹⁶, independently of each other, are selected from the group consisting of hydrogen, C₁-C₄-alkyl and C₁-C₄-alkylcarbonyl;
   R¹⁷ is C₁-C₄-alkyl;
   each R¹⁸ is independently selected from the group consisting of halogen, C₁-C₆-alkyl which may carry one substituent NR²³R²⁴; C₃-C₆-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, and C₁-C₆-alkylcarbonyl;
   or two radicals R¹⁸ bound on adjacent ring atoms, together with the ring atoms they are bound to, may form a saturated 5- or 6-membered heterocyclic ring containing one nitrogen ring atom or one or two oxygen atoms as ring members, where the heterocyclic ring may be substituted by an oxo group; and
      R²³ and R²⁴, independently of each other and independently of each occurrence, are selected from the group consisting of hydrogen and C₁-C₄-alkylcarbonyl.

In another specific embodiment of the invention, A is a 5-membered heteroaromatic ring containing one nitrogen atom and one further heteroatom selected from the group consisting of N and S as ring members (i.e. A is a pyrazole, imidazole, thiazole or isothiazole ring), where the heterocyclic ring may carry one or more substituents R¹⁰; where R¹⁰ has one of the above general or, in particular, one of the above or below preferred meanings.

Preferably, however,
each R¹⁰ is independently selected from the group consisting of CN, C₁-C₄-alkyl which may carry one or more substituents R¹¹, C₁-C₄-haloalkyl, C(O)R¹⁷, C(O)OR¹³, phenyl which may carry one or two substituents R¹⁸, and a 5- or 6-membered heteroaromatic ring containing one heteroatom selected from the group consisting of O, N and S as ring members, where the heteroaromatic ring may carry one or more substituents R¹⁸;
or two radicals R¹⁰ bound on adjacent ring atoms form together a bridging group —CH═CH—CH═CH— or —CH₂CH₂CH₂—, where one of the hydrogen atoms in the bridging group may be substituted by a radical selected from the group consisting of methyl and methoxy; where
   each R¹¹ is independently selected from the group consisting of OH, C₁-C₄-alkoxy, C₁-C₄-haloalkoxy and NR¹⁵R¹⁶;
   each R¹³ is independently C₁-C₄-alkyl;
   R¹⁵ and R¹⁶, independently of each other, are selected from the group consisting of hydrogen, C₁-C₄-alkyl and C₁-C₄-alkylcarbonyl;
   R¹⁷ is C₁-C₄-alkyl;
   each R¹⁸ is independently selected from the group consisting of halogen, C₁-C₆-alkyl which may carry one substituent NR²³R²⁴; C₃-C₆-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, and C₁-C₆-alkylcarbonyl;
   or two radicals R¹⁸ bound on adjacent ring atoms, together with the ring atoms they are bound to, may form a saturated 5- or 6-membered heterocyclic ring containing one nitrogen ring atom or one or two oxygen atoms as ring members, where the heterocyclic ring may be substituted by an oxo group; and
   R²³ and R²⁴, independently of each other and independently of each occurrence, are selected from the group consisting of hydrogen and C₁-C₄-alkylcarbonyl.

In this specific embodiment, A is in particular selected from imidazole and thiazole, where imidazole and thiazole may carry one or more substituents R¹⁰; where R¹⁰ has one of the above general or, in particular, one of the above or below preferred meanings.

In an alternatively preferred embodiment, L²-A forms a group C₁-C₆-alkylene-NR¹⁵R¹⁶; where R¹⁵ and R¹⁶ have one of the above general meanings. Preferably, however, in this context,
R¹⁵ and R¹⁶, independently of each other, are selected from the group consisting of hydrogen, C₁-C₆-alkyl which may carry one or more substituents R¹⁹, C₁-C₆-haloalkyl, C₃-C₆-cycloalkyl, C₃-C₆-halocycloalkyl, C₁-C₆-alkylcarbonyl and C₁-C₆-haloalkylcarbonyl;
or R¹⁵ and R¹⁶, together with the nitrogen atom they are bound to, form a saturated, partially unsaturated or maximally unsaturated 3-, 4-, 5- or 6-membered heterocyclic ring, where the heterocyclic ring may additionally contain 1 or 2 further heteroatoms or heteroatom-containing groups selected from the group consisting of O, N, S, NO, SO and SO₂ as ring members, where the heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo.

More preferably, in this context, R¹⁵ and R¹⁶, independently of each other, are selected from the group consisting of hydrogen, C₁-C₄-alkyl and C₁-C₄-alkylcarbonyl and in particular from hydrogen and C₁-C₄-alkyl. Specifically, they are both hydrogen.

In particular, L²-A forms a group CH₂CH₂—NR¹⁵R¹⁶; where R¹⁵ and R¹⁶ have one of the above general or, in particular, one of the above preferred meanings. Preferably, in this context, R¹⁵ and R¹⁶, independently of each other, are selected from the group consisting of hydrogen, C₁-C₄-alkyl and C₁-C₄-alkylcarbonyl and in particular from hydrogen and C₁-C₄-alkyl. Specifically, they are both hydrogen.

In a preferred embodiment, in compounds I
X¹ is CR¹, X² is CR², X³ is CR³ and X⁴ is CR⁴; or
X¹ is N, X² is CR², X³ is CR³ and X⁴ is CR⁴; or
X¹ is CR¹, X² is N, X³ is CR³ and X⁴ is CR⁴; or
X¹ is CR¹, X² is CR², X³ is N and X⁴ is CR⁴; or
X¹ is CR¹, X² is CR², X³ is CR³ and X⁴ is N; or
X¹ is N, X² is CR², X³ is N and X⁴ is CR⁴; or
X¹ is CR¹, X² is N, X³ is CR³ and X⁴ is N;
L¹ is C₁-C₆-alkylene which may carry one or more substituents R⁷;
L² is a bond, C₁-C₆-alkylene or C₁-C₆-alkylene-NR¹⁵, where the alkylene moiety in the two last-mentioned radicals may carry one or more substituents R⁷;
A is C₅-C₆-cycloalkyl which may carry 1 or two substituents R⁹, or is a 5-membered partially unsaturated or aromatic heterocyclic ring containing 1 or 2 heteroatoms selected from the group consisting of O, N and S as ring members, where the heterocyclic ring may carry one or more substituents R¹⁰;
or L²-A forms a group C₁-C₆-alkylene-NR¹⁵R¹⁶;
R¹ and R², independently of each other, are selected from the group consisting of hydrogen, halogen, CN, C₁-C₆-alkyl, C₁-C₆-haloalkyl, C₃-C₈-cycloalkyl, C₃-C₈-halocycloalkyl, C₁-C₆-alkoxy, C₁-C₆-haloalkoxy, C₁-C₆-alkylthio, C₁-C₆-haloalkylthio, phenyl which may carry one or more substituents R¹⁸, and a 5- or 6-membered saturated, partially unsaturated or maximally unsaturated heterocyclic ring containing 1, 2, 3 or 4 heteroatoms or heteroatom-containing groups selected from the group consisting of O, N, S, NO, SO and SO, as ring members, where the heterocyclic ring may carry one or more substituents R¹⁸;
R³ and R⁴, independently of each other, are selected from the group consisting of hydrogen, halogen, CN, C₁-C₆-alkyl, C₁-C₆-haloalkyl, C₁-C₄-alkoxy and C₁-C₄-haloalkoxy (where R⁴ is in particular hydrogen, F or methyl, more particularly hydrogen or methyl and specifically hydrogen);
or R¹ and R², or R² and R³, together with the carbon atoms they are bound to, form a 5- or 6-membered saturated, partially unsaturated or maximally unsaturated carbocyclic or heterocyclic ring, where the heterocyclic ring contains 1, 2 or 3 heteroatoms or heteroatom-containing groups selected from the group consisting of O, N, S, NO, SO and SO, as ring members;
R⁵ is hydrogen;
R⁶ is selected from the group consisting of hydrogen, C₁-C₆-alkyl which may carry one substituent R¹¹, C₂-C₆-alkenyl, and phenyl which may carry one or more substituents R¹⁸;
each R⁷ is independently selected from the group consisting of F, CN, OH, C₁-C₄-alkyl, C₁-C₄-haloalkyl, C₃-C₆-cycloalkyl, C₃-C₆-halocycloalkyl, C₁-C₄-alkoxy, C₁-C₄-haloalkoxy and phenyl which may carry one or more substituents R¹⁸; or two radicals R⁷ bound on the same carbon atom of the alkylene group, form together a group ═O;
each R⁹ is independently selected from the group consisting of halogen, C₁-C₆-alkyl which may carry one or more substituents R¹¹, and C₁-C₆-haloalkyl,
or two radicals R⁹ bound on adjacent ring atoms, together with the ring atoms they are bound to, may form a maximally unsaturated 5- or 6-membered carbocyclic ring;
or two radicals R⁹ bound on non-adjacent ring atoms may form a bridge —CH₂—;
each R¹⁰ is independently selected from the group consisting of CN, C₁-C₆-alkyl which may carry one or more substituents R¹¹, C₁-C₆-haloalkyl, C₁-C₆-alkoxy, C₁-C₆-haloalkoxy, S(O)₂R¹⁴, C(O)R¹⁷, C(O)OR¹³, C(O)NR¹⁵R¹⁶, aryl which may carry one or more substituents R¹⁸, and a 5- or 6-membered heteroaromatic ring containing 1, 2, 3 or 4 heteroatoms groups selected from the group consisting of O, N and S as ring members, where the heteroaromatic ring may carry one or more substituents R¹⁸;
or two radicals R¹⁰ bound on adjacent ring atoms, together with the ring atoms they are bound to, may form a saturated, partially unsaturated or maximally unsaturated 5- or 6-membered carbocyclic or heterocyclic ring, where the heterocyclic ring contains 1, 2, 3 or 4 heteroatoms or heteroatom-containing groups selected from the group consisting of O, N, S, NO, SO and SO₂ as ring members, where the carbocyclic or heterocyclic ring may be substituted by one or more radicals selected from the group consisting of halogen, C₁-C₆-alkyl which may carry one or more substituents R¹¹, C₁-C₆-haloalkyl, C₁-C₆-alkoxy, C₁-C₆-haloalkoxy, C₁-C₆-alkylsulfonyl, C₁-C₆-haloalkylsulfonyl, and phenyl which may carry one or more substituents selected from the group consisting of halogen, C₁-C₆-alkyl, C₁-C₆-haloalkyl, C₁-C₆-alkoxy and C₁-C₆-haloalkoxy;
each R¹¹ is independently selected from the group consisting of OH, C₁-C₆-alkoxy, C₁-C₆-haloalkoxy, NR¹⁵R¹⁶, C(O)OR¹³, C(O)NR¹⁵R¹⁶, phenyl which may carry one or more substituents R¹⁸, and a 3-, 4-, 5-, 6-, 7- or 8-membered saturated heterocyclic ring containing 1 or 2 heteroatoms or heteroatom-containing groups selected from the group consisting of O, N, S, NO, SO and SO₂ as ring members, where the heterocyclic ring may carry one or more substituents R¹⁸;
each R¹³ is independently C₁-C₆-alkyl or C₁-C₆-haloalkyl;
R¹⁴ is phenyl which may carry one or more substituents R¹⁸;
R¹⁵ and R¹⁶, independently of each other and independently of each occurrence, are selected from the group consisting of hydrogen, C₁-C₆-alkyl which may carry one or more substituents R¹⁹, C₁-C₆-haloalkyl, C₃-C₆-cycloalkyl, C₃-C₆-halocycloalkyl, C₁-C₆-alkylcarbonyl and C₁-C₆-haloalkylcarbonyl;
or R¹⁵ and R¹⁶, together with the nitrogen atom they are bound to, form a saturated, partially unsaturated or maximally unsaturated 3-, 4-, 5- or 6-membered heterocyclic ring, where the heterocyclic ring may additionally contain 1 or 2 further heteroatoms or heteroatom-containing groups selected from the group consisting of O, N, S, NO, SO and SO₂ as ring members, where the heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo;
each R¹⁷ is independently C₁-C₆-alkyl or C₁-C₆-haloalkyl;
each R¹⁸ is independently selected from the group consisting of halogen, CN, nitro, OH, SH, C₁-C₆-alkyl which may carry one or more substituents NR²³R²⁴; C₁-C₆-haloalkyl, C₃-C₈-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, carboxyl, C₁-C₆-alkylcarbonyl and C₁-C₆-haloalkylcarbonyl;
or two radicals R¹⁸ bound on adjacent ring atoms, together with the ring atoms they are bound to, may form a saturated, partially unsaturated or maximally unsaturated 5- or 6-membered carbocyclic or heterocyclic ring, where the heterocyclic ring contains 1 or 2 heteroatoms or heteroatom-containing groups selected from the group consisting of O, N, S, NO, SO and SO₂ as ring members, where the carbocyclic or heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo;
each R¹⁹ is independently selected from the group consisting of CN, OH, C₁-C₆-alkoxy, C₁-C₆-haloalkoxy, SH, C₁-C₆-alkylthio, C₁-C₆-haloalkylthio, C₁-C₆-alkylsulfonyl, C₁-C₆-haloalkylsulfonyl, NR²³R²⁴ and phenyl which may carry one or more substituents R¹⁸, where R¹⁸ is in particular selected from the group consisting of halogen, CN, OH, C₁-C₆-alkyl, C₁-C₆-haloalkyl, C₁-C₆-alkoxy and C₁-C₆-haloalkoxy; and
R²³ and R²⁴, independently of each other and independently of each occurrence, are selected from the group consisting of hydrogen, C₁-C₆-alkyl, C₁-C₆-haloalkyl, C₃-C₈-cycloalkyl, C₃-C₈-halocycloalkyl, C₁-C₆-alkylcarbonyl, C₁-C₆-haloalkylcarbonyl, C₁-C₆-alkoxycarbonyl, C₁-C₆-haloalkoxycarbonyl, C₁-C₆-alkylsulfonyl, C₁-C₆-haloalkylsulfonyl, aryl and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where aryl or the heterocyclic ring may carry one or more substituents selected from the group consisting of halogen, CN, OH, C₁-C₆-alkyl, C₁-C₆-haloalkyl, C₁-C₆-alkoxy and C₁-C₆-haloalkoxy.

In a more preferred embodiment, in compounds I
X¹ is CR¹ or N; in particular CR¹;
X² is CR²;
X³ is CR³;
X⁴ is CR⁴ or N; in particular CR¹;
with the proviso that at most one of X¹ and X⁴ is N;
L¹ is CH₂, CH(CH₃) or CH₂CH₂;
L² is a bond or CH₂CH₂NH;
A is a 5-membered heteroaromatic ring containing one nitrogen atom and one further heteroatom selected from the group consisting of O, N and S as ring members, where the heterocyclic ring may carry one or more substituents R¹⁰;
R¹ and R², independently of each other, are selected from the group consisting of hydrogen, halogen, CN, C₁-C₄-alkyl, C₁-C₄-alkoxy and C₁-C₄-haloalkoxy;
R³ and R⁴, independently of each other, are selected from the group consisting of hydrogen, F, C₁-C₄-alkyl and C₁-C₄-alkoxy (where R⁴ is in particular hydrogen, F or methyl, more particularly hydrogen or methyl and specifically hydrogen);
or R¹ and R², or R² and R³ form together a bridging group —CH₂CH₂CH₂—, —CH₂CH₂CH₂CH₂—, or —O—CH₂—O—;
R⁵ is hydrogen;
R⁶ is selected from the group consisting of hydrogen, C₂-C₄-alkenyl, and phenyl which may carry one or more substituents R¹⁸;
each R¹⁰ is independently selected from the group consisting of CN, C₁-C₄-alkyl which may carry one or more substituents R¹¹, C₁-C₄-haloalkyl, C(O)R¹⁷, C(O)OR¹³, C(O)NR¹⁵R¹⁶, phenyl which may carry one or more substituents R¹⁸, and a 5- or 6-membered heteroaromatic ring containing one heteroatom selected from the group consisting of O, N and S as ring members, where the heteroaromatic ring may carry one or more substituents R¹⁸;
or two radicals R^{1′} bound on adjacent ring atoms form together a bridging group —CH═CH—CH═CH—, —CH₂CH₂CH₂— or —CH₂CH₂CH₂CH₂—, where one of the hydrogen atoms in the bridging group may be substituted by a radical selected from the group consisting of methyl and methoxy;
each R¹¹ is independently selected from the group consisting of OH, C₁-C₄-alkoxy, C₁-C₄-haloalkoxy, NR¹⁵R¹⁶ and C(O)NR¹⁵R¹⁶;
each R¹³ is independently C₁-C₄-alkyl;
R¹⁵ and R¹⁶, independently of each other and independently of each occurrence, are selected from the group consisting of hydrogen, C₁-C₄-alkyl and C₁-C₄-alkylcarbonyl;
R¹⁷ is C₁-C₄-alkyl;
each R¹⁸ is independently selected from the group consisting of halogen, C₁-C₆-alkyl which may carry one substituent NR²³R²⁴; C₃-C₈-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, and C₁-C₆-alkylcarbonyl;
or two radicals R¹⁸ bound on adjacent ring atoms, together with the ring atoms they are bound to, may form a saturated 5- or 6-membered heterocyclic ring containing 1 or 2 heteroatoms or heteroatom-containing groups selected from the group consisting of O, N, S, NO, SO and SO₂ as ring members, where the heterocyclic ring may be substituted by one or more radicals selected from the group consisting of halogen, C₁-C₄-alkyl, C₁-C₄-haloalkyl, C₁-C₄-alkoxy, C₁-C₄-haloalkoxy and oxo; and
R²³ and R²⁴, independently of each other and independently of each occurrence, are selected from the group consisting of hydrogen and C₁-C₄-alkylcarbonyl.

In an even more preferred embodiment, in compounds I
X¹ is CR¹ or N;
X² is CR²;
X³ is CR³;
X⁴ is CR⁴ or N;
with the proviso that at most one of X¹ and X⁴ is N;
L¹ is CH₂, CH(CH₃) or CH₂CH₂;
L² is a bond or CH₂CH₂NH;
A is a 5-membered heteroaromatic ring containing one nitrogen atom and one further heteroatom selected from the group consisting of N, O and S as ring members, where the heterocyclic ring may carry one or more substituents R¹⁰;
R¹ and R², independently of each other, are selected from the group consisting of hydrogen, halogen, CN, C₁-C₄-alkyl, C₁-C₄-alkoxy and C₁-C₄-haloalkoxy;
R³ is selected from the group consisting of hydrogen, C₁-C₄-alkyl and C₁-C₄-alkoxy;
or R² and R³ form together a bridging group —CH₂CH₂CH₂— or —O—CH₂—O—;
R⁴ is hydrogen;
R⁵ is hydrogen;
R⁶ is selected from the group consisting of hydrogen, C₃-C₄-alkenyl, and phenyl which carries a substituent R¹⁸;
each R¹⁰ is independently selected from the group consisting of CN, C₁-C₄-alkyl which may carry one or more substituents R¹¹, C₁-C₄-haloalkyl, C(O)R¹⁷, C(O)OR¹³, C(O)NR¹⁵R¹⁶, phenyl which may carry one or two substituents R¹⁸, and a 5- or 6-membered heteroaromatic ring containing one heteroatom selected from the group consisting of O, N and S as ring members, where the heteroaromatic ring may carry one or more substituents R¹⁸;
or two radicals R¹⁰ bound on adjacent ring atoms form together a bridging group —CH═CH—CH═CH— or —CH₂CH₂CH₂—, where one of the hydrogen atoms in the bridging group may be substituted by a radical selected from the group consisting of methyl and methoxy;
each R¹¹ is independently selected from the group consisting of OH, C₁-C₄-alkoxy, C₁-C₄-haloalkoxy, NR¹⁵R¹⁶ and C(O)NR¹⁵R¹⁶;
each R¹³ is independently C₁-C₄-alkyl;
R¹⁵ and R¹⁶, independently of each other, are selected from the group consisting of hydrogen, C₁-C₄-alkyl and C₁-C₄-alkylcarbonyl;
R¹⁷ is C₁-C₄-alkyl;
each R¹⁸ is independently selected from the group consisting of halogen, C₁-C₆-alkyl which may carry one substituent NR²³R²⁴; C₃-C₆-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, and C₁-C₆-alkylcarbonyl;
or two radicals R¹⁸ bound on adjacent ring atoms, together with the ring atoms they are bound to, may form a saturated 5- or 6-membered heterocyclic ring containing one nitrogen ring atom or one or two oxygen atoms as ring members, where the heterocyclic ring may be substituted by an oxo group; and
R²³ and R²⁴, independently of each other and independently of each occurrence, are selected from the group consisting of hydrogen and C₁-C₄-alkylcarbonyl.

In particular, in compounds I
X¹ is CR¹;
X² is CR²;
X³ is CR³;
X⁴ is CR⁴;
L¹ is CH₂, CH(CH₃) or CH₂CH₂;
L² is a bond or CH₂CH₂NH;
A is a 5-membered heteroaromatic ring containing one nitrogen atom and one further heteroatom selected from the group consisting of N and S as ring members, where the heterocyclic ring may carry one or more substituents R¹⁰;
R¹ and R², independently of each other, are selected from the group consisting of hydrogen, halogen, CN, C₁-C₄-alkyl, C₁-C₄-alkoxy and C₁-C₄-haloalkoxy;
R³ is selected from the group consisting of hydrogen, C₁-C₄-alkyl and C₁-C₄-alkoxy;
or R² and R³ form together a bridging group —CH₂CH₂CH₂— or —O—CH₂—O—;
R⁴ is hydrogen;
R⁵ is hydrogen;
R⁶ is selected from the group consisting of hydrogen, C₃-C₄-alkenyl, and phenyl which carries a substituent R¹⁸;
each R¹⁰ is independently selected from the group consisting of CN, C₁-C₄-alkyl which may carry one or more substituents R¹¹, C₁-C₄-haloalkyl, C(O)R¹⁷, C(O)OR¹³, phenyl which may carry one or two substituents R¹⁸, and a 5- or 6-membered heteroaromatic ring containing one heteroatom selected from the group consisting of O, N and S as ring members, where the heteroaromatic ring may carry one or more substituents R¹⁸;
or two radicals R^{1′} bound on adjacent ring atoms form together a bridging group —CH═CH—CH═CH— or —CH₂CH₂CH₂—, where one of the hydrogen atoms in the bridging group may be substituted by a radical selected from the group consisting of methyl and methoxy;
each R¹¹ is independently selected from the group consisting of OH, C₁-C₄-alkoxy, C₁-C₄-haloalkoxy and NR¹⁵R¹⁶;
each R¹³ is independently C₁-C₄-alkyl;
R¹¹ and R¹⁶, independently of each other, are selected from the group consisting of hydrogen, C₁-C₄-alkyl and C₁-C₄-alkylcarbonyl;
R¹⁷ is C₁-C₄-alkyl;
each R¹⁸ is independently selected from the group consisting of halogen, C₁-C₆-alkyl which may carry one substituent NR²³R²⁴; C₃-C₆-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, and C₁-C₆-alkylcarbonyl;
or two radicals R¹⁸ bound on adjacent ring atoms, together with the ring atoms they are bound to, may form a saturated 5- or 6-membered heterocyclic ring containing one nitrogen ring atom or one or two oxygen atoms as ring members, where the heterocyclic ring may be substituted by an oxo group; and
R²³ and R²⁴, independently of each other and independently of each occurrence, are selected from the group consisting of hydrogen and C₁-C₄-alkylcarbonyl.

In particular, the compound of formula I is a compound of formula I.a

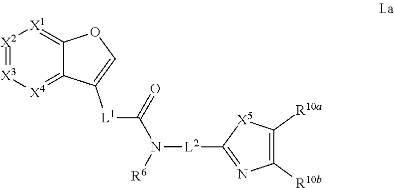

wherein
X¹ is CR¹, X² is CR², X³ is CR³ and X⁴ is CR⁴; or
X¹ is N, X² is CR², X³ is CR³ and X⁴ is CR⁴; or
X¹ is CR¹, X² is N, X³ is CR³ and X⁴ is CR⁴; or
X¹ is CR¹, X² is CR², X³ is N and X⁴ is CR⁴; or
X¹ is CR¹, X² is CR², X³ is CR³ and X⁴ is N;
L¹ is CH₂, CH(CH₃) or CH₂CH₂;
L² is a bond or CH₂CH₂NH;
X⁵ is O, S or NR^{x};
R¹¹ is hydrogen or C₁-C₄-alkyl;
R¹ and R², independently of each other, are selected from the group consisting of hydrogen, F, Cl, CN, C₁-C₄-alkyl, C₁-C₂-alkoxy and C₁-C₄-haloalkoxy;
R³ is selected from the group consisting of hydrogen, C₁-C₄-alkyl and C₁-C₄-alkoxy;
or R² and R³ form together a bridging group —CH₂CH₂CH₂— or —O—CH₂—O—;
R⁴ is hydrogen;
R⁶ is selected from the group consisting of hydrogen, C₃-C₄-alkenyl, and phenyl which carries a substituent R¹⁸;
R^{10a} is selected from the group consisting of hydrogen, CN, C₁-C₄-alkyl which may carry one substituent R¹¹; C₁-C₄-haloalkyl, C(O)OR¹³ and C(O)NR¹⁵R¹⁶;
R^{10b} is selected from the group consisting of hydrogen, C₁-C₄-alkyl which may carry one substituent R¹¹; C(O)R¹⁷, C(O)OR¹³, C(O)NR¹⁵R¹⁶, phenyl which may carry one or two substituents R¹⁸, and a 5- or 6-membered heteroaromatic ring containing one heteroatom selected from the group consisting of O, N and S as ring members, where the heteroaromatic ring may carry one or more substituents R¹⁸;
or R^{10a} and R^{10b} bound on adjacent ring atoms form together a bridging group —CH═CH—CH═CH— or —CH₂CH₂CH₂—, where one of the hydrogen atoms in the bridging group may be substituted by a radical selected from the group consisting of methyl and methoxy;
each R¹¹ is independently selected from the group consisting of OH, C₁-C₄-alkoxy and C(O)NR¹⁵R¹⁶;
each R¹³ is independently C₁-C₄-alkyl;
R¹⁵ and R¹⁶, independently of each other, are selected from the group consisting of hydrogen and C₁-C₄-alkyl;
R¹⁷ is C₁-C₄-alkyl;
each R¹⁸ is independently selected from the group consisting of halogen, C₁-C₆-alkyl which may carry one substituent NR²³R²⁴; C₃-C₆-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, and C₁-C₆-alkylcarbonyl;
or two radicals R¹⁸ bound on adjacent ring atoms, together with the ring atoms they are bound to, may form a saturated 5- or 6-membered heterocyclic ring containing one nitrogen ring atom or one or two oxygen atoms as ring members, where the heterocyclic ring may be substituted by an oxo group; and
R²³ and R²⁴, independently of each other and independently of each occurrence, are selected from the group consisting of hydrogen and C₁-C₄-alkylcarbonyl.

Preferably, in compounds I.a
X¹ is CR¹, X² is CR², X³ is CR³ and X⁴ is CR⁴; or
X¹ is N, X² is CR², X³ is CR³ and X⁴ is CR⁴; or
X¹ is CR¹, X² is CR², X³ is CR³ and X⁴ is N;
L¹ is CH₂, CH(CH₃) or CH₂CH₂;
L² is a bond or CH₂CH₂NH;
X⁵ is S or NR^{x};
R¹¹ is hydrogen or C₁-C₄-alkyl;
R¹ and R², independently of each other, are selected from the group consisting of hydrogen, F, Cl, CN, C₁-C₄-alkyl, C₁-C₂-alkoxy and C₁-C₄-haloalkoxy;
R³ is selected from the group consisting of hydrogen, C₁-C₄-alkyl and C₁-C₄-alkoxy;
or R² and R³ form together a bridging group —CH₂CH₂CH₂— or —O—CH₂—O—;
R⁴ is hydrogen;
R⁶ is selected from the group consisting of hydrogen, C₃-C₄-alkenyl, and phenyl which carries a substituent R¹⁸;
R^{10a} is selected from the group consisting of hydrogen, CN, C₁-C₄-alkyl which may carry one substituent R¹¹; C₁-C₄-haloalkyl, and C(O)OR¹³;
R^{10b} is selected from the group consisting of hydrogen, C₁-C₄-alkyl, phenyl which may carry one or two substituents R¹⁸, and a 5- or 6-membered heteroaromatic ring containing one heteroatom selected from the group consisting of O, N and S as ring members, where the heteroaromatic ring may carry one or more substituents R¹⁸;
or R^{10a} and R^{10b} bound on adjacent ring atoms form together a bridging group —CH═CH—CH═CH— or —CH₂CH₂CH₂—, where one of the hydrogen atoms in the bridging group may be substituted by a radical selected from the group consisting of methyl and methoxy;
each R¹¹ is independently selected from the group consisting of OH and C₁-C₄-alkoxy;
each R¹³ is independently C₁-C₄-alkyl;
each R¹⁸ is independently selected from the group consisting of halogen, C₁-C₆-alkyl which may carry one substituent NR²³R²⁴; C₃-C₆-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, and C₁-C₆-alkylcarbonyl;
or two radicals R¹⁸ bound on adjacent ring atoms, together with the ring atoms they are bound to, may form a saturated 5- or 6-membered heterocyclic ring containing one or two oxygen atoms as ring members; and
R²³ and R²⁴, independently of each other and independently of each occurrence, are selected from the group consisting of hydrogen and C₁-C₄-alkylcarbonyl.

More preferably, in compounds I.a
X¹ is CR¹, X² is CR², X³ is CR³ and X⁴ is CR⁴; or
X¹ is N, X² is CR², X³ is CR³ and X⁴ is CR⁴;
L¹ is CH₂, CH(CH₃) or CH₂CH₂;
L² is a bond or CH₂CH₂NH;
X⁵ is S or NR^{x};
R¹¹ is hydrogen or C₁-C₄-alkyl;
R¹ and R², independently of each other, are selected from the group consisting of hydrogen, F, C₁, CN, C₁-C₄-alkyl, C₁-C₂-alkoxy and C₁-C₄-haloalkoxy;
R³ is selected from the group consisting of hydrogen, C₁-C₄-alkyl and C₁-C₄-alkoxy;
or R² and R³ form together a bridging group —CH₂CH₂CH₂— or —O—CH₂—O—;
R⁴ is hydrogen;
R⁶ is selected from the group consisting of hydrogen, C₃-C₄-alkenyl, and phenyl which carries a substituent R¹⁸;
R^{10a} is selected from the group consisting of hydrogen, CN, C₁-C₄-alkyl which may carry one substituent R¹¹; C₁-C₄-haloalkyl, and C(O)OR¹³;
R^{10b} is selected from the group consisting of hydrogen, C₁-C₄-alkyl, phenyl which may carry one or two substituents R¹⁸, and a 5- or 6-membered heteroaromatic ring containing one heteroatom selected from the group consisting of O, N and S as ring members, where the heteroaromatic ring may carry one or more substituents R¹⁸;
or R^{10a} and R^{10b} bound on adjacent ring atoms form together a bridging group —CH═CH—CH═CH— or —CH₂CH₂CH₂—, where one of the hydrogen atoms in the bridging group may be substituted by a radical selected from the group consisting of methyl and methoxy;
each R¹¹ is independently selected from the group consisting of OH and C₁-C₄-alkoxy;
each R¹³ is independently C₁-C₄-alkyl;
each R¹⁸ is independently selected from the group consisting of halogen, C₁-C₆-alkyl which may carry one substituent NR²³R²⁴; C₃-C₆-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, and C₁-C₆-alkylcarbonyl;
or two radicals R¹⁸ bound on adjacent ring atoms, together with the ring atoms they are bound to, may form a saturated 5- or 6-membered heterocyclic ring containing one or two oxygen atoms as ring members; and
R²³ and R²⁴, independently of each other and independently of each occurrence, are selected from the group consisting of hydrogen and C₁-C₄-alkylcarbonyl.

Even more preferably, in compounds I.a
X¹ is CR¹, X² is CR², X³ is CR³ and X⁴ is CR⁴; or
X¹ is N, X² is CR², X³ is CR³ and X⁴ is CR⁴;
L¹ is CH₂, CH(CH₃) or CH₂CH₂;
L² is a bond or CH₂CH₂NH;
X⁵ is S;
R¹ and R², independently of each other, are selected from the group consisting of hydrogen, F, Cl, C₁-C₄-alkyl and C₁-C₂-alkoxy;
R³ is selected from the group consisting of hydrogen and C₁-C₄-alkyl;
or R² and R³ form together a bridging group —CH₂CH₂CH₂—;
R⁴ is hydrogen;
R⁶ is selected from the group consisting of hydrogen, C₃-C₄-alkenyl, and phenyl which carries a substituent R¹⁸; and is in particular hydrogen;
R^{10a} is selected from the group consisting of hydrogen, CN, C₁-C₄-alkyl which may carry one substituent R¹¹; and C₁-C₄-haloalkyl;
R^{10b} is selected from the group consisting of hydrogen and phenyl which may carry one or two substituents R¹⁸;
or R^{10a} and R^{10b} bound on adjacent ring atoms form together a bridging group —CH═CH—CH═CH—;
each R¹¹ is independently selected from the group consisting of OH and C₁-C₄-alkoxy;
each R¹⁸ is independently selected from the group consisting of halogen, C₃-C₆-cycloalkyl, C₁-C₄-alkoxy, C₁-C₄-haloalkoxy, C₁-C₄-alkylthio, C₁-C₄-haloalkylthio, C₁-C₄-alkylsulfonyl, C₁-C₄-haloalkylsulfonyl, and C₁-C₄-alkylcarbonyl;
or two radicals R¹⁸ bound on adjacent ring atoms, together with the ring atoms they are bound to, may form a saturated 5- or 6-membered heterocyclic ring containing one or two oxygen atoms as ring members.

In one embodiment of compounds I.a R⁶ is hydrogen. In another embodiment of compounds I.a R⁶ is C₃-C₄-alkenyl or phenyl which carries a substituent R¹⁸; where R¹⁸ has one of the above general or, in particular, one of the above preferred meanings. Preferably, in this context R¹⁸ is selected from the group consisting of halogen, C₃-C₆-cycloalkyl, C₁-C₄-alkoxy, C₁-C₄-haloalkoxy, C₁-C₄-alkylthio, C₁-C₄-haloalkylthio, C₁-C₄-alkylsulfonyl, C₁-C₄-haloalkylsulfonyl, and C₁-C₄-alkylcarbonyl; and is specifically C₁-C₄-alkylthio, C₁-C₄-haloalkylthio, or C₁-C₄-alkylcarbonyl.

Specifically, the compound of formula I.a is a compound of formula I.a.1

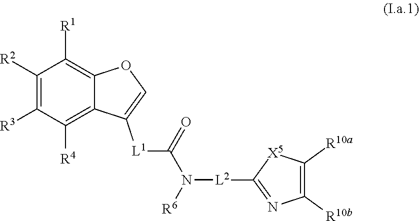

wherein R¹, R², R³, R⁴, R⁶, L¹ and L² have one of the above general or, in particular, one of the above preferred meanings; R^{10a} and R^{10b} are independently of each other hydrogen or have one of the general or, in particular, one of the preferred meanings given above for R¹⁰; and X⁵ is S or NR^{x}; where R^{x} is hydrogen or C₁-C₄-alkyl.

Preferably, however, in compounds I.a.1
L¹ is CH₂, CH(CH₃) or CH₂CH₂;
L² is a bond or CH₂CH₂NH;
X⁵ is S or NR^{x};
R^{x} is hydrogen or C₁-C₄-alkyl;
R¹ and R², independently of each other, are selected from the group consisting of hydrogen, F, Cl, CN, C₁-C₄-alkyl, C₁-C₂-alkoxy and C₁-C₄-haloalkoxy;
R³ is selected from the group consisting of hydrogen, C₁-C₄-alkyl and C₁-C₄-alkoxy;
or R² and R³ form together a bridging group —CH₂CH₂CH₂— or —O—CH₂—O—;
R⁴ is hydrogen;
R⁶ is selected from the group consisting of hydrogen, C₃-C₄-alkenyl, and phenyl which carries a substituent R¹⁸;
R^{10a} is selected from the group consisting of hydrogen, CN, C₁-C₄-alkyl which may carry one substituent R¹¹; C₁-C₄-haloalkyl, and C(O)OR¹³;
R^{10b} is selected from the group consisting of hydrogen, C₁-C₄-alkyl, phenyl which may carry one or two substituents R¹⁸, and a 5- or 6-membered heteroaromatic ring containing one heteroatom selected from the group consisting of O, N and S as ring members, where the heteroaromatic ring may carry one or more substituents R¹⁸;
or R^{10a} and R^{10b} bound on adjacent ring atoms form together a bridging group —CH═CH—CH═CH— or —CH₂CH₂CH₂—, where one of the hydrogen atoms in the bridging group may be substituted by a radical selected from the group consisting of methyl and methoxy;
each R¹¹ is independently selected from the group consisting of OH and C₁-C₄-alkoxy;
each R¹³ is independently C₁-C₄-alkyl;
each R¹⁸ is independently selected from the group consisting of halogen, C₁-C₆-alkyl which may carry one substituent NR²³R²⁴; C₃-C₆-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, and C₁-C₆-alkylcarbonyl;
or two radicals R¹⁸ bound on adjacent ring atoms, together with the ring atoms they are bound to, may form a saturated 5- or 6-membered heterocyclic ring containing one or two oxygen atoms as ring members; and
R²³ and R²⁴, independently of each other and independently of each occurrence, are selected from the group consisting of hydrogen and C₁-C₄-alkylcarbonyl.

More preferably, in compounds I.a.1
L¹ is CH₂, CH(CH₃) or CH₂CH₂;
L² is a bond or CH₂CH₂NH;
X⁵ is S;
R¹ and R², independently of each other, are selected from the group consisting of hydrogen, F, Cl, C₁-C₄-alkyl and C₁-C₂-alkoxy;
R³ is selected from the group consisting of hydrogen and C₁-C₄-alkyl;
or R² and R³ form together a bridging group —CH₂CH₂CH₂—;
R⁴ is hydrogen;
R⁶ is selected from the group consisting of hydrogen, C₃-C₄-alkenyl, and phenyl which carries a substituent R¹⁸; and is in particular hydrogen;
R^{10a} is selected from the group consisting of hydrogen, CN, C₁-C₄-alkyl which may carry one substituent R¹¹; and C₁-C₄-haloalkyl;
R^{10b} is selected from the group consisting of hydrogen and phenyl which may carry one or two substituents R¹⁸;
or R^{10a} and R^{10b} bound on adjacent ring atoms form together a bridging group —CH═CH—CH═CH—;
each R¹¹ is independently selected from the group consisting of OH and C₁-C₄-alkoxy;
each R¹⁸ is independently selected from the group consisting of halogen, C₃-C₆-cycloalkyl, C₁-C₄-alkoxy, C₁-C₄-haloalkoxy, C₁-C₄-alkylthio, C₁-C₄-haloalkylthio, C₁-C₄-alkylsulfonyl, C₁-C₄-haloalkylsulfonyl, and C₁-C₄-alkylcarbonyl;
or two radicals R¹⁸ bound on adjacent ring atoms, together with the ring atoms they are bound to, may form a saturated 5- or 6-membered heterocyclic ring containing one or two oxygen atoms as ring members.

In one embodiment of compounds I.a.1 R⁶ is hydrogen. In another embodiment of compounds I.a.1 R⁶ is C₃-C₄-alkenyl or phenyl which carries a substituent R¹⁸; where R¹⁸ has one of the above general or, in particular, one of the above preferred meanings. Preferably, in this context R¹⁸ is selected from the group consisting of halogen, C₃-C₆-cycloalkyl, C₁-C₄-alkoxy, C₁-C₄-haloalkoxy, C₁-C₄-alkylthio, C₁-C₄-haloalkylthio, C₁-C₄-alkylsulfonyl, C₁-C₄-haloalkylsulfonyl, and C₁-C₄-alkylcarbonyl; and is specifically C₁-C₄-alkylthio, C₁-C₄-haloalkylthio, or C₁-C₄-alkylcarbonyl.

In a specific embodiment, the invention relates to a pharmaceutical composition comprising compounds I selected from the compounds of the examples, either in form of free bases or of any pharmaceutically acceptable salt thereof or a stereoisomer, the racemate or any mixture of stereoisomers thereof or a tautomer or a tautomeric mixture or an N-oxide thereof.

Some compounds of formula I are novel, and thus the invention relates to these novel compounds. These are compounds of formula I.a.1

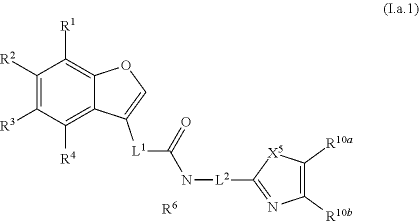

a tautomer, or a pharmaceutically acceptable salt thereof, wherein the variables for a single compound have the meanings given in one line of the following table:

| No. | R¹ | R² | R³ | R⁴ | L¹ | R⁶ | L² | X⁵ | R^{10a} | R^{10b} |
|---|---|---|---|---|---|---|---|---|---|---|
| 1 | H | H | CH₃ | H | CH₂ | H | bond | S | CF₃ | H |
| 2 | H | H | H | H | CH₂ | H | bond | S | CF₃ | H |
| 3 | CH₃ | H | H | H | CH₂ | H | bond | S | CH₃ | H |
| 4 | H | Cl | H | H | CH₂ | H | bond | S | CH₃ | H |
| 5 | H | —CH₂CH₂CH₂— | | H | CH₂ | H | bond | O | CH₃ | H |
| 6 | CH₃ | CH₃ | H | H | CH₂ | H | bond | NH | CH₃ | H |
| 7 | H | —CH₂CH₂CH₂— | | H | CH₂ | H | bond | NH | H | CH₃ |
| 8 | H | —O—CH₂—O— | | H | CH₂ | H | bond | S | CH₃ | H |
| 9 | H | Cl | H | H | CH₂ | H | bond | NH | CF₃ | H |
| 10 | F | F | H | H | CH₂ | H | bond | S | CF₃ | H |
| 11 | H | H | H | H | Et | H | bond | S | CF₃ | H |
| 13 | H | Cl | H | H | CH₂ | H | bond | S | CN | H |
| 14 | Cl | Cl | H | H | CH₂ | H | bond | NH | CF₃ | H |
| 15 | Cl | H | H | H | CH₂ | H | bond | S | CF₃ | H |
| 16 | CH₃ | CH₃ | H | H | CH₂ | H | bond | NH | CF₃ | H |
| 17 | H | Cl | H | H | CH₂ | H | bond | S | CH₂OCH₃ | H |
| 18 | Cl | CH₃ | H | H | CH₂ | H | bond | NH | CH₃ | H |
| 19 | Cl | CH₃ | H | H | CH₂ | H | bond | NH | CF₃ | H |
| 20 | Cl | CH₃ | H | H | CH₂ | H | bond | S | CN | H |
| 21 | H | Cl | H | H | CH₂ | H | bond | S | CF₃ | H |
| 22 | CH₃ | CH₃ | H | H | Et | H | bond | S | CF₃ | H |
| 23 | CH₃ | CH₃ | H | H | CH₂ | H | bond | S | CN | H |
| 24 | H | —CH₂CH₂CH₂— | | H | CH₂ | H | bond | S | CF₃ | H |
| 25 | H | —CH₂CH₂CH₂— | | H | CH₂ | H | bond | S | CH₂OCH₃ | H |
| 26 | H | Cl | H | H | CH(CH₃) | H | bond | S | CF₃ | H |
| 27 | CH₃ | H | H | H | CH₂ | H | bond | S | CF₃ | H |
| 28 | Cl | Cl | H | H | CH₂ | H | bond | S | CN | H |
| 29 | Cl | Cl | H | H | CH₂ | H | bond | S | CH₃ | H |
| 30 | CH₃ | OCH₃ | H | H | CH₂ | H | bond | S | CH₃ | H |
| 31 | H | —CH₂CH₂CH₂— | | H | CH₂ | H | EtNH | S | CH₂Oh | H |
| 32 | CH₃ | CH₃ | H | H | CH₂ | 4-SCHF₂—C₆H₄ | bond | S | —CH═CH—CH═CH— | |
| 33 | Cl | CH₃ | H | H | CH₂ | H | bond | S | CF₃ | H |
| 34 | H | —CH₂CH₂CH₂— | | H | CH₂ | H | bond | S | C₂H₅ | H |
| 35 | Cl | Cl | H | H | CH₂ | H | bond | S | CF₃ | H |
| 36 | OCH₃ | CH₃ | H | H | CH₂ | H | bond | S | CH₃ | H |
| 37 | CH₃ | CH₃ | H | H | CH₂ | H | bond | S | CH₂OCH₃ | H |
| 38 | CH₃ | CH₃ | H | H | CH₂ | H | bond | S | CH₃ | H |
| 39 | Cl | CH₃ | H | H | CH₂ | H | bond | S | CH₂OCH₃ | H |
| 40 | Cl | CH₃ | H | H | CH₂ | H | bond | S | CH₃ | H |
| 41 | CH₃ | OCH₃ | H | H | CH₂ | H | bond | S | CF₃ | H |
| 42 | CH₃ | Cl | H | H | CH₂ | H | bond | S | CH₃ | H |
| 43 | Cl | Cl | H | H | CH₂ | H | bond | S | CH₂OCH₃ | H |
| 44 | CH₃ | Cl | H | H | CH₂ | H | bond | S | CF₃ | H |
| 45 | CH₃ | CH₃ | H | H | CH₂ | H | bond | S | CH(CH₃)₂ | H |
| 46 | CH₃ | CH₃ | H | H | CH₂ | H | bond | S | CH₃ | H |
| 47 | OCH₃ | CH₃ | H | H | CH₂ | H | bond | S | CF₃ | H |
| 48 | CH₃ | CH₃ | H | H | CH₂ | H | bond | S | C₂H₅ | H |
| 49 | CH₃ | CH₃ | H | H | CH₂ | H | bond | S | CF₃ | H |
| 50 | H | —CH₂CH₂CH₂— | | H | CH₂ | H | bond | S | H | 5-am-furan-2-yl |
| 51 | H | CH₃ | CH₃ | H | CH₂ | H | bond | S | H | 4-am-phenyl |
| 52 | CH₃ | CH₃ | H | H | CH₂ | H | bond | S | CF₃ | C(O)—NH—CH₃ |

| | | | | | | | | | |
|---|---|---|---|---|---|---|---|---|---|
| 53 | H | —CH₂CH₂CH₂— | H | CH₂ | H | bond | S | H | 5-ac-am-furan-2-yl |

where the abbreviations have following meanings:
Et is CH₂CH₂;
EtNH is CH₂CH₂NH;
4-SCHF₂—C₆H₄ is 4-difluoromethylsulfanylphenyl;
4-OMe-CH₄ is 4-methoxyphenyl;
5-am-furan-2-yl is 5-aminomethylfuran-2-yl;
5-ac-am-furan-2-yl is 5-(acetylaminomethyl)-furan-2-yl; and
4-amphenyl is 4-aminomethylphenyl;
C(O)—NH—CH₃ is N-methyl-carboxamide;
or of formula I.b

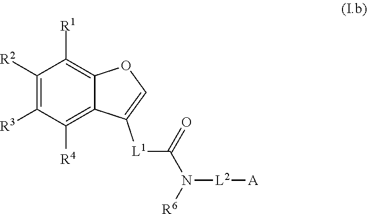

a tautomer, or a pharmaceutically acceptable salt thereof, wherein the variables for a single compound have the meanings given in one line of the following table:

| | | | | | | | | |
|---|---|---|---|---|---|---|---|---|
| No. | R¹ | R² | R³ | R⁴ | L¹ | R⁶ | L² | A |

| | | | | | | | |
|---|---|---|---|---|---|---|---|
| 54 | H | —CH₂CH₂—CH₂— | H | CH₂ | H | bond | 1-methyl- |
| | | | | | | | pyrazol-3-yl |

or of formula I.c

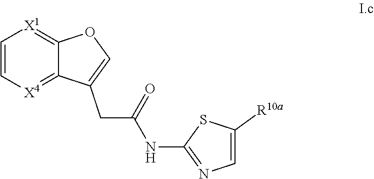

a tautomer, or a pharmaceutically acceptable salts thereof, wherein the variables for a single compound have the meanings given in one line of the following table:

| No. | X¹ | X⁴ | R^{10a} |
|---|---|---|---|
| 55 | N | C | CH₃ |
| 56 | C | N | CH₃ |
| 57 | N | C | CF₃ |

Some compounds I are commercially available. Those which are novel can be prepared by using routine techniques familiar to a skilled person. In particular, the compounds of the formula I can be prepared according to the following schemes, wherein the variables, if not stated otherwise, are as defined above.

The compounds I according to the invention can be prepared by analogy to methods known from the literature and as described in the examples of the present application. In particular, the compounds of the formula I can be prepared according to the following schemes, wherein the variables, if not stated otherwise, are as defined above. An important approach to the compounds according to the invention is the reaction of a benzofuran carboxylic acid compound 2 with an amine compound 3 to yield the compounds I according to the present invention, as depicted in scheme 1.

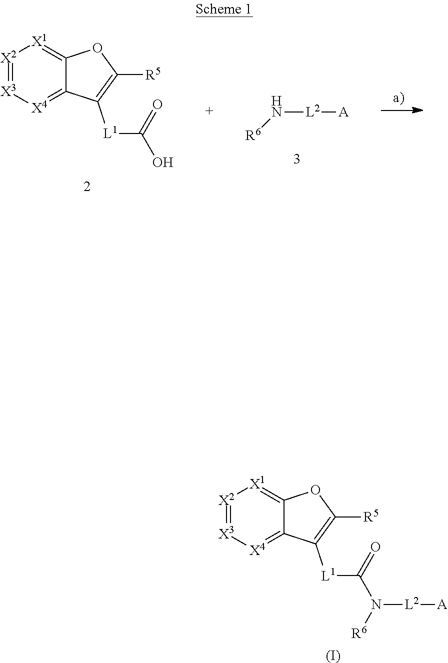

In step a) of scheme 1, the carboxylic acid of the formula 2 reacts with the amine group of compound 3 under conditions suitable for amide bond formation. The skilled person is familiar with the reaction conditions which are required for this type of reaction. Typically, the amide bond formation is carried out in the presence of a coupling reagent. Suitable coupling reagents (activators) are well known and are for instance selected from the group consisting of 1,1′-carbonyldiimidazole (CDI), carbodiimides, such as EDCI (1-ethyl-3-(3-dimethylaminopropyl)carbodiimide; also abbreviated as EDC), DCC (dicyclohexylcarbodiimide) and DIC (diisopropylcarbodiimide), benzotriazole derivatives, such as HOBt (1-hydroxybenzotriazole), HATU (O-(7-azabenzotriazol-1-yl)-N,N,N′,N′-tetramethyluronium hexafluorophosphate), HBTU ((O-benzotriazol-1-yl)-N,N,N′,N′-tetramethyluronium hexafluorophosphate) and HCTU (1H-benzotriazolium-1-[bis(dimethylamino)methylene]-5-chloro tetrafluoroborate), phosphonium-derived activators, such as BOP ((benzotriazol-1-yloxy)-tris(dimethylamino)phosphonium hexafluorophosphate), Py-BOP ((benzotriazol-1-yloxy)-tripyrrolidinphosphonium hexafluorophosphate) and Py-BrOP (bromotripyrrolidinphosphonium hexafluorophosphate), and others, such as COMU ((1-cyano-2-ethoxy-2-oxoethylidenaminooxy)dimethylamino-morpholino-carbenium-hexafluorophosphat). The above activators can also be used in combination with each other. Generally, the activator is used in at least equimolar amounts, with respect to that reactant not used in excess. The benzotriazole and phosphonium coupling reagents are generally used in a basic medium.

Alternatively, the carboxylic acid 2 can be first converted into a so-called active ester, which is obtained in a formal sense by the reaction of the carboxylic acid with an active ester-forming alcohol, such as p-nitrophenol, N-hydroxybenzotriazole (HOBt), N-hydroxysuccinimide or OPfp (pentafluorophenol). The active ester is then reacted with the amine 3 either in the presence or the absence of a coupling reagent.

Furthermore, the OH group of the carboxylic acid 2 can also first be converted into a suitable leaving group (LG), such as Cl, Br, I or a sulfonate, such as tosylate, mesylate, triflate or nonaflate, using reaction procedures that are known to the skilled person. The thus activated carboxylic acid 2 is then reacted with the amine 3. In this variant, the amide bond formation is generally carried out in the presence of a base to neutralize the acid formed during the reaction. Typically, organic bases are used for this purpose. Suitable organic bases are for example tertiary amines, e.g. trimethylamine, triethylamine, tripropylamine, ethyldiisopropylamine and the like, or basic N-heterocycles, such as morpholine, pyridine, lutidine, DABCO, DBU or DBN.

In some particular cases it may be necessary to use appropriate protecting groups in order to avoid side reactions with other reactive groups, which may be present in compound 2 and/or compound 3 and may compete in or disturb the reaction. Just by way of example, if one or more of R¹, R², R³, R⁴, R⁷ and R⁸ is or contains a group C(O)OH, NH₂ or OH and this group has a similar or even stronger reactivity than the desired reaction sites, it is expedient to protect these groups before the above-described amidation reaction is carried out. In these cases, additional deprotecting steps may be necessary to remove these protecting groups after amide bond formation. Suitable protecting groups and the methods for protecting and deprotecting different substituents using such suitable protecting groups are well known to those skilled in the art; examples of which may be found in T. Greene and P. Wuts, Protective Groups in Organic Synthesis (3^{rd} ed.), John Wiley & Sons, NY (1999).

In case that L² is not a bond, the compounds I (termed hereinafter compounds I′ can alternatively be prepared by the reaction of a benzofuran carboxylic acid compound 2 with a precursor amine 4 to yield the intermediate amide 5, as depicted in scheme 2, which is then further reacted with a compound 6 to yield the compounds I′, as depicted in scheme 3.

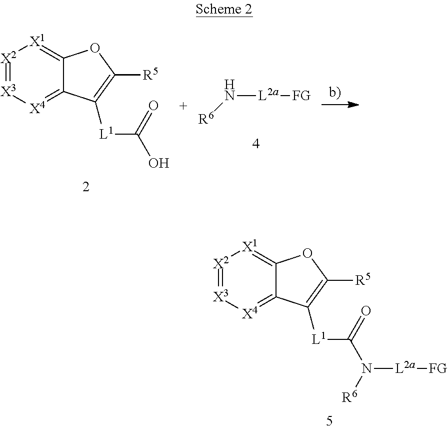

Typically, the amide bond formation in step b) of scheme 2 can be performed as described for step a). The intermediate amide compound 5 is then further reacted with a compound 6 to yield the compounds I′, as depicted in scheme 3.

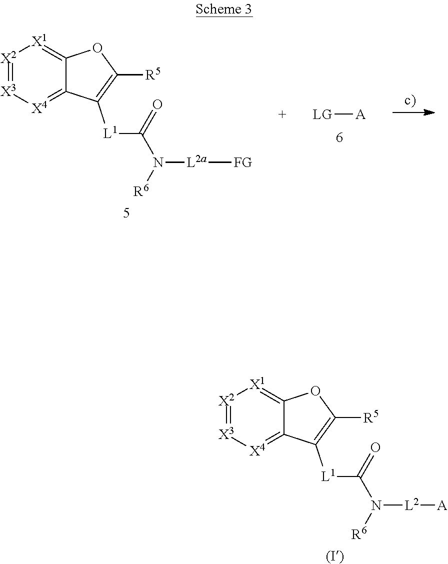

In scheme 3, L² in compound I′ has the aforementioned meanings, but for a bond. L^{2a} is selected from C₁-C₆-alkylene which may carry one or more substituents R⁷ and C₃-C₈-cycloalkylene which may carry one or more substituents R⁸. R⁷ and R⁸ are as defined above, under the provision that R⁷ and R⁸ are not selected from functional groups and/or do not comprise any functional groups that might interfere or disturb the reactions in steps b) and c), such as, in particular, halogen, haloalkyl, hydroxyl, CN, SF₅, primary or secondary amines, carboxylic acid or carboxylic acid esters. The choice of suitable R⁷ and R⁸ lies within the routine practice of the skilled person.

The precursor amine 4 carries a suitable functional group (FG) to allow the attachment of further building blocks, in particular to allow the attachment of the cyclic moiety A. For example, FG is selected from —OH, —SH and —N(R¹⁵)H, which may be protected with suitable protective groups, if required, to allow a selective amidation reaction in step b). Before step c), the protective group is of course removed. R¹⁵ is as defined above, under the provision that R¹⁵ is not selected from functional groups and/or does not comprise any functional groups that might interfere or disturb the reactions in steps b) and c). If in the reaction of compounds 4 (and downstream of compounds 5) FG is selected from —OH, —SH and —N(R¹⁵)H, this results in compounds I′ in which L² is C₁-C₆-alkylene-O, C₁-C₆-alkylene-S, C₁-C₆-alkylene-NR¹⁵, where the alkylene moiety in the three last-mentioned radicals may carry one or more substituents R⁷; C₃-C₈-cycloalkylene-O, C₃-C₈-cycloalkylene-S or C₃-C₈-cycloalkylene-NR¹⁵, where the cycloalkylene moiety in the three last-mentioned radicals may carry one or more substituents R⁸.

The compounds 6 comprise the group LG, which, in case that FG is —OH, —SH and —N(R¹⁵)H, is suitably a leaving group, such as those as defined above.

If FG is selected from —OH, —SH and —N(R¹⁵)H, the reaction in step c) is performed under conditions suitable for nucleophilic substitution reactions. Typically, this reaction is performed in the presence of a base. The skilled person is familiar with the reaction conditions which are required for this type of nucleophilic substitution reaction. In case that A is an aromatic or heteroaromatic ring, the exchange of substituents by nucleophilic reagents is however distinctly more difficult than in case of A being a saturated or partially unsaturated ring. It is essential that the leaving group LG in A forms an anion of low energy or an uncharged molecule or can be removed by an energetically advantageous process. Therefore, the leaving group LG is mostly a halide, a sulfonic acid group or a diazonium group in non-activated (hetero)aromatic compounds. Nucleophilic aromatic substitution on carboaromatic rings (phenyl, naphthyl etc.) is eased if the aromatic ring is activated, i.e. contains substituents with a −M effect in ortho and/or para position to the carbon atom carrying the leaving group. Substituents with a −M effect and which fall under the present substituents R¹⁰ are for example the nitro, cyano, formyl, or acetyl group. In this case, also less favoured leaving groups can react; e.g. even hydrogen atoms can be replaced (i.e. LG in 6 can in this case even be H). Electron-poor heteroaromatic rings, like the 6-membered heteroaromatic compounds (pyridine, pyridazine, pyrimidine, pyrazine, the triazines) or quinoline, also undergo readily nucleophilic substitution, even with poor leaving groups, like the hydrogen atom. In case the group FG in compound 5 is selected from —OH or —N(R¹⁵)H and A is an aromatic or heteroaromatic ring, the reaction in step c) can also be performed under conditions of transition metal-catalyzed C—O or C—N coupling reactions. Transition metal-catalyst C—O or C—N coupling reactions are well known to the skilled person. An important example is the Buchwald-Hartwig reaction. The Buchwald-Hartwig reaction is a transition metal-catalyzed, mostly a Pd catalyzed, C—N or C—O bond formation between an aryl or heteroaryl halogenide or sulfonate and a primary or secondary amine (for C—N bond formation) or an alcohol (for C—O bond formation), generally in the presence of a base. The skilled person is familiar with identifying suitable reaction conditions for the Buchwald-Hartwig reaction.

For preparing compounds I′, in which L² is C₁-C₆-alkylene-O, C₁-C₆-alkylene-S, C₁-C₆-alkylene-NR¹⁵, where the alkylene moiety in the three last-mentioned radicals may carry one or more substituents R⁷; C₃-C₈-cycloalkylene-O, C₃-C₈-cycloalkylene-S or C₃-C₈-cycloalkylene-NR¹⁵, where the cycloalkylene moiety in the three last-mentioned radicals may carry one or more substituents R⁸, it is alternatively possible to use compounds 5 in which FG is a leaving group, such as a halide atom (especially Cl, Br or I or a sulfonate (such as tosylate, mesylate, triflate or nonaflate), and compounds 6 in which LG is a group —OH, —SH or —N(R¹⁵)H. This reaction can be carried out under typical conditions for nucleophilic substitution.

Compounds of the formula 3 can either be purchased or can be readily synthesized using standard methods of heterocyclic chemistry, as for example described in Joule, J. A. and Mills, K. Heterocyclic Chemistry, 5th Edition. 2010, Wiley, Weinheim. ISBN: 978-1-4051-3300-5 and knowledge of functional group interconversion, as for example described in Larock, R. C. Comprehensive Organic Transformations, A Guide to Functional Group Preparations. 2017, Wiley, Weinheim. ISBN: 978-0-470-92795-3.

The compounds of formula 3 can also be synthesized, e.g. following the procedure as depicted in scheme 4.

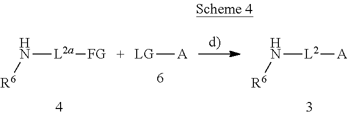

In scheme 4, L² in compound 3 has the aforementioned meanings, but for a bond. L^{2a}, FG and LG have the aforementioned meanings.

Typically, the reaction in step d) of scheme 4 is performed under conditions suitable for nucleophilic substitution reactions, as described for step c).

For obtaining compounds 3 in which L² is a bond, a compound N(R⁶)H₂ can be used instead of compound 4 for the reaction with 6 in scheme 4.

Compounds of the formula 2 can either be purchased or can be synthesized following different procedures that are described in the prior art. The selection of the appropriate synthetic route depends on the substitution pattern of the compounds of formula 2 and lies within the routine expertise of the skilled person.

For example, compounds of the general formula 2a, which represent a subset of the compounds of formula 2, can be prepared by the reaction of a hydroxy(hetero)aromatic compound 7 with a chloroacetoacetate compound 8 to the intermediate chloride 9, which is subsequently rearranged to yield the compounds 2a, as depicted in scheme 5.

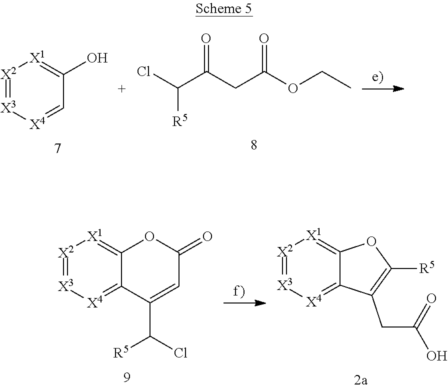

Step e) in scheme 5 is typically performed in the presence of an acid. Suitable acids are for example mineral acids, such as sulfuric acid, hydrochloric acid, hydrobromic acid or nitric acid, alkylsulfonic acids, such as methanesulfonic acid, ethanesulfonic acid or camphersulphonic acid, haloalkylsulfonic acids, such as trifluoromethanesulfonic acid, arylsulfonic acids, such as benzenesulfonic acid or para-toluenesulfonic acid, and carboxylic acids, such as trichloroacetic acid or trifluoroacetic acid. Generally, the intermediate chloride 9, obtained after the addition of the chloroacetoacetate compound 8 to the hydroxy(hetero)aromatic compound 7, is subjected to workup and/or purification procedures before it is subjected to the rearrangement reaction in step f).

Step f) in scheme 5 is typically performed in the presence of a base. Suitable bases can be inorganic or organic. Examples for suitable inorganic bases are alkali metal carbonates, e.g. Li₂CO₃, Na₂CO₃, K₂CO₃ or Cs₂CO₃, alkali metal hydroxides, e.g. LiOH, NaOH or KOH, or phosphates, e.g. Li₃PO₄, Na₃PO₄, K₃PO₄ or Cs₃PO₄. Examples for suitable organic bases are alkoxylates, e.g. sodium or potassium methanolate, ethanolate, propanolate, isopropanolate, butanolate or tert-butanolate, especially sterically hindered alkoxylates, such as sodium or potassium tert-butanolate.

Alternatively, compounds 2a can be prepared from precursors 10, which are first halogenated to the halogen compounds 11, then reacted with a cyanide to the nitrile compounds 12 and subsequently hydrolyzed to yield the compounds of formula 2a, as depicted in scheme 6.

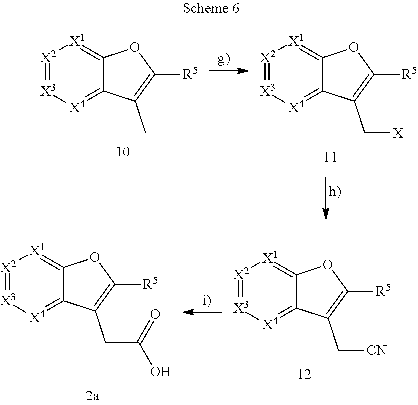

In scheme 6, X is selected from halogen, such as chlorine or bromine.

Step g) in scheme 6 is generally performed in the presence of a halogenation reagent. Suitable halogenation reagents are for example N-chlorosuccinimide (NCS), N-chlorophthalimid, trichloroisocyanuric acid, N-bromosuccinimide (NBS), N-bromophthalimid, dibromoisocyanuric acid, N-iodosuccinimide (NIS) or 1,3-Diodo-5,5′-dimethylhidantoin (DIH).

Step h) in scheme 6 is generally performed in the presence of a cyanide salt under conditions of a nucleophilic substitution reaction. Suitable cyanide salts are, for example, alkali metal cyanides and tetraalkylammonium cyanides. Examples include sodium cyanide, potassium cyanide, lithium cyanide, rubidium cyanide, tetraethylammonium cyanide and tetrabutylammonium cyanide.

Step i) in scheme 6 is performed under conditions suitable for hydrolyzing nitrile groups, i.e. in the presence of water under acidic or basic conditions. Suitable acids are for example mineral acids as mentioned above. Suitable bases are, for example, inorganic bases as mentioned above.

Furthermore, compounds 2a can also be prepared by reacting compounds 13 with a phosphonate compound 14 to give compounds 15, which are subsequently hydrolysed to yield the compounds of the general formula 2a, as depicted in scheme 7.

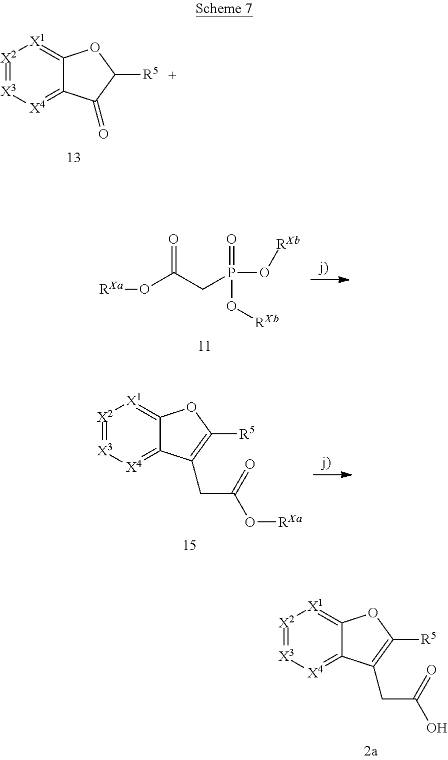

In scheme 7, R^{Xa} is selected from C₁-C₄-alkyl and R^{Xb} is selected from C₁-C₄-alkyl and C₁-C₃-haloalkyl.

The reaction of the compounds 13 with the phosphonate 14 in step j) of scheme 7 is typically performed under Horner-Wadsworth-Emmons reaction conditions, which involves the addition of a base to deprotonate the phosphonate 14.

The ester compound 15 obtained in step 7 is then subjected to ester hydrolysis conditions, i.e. step k) of scheme 7. The conditions for ester hydrolysis are well known to the skilled person. Ester hydrolysis is typically performed in the presence of water under basic conditions. Suitable bases are as defined above. The compounds 13 in turn can be prepared by reacting a phenol compound 7 with an α-halo-carboxylic acid compound 15 to the carboxylic acid intermediate 16, which is converted to the acid chloride 17 and subsequently subjected to an intramolecular Friedel-Crafts acylation to yield the benzofuranone compound 13, as depicted in scheme 8.

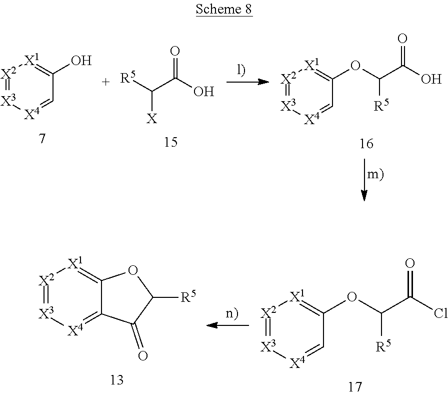

In scheme 8, X represents a halogen, such as chlorine or bromine.

Typically, step I) in scheme 8 is performed in the presence of a base and under reaction conditions suitable for nucleophilic substitution reactions.

For the conversion of the carboxylic acid intermediate 16 to the acid chloride 17 in step m), common chlorination agents are used that are well known to the skilled person. Suitable chlorination agents are for example SOCl₂, POCl₃, phosgene or triphosgene.

Step n) in scheme 8 is typically performed under reaction conditions suitable for Friedel-Crafts acylation reactions, which typically involves the addition of catalytic amounts of a Lewis acid, such as AlCl₃ or FeCl₃. Conditions for Friedel-Crafts acylation are well known to the skilled person.

Variations of the above described methods for the preparation of compounds 2a can be used for the preparation of compounds 2b,

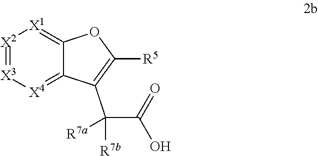

wherein R^{7a} and R^{7b} are independently of each other selected from hydrogen, C₁-C₆-alkyl, C₃-C₈-cycloalkyl and aryl, with the provision that at least one of the radicals R^{7a} or R^{7b} is not hydrogen.

The compounds 2b represent a subset of compounds of the formula 2.

Further methods for the synthesis of the compounds 2a and 2b, where at least one of the residues X¹, X², X³, X⁴ is a nitrogen atom, can be found in Shiotani, S. et al. Journal of Heterocyclic Chemistry (1995), 32(1) 129-39; Morita, H. et al. Journal of Heterocyclic Chemistry (1986), 23(5) 1465-9; Morita, H. et al. Journal of Heterocyclic Chemistry (1986), 23(2) 549-52; Shiotani, S. et al. Journal of Heterocyclic Chemistry (1986), 23(3) 665-8; and Cho, S. Y. et al., Heterocycles (1996), 43(8), 1641-1652.

Compounds of the general formula 2 in which L¹ is longer than one carbon atom can be generated by homologation of shorter intermediates. There are many methods for homologation know to the skilled person. Suitable methods are for example describes in Li, J. J. (Ed.) Name Reactions for Homologation, 2 Part Set. 2009, Wiley Weinheim, ISBN: 978-0-470-46721-3. For example, as can be seen from scheme 9, the compounds of formula 2a can be esterified under standard conditions to give the ester compounds 18, which are reduced to the alcohols of formula 19. Conversion of the alcohol to a leaving group (LG′), yields activated compounds 20, which can be alkylated with a cyanide to give nitrile compounds of formula 21. Hydrolysis then provides compounds of formula 2c. The compounds 2c are a subset of compounds of formula 2.

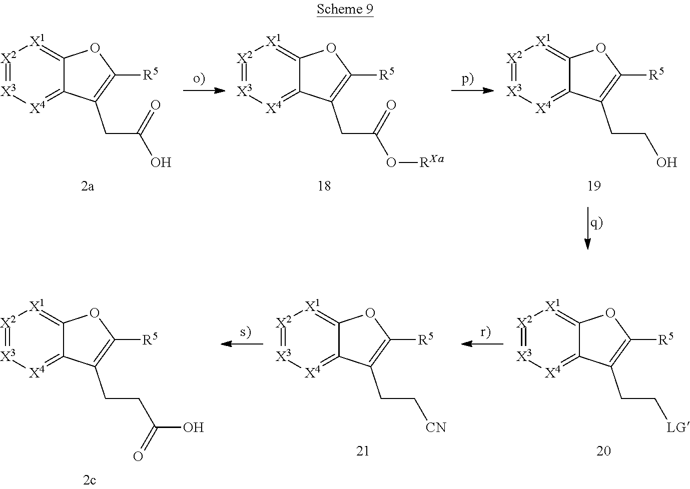

In scheme 9, R^{Xa} has the aforementioned meanings. LG′ is typically selected from sulfonates, such as tosylate, mesylate, triflate or nonaflate.

In step o) of scheme 9 standard esterification procedures can be applied that are well known to the skilled person.

The reduction in step p) of scheme 9 is typically performed in the presence of a reducing agent that is suitable for reducing carboxylic acid esters to the corresponding alcohols, such as LiAIH₄.

The conversion of the alcohol group into the leaving group (LG′) in step q) of scheme 9 is typically performed using reaction procedures that are well known to the skilled person.

Steps r) and s) of scheme 9 are performed following known standard procedures, as described above.

The same methodology can be applied using compounds 2b as starting compounds, which results in compounds 2d, as can be depicted from scheme 10.

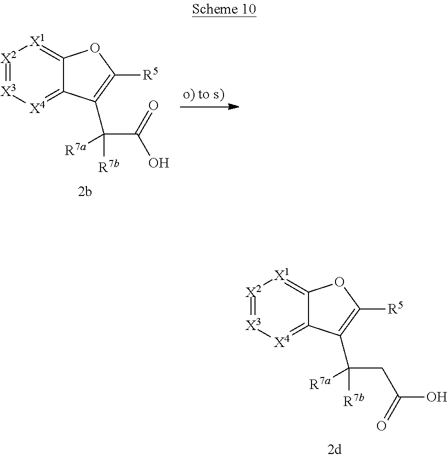

In scheme 10, R^{7a} and R^{7b} have the aforementioned meanings.

The synthesis of particular compounds 10a that can be used as building blocks for the preparation of compounds 2a, where one of the residues X¹, X² or X³ is a nitrogen atom and X⁴ is CR⁴, can be found in Cho, S. Y. et al., Heterocycles (1996), 43(8), 1641-1652. Cho, S. Y. et al. describe a palladium-catalyzed cyclization of iodopyridinyl allyl ethers 24 to generate 3-alkylfuropyridines 10a. The synthesis of particular compounds 10a following the procedure described in Cho, S. Y. et al. is illustrated in scheme 11.

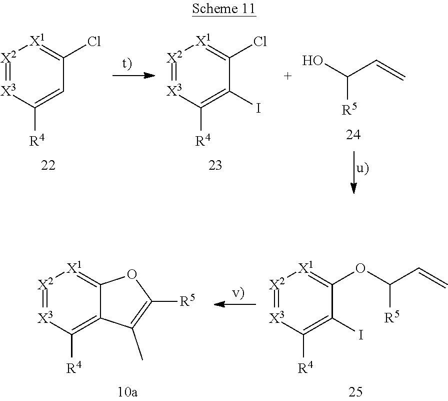

Readily accessible chloropyridines 22 are ortho-iodinated to give compounds 23. Substitution of the chloro residue with variously substituted allyl alcohol derivatives 24 gives compounds of the general formula 25. Finally palladium-catalyzed ring closure gives 3-alkylfuropyridines 10a. Other metal-catalyzed routes to benzofurans and aza-benzofurans, using, for example, alkyne building blocks are also known in the literature.

Another synthesis of particular compounds 10b that can be used as building blocks for the preparation of compounds 2a, can be found in Morita H. et al., Journal of Heterocyclic Chemistry, (1986), 23(2) 549-52. The synthesis is illustrated in scheme 12.

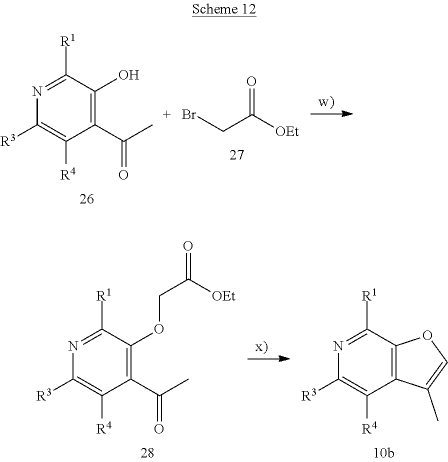

The ketone compounds 26 are alkylated to the corresponding compounds 28, using e.g. ethyl 2-bromoacetate 27. Compounds 28 are then subsequently cyclized to give compounds of the formula 10b.

The synthesis of particular azabenzofuranone compounds 13a can be found in in Morita H. et al., Journal of Heterocyclic Chemistry, (1986), 23(2) 549-52. The synthesis is illustrated in scheme 13.

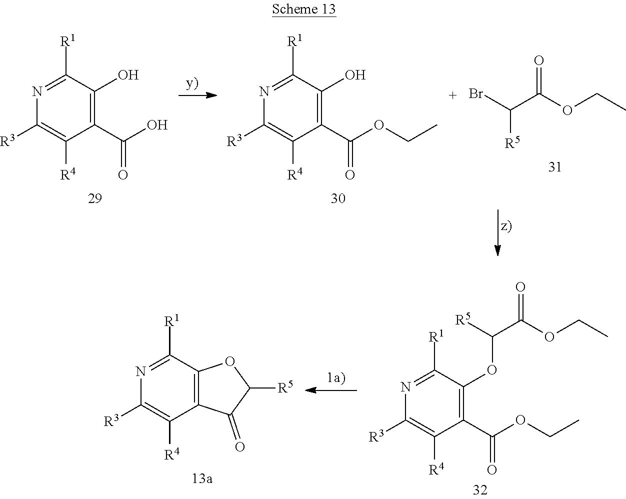

The 3-hydroxyisonicotinic acid compounds 29 are esterified to the corresponding ester compounds 30, which are alkylated to the compounds 32 using α-bromo acetic acid derivatives of formula 31. Compounds 32 are then cyclized to the azabenzofuranone compounds 13a.

Another synthesis of particular compounds 10c and/or 13b that can be used as building blocks for the preparation of compounds 2a can be found in Morita H. et al., Journal of Heterocyclic Chemistry, (1986), 23(2) 1495-9. The synthesis is illustrated in scheme 14.

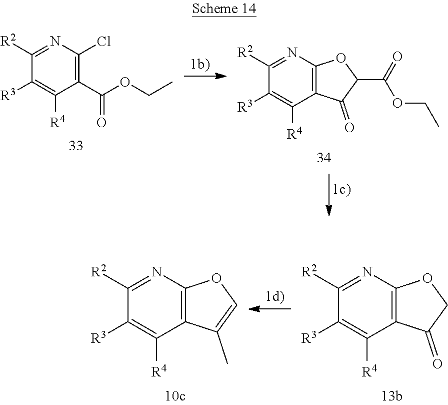

The readily available starting compound 33 is reacted with sodium 2-ethoxy-2-oxo-ethanolate to the azabenzofuranone intermediate 34, which is treated with a strong base, e.g. KOH, to give the azabenzofuranone compounds 13b. These azabenzofuranone compounds 13b can, if desired, be further converted to the azabenzofuran compounds 10c using standard reaction procedures.

Another route for the synthesis of compounds 2a, where at least one of the residues X¹, X², X³, X⁴ is a nitrogen atom, can be found in Shiotani, S. et al. Journal of Heterocyclic Chemistry (1995), 32(1) 129-39. The synthesis, which uses a variation on the Horner-Wadsworth-Emmons reaction, is illustrated in scheme 15.

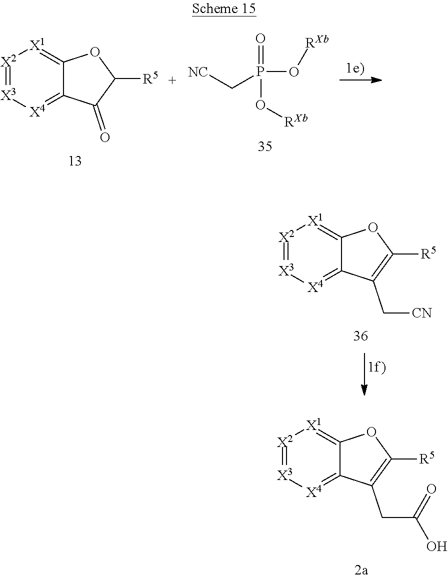

In scheme 15, R^{Xb} has the aforementioned meanings.

The furanones 13 are reacted with a diethyl cyanomethylphosphonates 35 to give nitrile compounds of formula 36, which are subsequently hydrolyzed to the benzofuran compounds 2a.

Furthermore, Shiotani, S. et al. describe the alkylation of the methylene linker of compounds 2a, where at least one of the residues X¹, X², X³, X⁴ is a nitrogen atom, to provide compounds of formula 2e, as depicted in scheme 16.

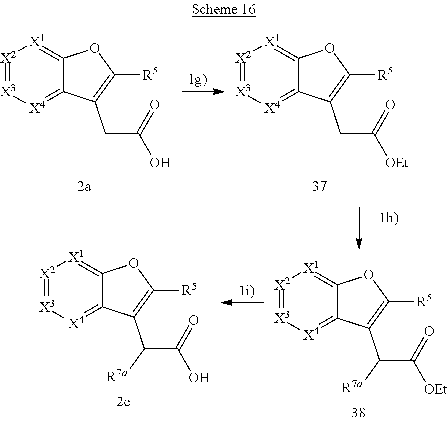

In scheme 16, R^{7a} has the aforementioned meanings.

The compounds 2a are esterified to compounds 37, which are then alkylated to the compounds 38 by using a strong base, e.g. lithiumdiisopropylamide (LDA), to deprotonate the hydrogen atom of the methylene linker followed by the addition of an alkyl-halide, such as methyl iodide, a cycloalkyl halide or an aryl halide. Saponification of compounds 38 yields 2e.

Furthermore, the synthesis of compounds 2f can be prepared following another procedure described by Shiotani at al., comprising the bromination of the precursors 39 at the C₃ carbon atom to give the bromo compounds 40, which can be subsequently converted to the nitrile compounds 41. Hydrolysis of the nitrile group yields compounds of formula 2f. The synthesis is illustrated in scheme 17.

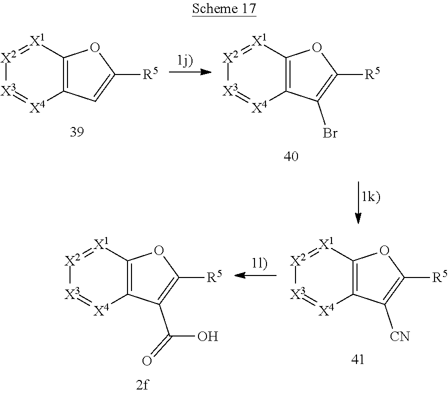

This procedure can also be used to synthesize compounds to assemble building blocks suitable for making compounds of formula I, in which L¹ is longer than one carbon atom (see for example Shiotani, S. et al., Journal of Heterocyclic Chemistry (1995), 32(1) 129-39.). For example, as depicted in scheme 18, compounds of formulae 44, 45 and 46 can be obtained from 3-halo-benzofurans or 3-halo-aza-benzofurans 43, by transition-metal catalyzed reactions using suitable alkenes or alkynes to generate a new carbon-carbon bond at the 3-position. Triflate or nonaflate leaving groups are also suitable in place of halogens.

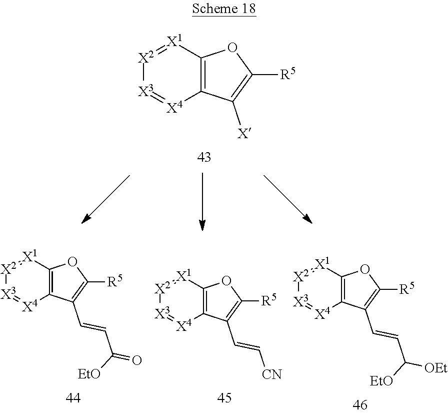

In scheme 18, X′ is selected from halogen, such as chloride or bromide, and sulfonates, such as tosylate, mesylate, triflate or nonaflate.

Further standard chemical transformation of the introduced functional groups of 44, 45 and 46 provide further compounds of formula 2, which can be used as building blocks for the synthesis of compounds I.

If not indicated otherwise, the above-described reactions are usually performed in an organic solvent, including aprotic organic solvent, e.g. substituted amides, lactams and ureas; such as dimethylformamide, dimethylacetamide, N-methylpyrrolidone, tetramethyl urea, cyclic ethers; such as dioxane, tetrahydrofuran, halogenated hydrocarbons; such as dichloromethane, and mixtures thereof as well as mixtures thereof with C₁-C₆-alkanols and/or water.

The reactions described above will be usually performed at temperatures between room temperature and the boiling temperature of the solvent employed, depending on the reactivity of the used compounds.

The reaction mixtures are worked up in a conventional way, e.g. by mixing with water, separating the phases and, where appropriate, purifying the crude products by chromatography. If the intermediates and final products are obtained as solids, the purification can also take place by recrystallization or digestion.

Routine experimentations, including appropriate manipulation of the reaction conditions, reagents and sequence of the synthetic route, protection of any chemical functionality that may not be compatible with the reaction conditions, and deprotection at a suitable point in the reaction sequence of the preparation methods are within routine techniques.

Synthesis of the compounds of the invention may be accomplished by methods analogous to those described in the synthetic schemes described hereinabove and in specific examples.

Starting materials, if not commercially available, may be prepared by procedures selected from standard organic chemical techniques, techniques that are analogous to the synthesis of known, structurally similar compounds, or techniques that are analogous to the above described schemes or the procedures described in the synthetic examples section.

The acid addition salts of compounds I are prepared in a customary manner by mixing the free base with a corresponding acid, where appropriate in solution in an organic solvent, for example acetonitrile, a lower alcohol, such as methanol, ethanol or propanol, an ether, such as diethyl ether, methyl tert-butyl ether or diisopropyl ether, a ketone, such as acetone or methyl ethyl ketone, an ester, such as ethyl acetate, mixtures thereof as well as mixtures thereof with water.

The pharmaceutical composition of the invention can contain one or more than one compound of formula I. It comprises moreover at least one pharmaceutically acceptable carrier and/or auxiliary substance.

Examples of suitable carriers and auxiliary substances for the various different forms of pharmaceutical compositions are well known and may be found in the “Handbook of Pharmaceutical Excipients”, 2nd Edition, (1994), Edited by A Wade and P J Weller or in Remington's Pharmaceutical Sciences, Mack Publishing Co. (A. R Gennaro edit. 1985).

For preparing pharmaceutical compositions from the compounds I, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material.

In powders, the carrier is a finely divided solid, which is in a mixture with the finely divided active component. In tablets, the active component is mixed with the carrier having the necessary binding properties in suitable proportions and compacted in the shape and size desired.

The powders and tablets preferably contain from 1% to 80%, more preferably from 5% to 60% of the active compound or active compounds. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. Liquid forms are particularly preferred for topical applications to the eye. For parenteral injection, liquid preparations can be formulated in solution as in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Examples for carriers are thus magnesium carbonate, magnesium stearate, talc, sugar, lactose, pectin, dextrin, starch, gelatin, tragacanth, methylcellulose, sodium carboxymethylcellulose, a low melting wax, cocoa butter, water, water/propylene glycol solutions, or water/polyethylene glycol solutions, and the like.

Examples for auxiliary substances for the present pharmaceutical composition are glidants; wetting agents; emulsifying and suspending agents; dispersants, preservatives; antioxidants; antiirritants; chelating agents; coating auxiliaries; emulsion stabilizers; film formers; gel formers; odor masking agents; flavors, taste corrigents; artificial and natural sweeteners, resin; hydrocolloids; solvents; solubilizers; neutralizing agents; buffers, diffusion accelerators; colorants, pigments; quaternary ammonium compounds; refatting and overfatting agents; raw materials for ointments, creams or oils; silicone derivatives; spreading auxiliaries; stabilizers; sterilants; binders, fillers, disintegrants, coatings; propellants; drying agents; opacifiers; thickeners; waxes; plasticizers, white mineral oils and the like.

The present invention further relates to the compound I as defined above, a stereoisomer, tautomer or pharmaceutically acceptable salt thereof for use as a medicament.

The invention moreover relates to the compound I as defined above, a stereoisomer, tautomer or pharmaceutically acceptable salt thereof for use in the treatment of conditions, disorders or diseases selected from the group consisting of inflammatory diseases, hyperproliferative diseases or disorders, a hypoxia related pathology and a disease characterized by pathophysiological hypervascularization. The invention also relates to the use of compounds I, a stereoisomer, tautomer or pharmaceutically acceptable salt thereof for preparing a medicament for the treatment of conditions, disorders or diseases selected from the group consisting of inflammatory diseases, hyperproliferative diseases or disorders, a hypoxia related pathology and a disease characterized by pathophysiological hypervascularization. The invention also relates to a method for treating conditions, disorders or diseases selected from the group consisting of inflammatory diseases, hyperproliferative diseases or disorders, a hypoxia related pathology and a disease characterized by pathophysiological hypervascularization, which method comprises administering to a patient in need thereof at least one compound I, a stereoisomer, tautomer or pharmaceutically acceptable salt thereof.

In preferred embodiments, the inflammatory disease is selected form the group consisting of atherosclerosis, rheumatoid arthritis, asthma, inflammatory bowel disease, psoriasis, in particular psoriasis vulgaris, psoriasis capitis, psoriasis guttata, psoriasis inversa; neurodermatitis; ichthyosis; alopecia areata; alopecia totalis; alopecia subtotalis; alopecia universalis; alopecia diffusa; atopic dermatitis; lupus erythematodes of the skin; dermatomyositis of the skin; atopic eczema; morphea; scleroderma; alopecia areata Ophiasis type; androgenic alopecia; allergic dermatitis; irritative contact dermatitis; contact dermatitis; pemphigus vulgaris; pemphigus foliaceus; pemphigus vegetans; scarring mucous membrane pemphigoid; bullous pemphigoid; mucous membrane pemphigoid; dermatitis; dermatitis herpetiformis Duhring; urticaria; necrobiosis lipoidica; erythema nodosum; prurigo simplex; prurigo nodularis; prurigo acuta; linear IgA dermatosis; polymorphic light dermatosis; erythema solaris; exanthema of the skin; drug exanthema; purpura chronica progressiva; dihydrotic eczema; eczema; fixed drug exanthema; photoallergic skin reaction; and perioral dermatitis.

In preferred embodiments, the hyperproliferative disease is selected from the group consisting of a tumor or cancer disease, precancerosis, dysplasia, histiocytosis, a vascular proliferative disease and a virus-induced proliferative disease. In particular, the hyperproliferative disease is a tumor or cancer disease selected from the group consisting of diffuse large B-cell lymphoma (DLBCL), T-cell lymphomas or leukemias, e.g., cutaneous T-cell lymphoma (CTCL), noncutaneous peripheral T-cell lymphoma, lymphoma associated with human T-cell lymphotrophic virus (HTLV), adult T-cell leukemia/lymphoma (ATLL), as well as acute lymphocytic leukemia, acute nonlymphocytic leukemia, acute myeloid leukemia, chronic lymphocytic leukemia, chronic myelogenous leukemia, Hodgkin's disease, non-Hodgkin's lymphoma, myeloma, multiple myeloma, mesothelioma, childhood solid tumors, glioma, bone cancer and soft-tissue sarcomas, common solid tumors of adults such as head and neck cancers (e.g., oral, laryngeal and esophageal), genitourinary cancers (e.g., prostate, bladder, renal (in particular malignant renal cell carcinoma (RCC)), uterine, ovarian, testicular, rectal, and colon), lung cancer (e.g., small cell carcinoma and non-small cell lung carcinoma, including squamous cell carcinoma and adenocarcinoma), breast cancer, pancreatic cancer, melanoma and other skin cancers, basal cell carcinoma, metastatic skin carcinoma, squamous cell carcinoma of both ulcerating and papillary type, stomach cancer, brain cancer, liver cancer, adrenal cancer, kidney cancer, thyroid cancer, medullary carcinoma, osteosarcoma, soft-tissue sarcoma, Ewing's sarcoma, veticulum cell sarcoma, and Kaposi's sarcoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, leiomyosarcoma, rhabdomyosarcoma, squamous cell carcinoma, adenocarcinoma, sweat gland carcinoma, sebaceous gland carcinoma, papillary carcinoma, glioblastoma, papillary adenocarcinomas, cystadenocarcinoma, bronchogenic carcinoma, seminoma, embryonal carcinoma, Wilms' tumor, small cell lung carcinoma, epithelial carcinoma, astrocytoma, medulloblastoma, craniopharyngioma, ependymoma, pinealoma, hemangioblastoma, acoustic neuroma, oligodendroglioma, meningioma, neuroblastoma, retinoblastoma, glaucoma, hemangioma, heavy chain disease and metastases.

The precancerosis are for example selected from the group consisting actinic keratosis, cutaneaous horn, actinic cheilitis, tar keratosis, arsenic keratosis, x-ray keratosis, Bowen's disease, bowenoid papulosis, lentigo maligna, lichen sclerosus, and lichen rubber mucosae; precancerosis of the digestive tract, in particular erythroplakia, leukoplakia, Barrett's esophagus, Plummer-Vinson syndrome, crural ulcer, gastropathia hypertrophica gigantea, borderline carcinoma, neoplastic intestinal polyp, rectal polyp, porcelain gallbladder; gynaecological precancerosis, in particular carcinoma ductale in situ (CDIS), cervical intraepithelial neoplasia (CIN), endometrial hyperplasia (grade III), vulvar dystrophy, vulvar intraepithelial neoplasia (VIN), hydatidiform mole; urologic precancerosis, in particular bladder papillomatosis, Queyrat's erythroplasia, testicular intraepithelial neoplasia (TIN), carcinoma in situ (CIS); precancerosis caused by chronic inflammation, in particular pyoderma, osteomyelitis, acne conglobata, lupus vulgaris, and fistula.

Dysplasia is frequently a forerunner of cancer, and is can be found in e.g. the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation. Dysplastic disorders which can be treated with the compounds of the present invention include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephaloophthalmic dysplasia, dysplasia epiphysialis heminelia, dysplasia epiphysialis multiplex, dysplasia epiphysalis punctata, epithelial dysplasia, faciodigitogenital dysplasia, familial fibrous dysplasia of jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysical dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, ophthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

A hypoxia related pathology is for example diabetic retinopathy, ischemic reperfusion injury, ischemic myocardial and limb disease, ischemic stroke, sepsis and septic shock (see, e.g. Liu F Q, et al., Exp Cell Res. 2008 Apr. 1; 314(6):1327-36).

A disease characterized by pathophysiological hyper-vascularization is for example angiogenesis in osteosarcoma (see, e.g.: Yang, Qing-cheng et al., Dier Junyi Daxue Xuebao (2008), 29(5), 504-508), macular degeneration, in particular, age-related macular degeneration and vasoproliferative retinopathy (see e.g. Kim J H, et al., J Cell Mol Med. 2008 Jan. 19).

The following examples serve to explain the present invention without limiting its scope.

### EXAMPLES

### A. Synthesis Examples

In the below examples the names of the synthesized target compounds as well as their structure are given. Any discrepancy between name and structure is unintentional; in this case the structure is decisive.

### Abbreviations

Boc for tert-butyloxycarbonyl; Boc₂O for di-tert-butyl dicarbonate; BuLi for buthyllithium; DCM for dichloromethane; DIPEA for N,N-diisopropylethylamine; DMF for dimethylformamide; DMSO for dimethylsulfoxide; EDC for 1-ethyl-3-(3-dimethylaminopropyl)carbodiimide; eq for equivalent; EtOH for ethanol, EtOAc for ethyl acetate; HOAt for 1-hydroxy-7-azabenzotriazole; i-PrOH for isopropanol; MeOH for methanol; Ms for mesityl; MTBE for methyl tertiary-butyl ether; PyBOP for benzotriazol-1-yl-oxytripyrrolidinophosphonium hexafluorophosphate; r.t. for room temperature; sat. for saturated, THF for tetrahydrofuran; TLC for thin layer chromatography.

Compounds can be characterized e.g. by melting point, ¹H-NMR, LC-MS and retention times. ¹H-NMR: The signals are characterized by chemical shift (ppm, δ [delta]) vs. tetramethylsilane, by their multiplicity and by their integral (relative number of hydrogen atoms given). The following abbreviations are used to characterize the multiplicity of the signals: m=multiplet, q=quartet, t=triplet, d=doublet and s=singlet.

### HPLC-MS Instrument Specifications:

Agilent 1100 Series LC/MSD system with DAD¥ELSD and Agilent LC¥MSD VL (G1956A), SL (G1956B) mass-spectrometer or Agilent 1200 Series LC/MSD system with DAD¥ELSD and Agilent LC¥MSD SL (G6130A), SL (G6140A) mass-spectrometer. All the LC/MS data were obtained using positive/negative mode switching.

### Acquisition Parameters:

Column: Zorbax SB-C18 1.8 μm 4.6×15 mm Rapid Resolution cartridge (PN 821975-932); Mobile phase: A—acetonitrile, 0.1% formic acid; B—water (0.1% formic acid); Flow rate: 3 mL/min; Gradient: 0 min—100% B; 0.01 min—100% B; 1.5 min—0% B; 1.8 min—0% B; 1.81 min—100% B; Injection volume: 1 μl; Ionization mode: atmospheric pressure chemical ionization (APCI); Scan range: m/z 80-1000.

### UPLC-MS Specifications

Agilent Infinity 1290 UPLC-MS System; Mass Spectrometer: Single Quadrupole, Electrospray Ionisation; Flow rate: 1 mL/min; inject volume 3 μl; runtime 3 min; Column: Acquity UPLC BEH C18; 1.7 μm; 2.1×50 mm; T=40° C.; Elution: A: Water plus 0.1% trifluoroacetic acid; B: CH₃CN plus 0.1% trifluoroacetic acid; 3 minute gradient: 0 min—5% B; 2.3 min—100% B; 2.5 min—100% B; 2.6 min—5% B; 3 min 5% B.

### HPLC Purification:

Purification was performed using HPLC (H₂O-MeOH, H₂O—CH₃CN; Agilent 1260 Infinity systems equipped with DAD and mass-detectors. Waters Sunfire C18 OBD Prep Column, 100A, 5 μm, 19 mm×100 mm with SunFire C18 Prep Guard Cartridge, 100A, 10 μm, 19 mm×10 mm) The material was dissolved in 0.7 mL DMSO. Flow: 30 mL/min. Purity of the obtained fractions was checked via the analytical LCMS. Spectra were recorded for each fraction as it was obtained straight after chromatography in the solution form. The solvent was evaporated in the flow of N₂ at 80° C. On the basis of post-chromatography LCMS analysis fractions were united. Solid fractions were dissolved in 0.5 mL MeOH/CH₃CN and transferred into a pre-weighted marked vials. Obtained solutions were again evaporated in the flow of N₂ at 80° C. After drying, products were finally characterized by LC-MS and ¹H NMR.

The procedures shown in the following general methods I, II, III and IV, respectively may be used to provide benzofuran-3-acetic acid compounds and 3-(benzofuran-3-yl)propanoic acid compounds, respectively. The acids may then be used in amination reactions with various amines to provide the compounds of formula (I) as outlined in the general methods A, B and C, respectively. In the general methods, the substituents and variables are as defined above for formula (I), if not otherwise specified.

### I. Preparation of Benzofuran-3-Acetic Acid Compounds

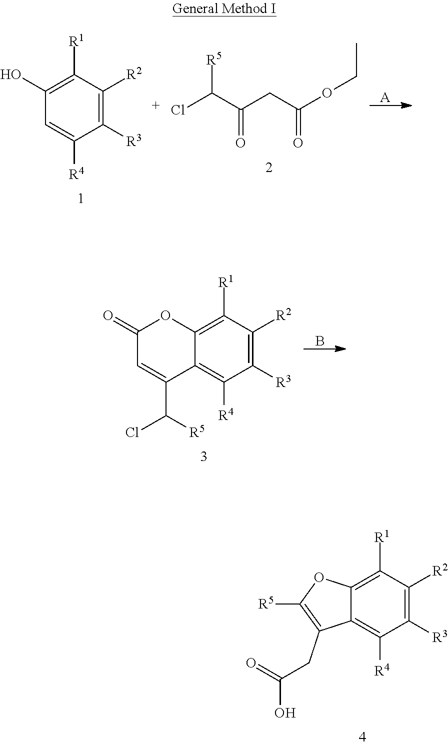

### Step A

Phenol compound (1) (100 mmol) was dissolved in ethyl chloroacetoacetate compound (2) (101 mmol) and the resulting solution was added dropwise to 50 mL of sulfuric acid (H₂SO₄) under stirring and ice cooling. The temperature was controlled within 0-10° C. The mixture was stirred for 8 hours at room temperature and then was poured into ice (200 g). The formed precipitate was filtered and washed with water (5×100 mL). Crude product was purified by crystallization. Yield 10-60%.

### Step B

To the solution of KOH in water (3 eq in 100 mL) compound (3) (0.1 mol) was added. The mixture was refluxed for 8-12 hours and then neutralized with hydrochloric acid. The precipitate was filtered and washed tree times with water (3×100 mL) and diethyl ether subsequently. The residue was recrystallized and dried to give the product 4 in yields 60-90%.

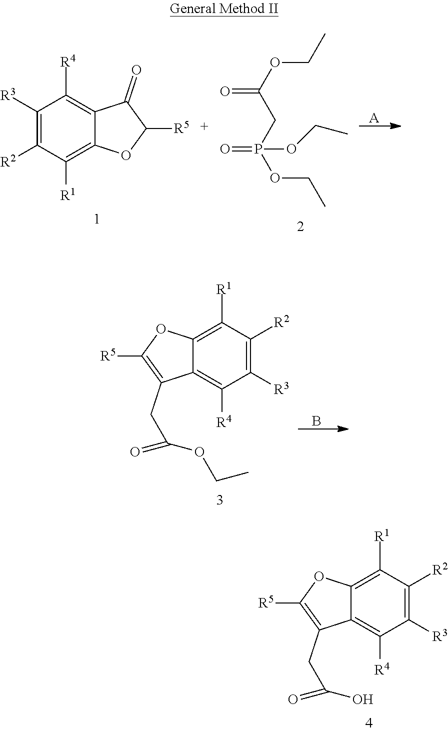

### Step A

To the solution of NaH (0.02 mol) in THF (50 mL) the solution of compound (1) (0.01 mol) and compound (2) (0.02 mol) in 20 mL of THF was added dropwise at ice cooling and stirring. The mixture was stirred with cooling for 6-8 hours, and poured into a mixture of ice (50 g) and water (50 g). The product was extracted with MTBE (3×75 mL); and the organic layer was washed with water (3×50 mL), dried and evaporated. The obtained compound (3) was used without purification in the next step. Yield 30-80%.

### Step B

To a solution of KOH (2 eq) in 50% aqueous methanol (50 mL) compound (3) was added. The mixture was refluxed for 1-2 hours, cooled and evaporated to dryness. The resulting salt was dissolved in water (30 mL) and impurities were extracted with MTBE (3×30 mL). The aqueous layer was neutralized with hydrochloric acid. The title product (4) was filtered, washed with water (3×30 mL) and dried. Yield 80-90%.

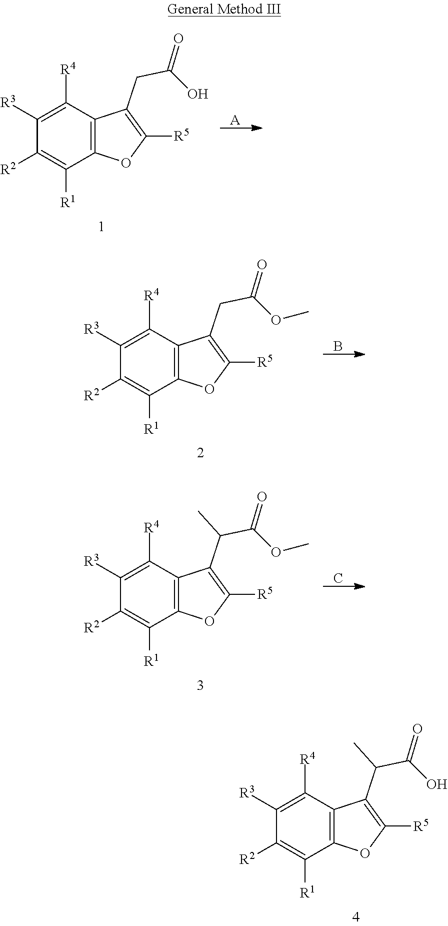

### Step A

5 g of acid (1) were dissolved in 40 mL of MeOH and cooled to −10° C. Then 3 eq of SOCl₂ were added dropwise. The obtained reaction mixture was allowed to warm to r.t. and stirred for an additional 30 min. Volatiles were removed at reduced pressure and the residue was partitioned between 50 mL of ethyl acetate and 50 mL of saturated solution of NaHCO₃. The aqueous phase was additionally extracted with 30 mL of ethyl acetate. Combined organic fractions were washed with 40 mL of saturated solution of NaCl, dried with Na₂SO₄ and evaporated in vacuo to afford 5.4 g of the title compound (2) as yellow oil. Yield: 100%.

### Step B

Diethylamine (1.2 eq) and 80 mL of THF were placed in a 250 mL round-bottom 3-necked flask equipped with dropping funnel. The solution was cooled to −50° C., then BuLi (2.4 M solution in hexane, 1.05 eq) was added dropwise. The obtained mixture was stirred at −50° C. for 30 min, then the solution was further cooled to −70° C. and ester (2) (1 eq) dissolved in 10 mL of THF was added dropwise. The resulting red solution was stirred for 1 h at −70-−60° C., then methyl iodide (1.2 eq) was added dropwise. The reaction was stirred at ambient temperature overnight, then cooled with an ice bath and quenched by addition of 50 mL of saturated NH₄Cl solution. Layers were separated and the aqueous phase was extracted with 80 mL of ethyl acetate. Combined organic fractions were washed successively with 50 mL of 7% solution of NaHSO₄, 50 mL of saturated solution of NaHCO₃, and 50 mL of saturated solution of NaCl, dried with Na₂SO₄ and evaporated in vacuo to afford the title compound (3) as a reddish oil. Yield: 85-91%.

### Step C

To a stirred solution of the methylated ester (3) (1 eq) in 60 mL of ethanol, a solution of KOH (1.5 eq) in 10 mL of water was added and the obtained solution was refluxed for 1 h. Volatiles were removed at reduced pressure and residue was dissolved in 50 mL of water. The solution was extracted with two portions of DCM (30 mL×2), then the aqueous phase was acidified using 3N aqueous HCl solution and extracted with two portions of EtOAc (50 mL×2). The combined EtOAc-fractions were washed with saturated solution of NaCl (60 mL), dried with Na₂SO₄ and evaporated in vacuo to afford crude product which was recrystallized from acetonitrile to give the pure title compound (4). Yield: 72%.

### II. Preparation of 3-(benzofuran-3-yl)propanoic Acid Compounds General Method IV

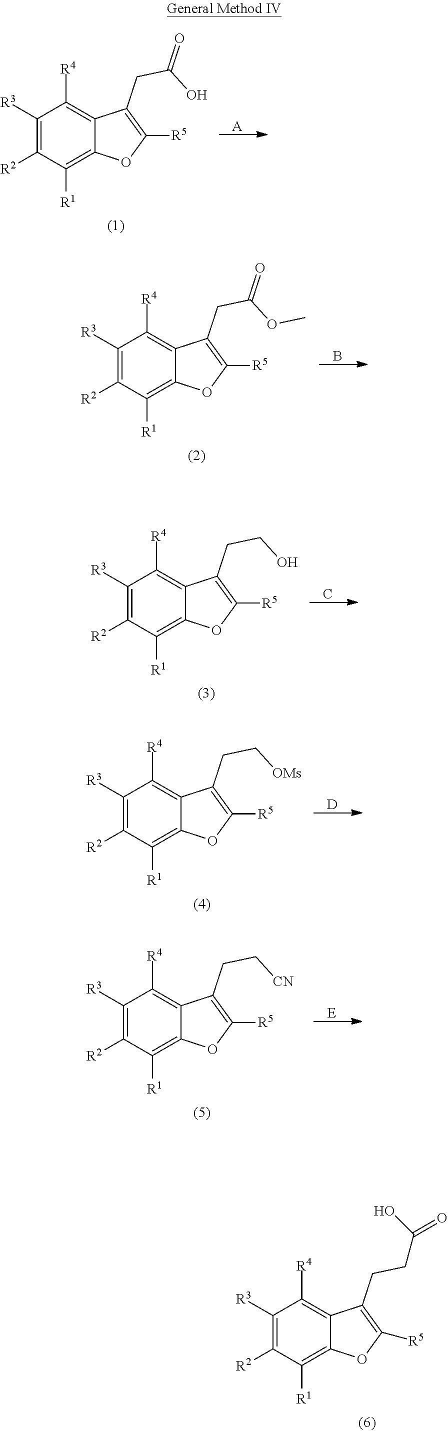

### Step A

5 g of acid (1) was dissolved in 40 mL of MeOH and cooled to −10° C. then 6 mL (3 eq) of SOCl₂ were added dropwise. Obtained reaction mixture was allowed to warm to r.t. and stirred for additional 30 min. Volatiles were removed at reduced pressure and residue was partitioned between 50 mL of ethyl acetate 50 mL of saturated solution of NaHCO₃, water fraction was additionally extracted with 30 mL of ethyl acetate, combined organic fractions were washed with 40 mL of saturated solution of NaCl, dried with Na₂SO₄ and evaporated in vacuum to afford compound (2).

### Step B

Lithium aluminium hydride (1.1 g, 1.0 eq) was suspended in 100 mL Et₂O and compound (2) was added dropwise. Mixture was stirred at ambient temperature for 1 h then quenched with 5 mL of water, solid was filtered off and ether was removed in vacuo to afford compound (3).

### Step C

Compound (3) was dissolved in 60 mL of DCM and 2.4 eq of Et₃N were added. Obtained solution was cooled to −40° C. and 1.2 eq of methanesulfonyl chloride dissolved in 5 mL of DCM was added dropwise in rate to keep internal temperature below −30° C. After the end of the addition the reaction mixture was allowed to warm to r.t. then diluted with DCM and washed with 7% solution of NaHSO₄, saturated solution of NaHCO₃, and of saturated solution of NaCl consequentially, dried with Na₂SO₄ and evaporated in vacuum to afford compound (4).

### Step D

Methanesulfonate compound (4) was dissolved in 70 mL of DMF and 1.5 eq of potassium cyanide was added. Obtained solution was heated at 80° C. for 14 h then cooled to 0° C. and poured in 100 mL of water. Obtained emulsion was extracted with two portions of EtOAc, combined organic fractions were washed with water (3×), and saturated solution of NaCl, dried with Na₂SO₄ and evaporated in vacuum to afford compound (5).

### Step E

The starting nitrile (5) was dissolved in MeOH and 3.0 eq of sodium hydroxide dissolved in water was added. Obtained solution was heated at reflux for 8 h then cooled to r. t. Volatiles were removed at reduced pressure and residue was dissolved in water. Obtained solution was extracted with two portions of MTBE (2×) then water fraction was acidified using 3 N HCl to pH 1 and extracted with two portions of EtOAc, combined EtOAc-fractions were washed with saturated solution of NaCl, dried with Na₂SO₄ and evaporated in vacuum to afford compound (6).

### III Preparation of Compounds of Formula (I)

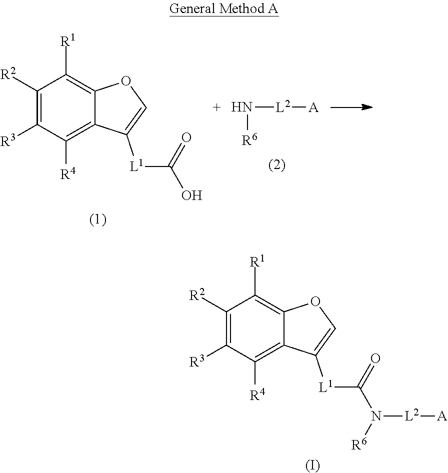

The carboxylic acid (1) (1.0 mmol) was dissolved in 30 mL of acetonitrile and then 1,1′-carbonyldiimidazole (1.2 mmol) was added. The resulting solution was heated under reflux for 1 h. Thereafter, the amine (2) (1.0 mmol) was added and the reaction mixture heated to reflux for 3 hour. Conversion of the starting materials was controlled by TLC. The solution was cooled to room temperature and solvent evaporated in vacuo. The residue was dissolved in water and the resulting precipitate was filtered off and washed twice with dilute aqueous hydrochloric acid and subsequently with aqueous sodium hydrogen carbonate and water. The crude title product (I) was purified by recrystallization from isopropyl alcohol or by HPLC chromatography. Yield: 60-90

### General Method B

The carboxylic acid (1) (2 mmol) and 1,1′-carbonyldiimidazole (2.4 mmol) were dissolved in acetonitrile and stirred for 1 hour. The amine (2) (2 mmol) was added to the reaction mixture and the mixture was refluxed overnight. After TLC control the suspension was cooled and the solvent evaporated under reduced pressure. The residue was treated with water and formed precipitate filtered out, washed with diluted hydrochloric acid, sodium hydrogen carbonate and then again with water. The crude product (I) was purified by flash chromatography. Yield: 30-50%.

### General Method C

The carboxylic acid (1) (1 eq.) was dissolved in DMF, then the amine (2) (1.1 eq.), 1-hydroxy-7-azabenzotriazole (HOAt) (1.2 eq) and 1.2 eq. of 1-ethyl-3-(3-dimethylaminopropyl)carbodiimide (EDC) were added sequentially. Resulting mixture was stirred at room temperature for overnight. Thereafter solvent and other volatiles were removed under reduced pressure. Residue was partitioned between water and ethyl acetate 50:50 and organic layer was then separated. Water layer was extracted with additional portions of ethyl acetate. Combined organic fraction was washed with citric acid solution (10%), saturated solution of NaHCO₃ and brine, then dried over Na₂SO₄ and evaporated in vacuo to give a crude product (I). The crude title product (I) was purified by recrystallization or with column chromatography. Yield: 40-80%.

### Example 1

### N-[4-[5-(Acetamidomethyl)-2-furyl]thiazol-2-yl]-2-(5-isopropyl-6-methyl-benzofuran-3-yl)acetamide

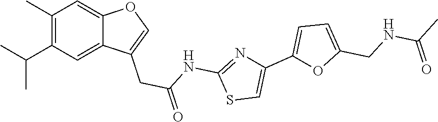 [SMILES: CC(=O)NCc1ccc(-c2csc(NC(=O)Cc3coc4cc(C)c(C(C)C)cc34)n2)o1]

The title compound is commercially available, e.g. from Enamine Ltd.

### Example 2

### 2-(6-Methoxybenzofuran-3-yl)-N-thiazol-2-yl-acetamide

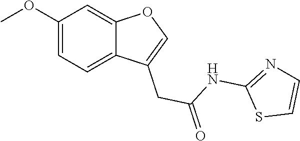 [SMILES: COc1ccc2c(CC(=O)Nc3nccs3)coc2c1]

The title compound is commercially available, e.g. from Enamine Ltd.

### Example 3

### 2-(6,7-Dihydro-5H-cyclopenta[f]benzofuran-3-yl)-N-(1-methylpyrazol-3-yl)acetamide

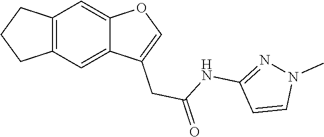 [SMILES: Cn1ccc(NC(=O)Cc2coc3cc4c(cc23)CCC4)n1]

2-(6,7-Dihydro-5H-cyclopenta[f]benzofuran-3-yl)acetic acid (300 mg, 1.39 mmol) was dissolved in DMF (10 mL). 1-Methylpyrazol-3-amine (0.12 mL, 1.53 mmol) and DIPEA (0.47 mL, 2.8 mmol) were added. PyBOP (794 mg, 1.53 mmol) was added last and the reaction was allowed to run over night at room temperature. The solvent was removed in vacuo. The residue was dissolved in EtOAc and washed twice with sat. aq. sodium bicarbonate solution, once with water and once with sat. sodium chloride solution. The organic phase was evaporated and the residue was purified by flash chromatography (eluting with DCM:EtOAc 1:1). The solvent was removed in vacuo and the title compound was obtained as a brownish oil (237 mg, 0.80 mmol, 58% yield). UPLC-MS (ES pos.) m/z 296 (M+H)⁺; retention time 1.542 min.

### Example 4

### 2-(5-Methylbenzofuran-3-yl)-N-[5-(trifluoromethyl)thiazol-2-yl]acetamide

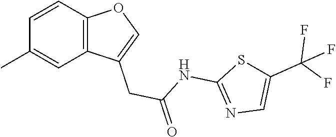 [SMILES: Cc1ccc2occ(CC(=O)Nc3ncc(C(F)(F)F)s3)c2c1]

The title compound was prepared according to General Method B using 2-(5-methylbenzofuran-3-yl)acetic acid and 5-(trifluoromethyl)thiazol-2-amine. Yield 42%.

¹H NMR (400 MHz, DMSO-d₆): δ=13.02 (s, 1H), 8.12 (s, 1H), 7.88 (s, 1H), 7.44 (d, J=8.4 Hz, 1H), 7.41 (s, 1H), 7.13 (d, J=8.1 Hz, 1H), 3.94 (s, 2H), 2.39 (s, 3H). LC-MS (positive mode) m/z 341 (M+H)⁺. Retention time 1.543 min.

### Example 5

### 2-(6-Ethylbenzofuran-3-yl)-N-[4-(2-pyridyl)thiazol-2-yl]acetamide

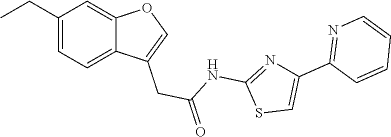 [SMILES: CCc1ccc2c(CC(=O)Nc3nc(-c4ccccn4)cs3)coc2c1]

The title compound is commercially available, e.g. from Enamine Ltd.

### Example 6

### 2-(Benzofuran-3-yl)-N-[5-(trifluoromethyl)thiazol-2-yl]acetamide

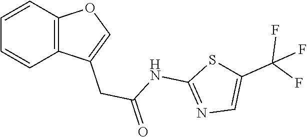 [SMILES: O=C(Cc1coc2ccccc12)Nc1ncc(C(F)(F)F)s1]

The title compound was prepared according to General Method B using 2-(benzofuran-3-yl)acetic acid and 5-(trifluoromethyl)thiazol-2-amine. UPLC-MS (positive mode) m/z 327 (M+H)⁺. Retention time 1.700 min.

### Example 7

### N-[4-(2-Amino-2-oxo-ethyl)thiazol-2-yl]-2-(6-methoxybenzofuran-3-yl)acetamide

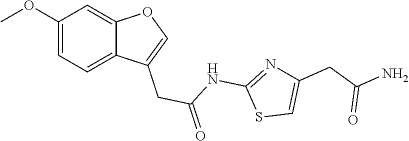 [SMILES: COc1ccc2c(CC(=O)Nc3nc(CC(N)=O)cs3)coc2c1]

The title compound is commercially available, e. g. from Enamine Ltd.

### Example 8

### N-[4-[5-(Acetamidomethyl)-2-furyl]thiazol-2-yl]-2-(6-methoxybenzofuran-3-yl)acetamide

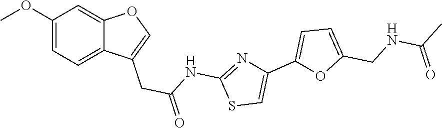 [SMILES: COc1ccc2c(CC(=O)Nc3nc(-c4ccc(CNC(C)=O)o4)cs3)coc2c1]

The title compound is commercially available, e. g. from Enamine Ltd.

### Example 9

### 2-(5,6-Dimethylbenzofuran-3-yl)-N-[4-(2-pyridyl)thiazol-2-yl]acetamide

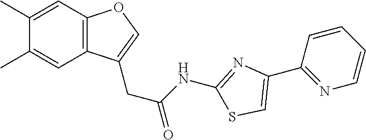 [SMILES: Cc1cc2occ(CC(=O)Nc3nc(-c4ccccn4)cs3)c2cc1C]

The title compound is commercially available, e. g. from UORSY.

### Example 10

### 2-(7-Methylbenzofuran-3-yl)-N-(5-methylthiazol-2-yl)acetamide

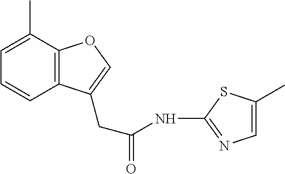 [SMILES: Cc1cnc(NC(=O)Cc2coc3c(C)cccc23)s1]

### 10.1 2-(7-methylbenzofuran-3-yl)acetic acid

The title compound was prepared according to General Method II.

### 10.2 2-(7-methylbenzofuran-3-yl)-N-(5-methylthiazol-2-yl)acetamide

The title compound was prepared according to General Method A using 2-(7-methylbenzofuran-3-yl)acetic acid and 5-methylthiazol-2-amine. Yield: 80%. ¹H NMR (400 MHz, DMSO-d₆): δ=2.30 (s, 6H), 3.86 (s, 2H), 7.18 (m, 3H), 7.42 (d, J=5.2 Hz, 1H), 7.89 (s, 1H), 12.20 (br. s, 1H). LC-MS (Positive mode) m/z 287 (M+H)⁺. HPLC retention time 1.318 min

### Example 11

### N-[4-(2,5-Dimethoxyphenyl)thiazol-2-yl]-2-(6-methylbenzofuran-3-yl)acetamide

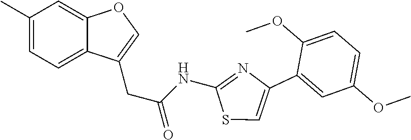

The title compound is commercially available, e. g. from Enamine Ltd.

### Example 12

### 2-(6-Methoxybenzofuran-3-yl)-N-(4-methylthiazol-2-yl)acetamide

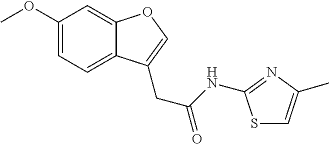

The title compound is commercially available, e. g. from Enamine Ltd.

### Example 13

### Ethyl 2-[[2-(6-methylbenzofuran-3-yl)acetyl]amino]thiazole-4-carboxylate

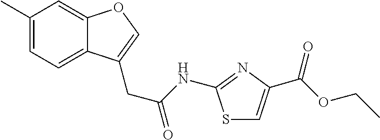 [SMILES: CCOC(=O)c1csc(NC(=O)Cc2coc3cc(C)ccc23)n1]

The title compound is commercially available, e. g. from Enamine Ltd.

### Example 14

### N-[4-(2,5-Dimethoxyphenyl)thiazol-2-yl]-2-(6-methoxybenzofuran-3-yl)acetamide

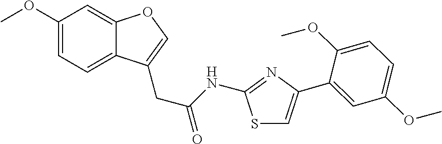

The title compound is commercially available, e. g. from Enamine Ltd.

### Example 15

### 2-(6-Chlorobenzofuran-3-yl)-N-(5-methylthiazol-2-yl)acetamide

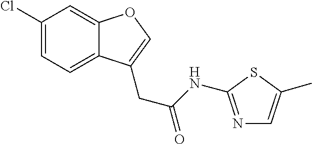 [SMILES: Cc1cnc(NC(=O)Cc2coc3cc(Cl)ccc23)s1]

### 15.1 2-(6-Chlorobenzofuran-3-yl)acetic acid

The title compound was prepared according to General Method I.

### 15.2 2-(6-Chlorobenzofuran-3-yl)-N-(5-methylthiazol-2-yl)acetamide

The title compound was prepared according to General Method A using 2-(6-chlorobenzofuran-3-yl)acetic acid and 5-methylthiazol-2-amine. Yield: 69%. ¹H NMR (400 MHz, DMSO-d₆): δ=12.22 (s, 1H), 7.96 (s, 1H), 7.75 (s, 1H), 7.64 (d, J=8.3 Hz, 1H), 7.33 (d, J=8.3 Hz, 1H), 7.13 (s, 1H), 3.87 (s, 2H), 2.32 (s, 3H). HPLC-MS (Positive mode) m/z 307/309 (M+H)⁺. Retention time 1.362 min.

### Example 16

### N-(1,3-Benzothiazol-2-yl)-N-(4-fluorophenyl)-2-(5-isopropyl-6-methyl-benzofuran-3-yl)acetamide

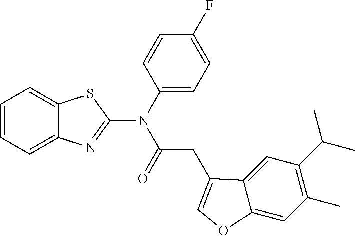 [SMILES: Cc1cc2occ(CC(=O)N(c3ccc(F)cc3)c3nc4ccccc4s3)c2cc1C(C)C]

The title compound is commercially available, e.g. from Enamine Ltd.

### Example 17

### 2-(6,7-Dihydro-5H-cyclopenta[f]benzofuran-3-yl)-N-(5-methyloxazol-2-yl)acetamide

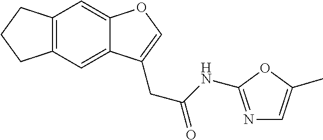 [SMILES: Cc1cnc(NC(=O)Cc2coc3cc4c(cc23)CCC4)o1]

This compound was prepared according to General Method A using 2-(6,7-dihydro-5H-cyclopenta[f]benzofuran-3-yl)acetic acid and 5-methyloxazol-2-amine. Yield 53%.

¹H NMR (400 MHz, DMSO-d₆): δ=2.08 (m, 2H), 2.26 (s, 3H), 2.94 (m, 4H), 3.78 (br. s, 2H), 6.50 (s, 1H), 7.29 (s, 1H), 7.37 (s, 1H), 7.56 (s, 1H), 10.18 (br. S, 1H). HPLC-MS (Positive mode) m/z 297 (M+H)⁺. Retention time 1.264 min.

### Example 18

### 2-[[2-(6,7-Dihydro-5H-cyclopenta[f]benzofuran-3-yl)acetyl]amino]thiazole-5-carboxamide

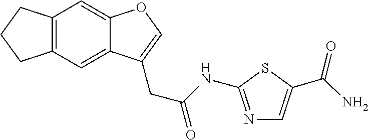 [SMILES: NC(=O)c1cnc(NC(=O)Cc2coc3cc4c(cc23)CCC4)s1]

The title compound is commercially available, e.g. from Enamine Ltd.

### Example 19

### N-[4-[5-(Acetamidomethyl)-2-furyl]thiazol-2-yl]-2-(6,7-dihydro-5H-cyclopenta[f]benzofuran-3-yl)acetamide

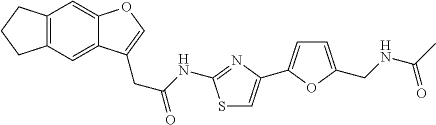 [SMILES: CC(=O)NCc1ccc(-c2csc(NC(=O)Cc3coc4cc5c(cc34)CCC5)n2)o1]

The title compound is commercially available, e.g. from Enamine Ltd.

### Example 20

### 2-(6-Ethylbenzofuran-3-yl)-N-thiazol-2-yl-acetamide

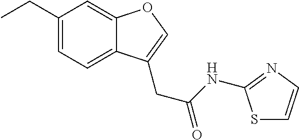 [SMILES: CCc1ccc2c(CC(=O)Nc3nccs3)coc2c1]

The title compound is commercially available, e.g. from Enamine Ltd.

### Example 21

### 2-(6-Methylbenzofuran-3-yl)-N-[4-(2-pyridyl)thiazol-2-yl]acetamide

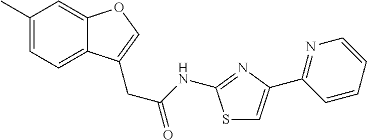 [SMILES: Cc1ccc2c(CC(=O)Nc3nc(-c4ccccn4)cs3)coc2c1]

The title compound is commercially available, e.g. from Enamine Ltd.

### Example 22

### 2-(6,7-Dimethylbenzofuran-3-yl)-N-(5-methyl-1H-imidazol-2-yl)acetamide

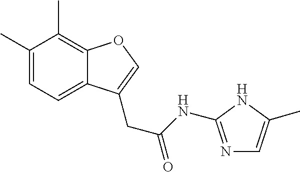

The title compound was prepared according to General Method A using 2-(6,7-dimethylbenzofuran-3-yl)acetic acid and 5-methyl-1H-imidazol-2-amine. Yield 64%. ¹H NMR (400 MHz, DMSO-d₆): δ=11.23 (s, 2H), 7.80 (s, 1H), 7.34 (d, J=5.3 Hz, 1H), 7.05 (d, J=6.1 Hz, 1H), 6.38 (s, 1H), 3.70 (s, 2H), 2.35 (s, 3H), 2.32 (s, 3H), 2.05 (s, 3H). HPLC-MS (Positive mode) m/z 284 (M+H)⁺. Retention time 1.028 min.

### Example 23

### 2-(6,7-dihydro-5H-cyclopenta[f]benzofuran-3-yl)-N-(5-methyl-1H-imidazol-2-yl)acetamide

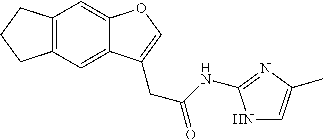 [SMILES: Cc1c[nH]c(NC(=O)Cc2coc3cc4c(cc23)CCC4)n1]

The title compound was prepared according to General Method A using 2-(6,7-dihydro-5H-cyclopenta[f]benzofuran-3-yl)acetic acid and 5-methyl-1H-imidazol-2-amine. Yield: 79%. ¹H NMR (400 MHz, DMSO-d₆): δ=2.10 (m, 5H), 2.96 (m, 4H), 3.67 (s, 2H), 6.30 (s, 1H), 7.23 (s, 1H), 7.45 (s, 1H), 7.62 (s, 1H), 11.14 (br. d, 2H). HPLC-MS (Positive mode) m/z 296 (M+H)⁺. Retention time 1.087 min.

### Example 24

### N-(4-Acetylthiazol-2-yl)-2-(6-methylbenzofuran-3-yl)acetamide

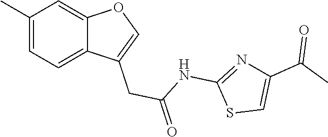 [SMILES: CC(=O)c1csc(NC(=O)Cc2coc3cc(C)ccc23)n1]

The title compound is commercially available, e.g. from Enamine Ltd.

### Example 25

### 2-(6-Methoxybenzofuran-3-yl)-N-[4-(4-pyridyl)thiazol-2-yl]acetamide

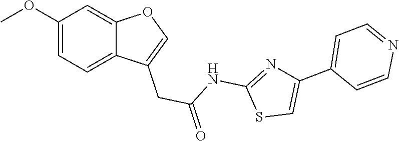 [SMILES: COc1ccc2c(CC(=O)Nc3nc(-c4ccncc4)cs3)coc2c1]

The title compound is commercially available, e.g. from Enamine Ltd.

### Example 26

### 2-(6-Methoxybenzofuran-3-yl)-N-[4-(2-pyridyl)thiazol-2-yl]acetamide

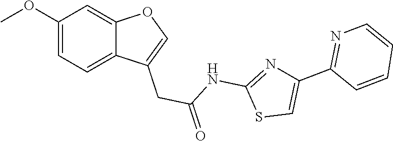 [SMILES: COc1ccc2c(CC(=O)Nc3nc(-c4ccccn4)cs3)coc2c1]

The title compound is commercially available, e.g. from Enamine Ltd.

### Example 27

### 2-(6-Methylbenzofuran-3-yl)-N-[4-(2-thienyl)thiazol-2-yl]acetamide

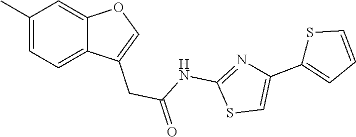 [SMILES: Cc1ccc2c(CC(=O)Nc3nc(-c4cccs4)cs3)coc2c1]

The title compound is commercially available, e.g. from Enamine Ltd.

### Example 28

### N-[4-[5-(2-Acetamidoethyl)-2-thienyl]thiazol-2-yl]-2-(6-methylbenzofuran-3-yl)acetamide

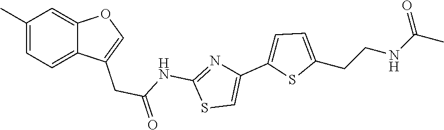 [SMILES: CC(=O)NCCc1ccc(-c2csc(NC(=O)Cc3coc4cc(C)ccc34)n2)s1]

The title compound is commercially available, e.g. from Enamine Ltd.

### Example 29

### 2-(6-Methoxybenzofuran-3-yl)-N-[4-(2-oxo-3,4-dihydro-1H-quinolin-6-yl)thiazol-2-yl]acetamide

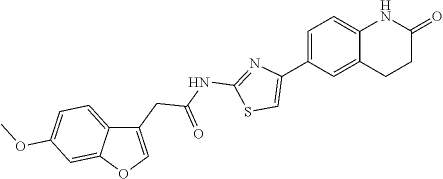 [SMILES: COc1ccc2c(CC(=O)Nc3nc(-c4ccc5c(c4)CCC(=O)N5)cs3)coc2c1]

The title compound is commercially available, e.g. from Enamine Ltd.

### Example 30

### N-[4-[4-(Acetamidomethyl)phenyl]thiazol-2-yl]-2-(6-ethylbenzofuran-3-yl)acetamide

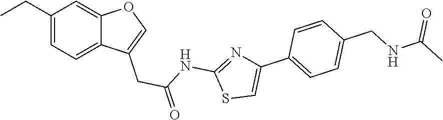 [SMILES: CCc1ccc2c(CC(=O)Nc3nc(-c4ccc(CNC(C)=O)cc4)cs3)coc2c1]

The title compound is commercially available, e.g. from Enamine Ltd.

### Example 31

### 2-(6-Methylbenzofuran-3-yl)-N-[4-(4-pyridyl)thiazol-2-yl]acetamide

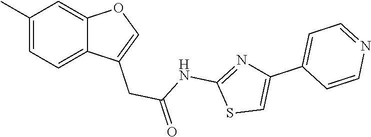 [SMILES: Cc1ccc2c(CC(=O)Nc3nc(-c4ccncc4)cs3)coc2c1]

The title compound is commercially available, e.g. from Enamine Ltd.

### Example 32

### 2-(6,7-Dihydro-5H-cyclopenta[f]benzofuran-3-yl)-N-(6-methoxy-1,3-benzothiazol-2-yl)acetamide

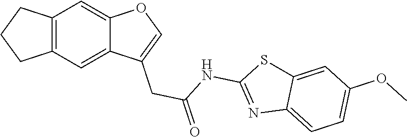 [SMILES: COc1ccc2nc(NC(=O)Cc3coc4cc5c(cc34)CCC5)sc2c1]

The title compound is commercially available, e.g. from Enamine Ltd.

### Example 33

### 2-(6-Methylbenzofuran-3-yl)-N-(4-methylthiazol-2-yl)acetamide

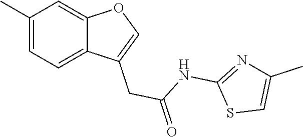

The title compound is commercially available, e.g. from Enamine Ltd.

### Example 34

### 2-Furo[3,2-f][1,3]benzodioxol-7-yl-N-(5-methylthiazol-2-yl)acetamide

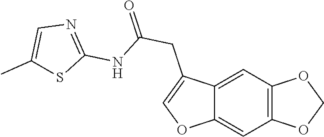 [SMILES: Cc1cnc(NC(=O)Cc2coc3cc4c(cc23)OCO4)s1]

### 34.1 8-(Chloromethyl)-[1,3]dioxolo[4,5-g]chromen-6-one

1,3-Benzodioxol-5-ol (1.38 g, 10.0 mmol) and ethyl 4-chloro-3-oxo-butanoate (1.81 g, 11.0 mmol) were added to H₂SO₄ (10 mL; cooled to −10° C.) and the mixture was stirred at 0° C. for 3 h. Then it was left overnight at +4° C. and poured onto crushed ice. The resulting precipitate was filtered, washed with water (5×30 mL), and dried to obtain 2.10 g (8.80 mmol, 88%) of the title compound.

### 34.2 2-Furo[2,3-f][1,3]benzodioxol-7-ylacetic acid

To a solution of 8-(chloromethyl)-[1,3]dioxolo[4,5-g]chromen-6-one (1.19 g, 5.00 mmol) in methanol (30 mL) a solution of KOH (0.840 g, 15.0 mmol) in H₂O (10 mL) was added. The mixture was refluxed for 3 h, concentrated to ½ of the initial volume, and neutralized with hydrochloric acid. The precipitated solid was filtered, washed with water (3×20 mL), and re-crystallized from i-PrOH to obtain 0.450 g (2.04 mmol, 41%) of the title compound.

### 34.3 2-Furo[3,2-f][1,3]benzodioxol-7-yl-N-(5-methylthiazol-2-yl)acetamide

A mixture of 2-furo[2,3-f][1,3]benzodioxol-7-ylacetic acid (0.220 g, 1.00 mmol), SOCl₂ (0.16 mL), and hexane (10 mL) was stirred at 40-50° C. for 3 h, cooled to room temperature and evaporated to dryness under reduced pressure. The solid was re-crystallized from hexane to give 0.200 g of corresponding acid chloride. It was dissolved in acetonitrile (10 mL) and mixed with 5-methyl-thiazol-2-ylamine (0.114 g, 1.00 mmol) and triethylamine (1.4 mL). The reaction was refluxed for 1 h, cooled to room temperature and filtered. The filtrate was washed with water to give 0.070 g (0.221 mmol, 22%) of title compound. ¹H NMR (400 MHz, DMSO-d₆): δ=2.32 (s, 3H), 3.77 (s, 2H), 6.03 (s, 2H), 7.09 (s, 1H), 7.13 (s, 1H), 7.22 (s, 1H), 7.77 (s, 1H), 12.18 (br. s, 1H).

HPLC-MS (Positive mode) m/z 317 (M+H)⁺. Retention time 1.226 min.

### Example 35

### 2-(6-Ethylbenzofuran-3-yl)-N-(4-methylthiazol-2-yl)acetamide

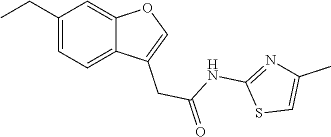 [SMILES: CCc1ccc2c(CC(=O)Nc3nc(C)cs3)coc2c1]

The title compound is commercially available, e.g. from Enamine Ltd.

### Example 36

### 2-(6-Methylbenzofuran-3-yl)-N-[4-(5-methyl-2-furyl)thiazol-2-yl]acetamide

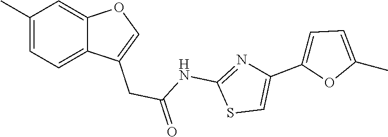

The title compound is commercially available, e.g. from Enamine Ltd.

### Example 37

### 2-(6-Methoxybenzofuran-3-yl)-N-[4-(2-thienyl)thiazol-2-yl]acetamide

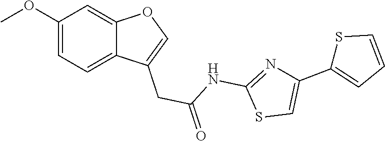 [SMILES: COc1ccc2c(CC(=O)Nc3nc(-c4cccs4)cs3)coc2c1]

The title compound is commercially available, e.g. from Enamine Ltd.

### Example 38

### 2-(6-Chlorobenzofuran-3-yl)-N-[5-(trifluoromethyl)-1H-imidazol-2-yl]acetamide

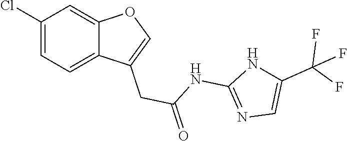 [SMILES: O=C(Cc1coc2cc(Cl)ccc12)Nc1ncc(C(F)(F)F)[nH]1]

### 38.1 2-(6-chlorobenzofuran-3-yl)acetic acid

The title compound was prepared according to General Method I.

### 38.2 2-(6-chlorobenzofuran-3-yl)-N-[5-(trifluoromethyl)-1H-imidazol-2-yl]acetamide

The title compound was prepared according to General Method B using 2-(6-chlorobenzofuran-3-yl)acetic acid and 5-(trifluoromethyl)-1H-imidazol-2-amine. Yield: 41%. ¹H NMR (400 MHz, DMSO-d6): δ=3.82 (s, 2H), 7.34 (br. s, 2H), 7.68 (d, J=8.0 Hz, 1H), 7.76 (s, 1H), 7.97 (s, 1H), 11.69 (s, 1H), 12.17 (br. s, 1H). HPLC-MS (Positive mode) m/z 344/346 (M+H)⁺. Retention time 1.363 min.

### Example 39

### 2-(6,7-Difluorobenzofuran-3-yl)-N-[5-(trifluoromethyl)thiazol-2-yl]acetamide

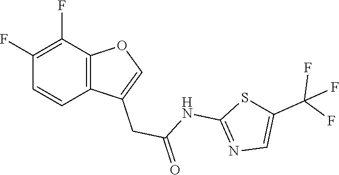 [SMILES: O=C(Cc1coc2c(F)c(F)ccc12)Nc1ncc(C(F)(F)F)s1]

The title compound was prepared according to General Method B using 2-(6,7-difluorobenzofuran-3-yl)acetic acid and 5-(trifluoromethyl)thiazol-2-amine. Yield: 30%.

¹H NMR (400 MHz, CDCl₃): δ=9.39-9.20 (br. s, 1H), 7.75 (s, 1H), 7.70 (s, 1H), 7.21-7.09 (m, 2H), 3.91 (s, 2H). HPLC-MS (Positive mode) m/z 363 (M+H)⁺. Retention time 1.484 min.

### Example 40

### 2-(6-Ethylbenzofuran-3-yl)-N-(5-methylthiazol-2-yl)acetamide

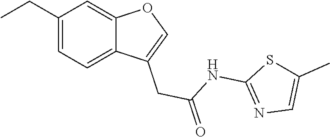 [SMILES: CCc1ccc2c(CC(=O)Nc3ncc(C)s3)coc2c1]

The title compound is commercially available, e.g. from Enamine Ltd.

### Example 41

### N-(6-Methoxy-1,3-benzothiazol-2-yl)-2-(6-methylbenzofuran-3-yl)acetamide

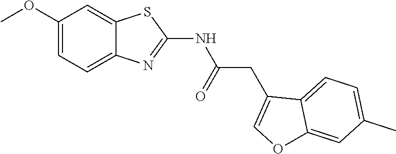

The title compound is commercially available, e.g. from Enamine Ltd.

### Example 42

### 2-(6-Methylbenzofuran-3-yl)-N-thiazol-2-yl-acetamide

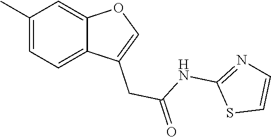 [SMILES: Cc1ccc2c(CC(=O)Nc3nccs3)coc2c1]

The title compound is commercially available, e.g. from Enamine Ltd.

### Example 43

### 2-(6-Methoxybenzofuran-3-yl)-N-(5-methylthiazol-2-yl)acetamide

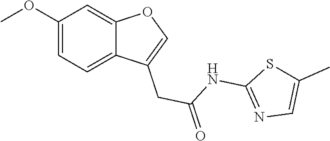

The title compound is commercially available, e.g. from Enamine Ltd.

### Example 44

### 3-(Benzofuran-3-yl)-N-[5-(trifluoromethyl)thiazol-2-yl]propanamide

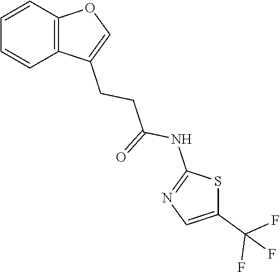 [SMILES: O=C(CCc1coc2ccccc12)Nc1ncc(C(F)(F)F)s1]

### 44.1 Methyl 2-(benzofuran-3-yl)acetate

The title compound was prepared according to General Method IV, step A and obtained as yellow oil. Yield: 100%. ¹H NMR (400 MHz, CDCl₃): δ=3.68 (s, 2H), 3.72 (s, 3H), 7.23 (d, J=8.8 Hz, 1H), 7.46 (d, J=8.4 Hz, 1H), 7.48 (s, 1H), 7.61 (s, 1H).

### 44.2 2-(Benzofuran-3-yl)ethanol

The title compound was prepared according to General Method IV, step B and obtained as colorless liquid. Yield: 91%. ¹H NMR (400 MHz, CDCl₃): δ=1.55 (t, J=5.8 Hz, 1H), 2.96 (t, J=6.2 Hz, 2H), 3.94 (q, J=6.4 Hz, 2H), 7.33-7.24 (m, 2H), 7.49 (d, J=8.0 Hz, 1H), 7.52 (s, 1H), 7.58 (d, J=8.0 Hz, 1H).

### 44.3 2-(Benzofuran-3-yl)ethyl methanesulfonate

The title compound was prepared according to General Method IV, step C and obtained as colorless oil. Yield: 97%. ¹H NMR (400 MHz, CDCl₃): δ=2.90 (s, 3H), 3.14 (t, J=6.8 Hz, 2H), 4.47 (t, J=6.6 Hz, 2H), 7.33-7.24 (m, 2H), 7.47 (d, J=8.4 Hz, 1H), 7.53 (s, 1H), 7.55 (d, J=7.0 Hz, 1H).

### 44.4 3-(Benzofuran-3-yl)propanenitrile

The title compound was prepared according to General Method IV, step D and obtained as orange oil. Yield: 98%. ¹H NMR (400 MHz, CDCl₃): δ=2.71 (t, J=7.2 Hz, 2H), 3.06 (t, J=7.4 Hz, 2H), 7.34-7.24 (m, 2H), 7.52-7.48 (m, 2H), 7.56 (s, 1H).

### 44.5 3-(Benzofuran-3-yl)propanoic acid

The title compound was prepared according to General Method IV, step E and obtained as beige powder. Yield: 84%. ¹H NMR (400 MHz, CDCl₃δ=2.76 (t, J=7.4 Hz, 2H), 3.02 (t, J=7.4 Hz, 2H), 7.31-7.23 (m, 2H), 7.46-7.45 (m, 2H), 7.54 (d, J=7.2 Hz, 1H).

### 44.6 3-(Benzofuran-3-yl)-N-[5-(trifluoromethyl)thiazol-2-yl]propanamide

The title compound was prepared according to General Method C using 3-(benzofuran-3-yl)propanoic acid and 5-(trifluoromethyl)thiazol-2-amine. ¹H NMR (400 MHz, DMSO-d6): δ=2.91 (t, J=7.4 Hz, 2H), 3.03 (t, J=7.4 Hz, 2H), 7.33-7.24 (m, 2H), 7.54 (d, J=7.6 Hz, 1H), 7.70 (d, J=8.0 Hz, 1H), 7.79 (s, 1H), 8.09 (s, 1H), 12.78 (br. s, 1H). HPLC-MS (Positive mode) m/z 341 (M+H)⁺. Retention time 1.538 min.

### Example 45

### 2-(5,6-Dimethylbenzofuran-3-yl)-N-(5-methylthiazol-2-yl)acetamide

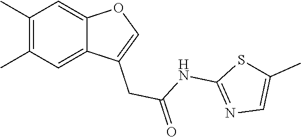

The title compound was prepared according to General Method A using 2-(5,6-dimethylbenzofuran-3-yl)acetic acid and 5-methylthiazol-2-amine. Yield: 92%. ¹H NMR (400 MHz, DMSO-d₆): δ=2.31 (t, 9H), 3.78 (s, 2H), 7.12 (s, 1H), 7.33 (s, 1H), 7.36 (s, 1H), 7.75 (s, 1H), 12.18 (br s, 1H). HPLC-MS (Positive mode) m/z 301 (M+H)⁺. Retention time 0.678 min.

### Example 46

### 2-(6-Chlorobenzofuran-3-yl)-N-(5-cyanothiazol-2-yl)acetamide

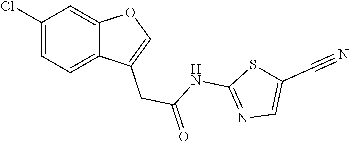 [SMILES: N#Cc1cnc(NC(=O)Cc2coc3cc(Cl)ccc23)s1]

### 46.1 2-(6-Chlorobenzofuran-3-yl)acetic acid

The title compound was prepared according to General Method I.

### 46.2 2-(6-Chlorobenzofuran-3-yl)-N-(5-cyanothiazol-2-yl)acetamide

The title compound was prepared according to General Method B using 2-(6-chlorobenzofuran-3-yl)acetic acid and 2-aminothiazole-5-carbonitrile. Yield 21%. ¹H NMR (400 MHz, DMSO-d6): δ=13.25 (s, 1H), 8.39 (s, 1H), 7.99 (s, 1H), 7.77 (s, 1H), 7.65 (d, J=8.3 Hz, 1H), 7.33 (d, J=7.9 Hz, 1H), 3.99 (s, 2H). HPLC-MS (Positive mode) m/z 318 (M+H)⁺. Retention time 1.360 min.

### Example 47

### Methyl 2-[[2-(6,7-dimethylbenzofuran-3-yl)acetyl]amino]thiazole-5-carboxylate

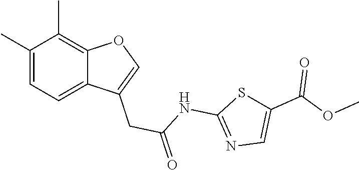

The title compound is commercially available, e.g. from Enamine Ltd.

### Example 48

### 2-(6-Methylbenzofuran-3-yl)-N-(5-methylthiazol-2-yl)acetamide

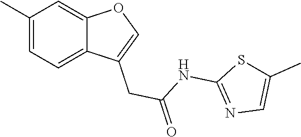

The title compound is commercially available, e.g. from Enamine Ltd.

### Example 49

### 2-(6,7-Dichlorobenzofuran-3-yl)-N-[5-(trifluoromethyl)-1H-imidazol-2-yl]acetamide

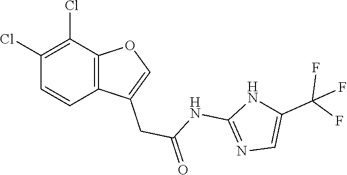 [SMILES: O=C(Cc1coc2c(Cl)c(Cl)ccc12)Nc1ncc(C(F)(F)F)[nH]1]

### 49.1 2-(6,7-Dichlorobenzofuran-3-yl)acetic acid

The title compound was prepared according to General Method II. HPLC-MS (Negative mode) m/z 245 (M−H). Retention time 1.365 min.

### 49.2 2-(6,7-Dichlorobenzofuran-3-yl)-N-[5-(trifluoromethyl)-1H-imidazol-2-yl]acetamide

The title compound was prepared according to General Method B using 2-(6,7-dichlorobenzofuran-3-yl)acetic acid and 5-(trifluoromethyl)-1H-imidazol-2-amine. Yield: 57%. ¹H NMR (400 MHz, DMSO-d6): δ=12.17 (s, 1H), 11.70 (s, 1H), 8.10 (s, 1H), 7.67 (d, J=8.3 Hz, 1H), 7.53 (d, J=8.3 Hz, 1H), 7.34 (s, 1H), 3.85 (s, 2H). HPLC-MS (Positive mode) m/z 378/380 (M+H)⁺. Retention time 1.491 min.

### Example 50

### N-[4-(3,4-Dimethoxyphenyl)thiazol-2-yl]-2-(6-methylbenzofuran-3-yl)acetamide

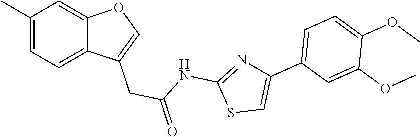

The title compound is commercially available, e.g. from Enamine Ltd.

### Example 51

### N-[4-[4-(3-Acetamidobutyl)phenyl]thiazol-2-yl]-2-(6-methoxybenzofuran-3-yl)acetamide

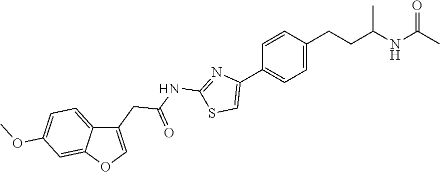 [SMILES: COc1ccc2c(CC(=O)Nc3nc(-c4ccc(CCC(C)NC(C)=O)cc4)cs3)coc2c1]

The title compound is commercially available, e.g. from Enamine Ltd.

### Example 52

### N-[4-(4-Acetamidophenyl)thiazol-2-yl]-2-(6-ethylbenzofuran-3-yl)acetamide

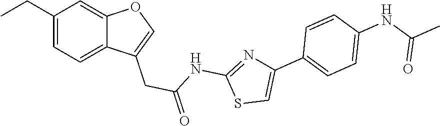 [SMILES: CCc1ccc2c(CC(=O)Nc3nc(-c4ccc(NC(C)=O)cc4)cs3)coc2c1]

The title compound is commercially available, e.g. from Enamine Ltd.

### Example 53

### 2-(7-Chlorobenzofuran-3-yl)-N-[5-(trifluoromethyl)thiazol-2-yl]acetamide

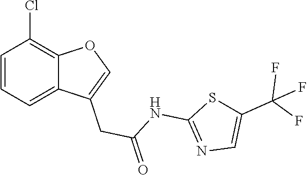 [SMILES: O=C(Cc1coc2c(Cl)cccc12)Nc1ncc(C(F)(F)F)s1]

### 53.1 2-(7-Chlorobenzofuran-3-yl)acetic acid

The title compound was prepared according to General Method II.

### 53.2 2-(7-Chlorobenzofuran-3-yl)-N-[5-(trifluoromethyl)thiazol-2-yl]acetamide

The title compound was prepared according to General Method B using 2-(7-chlorobenzofuran-3-yl)acetic acid and 5-(trifluoromethyl)thiazol-2-amine. Yield 33%. ¹H NMR (400 MHz, DMSO-d₆): δ=13.05 (s, 1H), 8.12 (s, 1H), 8.08 (s, 1H), 7.62 (d, J=7.7 Hz, 1H), 7.44 (d, J=7.7 Hz, 1H), 7.29 (t, J=7.7 Hz, 1H), 4.01 (s, 2H). HPLC-MS (Positive mode) m/z 361/362 (M+H)+. Retention time 1.476 min.

### Example 54

### N-(5,6-Dihydro-4H-cyclopenta[d]thiazol-2-yl)-2-(6-methoxybenzofuran-3-yl)acetamide

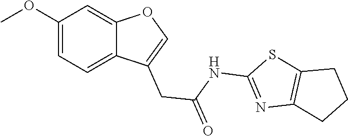 [SMILES: COc1ccc2c(CC(=O)Nc3nc4c(s3)CCC4)coc2c1]

The title compound is commercially available, e.g. from Enamine Ltd.

### Example 55

### 2-(6,7-Dimethylbenzofuran-3-yl)-N-[5-(trifluoromethyl)-1H-imidazol-2-yl]acetamide

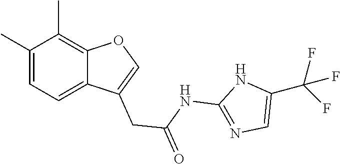 [SMILES: Cc1ccc2c(CC(=O)Nc3ncc(C(F)(F)F)[nH]3)coc2c1C]

The title compound was prepared according to General Method C using 2-(6,7-dimethylbenzofuran-3-yl)acetic acid and 5-(trifluoromethyl)-1H-imidazol-2-amine. Yield 52%. 1H NMR (400 MHz, DMSO-d₆): δ=12.14 (s, 1H), 11.64 (s, 1H), 7.83 (s, 1H), 7.33 (s, 2H), 7.06 (d, J=7.1 Hz, 1H), 3.76 (s, 2H), 2.36 (s, 3H), 2.33 (s, 3H). HPLC-MS (Positive mode) m/z 338 (M+H)+. Retention time 1.420 min.

### Example 56

### 2-(6-Chlorobenzofuran-3-yl)-N-[5-(2-methoxyethyl)thiazol-2-yl]acetamide

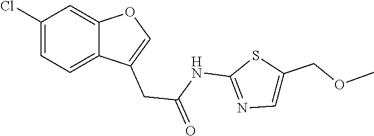 [SMILES: COCc1cnc(NC(=O)Cc2coc3cc(Cl)ccc23)s1]

### 56.1 2-(6-Chlorobenzofuran-3-yl)acetic acid

The title compound was prepared according to General Method I.

### 56.2 2-(6-Chlorobenzofuran-3-yl)-N-[5-(2-methoxyethyl)thiazol-2-yl]acetamide

The title compound was prepared according to General Method C using 2-(6-chlorobenzofuran-3-yl)acetic acid and 5-(methoxymethyl)thiazol-2-amine. Yield 81%. ¹H NMR (400 MHz, DMSO-d6): δ=12.40 (s, 1H), 7.97 (s, 1H), 7.76 (s, 1H), 7.65 (d, J=8.3 Hz, 1H), 7.40 (s, 1H), 7.33 (d, J=8.3 Hz, 1H), 4.52 (s, 2H), 3.90 (s, 2H), 3.22 (s, 3H). HPLC-MS (Positive mode) m/z 337/339 (M+H)⁺. Retention time 1.329 min.

### Example 57

### 2-(7-Chloro-6-methyl-benzofuran-3-yl)-N-(5-methyl-1H-imidazol-2-yl)acetamide

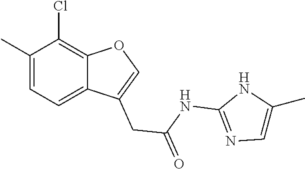

### 57.1 2-(7-Chloro-6-methyl-benzofuran-3-yl)acetic acid

The title compound was prepared according to General Method I.

### 57.2 2-(7-Chloro-6-methyl-benzofuran-3-yl)-N-(5-methyl-1H-imidazol-2-yl)acetamide

The title compound was prepared according to General Method A using 2-(7-chloro-6-methyl-benzofuran-3-yl)acetic acid and 5-methyl-1H-imidazol-2-amine.

Yield 32%. ¹H NMR (500 MHz, DMSO-d6): δ=11.24 (s, 1H), 7.94 (s, 1H), 7.52 (d, J=7.9 Hz, 1H), 7.25 (d, J=7.9 Hz, 1H), 6.40 (s, 1H), 3.75 (s, 2H), 2.44 (s, 3H), 2.05 (s, 3H).

HPLC-MS (Positive mode) m/z 304/306 (M+H)⁺. Retention time 1.073 min.

### Example 58

### 2-(7-Chloro-6-methyl-benzofuran-3-yl)-N-[5-(trifluoromethyl)-1H-imidazol-2-yl]acetamide

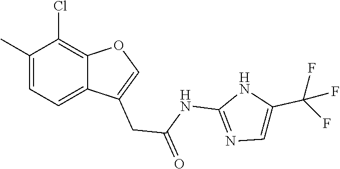 [SMILES: Cc1ccc2c(CC(=O)Nc3ncc(C(F)(F)F)[nH]3)coc2c1Cl]

### 58.1 2-(7-Chloro-6-methyl-benzofuran-3-yl)acetic acid

The title compound was prepared according to General Method I.

### 58.2 2-(7-Chloro-6-methyl-benzofuran-3-yl)-N-[5-(trifluoromethyl)-1H-imidazol-2-yl]acetamide

The title compound was prepared according to General Method B using 2-(7-chloro-6-methyl-benzofuran-3-yl)acetic acid and 5-(trifluoromethyl)-1H-imidazol-2-amine. Yield 51%. ¹H NMR (400 MHz, DMSO-d₆): δ=12.16 (s, 1H), 11.68 (s, 1H), 7.97 (s, 1H), 7.52 (d, J=7.8 Hz, 1H), 7.34 (s, 1H), 7.25 (d, J=7.9 Hz, 1H), 3.81 (s, 2H), 2.44 (s, 3H). HPLC-MS (Positive mode) m/z 358/360 (M+H)⁺. Retention time 1.473 min.

### Example 59

### 2-(7-Chloro-6-methyl-benzofuran-3-yl)-N-(5-cyano-1H-thiazol-2-yl)acetamide

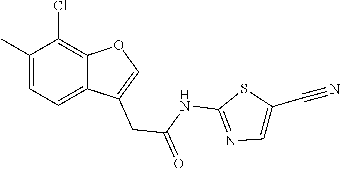 [SMILES: Cc1ccc2c(CC(=O)Nc3ncc(C#N)s3)coc2c1Cl]

### 59.1 2-(7-Chloro-6-methyl-benzofuran-3-yl)acetic acid

The title compound was prepared according to General Method I.

### 59.2 2-(7-Chloro-6-methyl-benzofuran-3-yl)-N-(5-cyano-1H-thiazol-2-yl)acetamide

The title compound was prepared according to General Method B using 2-(7-chloro-6-methyl-benzofuran-3-yl)acetic acid and 2-aminothiazole-5-carbonitrile. Yield 49%.

¹H NMR (400 MHz, DMSO-d₆): δ=2.44 (s, 3H), 3.94 (s, 2H), 7.25 (d, J=7.8 Hz, 1H), 7.49 (d, J=7.8 Hz, 1H), 7.98 (s, 1H), 8.32 (s, 1H).

HPLC-MS (Positive mode) m/z 332 (M+H)⁺. Retention time 1.405 min.

### Example 60

### 2-(6-Chlorobenzofuran-3-yl)-N-[5-(trifluoromethyl)thiazol-2-yl]acetamide

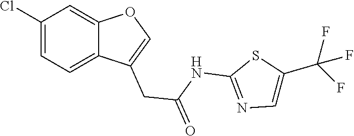 [SMILES: O=C(Cc1coc2cc(Cl)ccc12)Nc1ncc(C(F)(F)F)s1]

### 60.1 2-(6-Chlorobenzofuran-3-yl)acetic acid

The title compound was prepared according to General Method I.

### 60.2 2-(6-Chlorobenzofuran-3-yl)-N-[5-(trifluoromethyl)thiazol-2-yl]acetamide

The title compound was prepared according to General Method B using 2-(6-chlorobenzofuran-3-yl)acetic acid and 5-(trifluoromethyl)thiazol-2-amine. Yield: 41%. ¹H NMR (400 MHz, DMSO-d₆): δ=12.93 (s, 1H), 8.10 (s, 1H), 7.98 (s, 1H), 7.76 (s, 1H), 7.65 (d, J=8.2 Hz, 1H), 7.33 (d, J=8.2 Hz, 1H), 3.97 (s, 2H).

HPLC-MS (Positive mode) m/z 361/363 (M+H)⁺. Retention time 1.468 min.

### Example 61

### 3-(6,7-Dimethylbenzofuran-3-yl)-N-[5-(trifluoromethyl)thiazol-2-yl]propanamide

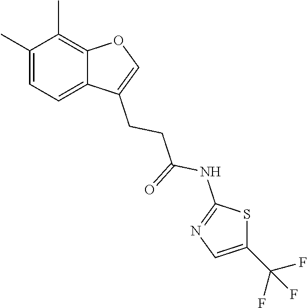 [SMILES: Cc1ccc2c(CCC(=O)Nc3ncc(C(F)(F)F)s3)coc2c1C]

This compound was synthesized analogously to example 44 using 2-(6,7-dimethylbenzofuran-3-yl)acetic acid as the starting material. Yield of the last step: 76%. ¹H NMR (400 MHz, DMSO-d6): δ=2.33 (s, 3H), 2.34 (s, 3H), 2.88 (t, J=7.4 Hz, 2H), 2.98 (t, J=7.4 Hz, 2H), 7.06 (d, J=8.0 Hz, 1H), 7.37 (d, J=8.0 Hz, 1H), 7.69 (s, 1H), 8.08 (s, 1H), 12.76 (br. s, 1H). HPLC-MS (Positive mode) m/z 369 (M+H)⁺. Retention time 1.670 min.

### Example 62

### N-(5-Cyanothiazol-2-yl)-2-(6,7-dimethylbenzofuran-3-yl)acetamide

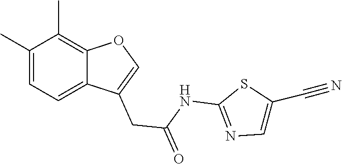 [SMILES: Cc1ccc2c(CC(=O)Nc3ncc(C#N)s3)coc2c1C]

The title compound was prepared according to General Method B using 2-(6,7-dimethylbenzofuran-3-yl)acetic acid and 2-aminothiazole-5-carbonitrile. UPLC-MS (Positive mode) m/z 312 (M+H)⁺. Retention time 1.683 min.

### Example 63

### 2-(6,7-Dihydro-5H-cyclopenta[f]benzofuran-3-yl)-N-[5-(trifluoromethyl)thiazol-2-yl]acetamide

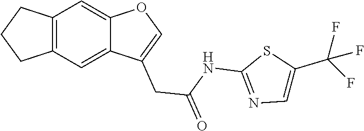 [SMILES: O=C(Cc1coc2cc3c(cc12)CCC3)Nc1ncc(C(F)(F)F)s1]

The title compound was prepared according to General Method B using 2-(6,7-dihydro-5H-cyclopenta[f]benzofuran-3-yl)acetic acid and 5-(trifluoromethyl)thiazol-2-amine. HPLC-MS (Positive mode) m/z 367 (M+H)⁺. Retention time 1.651 min.

### Example 64

### 2-(6,7-Dihydro-5H-cyclopenta[f]benzofuran-3-yl)-N-(5-methylthiazol-2-yl)acetamide

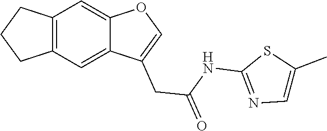 [SMILES: Cc1cnc(NC(=O)Cc2coc3cc4c(cc23)CCC4)s1]

The title compound is commercially available, e.g. from Enamine Ltd.

### Example 65

### 2-(6,7-Dihydro-5H-cyclopenta[f]benzofuran-3-yl)-N-[5-(methoxymethyl)thiazol-2-yl]acetamide

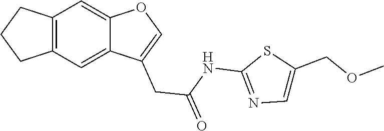 [SMILES: COCc1cnc(NC(=O)Cc2coc3cc4c(cc23)CCC4)s1]

The title compound was prepared according to General Method A using 2-(6,7-dihydro-5H-cyclopenta[f]benzofuran-3-yl)acetic acid and 5-(methoxymethyl)thiazol-2-amine. Yield: 25%. ¹H NMR (400 MHz, DMSO-d₆): δ=2.12 (t, J=7.4 Hz, 2H), 2.96 (s, 4H), 3.27 (s, 3H), 3.77 (s, 2H), 4.51 (s, 2H), 7.23 (s, 2H), 7.41 (s, 1H), 7.66 (s, 1H), 12.20 (br. s, 1H). HPLC-MS (Positive mode) m/z 343 (M+H)⁺. Retention time 1.400 min.

### Example 66

### 2-(6-Chlorobenzofuran-3-yl)-N-[5-(trifluoromethyl)thiazol-2-yl]propanamide

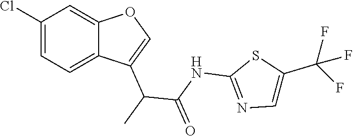 [SMILES: CC(C(=O)Nc1ncc(C(F)(F)F)s1)c1coc2cc(Cl)ccc12]

### 66.1 2-(6-Chlorobenzofuran-3-yl)propanoic acid

The title compound was synthesized according to General Method III.

### 66.2 2-(6-Chlorobenzofuran-3-yl)-N-[5-(trifluoromethyl)thiazol-2-yl]propanamide

The title compound was prepared according to General Method C using 2-(6-chlorobenzofuran-3-yl)propanoic acid and 5-(trifluoromethyl)thiazol-2-amine. Yield: 62%. HPLC-MS (Positive mode) m/z 375/376 (M+H)⁺. Retention time 1.624 min. ¹H NMR (400 MHz, DMSO-d6): δ=1.58 (d, J=6.8 Hz, 3H), 4.23 (q, J=7.2 Hz, 1H), 7.33 (d, J=8.4 Hz, 1H), 7.68 (d, J=8.4 Hz, 1H), 7.76 (s, 1H), 7.99 (s, 1H), 8.11 (s, 1H), 13.03 (br. s, 1H).

### Example 67

### N-[4-(3-Chloro-4-methoxy-phenyl)thiazol-2-yl]-2-(6-methylbenzofuran-3-yl)acetamide

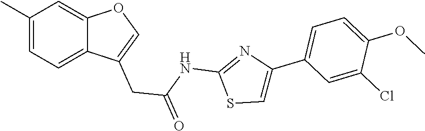

The title compound is commercially available, e.g. from Enamine Ltd.

### Example 68

### N-(4-Acetylphenyl)-N-(1,3-benzothiazol-2-yl)-2-(6-methoxybenzofuran-3-yl)acetamide

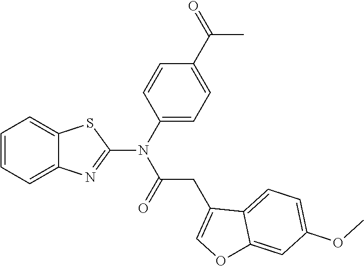 [SMILES: COc1ccc2c(CC(=O)N(c3ccc(C(C)=O)cc3)c3nc4ccccc4s3)coc2c1]

The title compound is commercially available, e.g. from Enamine Ltd.

### Example 69

### N-(4-Acetylphenyl)-N-(1,3-benzothiazol-2-yl)-2-(6-ethylbenzofuran-3-yl)acetamide

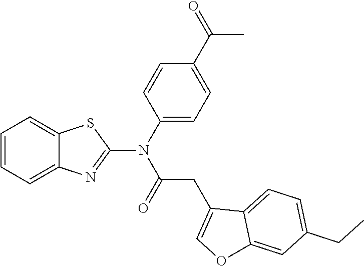 [SMILES: CCc1ccc2c(CC(=O)N(c3ccc(C(C)=O)cc3)c3nc4ccccc4s3)coc2c1]

The title compound is commercially available, e.g. from Enamine Ltd.

### Example 70

### N-Allyl-2-(6-methylbenzofuran-3-yl)-N-[4-(4-methylsulfonylphenyl)thiazol-2-yl]acetamide

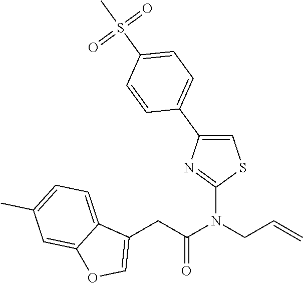 [SMILES: C=CCN(C(=O)Cc1coc2cc(C)ccc12)c1nc(-c2ccc(S(C)(=O)=O)cc2)cs1]

The title compound is commercially available, e.g. from Enamine Ltd.

### Example 71

### 2-(6-Methoxybenzofuran-3-yl)-N-[4-(4-methoxyphenyl)thiazol-2-yl]acetamide

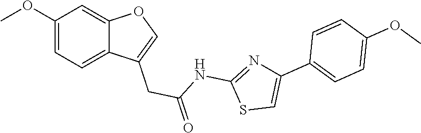

The title compound is commercially available, e.g. from Enamine Ltd.

### Example 72: 2-(7-Methylbenzofuran-3-yl)-N-[5-(trifluoromethyl)thiazol-2-yl]acetamide

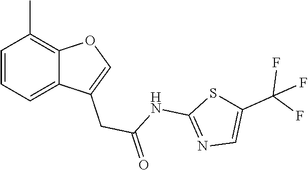 [SMILES: Cc1cccc2c(CC(=O)Nc3ncc(C(F)(F)F)s3)coc12]

### 72.1 2-(7-Methylbenzofuran-3-yl)acetic acid

The title compound was prepared according to General Method II.

### 72.2 2-(7-Methylbenzofuran-3-yl)-N-[5-(trifluoromethyl)thiazol-2-yl]acetamide

The title compound was prepared according to General Method B using 2-(7-methylbenzofuran-3-yl)acetic acid and 5-(trifluoromethyl)thiazol-2-amine. Yield: 46%. ¹H NMR (400 MHz, DMSO-d₆): δ=13.02 (s, 1H), 8.11 (s, 1H), 7.93 (s, 1H), 7.43 (d, J=8.4 Hz, 1H), 7.15 (m, 2H), 3.95 (s, 2H), 2.45 (s, 3H). HPLC-MS (Positive mode) m/z 341 (M+H)⁺. Retention time 1.527 min.

### Example 73

### N-(5-Cyanothiazol-2-yl)-2-(6,7-dichlorobenzofuran-3-yl)acetamide

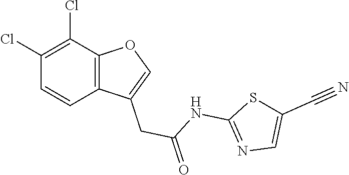 [SMILES: N#Cc1cnc(NC(=O)Cc2coc3c(Cl)c(Cl)ccc23)s1]

### 73.1 2-(6,7-Dichlorobenzofuran-3-yl)acetic acid

The title compound was prepared according to General Method II.

### 73.2 N-(5-Cyanothiazol-2-yl)-2-(6,7-dichlorobenzofuran-3-yl)acetamide

The title compound was prepared according to General Method B using 2-(6,7-dichlorobenzofuran-3-yl)acetic acid and 2-aminothiazole-5-carbonitrile. Yield: 30%. ¹H NMR (400 MHz, DMSO-d₆): δ=9.71 (s, 1H), 8.01 (s, 1H), 7.87 (s, 1H), 7.64 (d, J=8.2 Hz, 1H), 7.46 (d, J=8.3 Hz, 1H), 3.65 (s, 2H). HPLC-MS (Negative mode) m/z 352/350 (M−2H⁺)⁻. Retention time 1.471 min.

### Example 74

### N-[4-(2,3-Dihydrobenzofuran-5-yl)thiazol-2-yl]-2-(6-methoxybenzofuran-3-yl)acetamide

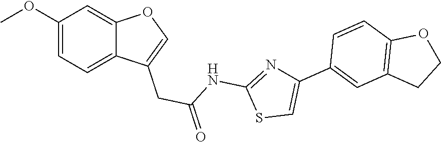 [SMILES: COc1ccc2c(CC(=O)Nc3nc(-c4ccc5c(c4)CCO5)cs3)coc2c1]

The title compound is commercially available, e.g. from Enamine Ltd.

### Example 75

### 2-(6,7-Dichlorobenzofuran-3-yl)-N-(5-methylthiazol-2-yl)acetamide

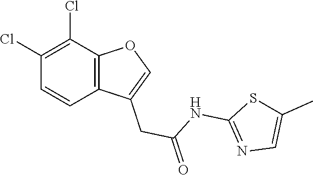 [SMILES: Cc1cnc(NC(=O)Cc2coc3c(Cl)c(Cl)ccc23)s1]

### 75.1 2-(6,7-Dichlorobenzofuran-3-yl)acetic acid

The title compound was prepared according to General Method II.

### 75.2 2-(6,7-Dichlorobenzofuran-3-yl)-N-(5-methylthiazol-2-yl)acetamide

The title compound was prepared according to General Method A using 2-(6,7-dichlorobenzofuran-3-yl)acetic acid and 5-methylthiazol-2-amine. Yield: 69%. ¹H NMR (400 MHz, DMSO-d₆): δ=12.22 (s, 1H), 8.09 (s, 1H), 7.63 (d, J=8.4 Hz, 1H), 7.53 (d, J=8.4 Hz, 1H), 7.13 (s, 1H), 3.89 (s, 2H), 2.31 (s, 3H). HPLC-MS (Positive mode) m/z 341/343 (M+H)⁺. Retention time 1.420 min.

### Example 76

### N-[4-(4-Cyclohexylphenyl)thiazol-2-yl]-2-(6-methoxybenzofuran-3-yl)acetamide

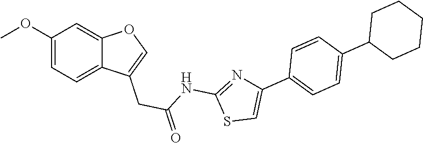 [SMILES: COc1ccc2c(CC(=O)Nc3nc(-c4ccc(C5CCCCC5)cc4)cs3)coc2c1]

The title compound is commercially available, e.g. from Enamine Ltd.

### Example 77

### N-(1,3-Benzothiazol-2-yl)-N-[4-(difluoromethylsulfanyl)phenyl]-2-(6-methoxybenzofuran-3-yl)acetamide

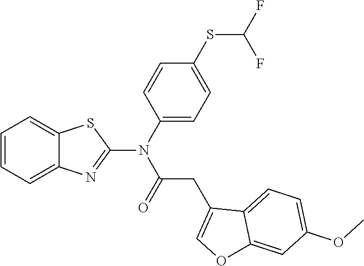 [SMILES: COc1ccc2c(CC(=O)N(c3ccc(SC(F)F)cc3)c3nc4ccccc4s3)coc2c1]

The title compound is commercially available, e.g. from Enamine Ltd.

### Example 78

### N-[4-(1,3-Benzodioxol-5-yl)thiazol-2-yl]-2-(6-methoxybenzofuran-3-yl)acetamide

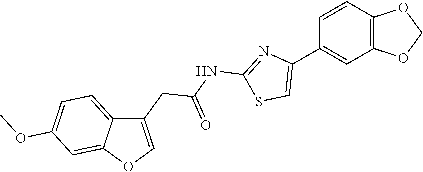 [SMILES: COc1ccc2c(CC(=O)Nc3nc(-c4ccc5c(c4)OCO5)cs3)coc2c1]

The title compound is commercially available, e.g. from Enamine Ltd.

### Example 79: 2-(6-Methoxy-7-methyl-benzofuran-3-yl)-N-(5-methylthiazol-2-yl)acetamide

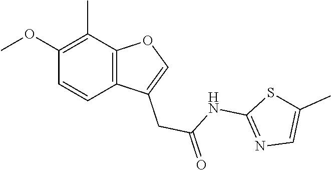

### 79.1 2-(6-Hydroxy-7-methyl-benzofuran-3-yl)acetic acid

The title compound was prepared according to General Method I starting from 2-methylbenzene-1,3-diol and chloroacetoacetate.

### 79.2 Methyl 2-(6-methoxy-7-methyl-benzofuran-3-yl)acetate

2-(6-hydroxy-7-methyl-benzofuran-3-yl)acetic acid (0.025 mmol) was dissolved in DMF then anhydrous potassium carbonate (3 equiv., 0.08 mmol) was added. The resulting solution was stirred for 20 min, after which methyl iodide was added dropwise (3 equiv., 0.075 mmol). The reaction mixture was heated to 100° C. and stirred for 8 h. Thereafter the mixture was cooled to r.t. and the precipitate filtered off. The remaining solution was concentrated in vacuo. The residue was taken-up in 100 mL of water and extracted with dichloromethane (3×50 mL). The combined organic layer was washed with water (3×25 mL), dried over sodium sulfate and filtered. After evaporation of solvents the compound was purified by flash chromatography.

### 79.3 2-(6-Methoxy-7-methyl-benzofuran-3-yl)acetic acid

The benzofuran acetic acid methyl ester (0.02 mmol) from example 79.2 was dissolved in ethanol-water solution (50:50) and potassium hydroxide (2 equiv., 0.04 mmol) was added. The solution was heated under reflux for 3 h. The reaction mixture was cooled and solvents were removed at reduced pressure. The residue was dissolved in 100 mL of water and extracted with DCM (50 mL×3). The aqueous phase was acidified using 3N aqueous HCl solution and extracted with EtOAc (50 mL×3). The combined organic fractions were washed with saturated brine (60 mL), dried over sodium sulfate and evaporated in vacuo to afford crude product. Corresponding crude acid was recrystallized from isopropanol to give 2-(6-methoxy-7-methyl-benzofuran-3-yl)acetic acid. Yield: 95%. HPLC-MS (Positive mode) m/z 221 (M+H)⁺. Retention time 1.242 min.

### 79.4 2-(6-Methoxy-7-methyl-benzofuran-3-yl)-N-(5-methylthiazol-2-yl)acetamide

The title compound was prepared according to General Method A using 2-(6-methoxy-7-methyl-benzofuran-3-yl)acetic acid and 5-methylthiazol-2-amine. Yield: 73%. ¹H NMR (400 MHz, DMSO-d₆): δ=12.19 (s, 1H), 7.78 (s, 1H), 7.37 (d, J=8.3 Hz, 1H), 7.12 (s, 1H), 6.97 (d, J=8.4 Hz, 1H), 3.82 (s, 3H), 3.79 (s, 2H), 2.31 (s, 3H), 2.27 (s, 3H). HPLC-MS (Positive mode) m/z 317 (M+H)⁺. Retention time 1.352 min.

### Example 80

### 2-(6,7-Dihydro-5H-cyclopenta[f]benzofuran-3-yl)-N-[2-[[5-(hydroxymethyl)thiazol-2-yl]amino]ethyl]acetamide

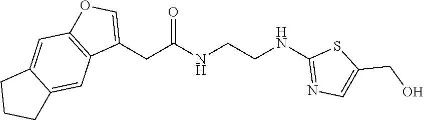 [SMILES: O=C(Cc1coc2cc3c(cc12)CCC3)NCCNc1ncc(CO)s1]

The title compound was prepared as outlined below:

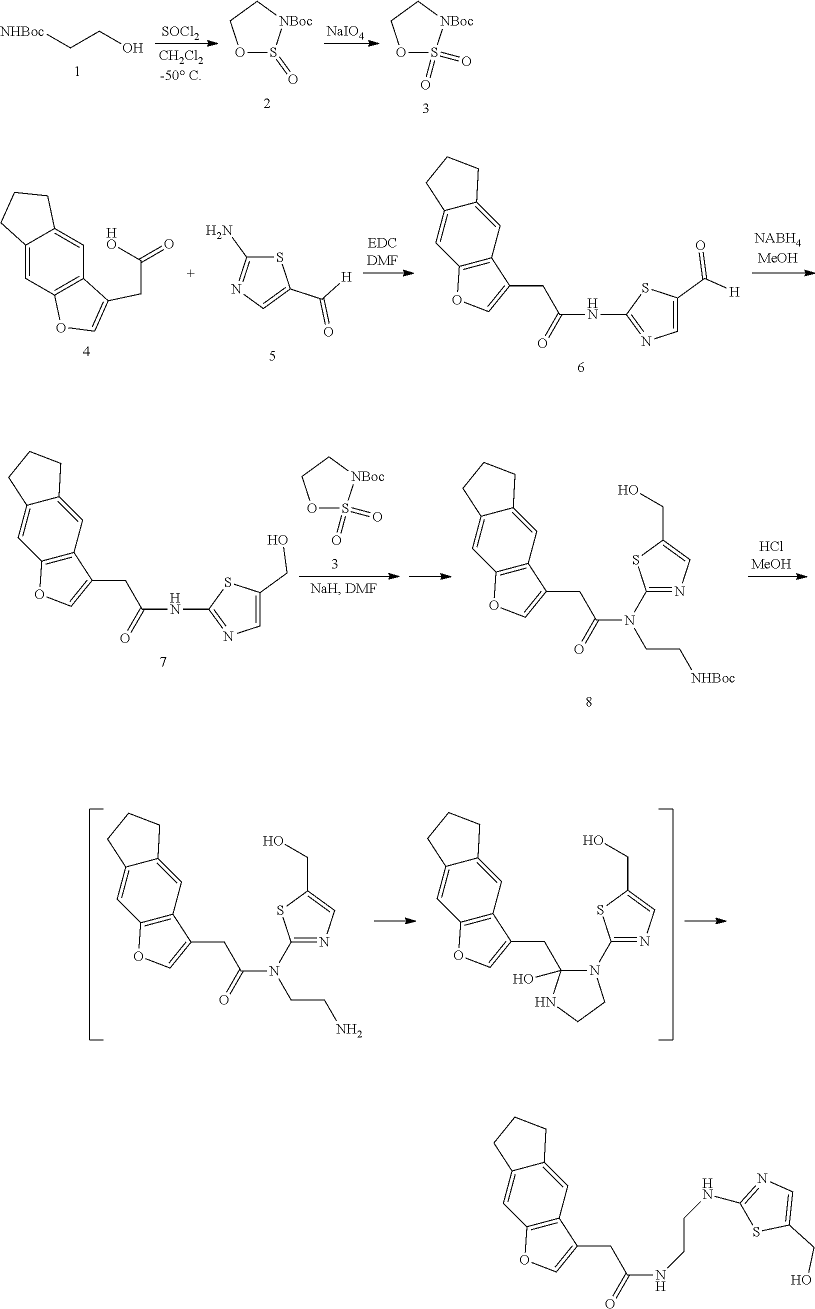

Compound 3 was obtained as colorless crystals according to literature procedure, e.g. Zeng J.-L. et al. Organic Letters, 2017, 19(8), 1974-1977.

Compounds 6 and 7 were obtained by the standard procedures as outlined above without purification of aldehyde 6. Compound 7 was purified with flash-chromatography with 11% overall Yield: over 2 stages after purification. Alkylation of 7 with 3 was conducted in DMF at room temperature with 1.2 eq of NaH (mixed at 0° C. then stirred at ambient temperature for 18 h). Compound 8 was purified by flash-chromatography (hexane-ethyl acetate 1:3) with resulted 100% purity; yield: 32%. After hydrolysis of the Boc-protection the resulting title compound was purified with HPLC chromatography; yield: 74%. HPLC-MS (Positive mode) m/z 372 (M+H)⁺. Retention time 1.083 min. ¹H NMR (400 MHz, DMSO-d₆): δ=2.05 (quint, J=8.0 Hz, 2H), 2.90 (m, 4H), 3.24 (br., s, 4H), 3.45 (s, 2H), 4.41 (d, J=5.6 Hz, 2H), 5.12 (t, J=5.6 Hz, 1H), 6.82 (s, 1H), 7.35 (s, 1H), 7.39 (s, 1H), 7.47 (br., s. 1H), 7.69 (s, 1H), 8.19 (br., s, 1H).

### Example 81

### N-(1,3-Benzothiazol-2-yl)-N-[4-(difluoromethylsulfanyl)phenyl]-2-(5-isopropyl-6-methyl-benzofuran-3-yl)acetamide

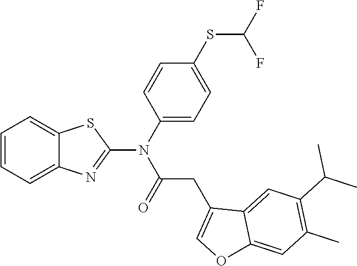 [SMILES: Cc1cc2occ(CC(=O)N(c3ccc(SC(F)F)cc3)c3nc4ccccc4s3)c2cc1C(C)C]

The title compound is commercially available, e.g. from Enamine Ltd.

### Example 82

### N-(1,3-Benzothiazol-2-yl)-N-[4-(difluoromethylsulfanyl)phenyl]-2-(6,7-dimethylbenzofuran-3-yl)acetamide

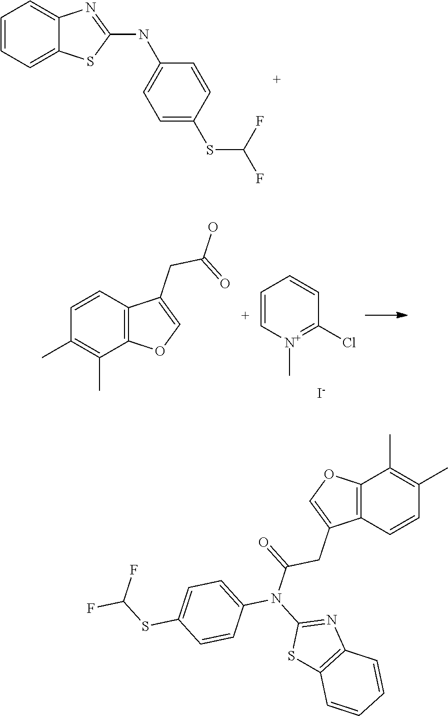

A mixture of N-[4-(difluoromethylsulfanyl)phenyl]-1,3-benzothiazol-2-amine (312 mg, 1.01 mmol), 2-(6,7-dimethylbenzofuran-3-yl)acetic acid (227 mg, 1.11 mmol), 2-chloro-1-methylpyridinium iodide (310 mg, 1.21 mmol) and DIPEA (327 mg, 2.53 mmol) was dissolved in 3 mL of acetonitrile and heated to 60° C. for 3 h. The reaction mixture was cooled to r.t. and 20 mL of water were added. The resulting slurry was extracted with dichloromethane (3×20 mL), the combined organic phase was washed with water (2×20 mL), dried over sodium sulfate and concentrated in vacuo. Crude title product was purified with HPLC chromatography (H₂O/MeOH, 70 100%, 0-6 min.) to give 109 mg of pure title compound. Yield: 22%. ¹H NMR (400 MHz, DMSO-d₆): δ=2.30 (s, 3H), 2.38 (s, 3H), 3.69 (s, 2H), 7.03 (d, J=4.8 Hz, 1H), 7.24 (d, J=4.8 Hz, 1H), 7.31 (t, 1H), 7.39 (t, 1H), 7.62 (m, 3H), 7.80 (m, 4H), 7.99 (s, J=8.0 Hz, 1H). HPLC-MS (Positive mode) m/z 495 (M+H)⁺. Retention time 1.808 min.

### Example 83

### 2-(7-Chloro-6-methyl-benzofuran-3-yl)-N-[5-(trifluoromethyl)thiazol-2-yl]acetamide

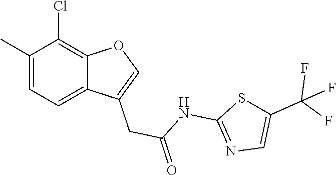 [SMILES: Cc1ccc2c(CC(=O)Nc3ncc(C(F)(F)F)s3)coc2c1Cl]

### 83.1 2-(7-Chloro-6-methyl-benzofuran-3-yl)acetic acid

The title compound was prepared according to General Method I.

### 83.2 2-(7-Chloro-6-methyl-benzofuran-3-yl)-N-[5-(trifluoromethyl)thiazol-2-yl]acetamide

The title compound was prepared according to General Method B using 2-(7-chloro-6-methyl-benzofuran-3-yl) acetic acid and 5-(trifluoromethyl)thiazol-2-amine. Yield: 34%. ¹H NMR (400 MHz, DMSO-d₆): δ=13.03 (s, 1H), 8.11 (s, 1H), 7.98 (s, 1H), 7.49 (d, J=7.9 Hz, 1H), 7.25 (d, J=7.8 Hz, 1H), 3.97 (s, 2H), 2.43 (s, 3H). HPLC-MS (Positive mode) m/z 375/377 (M+H)⁺. Retention time 1.625 min.

### Example 84

### 2-(6,7-Dihydro-5H-cyclopenta[f]benzofuran-3-yl)-N-(5-ethylthiazol-2-yl)acetamide

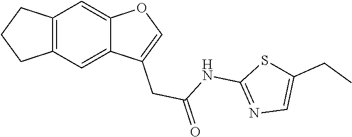 [SMILES: CCc1cnc(NC(=O)Cc2coc3cc4c(cc23)CCC4)s1]

2-(6,7-dihydro-5H-cyclopenta[f]benzofuran-3-yl)acetic acid (300 mg, 1.39 mmol) was dissolved in DMF (10 mL). 5-Ethylthiazol-2-amine (195 mg, 1.53 mmol) and DIPEA (0.47 ml, 2.8 mmol) were added. PyBOP (794 mg, 1.53 mmol) was added last and the reaction was allowed to run over night at room temperature. The solvent was removed in vacuo. The residue was dissolved in EtOAc and washed twice with sat. aq. sodium bicarbonate solution, once with water and once with sat. sodium chloride solution. The organic phase was evaporated and the residue was purified by flash chromatography (DCM:EtOAc 1:1). The solvent was removed in vacuo and the compound was obtained as a brownish powdery solid (201 mg, 0.62 mmol, 44% yield). UPLC-MS (Positive mode) m/z 327. Retention time 1.852 min.

### Example 85

### 2-(6,7-Dichlorobenzofuran-3-yl)-N-[5-(trifluoromethyl)thiazol-2-yl]acetamide

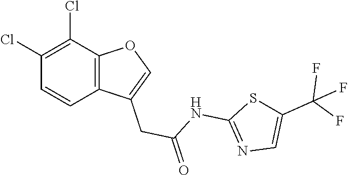 [SMILES: O=C(Cc1coc2c(Cl)c(Cl)ccc12)Nc1ncc(C(F)(F)F)s1]

### 85.1 2-(6,7-Dichlorobenzofuran-3-yl)acetic acid

The title compound was prepared according to General Method II.

### 85.2 2-(6,7-Dichlorobenzofuran-3-yl)-N-[5-(trifluoromethyl)thiazol-2-yl]acetamide

The title compound was prepared according to General Method B using 2-(6,7-dichlorobenzofuran-3-yl)acetic acid and 5-(trifluoromethyl)thiazol-2-amine. Yield: 22%. ¹H NMR (500 MHz, DMSO-d₆): δ=13.05 (s, 1H), 8.13 (s, 2H), 7.65 (d, J=8.5 Hz, 1H), 7.54 (d, J=8.5 Hz, 1H), 4.02 (s, 2H). LC-MS (Positive mode) m/z 395/396 (M+H)⁺. HPLC retention time 1.577 min.

### Example 86

### 2-(7-Methoxy-6-methyl-benzofuran-3-yl)-N-(5-methylthiazol-2-yl)acetamide

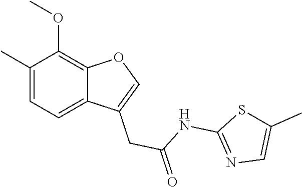

### 86.1 4-(Chloromethyl)-8-hydroxy-7-methyl-chromen-2-one

3-methylbenzene-1,2-diol (100 mmol) was dissolved in ethyl chloroacetoacetate (101 mmol) and the resulting solution was added dropwise to 50 mL of sulfuric acid (H₂SO₄) under stirring and ice cooling. The temperature was controlled within 0-10° C. The mixture was stirred for 8 hours at room temperature and then was poured into ice (200 g). The formed precipitate was filtered and washed with water (5×100 mL). Crude product was purified by crystallization. Yield: 60%

### 86.2 2-(7-Hydroxybenzofuran-3-yl)acetic acid

The product of example 86.1 (0.1 mol) was added to a solution of KOH in water (3 eq in 100 mL. The mixture was refluxed for 8-12 hours and then neutralized with hydrochloric acid. The precipitate was filtered and washed tree times with water (3×100 mL) and diethyl ether subsequently. Residue was recrystallized and dried to give the title product. Yield: 90%

### 86.3 Methyl 2-(7-methoxy-6-methyl-benzofuran-3-yl)acetate

The product of example 86.2 (0.025 mmol) was dissolved in DMF and then anhydrous potassium carbonate (3 equiv., 0.08 mmol) was added. The resulting solution was stirred for 20 min, after which methyl iodide was added dropwise (3 equiv., 0.075 mmol). The reaction mixture was heated to 100° C. and stirred for 8 h. Thereafter the mixture was cooled to r.t. and the precipitate filtered off. The remaining solution was concentrated in vacuo. The residue was taken-up in 100 mL of water and extracted with dichloromethane (3×50 mL). The combined organic layer was washed with water (3×25 mL), dried over sodium sulfate and filtered. After evaporation of solvents the compound was purified by flash chromatography.

### 86.4 2-(7-Methoxy-6-methyl-benzofuran-3-yl)acetic acid

The product from example 86.3 (0.02 mmol) was dissolved in ethanol-water solution (50:50) and potassium hydroxide (2 equiv., 0.04 mmol) was added. The solution was heated under reflux for 3 h. The reaction mixture was cooled and solvents were removed at reduced pressure. The residue was dissolved in 100 mL of water and extracted with DCM (50 mL×3). The aqueous phase was acidified using 3N aqueous HCl solution and extracted with EtOAc (50 mL×3). The combined organic fractions were washed with saturated brine (60 mL), dried over sodium sulfate and evaporated in vacuo to afford crude product. The crude acid was recrystallized from isopropanol to give 2-(7-methoxy-6-methyl-benzofuran-3-yl)acetic acid. Yield: 95%.

### 86.5 2-(7-Methoxy-6-methyl-benzofuran-3-yl)-N-(5-methylthiazol-2-yl)acetamide

The title compound was prepared according to General Method A using 2-(7-methoxy-6-methyl-benzofuran-3-yl)acetic acid and 5-methylthiazol-2-amine. Yield: 83%. ¹H NMR (500 MHz, DMSO-d₆): δ=12.20 (s, 1H), 7.85 (s, 1H), 7.17 (d, J=7.6 Hz, 1H), 7.13 (s, 1H), 7.05 (d, J=8.3 Hz, 1H), 4.04 (s, 3H), 3.82 (s, 2H), 2.32 (s, 3H), 2.27 (s, 3H). LC-MS (Positive mode) m/z 316/317 (M+H)⁺. HPLC retention time 1.379 min.

### Example 87

### 2-(6,7-Dimethylbenzofuran-3-yl)-N-[5-(methoxymethyl)thiazol-2-yl]acetamide

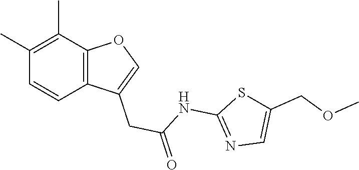

The title compound was prepared according to General Method A using 2-(6,7-dimethylbenzofuran-3-yl)acetic acid and 5-(methoxymethyl)thiazol-2-amine. Yield: 79%. ¹H NMR (500 MHz, CDCl3): δ=9.52 (s, 1H), 7.62 (s, 1H), 7.24 (m, 2H), 7.08 (d, J=7.8 Hz, 1H), 4.55 (s, 2H), 3.88 (s, 2H), 3.35 (s, 3H), 2.44 (s, 3H), 2.39 (s, 3H). LC-MS (Positive mode) m/z 331 (M+H)⁺. HPLC retention time 1.378 min.

### Example 88

### 2-(6,7-Dimethylbenzofuran-3-yl)-N-(5-methylthiazol-2-yl)acetamide

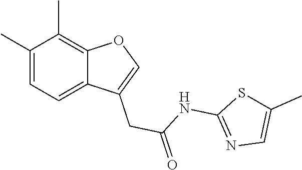

2-(6,7-Dimethylbenzofuran-3-yl)acetic acid (300 mg, 1.47 mmol) was dissolved in DMF (10 mL). 5-Methylthiazol-2-amine (184 mg, 1.62 mmol) and DIPEA (0.5 mL, 2.9 mmol) were added. PyBOP (841 mg, 1.62 mmol) was added last and the reaction was allowed to run over night at room temperature. The solvent was removed in vacuo. The residue was dissolved in EtOAc and washed twice with sat. aq. sodium bicarbonate solution, once with water and once with sat. sodium chloride solution. The organic phase was evaporated and the residue was purified by flash chromatography (DCM:EtOAc 1:1). The solvent was removed in vacuo and the compound was obtained as a brownish grey powder (212.7 mg, 0.71 mmol, 48% yield). UPLC-MS (Positive mode) m/z 301 (M+H)⁺. Retention time 1.651 min.

### Example 89

### 2-(7-Chloro-6-methyl-benzofuran-3-yl)-N-[5-(methoxymethyl)thiazol-2-yl]acetamide

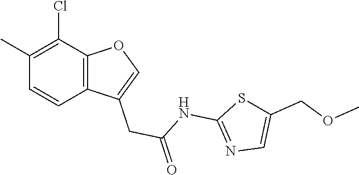

### 89.1 2-(7-Chloro-6-methyl-benzofuran-3-yl)acetic acid

The title compound was prepared according to General Method I.

### 89.2 2-(7-Chloro-6-methyl-benzofuran-3-yl)-N-[5-(methoxymethyl)thiazol-2-yl]acetamide

The title compound was prepared according to General Method A using 2-(7-chloro-6-methyl-benzofuran-3-yl)acetic acid and 5-(methoxymethyl)thiazol-2-amine. Yield: 75%. ¹H NMR (500 MHz, CDCl3): δ=10.85 (s, 1H), 7.68 (s, 1H), 7.33 (d, J=6.8 Hz, 1H), 7.27 (d, J=1.4 Hz, 1H), 7.14 (d, J=7.6 Hz, 1H), 4.56 (s, 2H), 3.90 (s, 2H), 3.36 (d, J=1.4 Hz, 3H), 2.49 (s, 3H). HPLC-MS (Positive mode) m/z 351/353 (M+H)⁺. Retention time 1.396 min.

### Example 90

### 2-(7-Chloro-6-methyl-benzofuran-3-yl)-N-(5-methylthiazol-2-yl)acetamide

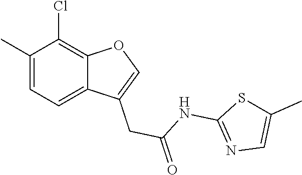

### 90.1 2-(7-Chloro-6-methyl-benzofuran-3-yl)acetic acid

The title compound was prepared according to General Method I.

### 90.2 2-(7-Chloro-6-methyl-benzofuran-3-yl)-N-(5-methylthiazol-2-yl)acetamide

The title compound was prepared according to General Method A using 2-(7-chloro-6-methyl-benzofuran-3-yl)acetic acid and 5-methylthiazol-2-amine. Yield: 81%. ¹H NMR (400 MHz, DMSO-d₆): δ=12.20 (s, 1H), 7.95 (s, 1H), 7.48 (d, J=7.9 Hz, 1H), 7.25 (d, J=7.9 Hz, 1H), 7.13 (s, 1H), 3.85 (s, 2H), 2.44 (s, 3H), 2.31 (s, 3H). LC-MS (Positive mode) m/z 321/323 (M+H)⁺. HPLC retention time 1.492 min.

### Example 91

### 2-(6-Methoxy-7-methyl-benzofuran-3-yl)-N-[5-(trifluoromethyl)thiazol-2-yl]acetamide

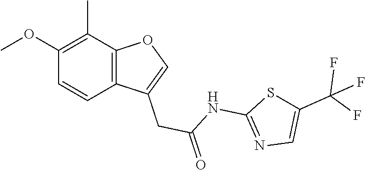 [SMILES: COc1ccc2c(CC(=O)Nc3ncc(C(F)(F)F)s3)coc2c1C]

The title compound was prepared according to General Method B using 2-(6-methoxy-7-methyl-benzofuran-3-yl)acetic acid and 5-(trifluoromethyl)thiazol-2-amine. Yield: 36%. ¹H NMR (400 MHz, DMSO-d₆): δ=13.02 (br s, 1H), 8.12 (s, 1H), 7.82 (s, 1H), 7.38 (d, J=8.5 Hz, 1H), 6.99 (d, J=8.4 Hz, 1H), 3.91 (s, 2H), 3.83 (s, 3H), 2.28 (s, 3H). HPLC-MS (Positive mode) m/z 371 (M+H)⁺. Retention time 1.542 min.

### Example 92

### 2-(6-Chloro-7-methyl-benzofuran-3-yl)-N-(5-methylthiazol-2-yl)acetamide

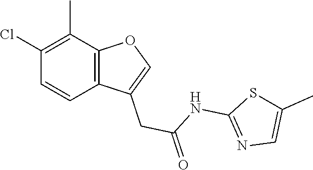 [SMILES: Cc1cnc(NC(=O)Cc2coc3c(C)c(Cl)ccc23)s1]

The title compound was prepared according to General Method A using 2-(6-chloro-7-methyl-benzofuran-3-yl)acetic acid and 5-methylthiazol-2-amine. Yield: 77%. ¹H NMR (400 MHz, DMSO-d₆): δ=12.21 (br s, 1H), 7.96 (s, 1H), 7.46 (d, J=8.2 Hz, 1H), 7.32 (d, J=8.3 Hz, 1H), 7.12 (s, 1H), 3.84 (s, 2H), 2.47 (s, 3H), 2.31 (s, 3H). HPLC-MS (Positive mode) m/z 321/323 (M+H)⁺. Retention time 1.458 min.

### Example 93

### 2-(6,7-Dichlorobenzofuran-3-yl)-N-[5-(methoxymethyl)thiazol-2-yl]acetamide

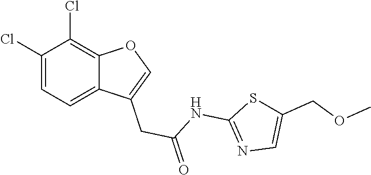 [SMILES: COCc1cnc(NC(=O)Cc2coc3c(Cl)c(Cl)ccc23)s1]

### 93.1 2-(6,7-Dichlorobenzofuran-3-yl)acetic acid

The title compound was prepared according to General Method II.

### 93.2 2-(6,7-Dichlorobenzofuran-3-yl)-N-[5-(methoxymethyl)thiazol-2-yl]acetamide

The title compound was prepared according to General Method A using 2-(6,7-dichlorobenzofuran-3-yl)acetic acid and 5-(methoxymethyl)thiazol-2-amine. Yield: 84%. ¹H NMR (400 MHz, DMSO-d₆): δ=3.22 (s, 3H), 3.93 (s, 2H), 4.52 (s, 2H), 7.40 (s, 1H), 7.54 (d, J=8.2 Hz, 1H), 7.64 (d, J=8.2 Hz, 1H), 8.11 (s, 1H), 12.39 (br s, 1H).

HPLC-MS (Positive mode) m/z 371/373(M+H)⁺. Retention time 1.430 min.

### Example 94

### 2-(6-Chloro-7-methyl-benzofuran-3-yl)-N-[5-(trifluoromethyl)thiazol-2-yl]acetamide

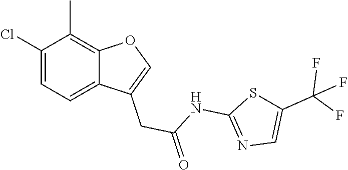 [SMILES: Cc1c(Cl)ccc2c(CC(=O)Nc3ncc(C(F)(F)F)s3)coc12]

The title compound was prepared according to General Method B using 2-(6-chloro-7-methyl-benzofuran-3-yl)acetic acid and 5-(trifluoromethyl)thiazol-2-amine. Yield: 39%. ¹H NMR (400 MHz, DMSO-d₆): δ=2.46 (s, 3H), 3.97 (s, 2H), 7.31 (d, J=8.2 Hz, 1H), 7.46 (d, J=8.2 Hz, 1H), 7.99 (s, 1H), 8.10 (s, 1H), 13.03 (br s, 1H). HPLC-MS (Positive mode) m/z 375/377(M+H)⁺. Retention time 1.599 min.

### Example 95

### 2-(6,7-Dimethylbenzofuran-3-yl)-N-(5-isopropylthiazol-2-yl)acetamide

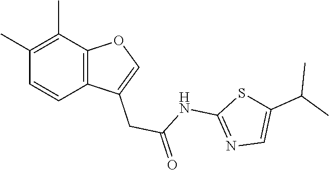 [SMILES: Cc1ccc2c(CC(=O)Nc3ncc(C(C)C)s3)coc2c1C]

2-(6,7-Dimethylbenzofuran-3-yl)acetic acid (300 mg, 1.47 mmol) was dissolved in DMF (10 mL). 5-Isopropylthiazol-2-amine (229.8 mg, 1.62 mmol) and DIPEA (0.5 mL, 2.94 mmol) were added. PyBOP (840.9 mg, 1.62 mmol) was added last and the reaction was allowed to run over night at room temperature. The solvent was removed in vacuo. The residue was dissolved in EtOAc and washed twice with sat. aq. sodium bicarbonate solution, once with water and once with sat. sodium chloride solution. The organic phase was evaporated and the residue was purified by flash chromatography (DCM:EtOAc 1:1). The solvent was removed in vacuo and the title compound was obtained as an orange powder (166.6 mg, 0.51 mmol, 35% yield). UPLC-MS (Positive mode) m/z 329(M+H)⁺. Retention time 1.785 min.

### Example 96

### 2-(6,7-Dimethylbenzofuran-3-yl)-N-(5-methylthiazol-2-yl)acetamide

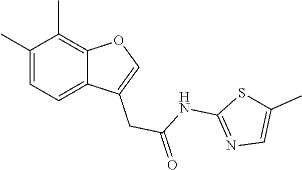

The title compound was prepared according to General Method A using 2-(6,7-dimethylbenzofuran-3-yl)acetic acid and 5-methylthiazol-2-amine. Yield: 90%. ¹H NMR (400 MHz, DMSO-d₆): δ=2.32 (s, 3H), 2.33 (s, 3H), 2.36 (s, 3H), 3.81 (s, 2H), 7.06 (d, J=8.2 Hz, 1H), 7.13 (s, 1H), 7.31 (d, J=8.2 Hz, 1H), 7.82 (s, 1H), 12.18 (br s, 1H).

HPLC-MS (Positive mode) m/z 301 (M+H)⁺. HPLC retention time 1.654 min.

### Example 97

### 2-(7-Methoxy-6-methyl-benzofuran-3-yl)-N-[5-(trifluoromethyl)thiazol-2-yl]acetamide

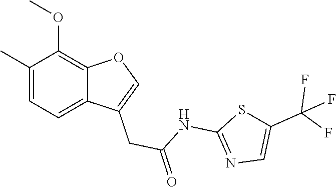 [SMILES: COc1c(C)ccc2c(CC(=O)Nc3ncc(C(F)(F)F)s3)coc12]

The title compound was prepared according to General Method B using 2-(7-methoxy-6-methyl-benzofuran-3-yl)acetic acid and 5-(trifluoromethyl)thiazol-2-amine. Yield: 48%. ¹H NMR (400 MHz, DMSO-d₆): δ=2.27 (s, 3H), 3.93 (s, 2H), 4.05 (s, 3H), 7.06 (d, J=7.8 Hz, 1H), 7.17 (d, J=7.8 Hz, 1H), 7.89 (s, 1H), 8.12 (s, 1H), 13.03 (br s, 1H).

HPLC-MS (Positive mode) m/z 371 (M+H)⁺. Retention time 1.583 min.

### Example 98

### 2-(6,7-Dimethylbenzofuran-3-yl)-N-(5-ethylthiazol-2-yl)acetamide

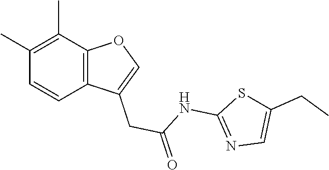 [SMILES: CCc1cnc(NC(=O)Cc2coc3c(C)c(C)ccc23)s1]

2-(6,7-Dimethylbenzofuran-3-yl)acetic acid (72.4 mg, 0.355 mmol) was dissolved in DMF (5 mL). 5-Ethylthiazol-2-amine (50 mg, 0.39 mmol) and DIPEA (0.121 mL, 0.7 mmol) were added. PyBOP (203 mg, 0.39 mmol) was added and the reaction was allowed to run over night at room temperature. The solvent was removed in vacuo. The residue was dissolved in EtOAc and washed twice with sat. aq. sodium bicarbonate solution, once with water and once with sat. sodium chloride solution. The organic phase was evaporated and the residue was purified by flash chromatography (heptane:EtOAc 1:1). The solvent was removed in vacuo and the compound was obtained as a white powder (46 mg, 0.146 mmol, 38% yield). UPLC-MS (Positive mode) m/z 315 (M+H)⁺. Retention time 1.731 min.

### Example 99

### 2-(6,7-Dimethylbenzofuran-3-yl)-N-[5-(trifluoromethyl)thiazol-2-yl]acetamide

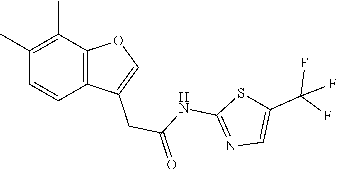 [SMILES: Cc1ccc2c(CC(=O)Nc3ncc(C(F)(F)F)s3)coc2c1C]

The title compound was prepared according to General Method B using 2-(6,7-dimethylbenzofuran-3-yl)acetic acid and 5-(trifluoromethyl)thiazol-2-amine. Yield: 44%. ¹H NMR (400 MHz, DMSO-d₆): δ=2.33 (s, 3H), 2.36 (s, 3H), 3.92 (s, 2H), 7.06 (d, J=7.8 Hz, 1H), 7.31 (d, J=7.8 Hz, 1H), 7.85 (s, 1H), 8.11 (s, 1H), 13.01 (br s, 1H).

UPLC-MS (Positive mode) m/z 355 (M+H)⁺. Retention time 1.836 min.

### Example 100

### N-[5-[5-(Aminomethyl)-2-furyl]thiazol-2-yl]-2-(6,7-dihydro-5H-cyclopenta[f]benzofuran-3-yl)acetamide, hydrochloride salt

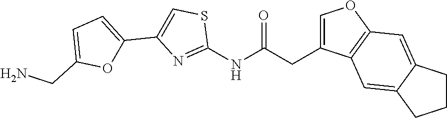 [SMILES: NCc1ccc(-c2csc(NC(=O)Cc3coc4cc5c(cc34)CCC5)n2)o1]

### 100.1 4-[5-(Aminomethyl)-2-furyl]thiazol-2-amine

1.21 g of N-[[5-(2-aminothiazol-4-yl)-2-furyl]methyl]acetamide (synthesized using the procedure reported in Bioorg. Med. Chem. Lett. 1998, 8, 1307-1312, Katsura Y. at all.) was refluxed in hydrochloric acid water solution (30 mL, 560 mg of HCl, 3 eq). After completion of the reaction (LCMS control), the mixture was cooled down and quenched with KOH. The precipitate was filtered, washed with water and dried in vacuo to afford the title compound (463 mg, 46%).

### 100.2 tert-Butyl N-[[5-(2-aminothiazol-4-yl)-2-furyl]methyl]carbamate

460 mg of the compound of example 100.1 were dissolved in MeOH, followed by addition of Boc₂O (479 mg). The mixture was allowed to stir at r.t. for 2 hours, and the solvents were distilled off to afford the title compound (700 mg, quant. yield).

### 100.3 N-[5-[5-(Aminomethyl)-2-furyl]thiazol-2-yl]-2-(6,7-dihydro-5H-cyclopenta[f]benzofuran-3-yl)acetamide, hydrochloride salt

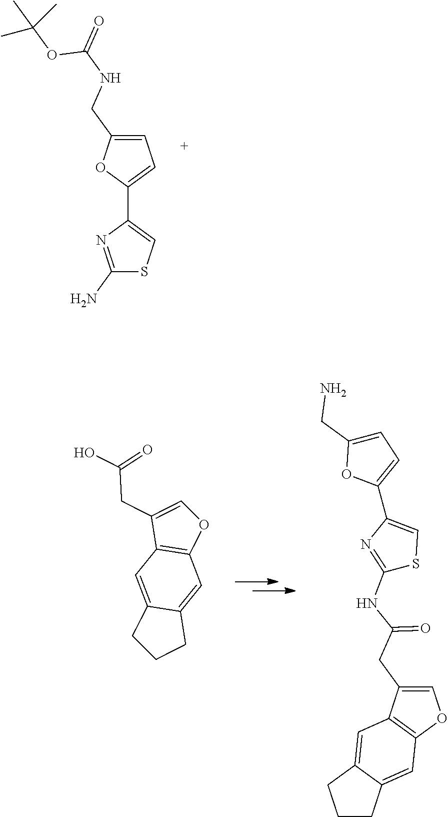 [SMILES: CC(C)(C)OC(=O)NCc1ccc(-c2csc(N)n2)o1 ; O=C(O)Cc1coc2cc3c(cc12)CCC3 ; NCc1ccc(-c2csc(NC(=O)Cc3coc4cc5c(cc34)CCC5)n2)o1]

To a cooled solution of compound from example 100.2 in DMF (203 mg), 2-(6,7-dihydro-5H-cyclopenta[f]benzofuran-3-yl)acetic acid (193 mg), HOAt (149 mg) and EDC (170 mg) were added sequentially. Resulting mixture was allowed to stir at r.t. for overnight. Then, the solution was poured into water and extracted with EtOAc. (3×30 mL). Organic layer was washed with water, brine, dried over Na₂SO₄, and evaporated in vacuo. The residue was dissolved in DCM, followed by addition of HCl in dioxane (4M). The formed precipitate was collected, washed with acetone and dried in vacuo to provide the title compound. (105 mg, 33%), overall Yield: in all steps 14%.

¹H NMR (400 MHz, DMSO-d₆): δ=2.05 (m, 2H), 2.90 (q., J¹=7.8 Hz, J²=7.4 Hz, 4H), 3.85 (s, 2H), 4.13 (d, J=5.2 Hz, 2H), 6.67 (dd, J¹=6.7 Hz, J²=2.7 Hz, 2H), 7.32 (s, 1H), 7.38 (s, 1H), 7.43 (s, 1H), 7.81 (s, 1H), 8.47 (br. s, 3H), 12.67 (s, 1H). HPLC-MS (Positive mode) m/z 394 (M+H)⁺. Retention time 1.144 min.

### Example 101

### N-[4-[4-(Aminomethyl)phenyl]thiazol-2-yl]-2-(5,6-dimethylbenzofuran-3-yl)acetamide, hydrochloride salt

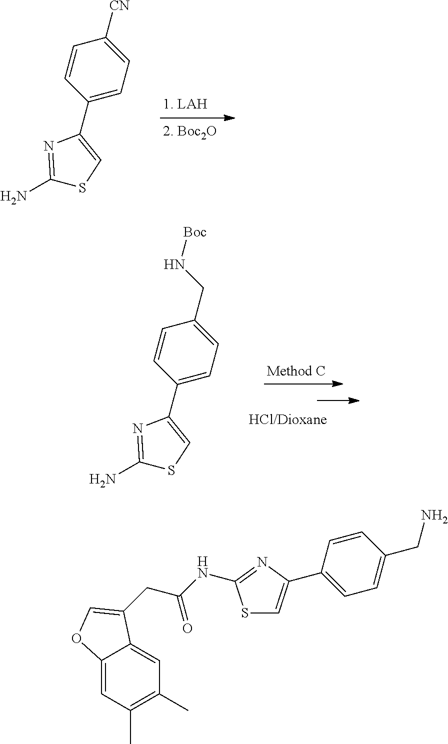 [SMILES: N#Cc1ccc(-c2csc(N)n2)cc1 ; CC(C)(C)OC(=O)NCc1ccc(-c2csc(N)n2)cc1 ; Cc1cc2occ(CC(=O)Nc3nc(-c4ccc(CN)cc4)cs3)c2cc1C]

The title compound was prepared according to general amide coupling Method C using 2-(5,6-dimethylbenzofuran-3-yl)acetic acid and 4-(2-aminothiazol-4-yl)-N(tert-butoxycarbonyl)-benzylamine.

To a mechanically-stirred slurry of lithium aluminum hydride (2.64 g, 69.7 mmol) in anhydrous dioxane (150 mL) at room temperature, a warmed slurry of 4-(2-aminothiazol-4-yl)-benzonitrile (4 g, 19.9 mmol) in dioxane (200 mL) was added portionwise. The reaction mixture was heated at 75° C. for 4 h and then cooled the to 0° C. Reaction was quenched with water (2.6 mL) and 15% aqueous NaOH (2.6 mL) was added (8 mL). Resulting mixture was stirred for 2 h at room temperature and slurry was then filtered over Celite®. Celite filter pad was washed few times with dioxane (500 mL) and filtrate concentrated in vacuo. Residue was dissolved in dioxane (300 mL) and then solution of di-tert-butyl dicarbonate (5.2 g, 23.8 mmol) in dioxane (100 mL) was added. Resulting mixture stirred at room temperature for 24 h and concentrated in vacuo. Residue dissolved in EtOAc (500 mL) and washed with saturated aqueous NaHCO₃(250 mL). The organic phase was dried over Na₂SO4, filtered and concentrated in vacuo. Crude product was purified with flash chromatography (EtOAc/hexane 2:3) to afford the desired intermediate (2.98 g, 49%) as a yellow solid.

Amide obtained after coupling with corresponding benzofuran acetic acid (80 mg, 0.16 mmol., 1 equiv.) was dissolved in dry dioxane (30 mL). Thereafter solution of hydrochloric acid (13%) in dioxane (80 mL) was added and resulting mixture was stirred for 1.5 h. After precipitate was formed the mixture was filtrated and dried under vacuum at +50° C. for 4 h. The product was obtained in amount of 45 mg, 0.115 mmol (Yield: 73%), overall Yield: in all steps 17%.

HPLC-MS (Positive mode) m/z 392 (M+H)⁺. Retention time 1.223 min.

¹H NMR (400 MHz, DMSO-d₆): δ=2.33 (s, 6H), 3.20 (s, 2H), 3.82 (s, 2H), 4.00 (d, J=3.8 Hz, 2H), 7.21 (s, 1H), 7.39 (d, J=4.1 Hz, 2H), 7.56 (d, J=7.6 Hz, 2H), 7.67 (s, 1H), 7.87 (d, J=7.6 Hz, 2H), 8.76 (s, 3H), 12.42 (s, 1H).

### Example 102

### N-(1,3-Benzothiazol-2-yl)-N-[4-(difluoromethylsulfanyl)phenyl]-2-(6-methylbenzofuran-3-yl)acetamide

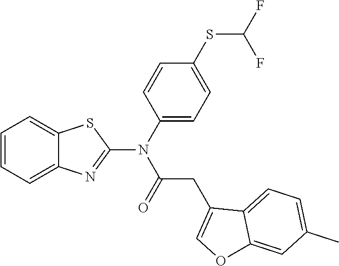 [SMILES: Cc1ccc2c(CC(=O)N(c3ccc(SC(F)F)cc3)c3nc4ccccc4s3)coc2c1]

The title compound is commercially available, e.g. from Enamine Ltd.

### Example 103

### 2-(2-(6,7-dimethylbenzofuran-3-yl)acetamido)-N-methyl-5-(trifluoromethyl)thiazole-4-carboxamide

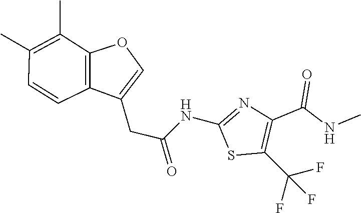

### 103.1 2-Amino-N-methyl-5-(trifluoromethyl)thiazole-4-carboxamide

A solution of 2-amino-5-(trifluoromethyl)thiazole-4-carboxylic acid (400 mg, 1.89 mmol) and methylamine (9.4 mL, 2 M solution in THF, 0.18 mmol) in DMF (5 mL) was treated with DIPEA (0.64 mL, 3.77 mmol) and benzotriazol-1-yl-oxytripyrrolidinophosphonium hexafluorophosphate (1.08 g, 2.07 mmol), stirred at 23° C. for 14 days and evaporated. Column chromatography (C₁₈; MeCN/H₂O 5:95->60:40) of the crude gave 2-amino-N-methyl-5-(trifluoromethyl)thiazole-4-carboxamide (369 mg, 87%) as a colorless solid.

MS (ESI+, H₂O/MeCN) m/z (%): 451.1 (22, [2M+H]⁺) 226.1 (100, [M+H]⁺)

### 103.2 2-(2-(6,7-dimethylbenzofuran-3-yl)acetamido)-N-methyl-5-(trifluoromethyl)thiazole-4-carboxamide

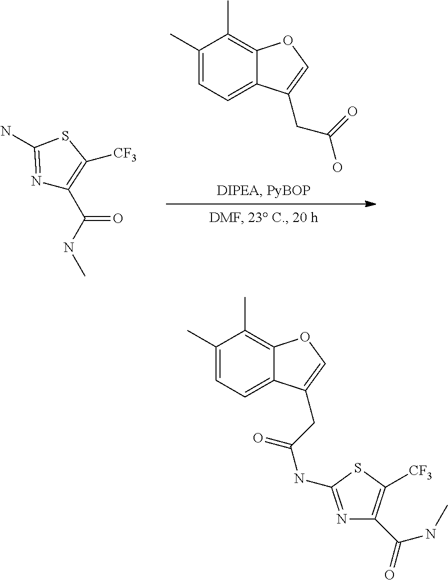

A solution of 2-(6,7-dimethylbenzofuran-3-yl)acetic acid (150 mg, 0.73 mmol) and 2-amino-N-methyl-5-(trifluoromethyl)thiazole-4-carboxamide (165 mg, 0.73 mmol) in DMF (5 mL) was treated with DIPEA (0.25 mL, 1.47 mmol) and benzotriazol-1-yl-oxytripyrrolidinophosphonium hexafluoro-phosphate (420 mg, 0.81 mmol) and stirred at 23° C. for 20 h. HPLC purification (1.0 mL, method B) gave 2-(2-(6,7-dimethylbenzofuran-3-yl)acetamido)-N-methyl-5-(trifluoromethyl)thiazole-4-carboxamide (12.4 mg, 21%) as colorless solid.

¹H NMR (400 MHz, DMSO-d₆) δ=13.01 (s, 1H, NH), 8.20 (br. s, J=4.9 Hz, 1H, NH), 7.80 (s, 1H, H—Ar), 7.24 (d, J=7.9 Hz, 1H, H—Ar), 7.00 (d, J=7.9 Hz, 1H, H—Ar), 3.88 (s, 2H, CH₂), 2.72 (d, J=4.8 Hz, 3H, N—CH₃), 2.30 (s, 3H, CH₃), 2.26 (s, 3H, CH₃) ppm.

MS (ESI+, H₂O/MeCN) m/z (%): 412 (100, [M+H]⁺).

### Example 104

### 2-(Furo[2,3-b]pyridin-3-yl)-N-(5-methylthiazol-2-yl)acetamide

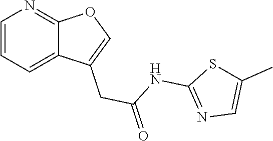 [SMILES: Cc1cnc(NC(=O)Cc2coc3ncccc23)s1]

### 104.1 Ethyl 3-hydroxyfuro[2,3-b]pyridine-2-carboxylate

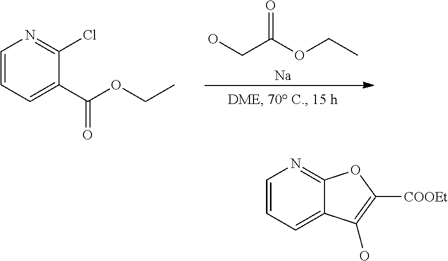

A suspension of sodium hydride (11.2 g, 60% dispersion in mineral oil, 280 mmol) in 1,2-dimethoxyethane (250 mL) was cooled to 0° C., treated dropwise with ethyl glycolate (25.5 mL, 269 mmol) and stirred at 23° C. for 30 min. Ethyl-2-chloronicotinate (20.0 g, 108 mmol) in 1,2-dimethoxyethane (40 mL) was added dropwise over 10 min and the mixture was stirred at 70° C. for 15 hours. The solvent was evaporated, the residue dissolved in water (500 mL) and washed with toluene. The aqueous layer was acidified with acetic acid (19 mL) to pH 5 and extracted five times with CH₂Cl₂ (5×100 mL). The combined organic layers were dried over anhydrous MgSO₄, filtered and the solvent evaporated. Column chromatography (SiO₂; EtOAc/Heptane, 20:80->50:50) of the crude gave ethyl 3-hydroxyfuro[2,3-b]pyridine-2-carboxylate (21.1 g, 94%) as a yellow solid.

¹H NMR (400 MHz, Chloroform-d) δ=8.52 (dd, J=4.9, 1.7 Hz, 1H, H—Ar), 8.12 (dd, J=7.8, 1.7 Hz, 1H, H—Ar), 7.31 (dd, J=7.8, 4.8 Hz, 1H, H—Ar), 4.47 (q, J=7.1 Hz, 2H, O—CH₂CH₃), 4.13 (s, 1H, OH), 1.44 (t, J=7.1 Hz, 3H, O—CH₂CH₃) ppm.

MS (ESI+, H₂O/MeCN) m/z (%): 208.0 (100, [M+H]⁺).

### 104.2 Furo[2,3-b]pyridin-3(2H)-one

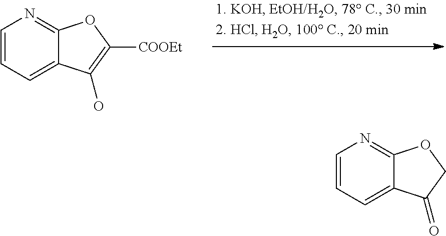 [SMILES: CCOC(=O)c1oc2ncccc2c1O ; O=C1COc2ncccc21]

A solution of ethyl 3-hydroxyfuro[2,3-b]pyridine-2-carboxylate (12.8 g, 62 mmol) in EtOH (100 mL) and water (10 mL) was treated with KOH (17.3° g, 309 mmol) and stirred at reflux for 20 min. The solvent was evaporated; the residue was dissolved in water (250 mL), acidified with conc. HCl (45 mL) and stirred at reflux for 10 minutes. The excess of HCl was evaporated and the residue dissolved in CH₂Cl₂, the organic phase was washed with water, dried over anhydrous MgSO₄, filtered and evaporated. Column chromatography (SiO₂; 0.5% Et₃N, EtOAc/Heptane 20:80->50:50) of the crude gave furo[2,3-b]pyridin-3(2H)-one (552 mg, 7%) as a colorless solid.

Alternatively furo[2,3-b]pyridin-3(2H)-one was prepared as follows:

A solution of ethyl 3-hydroxyfuro[2,3-b]pyridine-2-carboxylate (250 mg, 1.21 mmol) in EtOH (10 mL) and water (1 mL) was treated with KOH (17.3° g, 309 mmol) and stirred at reflux for 20 min. The solvent was evaporated; the residue was dissolved in water (5 mL), acidified with conc. HCl (0.9 mL) and stirred at reflux for 10 minutes. The excess of HCl was evaporated, column chromatography (SiO₂; 0.5% Et₃N, EtOAc/Heptane 20:80->50:50) of the crude gave furo[2,3-b]pyridin-3(2H)-one (48 mg, 29%) as a colorless solid.

¹H NMR (400 MHz, Chloroform-d) δ=8.52 (dd, J=4.9, 1.9 Hz, 1H, H—Ar), 7.99 (dd, J=7.5, 1.9 Hz, 1H, H—Ar), 7.09 (dd, J=7.5, 4.9 Hz, 1H, H—Ar), 4.69 (s, 2H, O—CH₂) ppm.

MS (ESI+, H₂O/MeCN) m/z (%): 136.0 (100, [M+H]⁺).

### 104.3 2-(Furo[2,3-b]pyridin-3-yl)acetonitrile

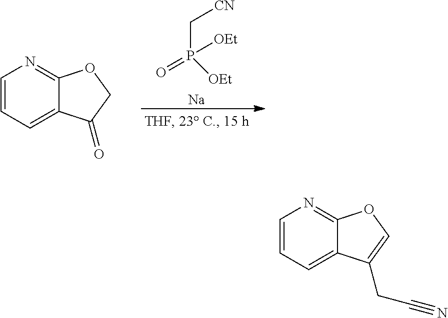

The reaction was performed under Ar atmosphere.

A suspension of sodium hydride (0.155 g, 60% dispersion in mineral oil, 3.89 mmol) in anhydrous tetrahydrofuran (4 mL) was treated dropwise with diethyl cyanomethylphosphonate (0.63 mL, 3.89 mmol) dissolved in anhydrous tetrahydrofuran (2 mL) and stirred at 23° C. for 30 min. The mixture was cooled to 0° C., treated with a solution of furo[2,3-b]pyridin-3(2H)-on (500 mg, 3.79 mmol) dissolved in anhydrous tetrahydrofuran (9 mL) and stirred at 23° C. for 15 h. The solvent was evaporated, the residue was dissolved in CH₂Cl₂ (50 mL), washed with water, dried over anhydrous MgSO4, filtered and evaporated to give 2-(Furo[2,3-b]pyridin-3-yl)acetonitrile (562 mg, 96%) as a yellow solid. The crude product was used directly in the next step without further purification.

¹H NMR (400 MHz, Chloroform-d) δ=8.42 (dd, J=4.9, 1.6 Hz, 1H, H—Ar), 8.01 (dd, J=7.7, 1.6 Hz, 1H, H—Ar), 7.77 (t, J=1.2, 1H, H—Ar), 7.33 (dd, J=7.7, 4.9 Hz, 1H, H—Ar), 3.80 (d, J=1.2, 2H, CH₂) ppm.

MS (ESI+, H₂O/MeCN) m/z (%): 159.0 (100, [M+H]⁺).

### 104.4 2-(Furo[2,3-b]pyridin-3-yl)acetic acid

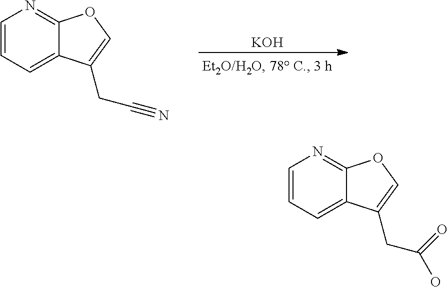 [SMILES: N#CCc1coc2ncccc12 ; "KOH" ; O=C(O)Cc1coc2ncccc12]

A solution of 2-(furo[2,3-b]pyridin-3-yl)acetonitrile (560 mg, 3.54 mmol) in ethanol (50 mL) and water (5 mL) was treated with KOH (500 mg, 8.91 mmol) and stirred at reflux for 3 h. The solvent was evaporated, the residue was dissolved in water (50 mL), washed with CH₂Cl₂ (3×30 mL) and incubated with Chelex 100 (1 g) for 1 h. Filtration and evaporation of the solvent gave 2-(Furo[2,3-b]pyridin-3-yl)acetic acid (620 mg, 99%) as a light brown solid. The crude product was used directly in the next step without further purification.

¹H NMR (400 MHz, Methanol-d₄) δ=8.24-8.11 (m, 2H, H—Ar), 7.74 (s, 1H, H—Ar), 7.30 (dd, J=7.7, 5.0 Hz, 1H, H—Ar), 3.53 (s, 2H, CH₂) ppm.

MS (ESI+, H₂O/MeCN) m/z (%): 178.1 (100, [M+H]⁺).

### 104.5 2-(Furo[2,3-b]pyridin-3-yl)-N-(5-methylthiazol-2-yl)acetamide

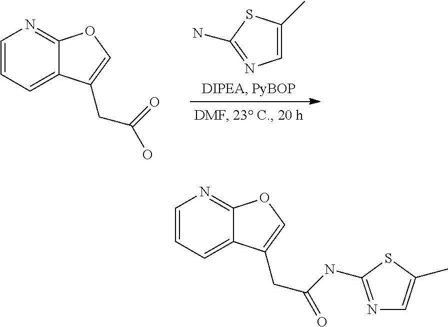

A solution of 2-(furo[2,3-b]pyridin-3-yl)acetic acid (150 mg, 0.85 mmol) and 5-methylthiazol-2-amine (106 mg, 0.93 mmol) in DMF (5 mL) was treated with DIPEA (0.29 mL, 1.69 mmol) and benzotriazol-1-yl-oxytripyrrolidinophosphonium hexafluorophosphate (485 mg, 0.93 mmol), stirred at 23° C. for 20 h and evaporated. The residue was dissolved in CH₂Cl₂, washed with brine, dried over anhydrous MgSO₄, filtered and evaporated. HPLC purification (method A) gave 2-(furo[2,3-b]pyridin-3-yl)-N-(5-methylthiazol-2-yl)acetamide (64 mg, 28%) as an off-white solid.

¹H NMR (400 MHz, Chloroform-d) δ=8.36 (d, J=4.8 Hz, 1H, H—Ar), 8.04 (d, J=7.6 Hz, 1H, H—Ar), 7.83 (s, 1H, H—Ar), 7.29 (s, 1H, H—Ar), 7.10 (s, 1H, H—Ar), 3.99 (s, 2H, CH₂), 2.44 (s, 3H, CH₃) ppm.

MS (ESI+, H₂O/MeCN) m/z (%): 274.0 (100, [M+H]⁺).

### Example 105

### 2-(Furo[3,2-b]pyridin-3-yl)-N-(5-methylthiazol-2-yl)acetamide

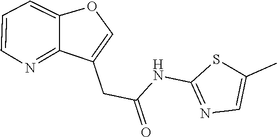 [SMILES: CC1=CCCN=C(NC(=O)Cc2coc3cccnc23)S1]

### 105.1 Ethyl 3-hydroxypicolinate

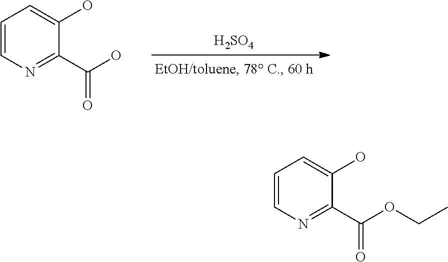

A solution of 3-hydroxypicolinic acid (2.50 g, 18.0 mmol) in EtOH (60 mL) and toluene (20 mL) was treated with conc. H₂SO₄ (1 mL) and stirred under reflux for 60 h with azeotropic removal of water via Dean-Stark trap. The solvent was evaporated, the residue was dissolved in water (50 mL) and carefully basified with a sat. Na—HCO₃ solution, upon which a white precipitate appeared. The mixture was diluted with EtOAc (30 mL) and the aqueous layer was extracted three times with EtOAc (3×20 mL). The combined organic layers were dried over anhydrous MgSO₄, filtered and evaporated to give ethyl 3-hydroxypicolinate (2.10 g, 70%) as a colorless liquid.

¹H NMR (400 MHz, Chloroform-d) δ=10.78 (s, 1H, OH), 8.30 (dd, J=4.2, 1.5 Hz, 1H, H—Ar), 7.47-7.34 (m, 2H, H—Ar), 4.54 (q, J=7.1 Hz, 2H, O—CH₂CH₃), 1.49 (t, J=7.1 Hz, 3H, O—CH₂CH₃) ppm.

MS (ESI+, H₂O/MeCN) m/z (%): 168.0 (100, [M+H]⁺).

### 105.2 Ethyl 3-(2-ethoxy-2-oxoethoxy)picolinate

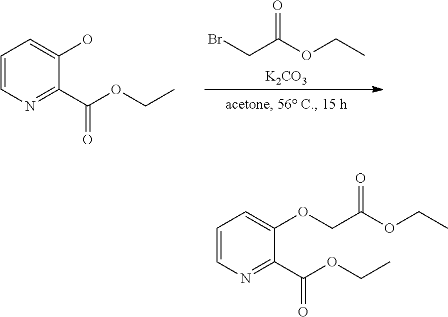

The reaction was performed under Ar atmosphere.

A solution of ethyl 3-hydroxypicolinate (2.10 g, 12.6 mmol) and ethyl bromoacetate (1.60 mL, 14.4 mmol) in anhydrous acetone (25 mL) was treated with anhydrous K₂CO₃, stirred under reflux for 15 h and cooled down to 23° C. The mixture was filtered and the solvent evaporated. The residue was dissolved in CH₂Cl₂ (100 mL), washed with water (3×50 mL), dried over anhydrous MgSO₄, filtered and evaporated. Column chromatography (SiO₂; EtOAc/Heptane 25:75->40:60) gave Ethyl 3-(2-ethoxy-2-oxoethoxy)picolinate (2.42 g, 76%) as a colorless oil.

¹H NMR (400 MHz, Chloroform-d) δ=8.36 (dd, J=4.5, 1.2 Hz, 1H, H—Ar), 7.39 (dd, J=8.5, 4.5 Hz, 1H, H—Ar), 7.29 (dd, J=8.6, 1.2 Hz, 1H, H—Ar), 4.74 (s, 2H, OCH₂C═O), 4.47 (q, J=7.1 Hz, 2H, O—CH₂CH₃), 4.27 (q, J=7.1 Hz, 2H, O—CH₂CH₃), 1.44 (t, J=7.1 Hz, 3H, O—CH₂CH₃), 1.29 (t, J=7.1 Hz, 3H, O—CH₂CH₃) ppm.

MS (ESI+, H₂O/MeCN) m/z (%): 254.0 (100, [M+H]⁺).

### 105.3 Ethyl 3-hydroxyfuro[3,2-b]pyridine-2-carboxylate

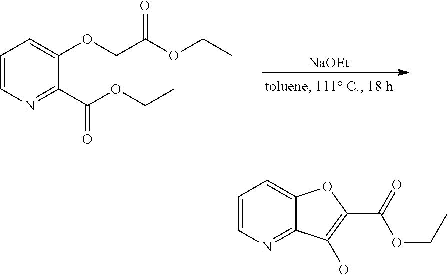 [SMILES: CCOC(=O)COc1cccnc1C(=O)OCC ; CCOC(=O)c1oc2cccnc2c1O ; "NaOEt"]

The reaction was performed under Ar atmosphere.

A solution of ethyl 3-(2-ethoxy-2-oxoethoxy)picolinate (2.10 g, 8.29 mmol) in anhydrous toluene (40 mL) was treated with sodium ethoxide (1.43 mL, 21 wt. % in EtOH, 18.2 mmol) and stirred under reflux for 18 h. The mixture was diluted with water (60 mL) and the organic layer was extracted with water (3×30 mL). The combined aqueous layers were acidified with acetic acid to pH 5 and extracted with CH₂Cl₂ (5×30 mL). The combined organic layers were dried over anhydrous MgSO₄, filtered and evaporated to give ethyl 3-hydroxyfuro[3,2-b]pyridine-2-carboxylate (1.26 g, 73%) as an off-white solid.

¹H NMR (400 MHz, Chloroform-d) δ=8.68 (dd, J=4.6, 1.3 Hz, 1H, H—Ar), 7.81 (dd, J=8.6, 1.3 Hz, 1H, H—Ar), 7.43 (dd, J=8.5, 4.6 Hz, 1H, H—Ar), 4.52 (q, J=7.1 Hz, 2H, O—CH₂CH₃), 1.48 (t, J=7.1 Hz, 3H, O—CH₂CH₃) ppm.

MS (ESI+, H₂O/MeCN) m/z (%): 208.0 (100, [M+H]⁺).

### 105.4 Furo[3,2-b]pyridin-3(2H)-one

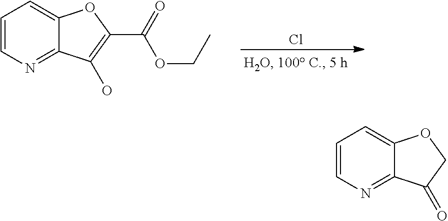

A solution of ethyl 3-hydroxyfuro[3,2-b]pyridine-2-carboxylate (522 mg, 2.52 mmol) in 10% hydrochloric acid (50 mL) was stirred under reflux for 5 h The excess of HCl was evaporated to give the crude hydrochloride of furo[3,2-b]pyridin-3(2H)-one (432 mg, quant.) as a brown solid which was used directly in the next step without further purification.

¹H NMR (400 MHz, Chloroform-d) δ=10.14 (br. s, 1H, OH), 8.49 (d, J=5.8 Hz, 1H, H—Ar), 8.34 (dd, J=8.4, 0.9 Hz, 1H, H—Ar), 8.00 (s, 1H, H—Ar), 7.71 (dd, J=8.4, 5.8 Hz, 1H, H—Ar) ppm.

MS (ESI+, H₂O/MeCN) m/z (%): 136.0 (100, [M+H]⁺).

### 105.5 2-(Furo[3,2-b]pyridin-3-yl)acetonitrile

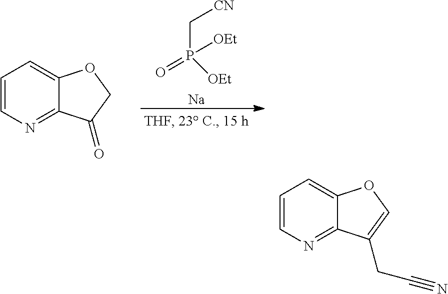

The reaction was performed under Ar atmosphere.

A suspension of sodium hydride (0.206 g, 60% dispersion in mineral oil, 5.16 mmol) in anhydrous tetrahydrofuran (5 mL) was treated dropwise with a solution of diethyl cyanomethylphosphonate (0.42 mL, 2.64 mmol) in anhydrous tetrahydrofuran (2 mL) and stirred at 23° C. for 30 min. The mixture was cooled to 0° C., treated with a suspension of furo[3,2-b]pyridin-3(2H)-one hydrochloride (432 mg, 2.52 mmol) in anhydrous tetrahydrofuran (15 mL) and stirred at 23° C. for 15 h. The solvent was evaporated and the residue dissolved in CH₂Cl₂ (50 mL) The organic phase was washed with water, dried over anhydrous MgSO4, filtered and the solvent evaporated to give 2-(furo[3,2-b]pyridin-3-yl)acetonitrile (204 mg, 51%) as a light brown solid. The crude product was used directly in the next step without further purification.

¹H NMR (400 MHz, Chloroform-d) δ=8.56 (dd, J=4.8, 1.2 Hz, 1H, H—Ar), 7.93 (t, J=1.3 Hz, 1H), 7.78 (dd, J=8.4, 1.3 Hz, 1H, H—Ar), 7.33-7.26 (m, 1H, H—Ar), 3.88 (d, J=1.3 Hz, 2H, CH₂) ppm.

MS (ESI+, H₂O/MeCN) m/z (%): 159.0 (100, [M+H]⁺).

### 105.6 2-(Furo[3,2-b]pyridin-3-yl)acetic acid

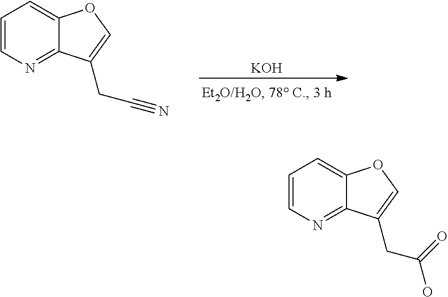 [SMILES: N#CCc1coc2cccnc12 ; "KOH" ; O=C(O)Cc1coc2cccnc12]

A solution of 2-(furo[3,2-b]pyridin-3-yl)acetonitrile (204 mg, 1.29 mmol) in ethanol (20 mL) and water (2 mL) was treated with KOH (200 mg, 3.56 mmol) and stirred at reflux for 3 h. The solvent was evaporated the residue was dissolved in water (30 mL). The aqueous solution was washed with CH₂Cl, (3×30 mL) and the aqueous phase was incubated with Chelex 100 (1 g) for 30 min. Filtration and evaporation of the solvent gave 2-(furo[3,2-b]pyridin-3-yl)acetic acid (220 mg, 96%) as purple solid. The crude product was used directly in the next step without further purification.

¹H NMR (400 MHz, Methanol-d₄) δ=8.30 (dd, J=4.9, 1.3 Hz, 1H, H—Ar), 7.84 (s, 1H, H—Ar), 7.75 (dd, J=8.4, 1.2 Hz, 1H, H—Ar), 7.18 (dd, J=8.3, 4.8 Hz, 1H, H—Ar), 3.52 (d, J=1.1 Hz, 2H, CH₂) ppm.

MS (ESI+, H₂O/MeCN) m/z (%): 178.0 (100, [M+H]⁺).

### 105.7 2-(Furo[3,2-b]pyridin-3-yl)-N-(5-methylthiazol-2-yl)acetamide

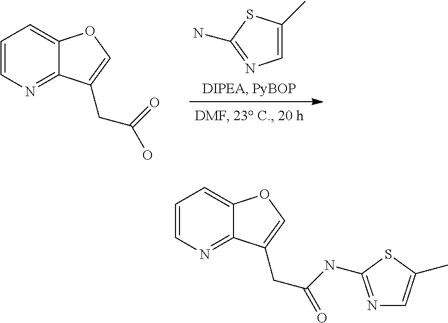

A solution of 2-(furo[3,2-b]pyridin-3-yl)acetic acid (220 mg crude, <1 mM) and 5-methylthiazol-2-amine (126 mg, 1.10 mmol) in DMF (6 mL) was treated with DIPEA (0.34 mL, 2.00 mmol) and benzotriazol-1-yl-oxytripyrrolidinophosphonium hexafluorophosphate (572 mg, 1.10 mmol) before it was stirred at 23° C. for 20 h. The solvent was evaporated and the residue was dissolved in EtOAc (50 mL), washed with a sat. NaHCO₃ solution (2×30 mL), water (30 mL) and brine (30 mL). The organic phase was dried over anhydrous MgSO₄, filtered and the solvent evaporated. HPLC purification (method A) gave 2-(furo[3,2-b]pyridin-3-yl)-N-(5-methylthiazol-2-yl)acetamide (38 mg, 14%) as a light brown solid.

¹H NMR (400 MHz, Chloroform-d) δ=8.69 (dd, J=5.0, 1.2 Hz, 1H, H—Ar), 8.03 7.93 (m, 2H, H—Ar), 7.42 (dd, J=8.4, 5.1 Hz, 1H, H—Ar), 7.12 (s, 1H, H—Ar), 4.20 (d, J=0.9 Hz, 2H, CH₂), 3.13 (br. s, 1H, NH), 2.42 (s, 3H, CH₃).

MS (ESI+, H₂O/MeCN) m/z (%): 274.0 (100, [M+H]⁺).

### Example 106

### 2-(Furo[2,3-b]pyridin-3-yl)-N-(5-(trifluoromethyl)thiazol-2-yl)acetamide

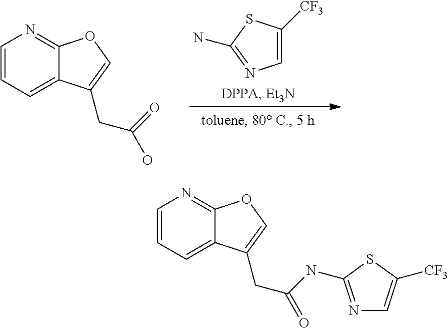

The reaction was performed under Ar atmosphere.

A solution of 2-(furo[2,3-b]pyridin-3-yl)acetic acid, prepared according to examples 104.1 to 104.4, (150 mg, 0.85 mmol) in anhydrous toluene (10 mL) was treated with diphenylphosphoryl azide (0.18 mL, 0.85 mmol) and Et₃N (0.09 mL, 0.68 mmol), stirred at reflux for 2 h and cooled to 23° C. The mixture was treated with a solution of 5-(trifluoromethyl)thiazol-2-amine (142 mg, 0.85 mmol) previously dissolved in anhydrous toluene (5 mL) and stirred at 80° C. for 5 h. The mixture was diluted with toluene (30 mL), washed with water and brine, dried over anhydrous MgSO₄, filtered and evaporated. HPLC purification (method A) gave 2-(furo[2,3-b]pyridin-3-yl)-N-(5-(trifluoromethyl)thiazol-2-yl)acetamide (1.5 mg, 1%) as colorless solid.

¹H NMR (400 MHz, DMSO-d₆) δ=13.08 (br. s, 1H, NH), 8.33 (dd, J=4.9, 1.7 Hz, 1H, H—Ar), 8.18-8.11 (m, 2H, H—Ar), 8.06 (s, 1H, H—Ar), 7.38 (dd, J=7.7, 4.8 Hz, 1H, H—Ar), 4.02 (d, J=1.0 Hz, 2H, CH₂) ppm.

MS (ESI+, H₂O/MeCN) m/z (%): 328.0 (100, [M+H]⁺).

### Example 107

### N-(4-(5-(aminomethyl)furan-2-yl)thiazol-2-yl)-2-(6,7-dihydro-5H-indeno[5,6-b]furan-3-yl)acetamide hydrochloride

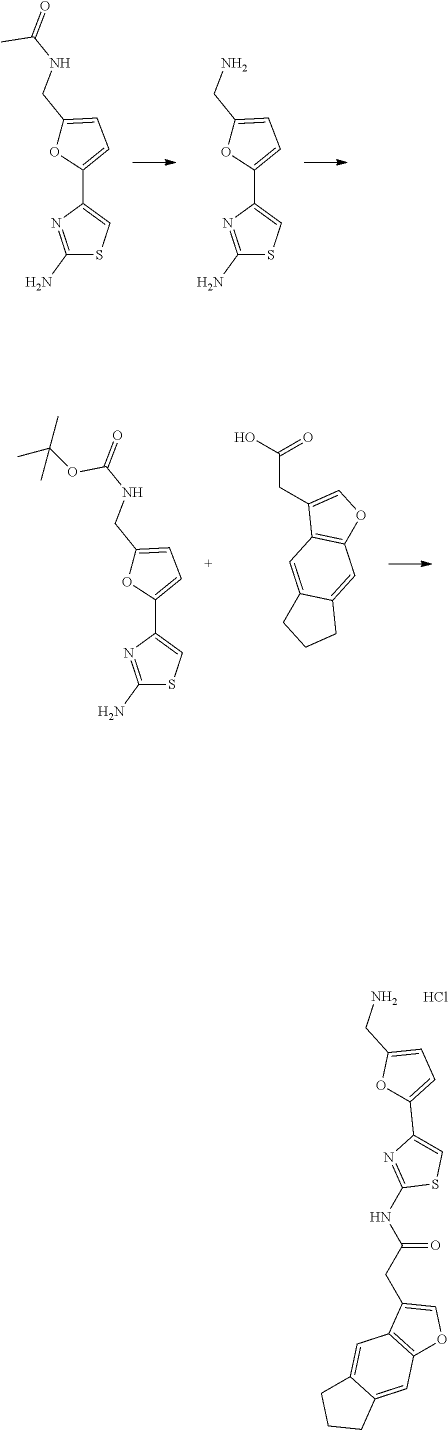 [SMILES: CC(=O)NCc1ccc(-c2csc(N)n2)o1 ; NCc1ccc(-c2csc(N)n2)o1 ; CC(C)(C)OC(=O)NCc1ccc(-c2csc(N)n2)o1 ; O=C(O)Cc1coc2cc3c(cc12)CCC3 ; NCc1ccc(-c2csc(NC(=O)Cc3coc4cc5c(cc34)CCC5)n2)o1 ; Cl]

### 107.1 4-[5-(aminomethyl)-2-furyl]thiazol-2-amine

Compound N-[[5-(2-aminothiazol-4-yl)-2-furyl]methyl]acetamide (1.21 g; 5.1 mmol) was refluxed in aqueous hydrochloric acid solution (30 mL, 3N) for 2 h. After completion of the reaction (LCMS control), the mixture was cooled and basified with KOH. The precipitate was collected, washed with water and dried in vacuo to afford compound 4-[5-(aminomethyl)-2-furyl]thiazol-2-amine (463 mg, 46%), which was used in the following step without further characterization.

### 107.2 tert-butyl N-[[5-(2-aminothiazol-4-yl)-2-furyl]methyl]carbamate

Compound 4-[5-(aminomethyl)-2-furyl]thiazol-2-amine (450 mg; 2.3 mmol) was dissolved in MeOH, followed by addition of Boc₂O (479 mg; 2.2 mmol). The mixture was allowed to stir at ambient temperature for 2 hours, and the solvents were distilled off to afford compound tert-butyl N-[[5-(2-aminothiazol-4-yl)-2-furyl]methyl]carbamate (700 mg, quant. yield), which was used in the following step without further characterization.

### 107.3 N-(4-(5-(aminomethyl)furan-2-yl)thiazol-2-yl)-2-(6,7-dihydro-5H-indeno[5,6-b]furan-3-yl)acetamide hydrochloride

To a cooled DMF solution of compound tert-butyl N-[[5-(2-aminothiazol-4-yl)-2-furyl]methyl]carbamate (203 mg; 0.69 mmol), compound 2-(6,7-dihydro-5H-cyclopenta[f]benzofuran-3-yl)acetic acid (193 mg; 0.89 mmol), 1-hydroxypyridotriazole (149 mg; 1.2 mmol)) and EDC (170 mg; 0.87 mmol) were added, and the mixture was allowed to stir at r.t. overnight. The solution was poured into water and extracted with EtOAc. The organic layer was washed with water, brine, dried over Na₂SO₄. and evaporated in vacuo. The residue was dissolved in DCM, followed by the addition of HCl in dioxane (4M). The resulting precipitate was collected, washed with acetone, dried in vacuo to provide the title compound in 105 mg (33%) yield.

HPLC-MS (Positive mode) m/z 394 (M+H)⁺ Retention time 1.112 min, purity 100%.

¹H NMR (400 MHz, DMSO d6): δ=2.05 (m, 2H), 2.91 (q, 7.0 Hz, 4H), 3.85 (s, 2H), 4.13 (q, 7.0 Hz, 2H), 6.67 (dd, 3.0 Hz, 2H), 7.32 (s, 1H), 7.38 (s, 1H), 7.43 (s, 1H), 7.81 (s, 1H) 8.46 (br.s, 3H), 12.67 (s, 1H).

### Example 108

### N-[4-[4-(acetamidomethyl)phenyl]thiazol-2-yl]-2-(5,6-dimethylbenzofuran-3-yl)acetamide

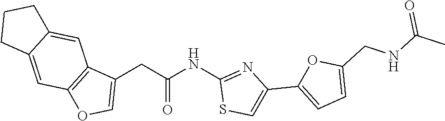 [SMILES: CC(=O)NCc1ccc(-c2csc(NC(=O)Cc3coc4cc5c(cc34)CCC5)n2)o1]

The title compound was obtained by acetylation of the compound of example 107.

### B. Biological Investigations

### Abbreviations

AUC area under curve
CLL chronic lymphocytic leucemia
DMEM Dulbecco's modified eagle medium
DMSO dimethyl sulfoxide
i.v. or IV intravenous
PBS phosphate buffered saline
PO peroral
QD once a day
Q7D4 4 injections in a 7 days interval
ThPA: N-{[4-(Benzyloxy)phenyl](methyl)-λ⁴-sulfanylidene}-4-methylbenzenesulfonamide (CAS Number: 21306-65-0; VWR, USA)
Tween 20: polysorbat 20

### General Methods

### Cell Culture

HeLa, A549 and HCT116 cells were grown in high-glucose Dulbecco's Modified Eagle's Medium (DMEM, Sigma)+10% FBS+1% penicillin and streptomycin+1% L-glutamine, at 37° C. with 5% CO₂ and 95% humidity. Cytotoxic screening of the ProQinase panel of 100 cell-lines was performed by ProQinase (Freiburg, Germany). Patient derived CLL isolates were prepared and screened as described by Dietrich et al. (S. Dietrich et al., J Clin Invest, 2018, 128(1), 427-445). Cell viability was determined after 48 hours using the ATP-based CellTiter Glo assay (Promega). Luminescence was measured with a Tecan Infinite F200 Microplate Reader (Tecan Group AG) and with an integration time of 0.2 seconds per well.

### Example B.1: Characterization of Compounds for their Influence on egr1 Expression

The compounds of the present invention can be characterized for their effect on expression of egr1 (early growth response protein 1) using an EGR1 reporter cell line.

EGR1 reporter cell lines can be generated, for example, by transfecting cells of a suitable cell line, e.g. HeLa cells, with an expression vector that comprises the coding sequence for at least one reporter, such as luciferase or a GFP (green fluorescent protein), under the control of the EGR1 promoter. This allows for reporter expression to be controlled by stimuli regulating EGR1 transcription (see, for example Gudernova et al., Elife. 6:e21536 (2017)). EGR1 reporter vectors are known in the art and are commercially available (e.g., pGL4[luc2P/hEGR1/Hygro] Vector from Promega Corporation, Madison, Wis., USA, and EGR-1-Luc Reporter Vector from Signosis, Inc., Santa Clara, Calif., USA).

Methods for determining luciferase activity are also well known in the art and generally rely on the measurement of bioluminescent light that is produced in the luciferase-catalyzed conversion of a luciferase substrate (luciferin) by ATP and oxygen in the presence of Mg²⁺ to produce oxyluciferin, AMP, PPᵢ, CO₂ and light. Luciferase assay kits are available, for example, from Promega Corporation, Madison, USA, and Perkin Elmer Inc., Waltham, Mass., USA.

### Generation of a Genomically Engineered EGR1 Reporter HeLa Cell-Line

The HeLa cell line was genetically modified to provide a simple, robust and highly reproducible cell-based assay reporting the activity of an endogenous EGR1 promoter. In brief, a construct encoding EGFP and luciferase proteins, separated by a self-cleaving P2A peptide was inserted, using CRISPR, immediately downstream (3′) to the promoter of endogenous EGR1. Upon treatment with compounds, cells express EGFP and luciferase from EGR1 promoter, which can be readily detected either in live cells using microscopy or cytometry, or through detection of luciferase activity in cell lysates.

To achieve stable genomic integration of an EGR1-promoter dual reporter, two plasmids were generated: one contained the reporter construct (eGFP-P2A-luciferase) flanked by homology arms that direct insertion into genomic DNA, by homologous recombination, of a break in genomic DNA generated by guide RNA targeted cleavage by Cas9 endonuclease. The gRNA expressing plasmid was based on px330 (56), into which a gRNA sequence that targets a break in gDNA close to the start codon of EGR1 was cloned. The left homology arm (encoding part of EGR1 promoter adjacent to its start codon) and right homology arm (encoding upstream of start codon of EGR1) were cloned from gDNA using the following primers:

Left HA-rev
(SEQ. ID NO: 1)
tcaccatTTGGACGAGCAGGCTGGA
Left HA for
(SEQ ID NO: 2)
gacggccagtgaattCTTCCCCAGCCTAGTTCACG
Right HA-rev
(SEQ ID NO: 3)
cgactctagaggatCCAGTGGCAGAGCCCATTTC
Right HA-for
(SEQ ID NO: 4)
tccccgcGGCCAAGGCCGAGATGC

The reporter construct was amplified from HIV-1SDm-CMV-eGFP-P2A-luc plasmid using the following primers:

Reporter-for
(SEQ ID NO: 5)
tcgtccaaatggtgagcaagggcgagga
Reporter-rev
(SEQ ID NO: 6)
ccttggccgcggggaggcggcccaaagg

The resulting PCR products were cloned into pUC19 vector using an InFusion kit from Clontech. Both vectors were transfected into HeLa cells and suitable derivatives were identified using flow cytometry

### Compound Testing

The present compounds can be tested, e.g. by using a HeLa cell line carrying an EGR1 reporter construct which allows for expression of luciferase and eGFP (enhanced GFP) controlled by the EGR1 promoter. For this reporter cells are seeded in the wells of a 384 well microtiter plate at a density of 2000 cells per well in 48 μl of DMEM supplemented with 4.5 g/l glucose, 2 mM glutamine and 10% FCS and are incubated for 24 hours at 37° C. with 5% CO₂ and 95% humidity. Then, an eleven point 1:3 serial dilution of each test compound, from an initial concentration of 100 μM, is prepared in DMSO and the dilutions are added to the cells in a volume of 2 μl per well. The cells are incubated for a further 24 hours, after which the luciferase activity of each well is determined by addition of 25 μl of luciferase substrate reaction mixture (Britelite™ plus, Perkin Elmer) and measuring the bioluminescence light output (EnVision Xcite plate reader, PerkinElmer). The results are shown in table 1.

The compound of example 64 served as a positive control for this EGR1 reporter assay. The compound of example 64 had been identified in an initial high throughput screening campaign. Moreover, massively parallel sequencing of RNA transcripts at multiple time-points from HeLa cells treated with the compound of example 64 demonstrated that EGR1 transcripts were upregulated at early time points.

TABLE 1

| Example No | EC₅₀ |
|---|---|
| 1 | B |
| 2 | B |
| 3 | B |
| 4 | B |
| 5 | B |
| 6 | B |
| 7 | B |
| 8 | B |
| 9 | B |
| 10 | B |
| 11 | B |
| 12 | B |
| 13 | B |
| 14 | B |
| 15 | B |
| 16 | B |
| 17 | B |
| 18 | B |
| 19 | B |
| 20 | B |
| 21 | B |
| 22 | B |
| 23 | A |
| 24 | A |
| 25 | A |
| 26 | A |
| 27 | A |
| 28 | A |
| 29 | A |
| 30 | A |
| 31 | A |
| 32 | A |
| 33 | A |
| 34 | A |
| 35 | A |
| 36 | A |
| 37 | A |
| 38 | A |
| 39 | A |
| 40 | A |
| 41 | A |
| 42 | A |
| 43 | A |
| 44 | A |
| 45 | A |
| 46 | A |
| 47 | A |
| 48 | A |
| 49 | A |
| 50 | A |
| 51 | A |
| 52 | A |
| 53 | A |
| 54 | A |
| 55 | A |
| 56 | A |
| 57 | A |
| 58 | A |
| 59 | A |
| 60 | A |
| 61 | A |
| 62 | A |
| 63 | A |
| 64 | A |
| 65 | A |
| 66 | A |
| 67 | A |
| 68 | A |
| 69 | A |
| 70 | A |
| 71 | A |
| 72 | A |
| 73 | A |
| 74 | A |
| 75 | A |
| 76 | A |
| 77 | A |
| 78 | A |
| 79 | A |
| 80 | A |
| 81 | A |
| 82 | A |
| 83 | A |
| 84 | A |
| 85 | A |
| 86 | A |
| 87 | A |
| 88 | A |
| 89 | A |
| 90 | A |
| 91 | A |
| 92 | A |
| 93 | A |
| 94 | A |
| 95 | A |
| 96 | A |
| 97 | A |
| 98 | A |
| 99 | A |
| 100 | A |
| 101 | A |
| 102 | A |
| 103 | B |
| 104 | B |
| 106 | A |
| 108 | B |

Key:
A: 10 nM to <10 μM;
B: 10 μM to <100 μM

### Example B.2: Surface Plasmon Resonance

Recombinant human pirin was produced in E. coli with an N-terminal hexahistidine tag and a C-terminal strep tag using a commercially available plasmid construct (pQStrep2-PIR, Addgene Plasmid #31570; Büssow et al., Microbial Cell Factories 4:21 (2005)).

Pirin was covalently linked to a Biacore Series S CM7 chip (GE Healthcare) via amine chemistry in 10 mM acetate buffer, pH 5.5 using 25 μg per ml pirin in the presence of ThPA, a known pirin ligand (Miyazaki et al., Nat. Chem. Biol. 6:667 (2010)) whose presence was included to protect the active site of pirin. A control chip was also prepared under identical condition but without including pirin in the reaction. The sensorgram produced during immobilization demonstrated that pirin was specifically coupled to the surface of the CM7 chip in sufficient amounts to generate a robust signal. A series of increasing concentrations of compound, either the control ThPA or a compound of the present invention is then applied to the pirin modified CM7 chip in phosphate buffered saline containing 2% DMSO and 0.05% tween 20 and sensorgrams are recorded covering the association, equilibrium and dissociation phases of the response.

As shown in FIG. 1, ThPA and compound of example 72 associates with pirin with Kd's of 380 nM and 1.6 μM, respectively.

### Example B.3: Nano Differential Scanning Fluorimetry (NanoDSF)

NanoDSF is an advanced Differential Scanning Fluorimetry method for measuring protein stability using intrinsic tryptophan or tyrosine fluorescence. The fluorescence of the tryptophans and tyrosines in a protein is strongly dependent on their close surroundings. Changes in protein structure typically affect both the intensity and the emission wavelength especially of tryptophan fluorescence. By measuring fluorescence intensity at 330 nm and 350 nm, the change in fluorescence intensity and the shift of the fluorescence maximum upon unfolding can be used to detect thermal melting of the protein. Proteins are stabilized when associated with ligands and show a shift in their melting temperatures. NanoDSF has the advantages of being label free and observing the protein in solution.

A 10 μM solution of pirin in phosphate buffered saline, with or without 20 μM test compound, is subject to thermal denaturation under fluorescence monitoring using a Prometheus NT.48 instrument of NanoTemper Technologies. Unliganded pirin has a complex biphasic melting curve. This may reflect independent melting of the two 8-domains within pirin. If the test compound is a ligand to pirin, it adopts a single thermal transition some 10° C. above that of apopirin. Association of either compounds of example 64, 85 or 101 with pirin induces an increase in the Tₘ of melting by approximately 11° C. Additionally, all active pirin ligands substantially increase the thermal stability of pirin by between 4 and 12° C. In addition to increasing the overall thermal stability of pirin, interaction with the benzofuran ligands result in pirin melting at a single temperature. This again indicates that the ligands of the present invention induce significant structural rearrangements to pirin upon binding.

### Example B.4: In Vitro Test Evaluating Growth Inhibition of the ProQinase 100 Cancer Cell-Line Panel

The ProQinase 100 cell-line panel is a cell proliferation assay service of ProQinase comprising the EC₅₀ determination of a test compound against a defined panel of 100 cancer cell lines from 18 different tissue types.

The inhibitory growth effect of the compound of example 85 on the ProQinase 100 cancer cell-line panel was evaluated by ProQinase. The obtained data show that the compound of example 85 inhibits the proliferation of all 100 cell-lines with a growth inhibition EC₅₀ in the range of from 0.58 to 16 μM and a median growth inhibition EC₅₀ of 3.2 μM.

### Example B.5: In Vitro Test Evaluating Growth Inhibition of Cells Derived from Patients with CLL

The response of 97 tumour samples derived from patients with CLL was investigated. All samples tumor cells were obtained from whole blood, subjected to Ficoll-Isopaque density centrifugation. CD19+B and CD3+ T cells were isolated by positive magnetic cell separation (Miltenyi Biotec). Sorted cells were checked for purity by fluorescence-activated cell sorting (FACS) with CD19/CD20 for healthy control samples and CD19/CD20/CD5 for CLL samples (BD Biosciences). Following sorting, all samples with a CD19/CD20/CD5 purity <98% were subjected to additional sorting, and the average final purity of all sorted samples was >99%. CLL samples with >100×10⁶ WBC/μL were not subject to purification.

Cells are incubated for three days with an eight-point three-fold titration series of the test compound from an initial concentration of 30 μM (2000 cells per well in a volume of 50 μl). Cellular viability is estimated by the addition of 25 μL of ATPlite (Perkin Elmer) with the resulting luminescence measured using an EnVision Xcite plate reader (Perkin Elmer).

The compound of example 72 has a cytotoxic effect on most patient derived CLL samples (90; 93%) with 45 (46%) patient samples killed with an EC₅₀ less than 10 μM.

In addition, the growth inhibitory response of a selection of 27 patient derived CLL isolates (S. Dietrich et al., J Clin Invest, 2018, 128(1), 427-445) against the compounds of examples 64 and 85 was evaluated using the procedure as described above. Both compounds were active against all isolates with median EC₅₀ values of 6.0 and 1.67, respectively.

### Example B.6: In Vivo Test Evaluating the Effects of Test Compounds on the Growth of A549 Cells in Nude Mice

The following test can be conducted for determining, if administration of compounds influences the growth of A549 cells in nude mice, in comparison to solvent only and to carboplatin, a standard of care. An i.p. route of administration is evaluated at 10 and 3 mg/kg delivered i.p., q.d. and compared with solvent control and carboplatin at 75 mg/kg delivered Q7D4 ip. Eight mice are used per study condition.

Compounds are supplied as a dry powder. Each compound is first dissolved in DMSO to yield an appropriate concentration then mixed with 9 volumes of a previously prepared solution of Cremophor-EL: 5% Mannitol (1:8, v/v) warmed to 37° C. while vigorously vortexing. This mixture is sonicated in an ultrasonic bath heated to 40° C. for 15-20 min. The formulations are stable for 24 hours at ambient temperature. A working formulation batch is prepared immediately prior to the in vivo study. A dose volume of 5 ml/kg is used for each concentration and route of administration.

NMRI-nu/nu nude mice are injected subcutaneously in one flank with 5×10⁶ A549 cells in 200 μl of DMEM prepared by trypsinizing an exponentially growing culture of cells. Tumours are allowed to develop to an approximate volume of 100 mm³, (approximately one week after initiation) and thereafter treatment commenced. Body weights and tumour volume are determined every two days. The study lasts for a maximum of a further 28 days, or until the tumour burden exceeded 1000 mm³. At the end of the study, tumours are excised, weighed and then preserved by snap freezing in liquid nitrogen.

As shown in FIG. 2, treatment at 3 mg/kg and 10 mg/kg of the compound of example 64 by i.p. administration QD substantially prevented the growth of A549 lung adenocarcinoma cells in nude mice and performed as well at the current clinical standard of care, carboplatin. No significant changes in body weight or signs of toxicity were apparent

### Example B.7: Microsomal Stability

Mouse hepatic microsomes were isolated from pooled (50), perfused livers of Balb/c male mice according to the standard protocol (Hill, J. R. in Current Protocols in Pharmacology 7.8.1-7.8.11, Wiley Interscience, 2003). The batch of microsomes was tested for quality control using Imipramine, Propranolol and Verapamil as reference compounds. Microsomal incubations were carried out in 96-well plates in 5 aliquots of 40 μL each (one for each time point). Liver microsomal incubation medium contained PBS (100 mM, pH 7.4), MgCl₂ (3.3 mM), NADPH (3 mM), glucose-6-phosphate (5.3 mM), glucose-6-phosphate dehydrogenase (0.67 units/ml) with 0.42 mg of liver microsomal protein per ml. Control incubations were performed replacing the NADPH-cofactor system with PBS.

Test compound (2 μM, final solvent concentration 1.6%) is incubated with microsomes at 37° C., shaking at 100 rpm. Incubations are performed in duplicates. Five time points over 40 minutes are analyzed. The reactions are stopped by adding 12 volumes of 90% acetonitrile-water to incubation aliquots, followed by protein sedimentation by centrifuging at 5500 rpm for 3 minutes. Supernatants are analyzed using the HPLC system coupled with tandem mass spectrometer. The elimination constant (kₑₗ), half-life (t½) and intrinsic clearance (Clint) is determined in plot of In(AUC) versus time, using linear regression analysis.

### Example B.8: Bioavalability

Male Balb/c mice (11-12 weeks old, body weight 23.7 to 30.6 g and average body weight across all groups 26.5 g, SD=1.6 g) are used in this study. The animals are randomly assigned to the treatment groups before the pharmacokinetic study; all animals are fasted for 3 h before dosing. Six time points (IV: 5, 15, 30, 60, 120 and 240 min, and PO: 15, 30, 60, 120, 240, and 360 min) are used in this pharmacokinetic study. Each of the PO and IV time point treatment groups includes 4 animals; there is also control group of 2 animals. Dosing is done according to the treatment schedules outlined in the Table 2. Mice are injected IV with tribrometanol at the dose of 150 mg/kg prior to taking blood. Blood samples are withdrawn from retroorbital sinus and are collected in microcontainers containing K₂EDTA. All samples are immediately prepared, flash-frozen and stored at −70° C. until subsequent bioanalysis.

TABLE 2

| | | | | | Target | |
|---|---|---|---|---|---|---|
| | | | | Target | Dose | Target |
| Number of | | | | Dose | Concen- | Dose |
| Mice | Test | Formu- | Delivery | Level | tration | Volume |
| (male) | compound | lation | Route | (mg/kg) | (mg/ml) | (ml/kg) |
| 24 | yes | 1 | PO | 30 | 6 | 5 |
| 24 | yes | 1 | IV | 10 | 2 | 5 |
| 2 | no | 1 | IV | 0 | 0 | 5 |

Formulation 1: DMSO - Cremophor EL - 5% aqueous solution of Mannitol (10%:10%:80%)

Plasma samples (50 μl) are mixed with 200 μl of IS solution (100 ng/ml in acetonitrile-methanol mixture 1:1, v/v). After mixing by pipetting and centrifuging for 4 min at 6,000 rpm, 2 μl of each supernatant is injected into a LC-MS/MS system.

The concentrations of test compound are determined using a high performance liquid chromatography/tandem mass spectrometry (HPLC-MS/MS) method. A Shimadzu HPLC system comprised of 2 isocratic pumps LC-10Advp, an autosampler SIL-HTc, a sub-controller FCV-14AH and a degasser DGU-14A. Mass spectrometric analysis is performed using an API 3000 (triple-quadrupole) instrument from AB Sciex (Canada) with an electro-spray (ESI) interface. The data acquisition and system control is performed using Analyst 1.5.2 software from AB Sciex.

The tests performed in examples B.7 and B.8 showed that the compound of example 64 has a microsomal stability of 10 minutes and an oral bioavalability of 9.9% and that the compound of example 85 has a microsomal stability of 113 minutes and an oral bioavalability of 40%.

### Example B.9: Benzofuran Pirin Ligands Compromise the Warburg Effect in Tumor Cells

Cheeseman et a., through deconvolution of their phenotypic screen, established a link between pirin and HSF1 (M. D. Cheeseman et al., J Med Chem, 2017, 60(1), 180-201), with their bisamide pirin ligand compromising the activity of HSF1. As HSF1 is a key driver of malignant metabolism, the effect of compound of example 64 on selected key components was evaluated by RNAseq and by western blot.

### RNAseq

Total RNA was isolated using TRIzol (Thermofisher) following the manufacturer's instructions. Barcoded stranded mRNA-seq libraries were prepared using the Illumina TruSeq RNA Sample Preparation v2 Kit (Illumina, San Diego, Calif., USA) implemented on the liquid handling robot Beckman FXP2. The resulting libraries were pooled in equimolar amounts; 1.8 pM solution of this pool was loaded on the Illumina sequencer NextSeq 500 and sequenced uni-directionally, generating ˜500 million reads 85 bases long. Sequencing reads were aligned using STAR aligner (version 2.5, (92) against the human genome reference (GRCh37/hg19 with UCSC annotation). Reads mapping to regions described as “exon” in the reference were counted during the alignment (-quantMode GeneCounts option in STAR).

### Western Blot

HeLa cells were grown in high-glucose DMEM medium containing 10% FBS, 1% penicillin and streptomycin and 1% L-glutamine. Treated cells were washed twice with PBS, pelleted and resuspended in Laemmli loading buffer. After a brief sonication to reduce viscosity, the samples were electrophoresed on a 12.5% SDS gel and subsequently blotted onto PVDF membranes for 1 hour at 100 V at 4° C. The membranes were blocked in 5% BSA in TBST buffer for 1 hour, and incubation with primary antibodies (pirin, (Sigma, 0.2 μg/ml); HSF1, (Cell Signaling, 0.2 μg/ml); LAT1, (Sigma, 0.2 μg/ml); GLUT1, (Abcam, 0.2 μg/ml) was performed overnight at 4° C. After three washes with TBST, appropriate secondary antibodies conjugated to horse radish peroxidase (Sigma, 0.1 μg/ml) were incubated for 45 minutes after which the membranes were washed a further three times. Immuno-stained bands were visualized with ECL reagent (Invitrogen).

### Results:

While EGR1 and EGR2 mRNA levels rose to around 30 fold higher some hours after treatment with compound of example 64, pirin, HSF1, SLC2A1 and SLC7A5 mRNA and protein levels were substantially reduced, with kinetics mirroring EGR1 induction. Moreover, RNAseq analysis demonstrated that of the 170 solute carrier transcripts expressed in HeLa cells, 121 are downregulated greater than 4-fold with only one, SLC6A14, upregulated. Additionally, and presumably in consequence of down-regulation of HSF1, the expression of heat-shock proteins HSPA12A, HSPB8, HSPBP1, HSPA4, HSPD1 and HSPA14 are concomitantly down-regulated greater than 10-fold upon treatment with the compound of example 64.

Prompted by these observations, the glucose uptake and lactate excretion in HeLa cells treated for 16 hours with the compound of examples 64 was evaluated. The obtained data showed that glucose uptake was reduced by approximately 80% in the presence of the compound of example 64, from concentrations at and above those that result in maximal expression of EGR1, with a concomitant reduction in lactic acid secretion. A reduction in glucose uptake was also observed in A549 (lung adenocarcinoma) and HCT116 (colon adenocarcinoma) cells.

### Example B.10: Benzofuran Pirin Ligands Suppress Expression of Multiple Transcripts Involved in Aerobic Glycolysis

Next, the effect of the compound of example 64 on the expression of components of glycolysis and on the PIK3K-Akt-mTOR signalling pathway was explored. RNAseq at multiple time points demonstrates that treatment of HeLa cells with 10 μM of compound of example 64 results in the down-regulation of multiple components of the glycolytic pathway and moreover, of tyrosine kinases known to activate PI3K (EGFR and IGF1R are expressed in HeLa cells), of PIK3K, of Akt isoforms and of mTOR. Collectively, these data indicate that benzofuran pirin ligands act to supress the Akt signalling axis and aerobic glycolysis in transformed cells.

### DESCRIPTION OF FIGURES

FIG. 1 shows sensorgrams obtained from a biacore chip onto which pirin was immobilized demonstrate specific and high affinity interactions between pirin, ThPA and the compound of Example 72. A control chip showed no response.

FIG. 2 shows the tumor growth, i.e. the tumor volume vs. the time after tumor transplantation for vehicle, for dosage of the compound of example 64 at 3 mg/kg QD (i.p.) and 10 mg/kg QD (i.p.) of the compound of example 64 and for dosage of carboplatin at 75 mg/kg Q7D4 (i.p.).

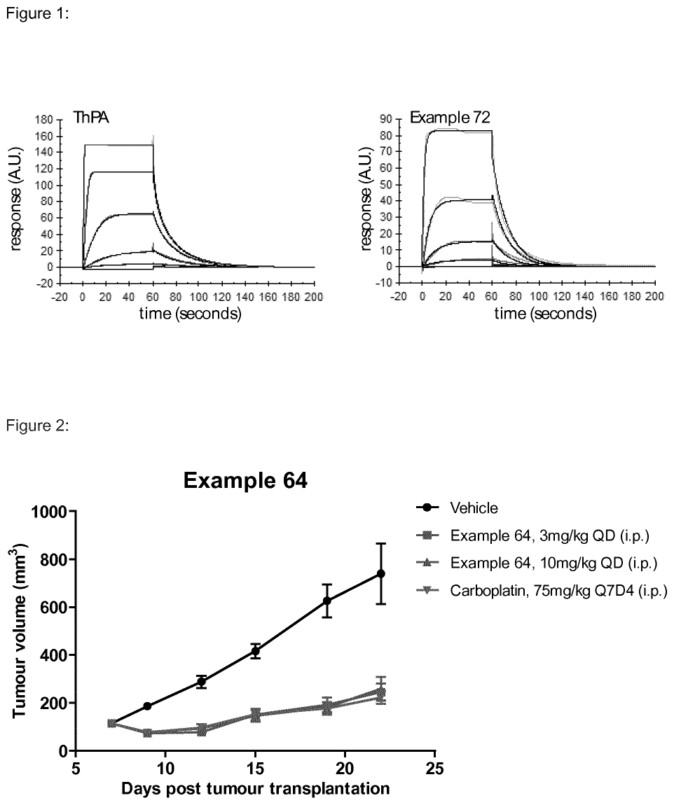

1. A pharmaceutical composition comprising a compound of the formula I or a tautomer or a pharmaceutically acceptable salt thereof 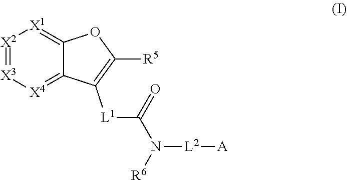
wherein
X¹ is CR¹ or N;
X² is CR² or N;
X³ is CR³ or N;
X⁴ is CR⁴ or N;
with the proviso that at most two of X¹, X², X³ and X⁴ are N;
L¹ is a bond, C₁-C₆-alkylene which may carry one or more substituents R⁷, or C₃-C₈-cycloalkylene which may carry one or more substituents R⁸;
L² is a bond, C₁-C₆-alkylene which may carry one or more substituents R⁷, C₃-C₈-cycloalkylene which may carry one or more substituents R⁸, C₁-C₆-alkylene-O, C₁-C₆-alkylene-S, C₁-C₆-alkylene-NR¹⁵, where the alkylene moiety in the three last-mentioned radicals may carry one or more substituents R⁷; C₃-C₈-cycloalkylene-O, C₃-C₈-cycloalkylene-S or C₃-C₈-cycloalkylene-NR¹⁵, where the cycloalkylene moiety in the three last-mentioned radicals may carry one or more substituents R⁸;
A is 3-, 4-, 5-, 6-, 7- or 8-membered saturated, partially unsaturated or maximally unsaturated carbocyclic ring which may carry one or more substituents R⁹; or a 3-, 4, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁰;
or L²-A forms a group C₁-C₆-alkylene-OR¹³, C₁-C₆-alkylene-SR¹⁴ or C₁-C₆-alkylene-NR¹⁵R¹⁶;
R¹, R², R³ and R⁴, independently of each other, are selected from the group consisting of hydrogen, halogen, CN, nitro, SF₅, C₁-C₆-alkyl which may carry one or more substituents R¹¹, C₁-C₆-haloalkyl, C₃-C₈-cycloalkyl which may carry one or more substituents R¹², OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
or R¹ and R², or R² and R³, or R³ and R⁴, together with the carbon atoms they are bound to, form a 3-, 4-, 5-, 6- or 7-membered partially unsaturated or maximally unsaturated carbocyclic or heterocyclic ring, where the heterocyclic ring contains 1, 2 or 3 heteroatoms or heteroatom-containing groups selected from the group consisting of O, N, S, NO, SO and SO₂ as ring members, where the carbocyclic or heterocyclic ring may carry one or more substituents R¹⁸;
R⁵ is selected from the group consisting of hydrogen, C₁-C₆-alkyl, C₁-C₆-haloalkyl, aryl, aryl-C₁-C₃-alkyl, where the aryl moiety in the two last-mentioned radicals may carry one or more substituents R¹⁸; hetaryl and hetaryl-C₁-C₃-alkyl, where hetaryl is a 5- or 6-membered heteroaromatic ring containing 1, 2, 3, or 4 heteroatoms selected from the group consisting of O, S and N as ring members, where the heteroaromatic ring may carry one or more substituents R¹⁸;
R⁶ is selected from the group consisting of hydrogen, C₁-C₆-alkyl which may carry one or more substituents R¹¹, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-cycloalkyl-C₁-C₄-alkyl, where cycloalkyl in the two last-mentioned radicals may carry one or more substituents R¹²; C₁-C₆-alkoxy, C₁-C₆-haloalkoxy, aryl, aryl-C₁-C₃-alkyl, where the aryl moiety in the two last-mentioned radicals may carry one or more substituents R¹⁸; heterocyclyl and heterocyclyl-C₁-C₃-alkyl, where heterocyclyl is a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
R⁷ and R⁸, independently of each other and independently of each occurrence, are selected from the group consisting of F, CN, nitro, SF₅, C₁-C₆-alkyl which may carry one or more substituents R¹¹, C₁-C₆-haloalkyl, C₃-C₈-cycloalkyl which may carry one or more substituents R¹², OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
or two radicals R⁷ bound on the same carbon atom of the alkylene group, or two radicals R⁸ bound on the same carbon atom of the cycloalkylene group form together a group ═O or ═S;
each R⁹ is independently selected from the group consisting of halogen, CN, nitro, SF₅, C₁-C₆-alkyl which may carry one or more substituents R¹¹, C₁-C₆-haloalkyl, C₃-C₈-cycloalkyl which may carry one or more substituents R¹², OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
or two radicals R⁹ bound on adjacent ring atoms, together with the ring atoms they are bound to, may form a saturated, partially unsaturated or maximally unsaturated 3-, 4, 5- or 6-membered carbocyclic ring which may be substituted by one or more radicals selected from the group consisting of halogen, CN, nitro, SF₅, C₁-C₆-alkyl which may carry one or more substituents R¹¹, C₁-C₆-haloalkyl, C₃-C₈-cycloalkyl which may carry one or more substituents R¹², OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
or two radicals R⁹ bound on non-adjacent ring atoms may form a bridge —CH₂— or —(CH₂)₂—;
each R¹⁰ is independently selected from the group consisting of halogen, CN, nitro, SF₅, C₁-C₆-alkyl which may carry one or more substituents R¹¹, C₁-C₆-haloalkyl, C₃-C₈-cycloalkyl which may carry one or more substituents R¹², OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
or two radicals R¹⁰ bound on adjacent ring atoms, together with the ring atoms they are bound to, may form a saturated, partially unsaturated or maximally unsaturated 3-, 4, 5- or 6-membered carbocyclic or heterocyclic ring, where the heterocyclic ring contains 1, 2, 3 or 4 heteroatoms or heteroatom-containing groups selected from the group consisting of O, N, S, NO, SO and SO₂ as ring members, where the carbocyclic or heterocyclic ring may be substituted by one or more radicals selected from the group consisting of halogen, CN, nitro, SF₅, C₁-C₆-alkyl which may carry one or more substituents R¹¹, C₁-C₆-haloalkyl, C₃-C₈-cycloalkyl which may carry one or more substituents R¹², OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
each R¹¹ is independently selected from the group consisting of CN, nitro, SF₅, C₃-C₈-cycloalkyl which may carry one or more substituents R¹², OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
each R¹² is independently selected from the group consisting of halogen, CN, nitro, SF₅, C₁-C₆-alkyl, C₁-C₆-haloalkyl, C₃-C₈-cycloalkyl, C₃-C₈-halocycloalkyl, OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
each R¹³ is independently selected from the group consisting of hydrogen, C₁-C₆-alkyl which may carry one or more substituents R¹⁹, C₁-C₆-haloalkyl, C₃-C₈-cycloalkyl which may carry one or more substituents R²⁰, S(O)ₘR¹⁴, C(O)R¹⁷, C(O)OR²¹, C(O)NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
each R¹⁴ is independently selected from the group consisting of hydrogen, C₁-C₆-alkyl which may carry one or more substituents R¹⁹, C₁-C₆-haloalkyl, C₃-C₈-cycloalkyl which may carry one or more substituents R²⁰, OR²¹, NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
R¹⁵ and R¹⁶, independently of each other and independently of each occurrence, are selected from the group consisting of hydrogen, C₁-C₆-alkyl which may carry one or more substituents R¹⁹, C₁-C₆-haloalkyl, C₃-C₈-cycloalkyl which may carry one or more substituents R²⁰, OR²¹, S(O)ₘR²², C(O)R¹⁷, C(O)OR²¹, C(O)NR²³R²⁴, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
or R¹⁵ and R¹⁶, together with the nitrogen atom they are bound to, form a saturated, partially unsaturated or maximally unsaturated 3-, 4-, 5- or 6-membered heterocyclic ring, where the heterocyclic ring may additionally contain 1 or 2 further heteroatoms or heteroatom-containing groups selected from the group consisting of O, N, S, NO, SO and SO₂ as ring members, where the heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo;
each R¹⁷ is independently selected from the group consisting of hydrogen, C₁-C₆-alkyl which may carry one or more substituents R¹⁹, C₁-C₆-haloalkyl, C₃-C₈-cycloalkyl which may carry one or more substituents R²⁰, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
each R¹⁸ is independently selected from the group consisting of halogen, CN, nitro, OH, SH, SF₅, C₁-C₆-alkyl which may carry one or more substituents selected from the group consisting of CN, OH, C₁-C₆-alkoxy, C₁-C₆-haloalkoxy, SH, C₁-C₆-alkylthio, C₁-C₆-haloalkylthio, C₁-C₆-alkylsulfonyl, C₁-C₆-haloalkylsulfonyl, NR²³R²⁴ and phenyl; C₁-C₆-haloalkyl, C₃-C₈-cycloalkyl which may carry one or more substituents selected from the group consisting of halogen, CN, OH, C₁-C₆-alkyl, C₁-C₆-haloalkyl, C₁-C₆-alkoxy, C₁-C₆-haloalkoxy, SH, C₁-C₆-alkylthio, C₁-C₆-haloalkylthio, C₁-C₆-alkylsulfonyl, C₁-C₆-haloalkylsulfonyl and phenyl; C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, carboxyl, C₁-C₆-alkylcarbonyl, C₁-C₆-haloalkylcarbonyl, C₁-C₆-alkoxycarbonyl, C₁-C₆-haloalkoxycarbonyl, aryl and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where aryl or the heterocyclic ring may carry one or more substituents selected from the group consisting of halogen, CN, OH, C₁-C₆-alkyl, C₁-C₆-haloalkyl, C₁-C₆-alkoxy and C₁-C₆-haloalkoxy;
or two radicals R¹⁸ bound on adjacent ring atoms, together with the ring atoms they are bound to, may form a saturated, partially unsaturated or maximally unsaturated 3-, 4, 5- or 6-membered carbocyclic or heterocyclic ring, where the heterocyclic ring contains 1, 2, 3 or 4 heteroatoms or heteroatom-containing groups selected from the group consisting of O, N, S, NO, SO and SO₂ as ring members, where the carbocyclic or heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo;
each R¹⁹ is independently selected from the group consisting of CN, OH, C₃-C₈-cycloalkyl, C₃-C₈-halocycloalkyl, C₁-C₆-alkoxy, C₁-C₆-haloalkoxy, SH, C₁-C₆-alkylthio, C₁-C₆-haloalkylthio, C₁-C₆-alkylsulfonyl, C₁-C₆-haloalkylsulfonyl, NR²³R²⁴, aryl and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where aryl or the heterocyclic ring may carry one or more substituents R¹⁸;
each R²⁰ is independently selected from the group consisting of halogen, CN, OH, C₁-C₆-alkyl, C₁-C₆-haloalkyl, C₁-C₆-alkoxy, C₁-C₆-haloalkoxy, SH, C₁-C₆-alkylthio, C₁-C₆-haloalkylthio, C₁-C₆-alkylsulfonyl, C₁-C₆-haloalkylsulfonyl and phenyl;
R²¹ and R²², independently of each other and independently of each occurrence, are selected from the group consisting of hydrogen, C₁-C₆-alkyl which may carry one or more substituents R¹⁹, C₁-C₆-haloalkyl, C₃-C₈-cycloalkyl, C₃-C₈-halocycloalkyl, aryl and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where aryl or the heterocyclic ring may carry one or more substituents selected from the group consisting of halogen, CN, OH, C₁-C₆-alkyl, C₁-C₆-haloalkyl, C₁-C₆-alkoxy and C₁-C₆-haloalkoxy;
R²³ and R²⁴, independently of each other and independently of each occurrence, are selected from the group consisting of hydrogen, C₁-C₆-alkyl, C₁-C₆-haloalkyl, C₃-C₈-cycloalkyl, C₃-C₈-halocycloalkyl, C₁-C₆-alkylcarbonyl, C₁-C₆-haloalkylcarbonyl, C₁-C₆-alkoxycarbonyl, C₁-C₆-haloalkoxycarbonyl, C₁-C₆-alkylsulfonyl, C₁-C₆-haloalkylsulfonyl, aryl and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where aryl or the heterocyclic ring may carry one or more substituents selected from the group consisting of halogen, CN, OH, C₁-C₆-alkyl, C₁-C₆-haloalkyl, C₁-C₆-alkoxy and C₁-C₆-haloalkoxy;
m is 1 or 2; and
n is 0, 1 or 2;
and
at least one pharmaceutically acceptable carrier and/or auxiliary substance.

2. The pharmaceutical composition as claimed in claim 1, wherein
X¹ is CR¹, X² is CR², X³ is CR³ and X⁴ is CR⁴; or
X¹ is N, X² is CR², X³ is CR³ and X⁴ is CR⁴; or
X¹ is CR¹, X² is N, X³ is CR³ and X⁴ is CR⁴; or
X¹ is CR¹, X² is CR², X³ is N and X⁴ is CR⁴; or
X¹ is CR¹, X² is CR², X³ is CR³ and X⁴ is N; or
X¹ is N, X² is CR², X³ is N and X⁴ is CR⁴; or
X¹ is CR¹, X² is N, X³ is CR³ and X⁴ is N;
L¹ is C₁-C₆-alkylene which may carry one or more substituents R⁷;
L² is a bond, C₁-C₆-alkylene or C₁-C₆-alkylene-NR¹⁵, where the alkylene moiety in the two last-mentioned radicals may carry one or more substituents R⁷;
A is C₅-C₆-cycloalkyl which may carry one or two substituents R⁹, or is a 5-membered saturated, partially unsaturated or aromatic heterocyclic ring containing 1 or 2 heteroatoms selected from the group consisting of O, N and S as ring members, where the heterocyclic ring may carry one or more substituents R¹⁰;
or L²-A forms a group C₁-C₆-alkylene-NR¹⁵R¹⁶;
R¹ and R², independently of each other, are selected from the group consisting of hydrogen, halogen, CN, C₁-C₆-alkyl, C₁-C₆-haloalkyl, C₃-C₈-cycloalkyl, C₃-C₈-halocycloalkyl, C₁-C₆-alkoxy, C₁-C₆-haloalkoxy, C₁-C₆-alkylthio, C₁-C₆-haloalkylthio, phenyl which may carry one or more substituents R¹⁸, and a 5- or 6-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
R³ and R⁴, independently of each other, are selected from the group consisting of hydrogen, halogen, CN, C₁-C₆-alkyl, C₁-C₆-haloalkyl, C₁-C₄-alkoxy and C₁-C₄-haloalkoxy;
or R¹ and R², or R² and R³, together with the carbon atoms they are bound to, form a 5- or 6-membered saturated, partially unsaturated or maximally unsaturated carbocyclic or heterocyclic ring, where the heterocyclic ring contains 1, 2 or 3 heteroatoms or heteroatom-containing groups selected from the group consisting of O, N, S, NO, SO and SO₂ as ring members;
R⁵ is hydrogen;
R⁶ is selected from the group consisting of hydrogen, C₁-C₆-alkyl which may carry one substituent R¹¹, C₂-C₆-alkenyl, and phenyl which may carry one or more substituents R¹⁸;
each R⁷ is independently selected from the group consisting of F, CN, OH, C₁-C₄-alkyl, C₁-C₄-haloalkyl, C₃-C₆-cycloalkyl, C₃-C₆-halocycloalkyl, C₁-C₄-alkoxy, C₁-C₄-haloalkoxy and phenyl which may carry one or more substituents R¹⁸;
or two radicals R⁷ bound on the same carbon atom of the alkylene group, form together a group ═O;
each R⁹ is independently selected from the group consisting of halogen, C₁-C₆-alkyl which may carry one or more substituents R¹¹, and C₁-C₆-haloalkyl,
or two radicals R⁹ bound on adjacent ring atoms, together with the ring atoms they are bound to, may form a maximally unsaturated 5- or 6-membered carbocyclic ring;
or two radicals R⁹ bound on non-adjacent ring atoms may form a bridge —CH₂—;
each R¹⁰ is independently selected from the group consisting of CN, C₁-C₆-alkyl which may carry one or more substituents R¹¹, C₁-C₆-haloalkyl, C₁-C₆-alkoxy, C₁-C₆-haloalkoxy, S(O)₂R¹⁴, C(O)R¹⁷, C(O)OR¹³, C(O)NR¹⁵R¹⁶, aryl which may carry one or more substituents R¹⁸, and a 5- or 6-membered heteroaromatic ring containing 1, 2, 3 or 4 heteroatoms groups selected from the group consisting of O, N and S as ring members, where the heteroaromatic ring may carry one or more substituents R¹⁸;
or two radicals R¹⁰ bound on adjacent ring atoms, together with the ring atoms they are bound to, may form a saturated, partially unsaturated or maximally unsaturated 5- or 6-membered carbocyclic or heterocyclic ring, where the heterocyclic ring contains 1, 2, 3 or 4 heteroatoms or heteroatom-containing groups selected from the group consisting of O, N, S, NO, SO and SO₂ as ring members, where the carbocyclic or heterocyclic ring may be substituted by one or more radicals selected from the group consisting of halogen, C₁-C₆-alkyl which may carry one or more substituents R¹¹, C₁-C₆-haloalkyl, C₁-C₆-alkoxy, C₁-C₆-haloalkoxy, C₁-C₆-alkylsulfonyl, C₁-C₆-haloalkylsulfonyl, and phenyl which may carry one or more substituents selected from the group consisting of halogen, C₁-C₆-alkyl, C₁-C₆-haloalkyl, C₁-C₆-alkoxy and C₁-C₆-haloalkoxy;
each R¹¹ is independently selected from the group consisting of OH, C₁-C₆-alkoxy, C₁-C₆-haloalkoxy, NR¹⁵R¹⁶, C(O)OR¹³, C(O)NR¹⁵R¹⁶, phenyl which may carry one or more substituents R¹⁸, and a 3-, 4-, 5-, 6-, 7- or 8-membered saturated heterocyclic ring containing 1 or 2 heteroatoms or heteroatom-containing groups selected from the group consisting of O, N, S, NO, SO and SO₂ as ring members, where the heterocyclic ring may carry one or more substituents R¹⁸;
each R¹³ is independently C₁-C₆-alkyl or C₁-C₆-haloalkyl;
R¹⁴ is phenyl which may carry one or more substituents R¹⁸;
R¹⁵ and R¹⁶, independently of each other and independently of each occurrence, are selected from the group consisting of hydrogen, C₁-C₆-alkyl which may carry one or more substituents R¹⁹, C₁-C₆-haloalkyl, C₃-C₆-cycloalkyl, C₃-C₆-halocycloalkyl, C₁-C₆-alkylcarbonyl and C₁-C₆-haloalkylcarbonyl;
or R¹⁵ and R¹⁶, together with the nitrogen atom they are bound to, form a saturated, partially unsaturated or maximally unsaturated 3-, 4-, 5- or 6-membered heterocyclic ring, where the heterocyclic ring may additionally contain 1 or 2 further heteroatoms or heteroatom-containing groups selected from the group consisting of O, N, S, NO, SO and SO₂ as ring members, where the heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo;
each R¹⁷ is independently C₁-C₆-alkyl or C₁-C₆-haloalkyl;
each R¹⁸ is independently selected from the group consisting of halogen, CN, nitro, OH, SH, C₁-C₆-alkyl which may carry one or more substituents NR²³R²⁴; C₁-C₆-haloalkyl, C₃-C₈-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, carboxyl, C₁-C₆-alkylcarbonyl and C₁-C₆-haloalkylcarbonyl;
or two radicals R¹⁸ bound on adjacent ring atoms, together with the ring atoms they are bound to, may form a saturated, partially unsaturated or maximally unsaturated 5- or 6-membered carbocyclic or heterocyclic ring, where the heterocyclic ring contains 1 or 2 heteroatoms or heteroatom-containing groups selected from the group consisting of O, N, S, NO, SO and SO₂ as ring members, where the carbocyclic or heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo;
each R¹⁹ is independently selected from the group consisting of CN, OH, C₁-C₆-alkoxy, C₁-C₆-haloalkoxy, SH, C₁-C₆-alkylthio, C₁-C₆-haloalkylthio, C₁-C₆-alkylsulfonyl, C₁-C₆-haloalkylsulfonyl, NR²³R²⁴ and phenyl which may carry one or more substituents R¹⁸; and
R²³ and R²⁴, independently of each other and independently of each occurrence, are selected from the group consisting of hydrogen, C₁-C₆-alkyl, C₁-C₆-haloalkyl, C₃-C₈-cycloalkyl, C₃-C₈-halocycloalkyl, C₁-C₆-alkylcarbonyl, C₁-C₆-haloalkylcarbonyl, C₁-C₆-alkoxycarbonyl, C₁-C₆-haloalkoxycarbonyl, C₁-C₆-alkylsulfonyl, C₁-C₆-haloalkylsulfonyl, aryl and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where aryl or the heterocyclic ring may carry one or more substituents selected from the group consisting of halogen, CN, OH, C₁-C₆-alkyl, C₁-C₆-haloalkyl, C₁-C₆-alkoxy and C₁-C₆-haloalkoxy.

3. The pharmaceutical composition as claimed in claim 2, wherein
X¹ is CR¹ or N;
X² is CR²;
X³ is CR³;
X⁴ is CR⁴ or N;
with the proviso that at most one of X¹ and X⁴ is N;
L¹ is CH₂, CH(CH₃) or CH₂CH₂;
L² is a bond or CH₂CH₂NH;
A is a 5-membered heteroaromatic ring containing one nitrogen atom and one further heteroatom selected from the group consisting of O, N and S as ring members, where the heterocyclic ring may carry one or more substituents R¹⁰;
R¹ and R², independently of each other, are selected from the group consisting of hydrogen, halogen, CN, C₁-C₄-alkyl, C₁-C₄-alkoxy and C₁-C₄-haloalkoxy;
R³ and R⁴, independently of each other, are selected from the group consisting of hydrogen, F, C₁-C₄-alkyl and C₁-C₄-alkoxy;
or R¹ and R², or R² and R³ form together a bridging group —CH₂CH₂CH₂—, —CH₂CH₂CH₂CH₂—, or —O—CH₂—O—;
R⁵ is hydrogen;
R⁶ is selected from the group consisting of hydrogen, C₂-C₄-alkenyl, and phenyl which may carry one or more substituents R¹⁸;
each R¹⁰ is independently selected from the group consisting of CN, C₁-C₄-alkyl which may carry one or more substituents R¹¹, C₁-C₄-haloalkyl, C(O)R¹⁷, C(O)OR¹³, C(O)NR¹⁵R¹⁶, phenyl which may carry one or more substituents R¹⁸, and a 5- or 6-membered heteroaromatic ring containing one heteroatom selected from the group consisting of O, N and S as ring members, where the heteroaromatic ring may carry one or more substituents R¹⁸;
or two radicals R¹⁰ bound on adjacent ring atoms form together a bridging group —CH═CH—CH═CH—, —CH₂CH₂CH₂— or —CH₂CH₂CH₂CH₂—, where one of the hydrogen atoms in the bridging group may be substituted by a radical selected from the group consisting of methyl and methoxy;
each R¹¹ is independently selected from the group consisting of OH, C₁-C₄-alkoxy, C₁-C₄-haloalkoxy, NR¹⁵R¹⁶ and C(O)NR¹⁵R¹⁶;
each R¹³ is independently C₁-C₄-alkyl;
R¹⁵ and R¹⁶, independently of each other and independently of each occurrence, are selected from the group consisting of hydrogen, C₁-C₄-alkyl and C₁-C₄-alkylcarbonyl;
R¹⁷ is C₁-C₄-alkyl;
each R¹⁸ is independently selected from the group consisting of halogen, C₁-C₆-alkyl which may carry one substituent NR²³R²⁴; C₃-C₈-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, and C₁-C₆-alkylcarbonyl;
or two radicals R¹⁸ bound on adjacent ring atoms, together with the ring atoms they are bound to, may form a saturated 5- or 6-membered heterocyclic ring containing 1 or 2 heteroatoms or heteroatom-containing groups selected from the group consisting of O, N, S, NO, SO and SO₂ as ring members, where the heterocyclic ring may be substituted by one or more radicals selected from the group consisting of halogen, C₁-C₄-alkyl, C₁-C₄-haloalkyl, C₁-C₄-alkoxy, C₁-C₄-haloalkoxy and oxo; and
R²³ and R²⁴, independently of each other and independently of each occurrence, are selected from the group consisting of hydrogen and C₁-C₄-alkylcarbonyl.

4. The pharmaceutical composition as claimed in claim 1, wherein R⁴ is hydrogen.

5. The pharmaceutical composition as claimed in claim 1, wherein
X¹ is CR¹ or N;
X² is CR²;
X³ is CR³;
X⁴ is CR⁴ or N;
with the proviso that at most one of X¹ and X⁴ is N;
L¹ is CH₂, CH(CH₃) or CH₂CH₂;
L² is a bond or CH₂CH₂NH;
A is a 5-membered heteroaromatic ring containing one nitrogen atom and one further heteroatom selected from the group consisting of N, O and S as ring members, where the heterocyclic ring may carry one or more substituents R¹⁰;
R¹ and R², independently of each other, are selected from the group consisting of hydrogen, halogen, CN, C₁-C₄-alkyl, C₁-C₄-alkoxy and C₁-C₄-haloalkoxy;
R³ is selected from the group consisting of hydrogen, C₁-C₄-alkyl and C₁-C₄-alkoxy;
or R² and R³ form together a bridging group —CH₂CH₂CH₂— or —O—CH₂—O—;
R⁴ is hydrogen;
R⁵ is hydrogen;
R⁶ is selected from the group consisting of hydrogen, C₃-C₄-alkenyl, and phenyl which carries a substituent R¹⁸;
each R¹⁰ is independently selected from the group consisting of CN, C₁-C₄-alkyl which may carry one or more substituents R¹¹, C₁-C₄-haloalkyl, C(O)R¹⁷, C(O)OR¹³, C(O)NR¹⁵R¹⁶, phenyl which may carry one or two substituents R¹⁸, and a 5- or 6-membered heteroaromatic ring containing one heteroatom selected from the group consisting of O, N and S as ring members, where the heteroaromatic ring may carry one or more substituents R¹⁸;
or two radicals R¹⁰ bound on adjacent ring atoms form together a bridging group —CH═CH—CH═CH— or —CH₂CH₂CH₂—, where one of the hydrogen atoms in the bridging group may be substituted by a radical selected from the group consisting of methyl and methoxy;
each R¹¹ is independently selected from the group consisting of OH, C₁-C₄-alkoxy, C₁-C₄-haloalkoxy, NR¹⁵R¹⁶ and C(O)NR¹⁵R¹⁶;
each R¹³ is independently C₁-C₄-alkyl;
R¹⁵ and R¹⁶, independently of each other, are selected from the group consisting of hydrogen, C₁-C₄-alkyl and C₁-C₄-alkylcarbonyl;
R¹⁷ is C₁-C₄-alkyl;
each R¹⁸ is independently selected from the group consisting of halogen, C₁-C₆-alkyl which may carry one substituent NR²³R²⁴; C₃-C₆-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, and C₁-C₆-alkylcarbonyl;
or two radicals R¹⁸ bound on adjacent ring atoms, together with the ring atoms they are bound to, may form a saturated 5- or 6-membered heterocyclic ring containing one nitrogen ring atom or one or two oxygen atoms as ring members, where the heterocyclic ring may be substituted by an oxo group; and
R²³ and R²⁴, independently of each other and independently of each occurrence, are selected from the group consisting of hydrogen and C₁-C₄-alkylcarbonyl.

6. The pharmaceutical composition as claimed in claim 1, wherein A is selected from the group consisting of oxazol-2-yl, thiazol-2-yl and imidazol-2-yl, where oxazol-2-yl, thiazol-2-yl and imidazol-2-yl may carry one or more substituents R¹⁰.

7. The pharmaceutical composition as claimed in claim 2, wherein the compound of formula I is a compound of formula I.a 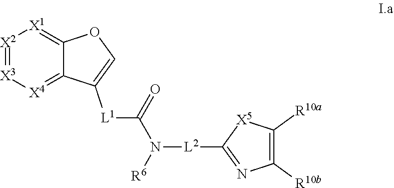
wherein
X¹ is CR¹, X² is CR², X³ is CR³ and X⁴ is CR⁴; or
X¹ is N, X² is CR², X³ is CR³ and X⁴ is CR⁴; or
X¹ is CR¹, X² is N, X³ is CR³ and X⁴ is CR⁴; or
X¹ is CR¹, X² is CR², X³ is N and X⁴ is CR⁴; or
X¹ is CR¹, X² is CR², X³ is CR³ and X⁴ is N;
L¹ is CH₂, CH(CH₃) or CH₂CH₂;
L² is a bond or CH₂CH₂NH;
X⁵ is O, S or NR^{x};
R^{x} is hydrogen or C₁-C₄-alkyl;
R¹ and R², independently of each other, are selected from the group consisting of hydrogen, F, Cl, CN, C₁-C₄-alkyl, C₁-C₂-alkoxy and C₁-C₄-haloalkoxy;
R³ is selected from the group consisting of hydrogen, C₁-C₄-alkyl and C₁-C₄-alkoxy;
or R² and R³ form together a bridging group —CH₂CH₂CH₂— or —O—CH₂—O—;
R⁴ is hydrogen;
R⁶ is selected from the group consisting of hydrogen, C₃-C₄-alkenyl, and phenyl which carries a substituent R¹⁸;
R^{10a} is selected from the group consisting of hydrogen, CN, C₁-C₄-alkyl which may carry one substituent R¹¹; C₁-C₄-haloalkyl, C(O)OR¹³ and C(O)NR¹⁵R¹⁶;
R^{10b} is selected from the group consisting of hydrogen, C₁-C₄-alkyl which may carry one substituent R¹¹; C(O)R¹⁷, C(O)OR¹³, C(O)NR¹⁵R¹⁶, phenyl which may carry one or two substituents R¹⁸, and a 5- or 6-membered heteroaromatic ring containing one heteroatom selected from the group consisting of O, N and S as ring members, where the heteroaromatic ring may carry one or more substituents R¹⁸;
or R^{10a} and R^{10b} bound on adjacent ring atoms form together a bridging group —CH═CH—CH═CH— or —CH₂CH₂CH₂—, where one of the hydrogen atoms in the bridging group may be substituted by a radical selected from the group consisting of methyl and methoxy;
each R¹¹ is independently selected from the group consisting of OH, C₁-C₄-alkoxy and C(O)NR¹⁵R¹⁶;
each R¹³ is independently C₁-C₄-alkyl;
R¹⁵ and R¹⁶, independently of each other, are selected from the group consisting of hydrogen and C₁-C₄-alkyl;
R¹⁷ is C₁-C₄-alkyl;
each R¹⁸ is independently selected from the group consisting of halogen, C₁-C₆-alkyl which may carry one substituent NR²³R²⁴; C₃-C₆-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, and C₁-C₆-alkylcarbonyl;
or two radicals R¹⁸ bound on adjacent ring atoms, together with the ring atoms they are bound to, may form a saturated 5- or 6-membered heterocyclic ring containing one nitrogen ring atom or one or two oxygen atoms as ring members, where the heterocyclic ring may be substituted by an oxo group; and
R²³ and R²⁴, independently of each other and independently of each occurrence, are selected from the group consisting of hydrogen and C₁-C₄-alkylcarbonyl.

8. The pharmaceutical composition as claimed in claim 7, wherein
X¹ is CR¹, X² is CR², X³ is CR³ and X⁴ is CR⁴; or
X¹ is N, X² is CR², X³ is CR³ and X⁴ is CR⁴; or
X¹ is CR¹, X² is CR², X³ is CR³ and X⁴ is N;
L¹ is CH₂, CH(CH₃) or CH₂CH₂;
L² is a bond or CH₂CH₂NH;
X⁵ is S or NR^{x};
R^{x} is hydrogen or C₁-C₄-alkyl;
R¹ and R², independently of each other, are selected from the group consisting of hydrogen, F, Cl, CN, C₁-C₄-alkyl, C₁-C₂-alkoxy and C₁-C₄-haloalkoxy;
R³ is selected from the group consisting of hydrogen, C₁-C₄-alkyl and C₁-C₄-alkoxy;
or R² and R³ form together a bridging group —CH₂CH₂CH₂— or —O—CH₂—O—;
R⁴ is hydrogen;
R⁶ is selected from the group consisting of hydrogen, C₃-C₄-alkenyl, and phenyl which carries a substituent R¹⁸;
R^{10a} is selected from the group consisting of hydrogen, CN, C₁-C₄-alkyl which may carry one substituent R¹¹; C₁-C₄-haloalkyl, and C(O)OR¹³;
R^{10b} is selected from the group consisting of hydrogen, C₁-C₄-alkyl, phenyl which may carry one or two substituents R¹⁸, and a 5- or 6-membered heteroaromatic ring containing one heteroatom selected from the group consisting of O, N and S as ring members, where the heteroaromatic ring may carry one or more substituents R¹⁸;
or R^{10a} and R^{10b} bound on adjacent ring atoms form together a bridging group —CH═CH—CH═CH— or —CH₂CH₂CH₂—, where one of the hydrogen atoms in the bridging group may be substituted by a radical selected from the group consisting of methyl and methoxy;
each R¹¹ is independently selected from the group consisting of OH and C₁-C₄-alkoxy;
each R¹³ is independently C₁-C₄-alkyl;
each R¹⁸ is independently selected from the group consisting of halogen, C₁-C₆-alkyl which may carry one substituent NR²³R²⁴; C₃-C₆-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, and C₁-C₆-alkylcarbonyl;
or two radicals R¹⁸ bound on adjacent ring atoms, together with the ring atoms they are bound to, may form a saturated 5- or 6-membered heterocyclic ring containing one or two oxygen atoms as ring members; and
R²³ and R²⁴, independently of each other and independently of each occurrence, are selected from the group consisting of hydrogen and C₁-C₄-alkylcarbonyl.

9. The pharmaceutical composition as claimed in claim 1, wherein
X¹ is CR¹, X² is CR², X³ is CR³ and X⁴ is CR⁴; or
X¹ is N, X² is CR², X³ is CR³ and X⁴ is CR⁴.

10. The pharmaceutical composition as claimed in claim 1, wherein the compound of formula I.a is a compound of formula I.a.1 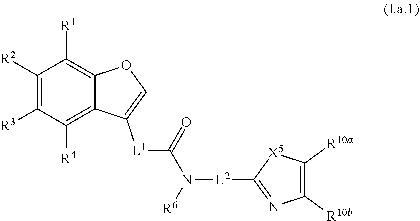
wherein
L¹ is CH₂, CH(CH₃) or CH₂CH₂;
L² is a bond or CH₂CH₂NH;
X⁵ is S or NR^{x};
R^{x} is hydrogen or C₁-C₄-alkyl;
R¹ and R², independently of each other, are selected from the group consisting of hydrogen, F, Cl, CN, C₁-C₄-alkyl, C₁-C₂-alkoxy and C₁-C₄-haloalkoxy;
R³ is selected from the group consisting of hydrogen, C₁-C₄-alkyl and C₁-C₄-alkoxy;
or R² and R³ form together a bridging group —CH₂CH₂CH₂— or —O—CH₂—O—;
R⁴ is hydrogen;
R⁶ is selected from the group consisting of hydrogen, C₃-C₄-alkenyl, and phenyl which carries a substituent R¹⁸;
R^{10a} is selected from the group consisting of hydrogen, CN, C₁-C₄-alkyl which may carry one substituent R¹¹; C₁-C₄-haloalkyl, and C(O)OR¹³;
R^{10b} is selected from the group consisting of hydrogen, C₁-C₄-alkyl, phenyl which may carry one or two substituents R¹⁸, and a 5- or 6-membered heteroaromatic ring containing one heteroatom selected from the group consisting of O, N and S as ring members, where the heteroaromatic ring may carry one or more substituents R¹⁸;
or R^{10a} and R^{10b} bound on adjacent ring atoms form together a bridging group —CH═CH—CH═CH— or —CH₂CH₂CH₂—, where one of the hydrogen atoms in the bridging group may be substituted by a radical selected from the group consisting of methyl and methoxy;
each R¹¹ is independently selected from the group consisting of OH and C₁-C₄-alkoxy;
each R¹³ is independently C₁-C₄-alkyl;
each R¹⁸ is independently selected from the group consisting of halogen, C₁-C₆-alkyl which may carry one substituent NR²³R²⁴; C₃-C₆-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, and C₁-C₆-alkylcarbonyl;
or two radicals R¹⁸ bound on adjacent ring atoms, together with the ring atoms they are bound to, may form a saturated 5- or 6-membered heterocyclic ring containing one or two oxygen atoms as ring members; and
R²³ and R²⁴, independently of each other and independently of each occurrence, are selected from the group consisting of hydrogen and C₁-C₄-alkylcarbonyl.

11. The pharmaceutical composition as claimed in claim 7, wherein
L¹ is CH₂, CH(CH₃) or CH₂CH₂;
L² is a bond or CH₂CH₂NH;
X⁵ is S;
R¹ and R², independently of each other, are selected from the group consisting of hydrogen, F, Cl, C₁-C₄-alkyl and C₁-C₂-alkoxy;
R³ is selected from the group consisting of hydrogen and C₁-C₄-alkyl;
or R² and R³ form together a bridging group —CH₂CH₂CH₂—;
R⁴ is hydrogen;
R⁶ is selected from the group consisting of hydrogen, C₃-C₄-alkenyl, and phenyl which carries a substituent R¹⁸;
R^{10a} is selected from the group consisting of hydrogen, CN, C₁-C₄-alkyl which may carry one substituent R¹¹; and C₁-C₄-haloalkyl;
R^{10b} is selected from the group consisting of hydrogen and phenyl which may carry one or two substituents R¹⁸;
or R^{10a} and R^{10b} bound on adjacent ring atoms form together a bridging group —CH═CH—CH═CH—;
each R¹¹ is independently selected from the group consisting of OH and C₁-C₄-alkoxy;
each R¹⁸ is independently selected from the group consisting of halogen, C₃-C₆-cycloalkyl, C₁-C₄-alkoxy, C₁-C₄-haloalkoxy, C₁-C₄-alkylthio, C₁-C₄-haloalkylthio, C₁-C₄-alkylsulfonyl, C₁-C₄-haloalkylsulfonyl, and C₁-C₄-alkylcarbonyl;
or two radicals R¹⁸ bound on adjacent ring atoms, together with the ring atoms they are bound to, may form a saturated 5- or 6-membered heterocyclic ring containing one or two oxygen atoms as ring members.

12. The pharmaceutical composition as claimed in claim 1, wherein R² and R³ do not form a bridging group —CH₂CH₂CH₂—.

13. The pharmaceutical composition as claimed in claim 1, wherein R⁶ is hydrogen.

14. The pharmaceutical composition as claimed in claim 1, wherein R⁶ is C₃-C₄-alkenyl or phenyl which carries a substituent R¹⁸.

15. (canceled)

16. A method to treat a condition, disorder or disease in a patient in need thereof comprising administering to the patient in need thereof a compound or a tautomer or a pharmaceutically acceptable salt thereof as described in claim 1, wherein the condition, disorder or disease is selected from the group consisting of inflammatory diseases, hyperproliferative diseases or disorders, a hypoxia related pathology and a disease characterized by pathophysiological hypervascularization.

17. The method of claim 16, wherein the condition, disorder or disease is selected from the group consisting of atherosclerosis, rheumatoid arthritis, asthma, inflammatory bowel disease, psoriasis, psoriasis capitis, psoriasis guttata, psoriasis inversa; neurodermatitis; ichthyosis; alopecia areata; alopecia totalis; alopecia subtotalis; alopecia universalis; alopecia diffusa; atopic dermatitis; lupus erythematodes of the skin; dermatomyositis; atopic eczema; morphea; scleroderma; alopecia areata Ophiasis type; androgenic alopecia; allergic dermatitis; irritative contact dermatitis; contact dermatitis; pemphigus vulgaris; pemphigus foliaceus; pemphigus vegetans; scarring mucous membrane pemphigoid; bullous pemphigoid; mucous membrane pemphigoid; dermatitis; dermatitis herpetiformis Duhring; urticaria; necrobiosis lipoidica; erythema nodosum; prurigo simplex; prurigo nodularis; prurigo acuta; linear IgA dermatosis; polymorphic light dermatosis; erythema solaris; exanthema of the skin; drug exanthema; purpura chronica progressiva; dihydrotic eczema; eczema; fixed drug exanthema; photoallergic skin reaction; and periorale dermatitis.

18. The method of claim 16, wherein the condition, disorder or disease is hyperproliferative disease which is selected from the group consisting of a tumor or cancer disease, precancerosis, dysplasia, histiocytosis, a vascular proliferative disease and a virus-induced proliferative disease.

19. The method of claim 18, wherein the condition, disorder or disease is a tumor or cancer disease which is selected from the group consisting of diffuse large B-cell lymphoma (DLBCL), T-cell lymphomas or leukemias, e.g., cutaneous T-cell lymphoma (CTCL), noncutaneous peripheral T-cell lymphoma, lymphoma associated with human T-cell lymphotrophic virus (HTLV), adult T-cell leukemia/lymphoma (ATLL), as well as acute lymphocytic leukemia, acute nonlymphocytic leukemia, acute myeloid leukemia, chronic lymphocytic leukemia, chronic myelogenous leukemia, Hodgkin's disease, non-Hodgkin's lymphoma, myeloma, multiple myeloma, mesothelioma, childhood solid tumors, glioma, bone cancer and soft-tissue sarcomas, common solid tumors of adults such as head and neck cancers (e.g., oral, laryngeal and esophageal), genitourinary cancers (e.g., prostate, bladder, renal, uterine, ovarian, testicular, rectal, and colon), lung cancer (e.g., small cell carcinoma and non-small cell lung carcinoma, including squamous cell carcinoma and adenocarcinoma), breast cancer, pancreatic cancer, melanoma and other skin cancers, basal cell carcinoma, metastatic skin carcinoma, squamous cell carcinoma of both ulcerating and papillary type, stomach cancer, brain cancer, liver cancer, adrenal cancer, kidney cancer, thyroid cancer, medullary carcinoma, osteosarcoma, soft-tissue sarcoma, Ewing's sarcoma, veticulum cell sarcoma, and Kaposi's sarcoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, leiomyosarcoma, rhabdomyosarcoma, squamous cell carcinoma, adenocarcinoma, sweat gland carcinoma, sebaceous gland carcinoma, papillary carcinoma, glioblastoma, papillary adenocarcinomas, cystadenocarcinoma, bronchogenic carcinoma, seminoma, embryonal carcinoma, Wilms' tumor, small cell lung carcinoma, epithelial carcinoma, astrocytoma, medulloblastoma, craniopharyngioma, ependymoma, pinealoma, hemangioblastoma, acoustic neuroma, oligodendroglioma, meningioma, neuroblastoma, retinoblastoma, glaucoma, hemangioma, heavy chain disease and metastases.

20. A compound of formula I.a.1 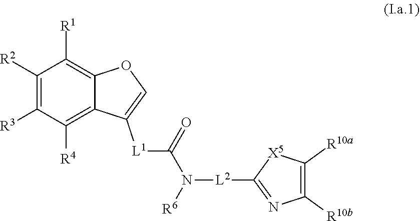
or a tautomer, or a pharmaceutically acceptable salt thereof, wherein the variables for a single compound have the meanings given in one line of the following table:

| No. | R¹ | R² | R³ | R⁴ | L¹ | R⁶ | L² | X⁵ | R^{10a} | R^{10b} |
|---|---|---|---|---|---|---|---|---|---|---|
| 1 | H | H | CH₃ | H | CH₂ | H | bond | S | CF₃ | H |
| 2 | H | H | H | H | CH₂ | H | bond | S | CF₃ | H |
| 3 | CH₃ | H | H | H | CH₂ | H | bond | S | CH₃ | H |
| 4 | H | Cl | H | H | CH₂ | H | bond | S | CH₃ | H |
| 5 | H | —CH₂CH₂CH₂— | | H | CH₂ | H | bond | O | CH₃ | H |
| 6 | CH₃ | CH₃ | H | H | CH₂ | H | bond | NH | CH₃ | H |
| 7 | H | —CH₂CH₂CH₂— | | H | CH₂ | H | bond | NH | H | CH₃ |
| 8 | H | —O—CH₂—O— | | H | CH₂ | H | bond | S | CH₃ | H |
| 9 | H | Cl | H | H | CH₂ | H | bond | NH | CF₃ | H |
| 10 | F | F | H | H | CH₂ | H | bond | S | CF₃ | H |
| 11 | H | H | H | H | Et | H | bond | S | CF₃ | H |
| 13 | H | Cl | H | H | CH₂ | H | bond | S | CN | H |
| 14 | Cl | Cl | H | H | CH₂ | H | bond | NH | CF₃ | H |
| 15 | Cl | H | H | H | CH₂ | H | bond | S | CF₃ | H |
| 16 | CH₃ | CH₃ | H | H | CH₂ | H | bond | NH | CF₃ | H |
| 17 | H | Cl | H | H | CH₂ | H | bond | S | CH₂OCH₃ | H |
| 18 | Cl | CH₃ | H | H | CH₂ | H | bond | NH | CH₃ | H |
| 19 | Cl | CH₃ | H | H | CH₂ | H | bond | NH | CF₃ | H |
| 20 | Cl | CH₃ | H | H | CH₂ | H | bond | S | CN | H |
| 21 | H | Cl | H | H | CH₂ | H | bond | S | CF₃ | H |
| 22 | CH₃ | CH₃ | H | H | Et | H | bond | S | CF₃ | H |
| 23 | CH₃ | CH₃ | H | H | CH₂ | H | bond | S | CN | H |
| 24 | H | —CH₂CH₂CH₂— | | H | CH₂ | H | bond | S | CF₃ | H |
| 25 | H | —CH₂CH₂CH₂— | | H | CH₂ | H | bond | S | CH₂OCH₃ | H |
| 26 | H | Cl | H | H | CH(CH₃) | H | bond | S | CF₃ | H |
| 27 | CH₃ | H | H | H | CH₂ | H | bond | S | CF₃ | H |
| 28 | Cl | Cl | H | H | CH₂ | H | bond | S | CN | H |
| 29 | Cl | Cl | H | H | CH₂ | H | bond | S | CH₃ | H |
| 30 | CH₃ | OCH₃ | H | H | CH₂ | H | bond | S | CH₃ | H |
| 31 | H | —CH₂CH₂CH₂— | | H | CH₂ | H | EtNH | S | CH₂OH | H |
| 32 | CH₃ | CH₃ | H | H | CH₂ | 4-SCHF₂—C₆H₄ | bond | S | —CH═CH—CH═CH— | |
| 33 | Cl | CH₃ | H | H | CH₂ | H | bond | S | CF₃ | H |
| 34 | H | —CH₂CH₂CH₂— | | H | CH₂ | H | bond | S | C₂H₅ | H |
| 35 | Cl | Cl | H | H | CH₂ | H | bond | S | CF₃ | H |
| 36 | OCH₃ | CH₃ | H | H | CH₂ | H | bond | S | CH₃ | H |
| 37 | CH₃ | CH₃ | H | H | CH₂ | H | bond | S | CH₂OCH₃ | H |
| 38 | CH₃ | CH₃ | H | H | CH₂ | H | bond | S | CH₃ | H |
| 39 | Cl | CH₃ | H | H | CH₂ | H | bond | S | CH₂OCH₃ | H |
| 40 | Cl | CH₃ | H | H | CH₂ | H | bond | S | CH₃ | H |
| 41 | CH₃ | OCH₃ | H | H | CH₂ | H | bond | S | CF₃ | H |
| 42 | CH₃ | Cl | H | H | CH₂ | H | bond | S | CH₃ | H |
| 43 | Cl | Cl | H | H | CH₂ | H | bond | S | CH₂OCH₃ | H |
| 44 | CH₃ | Cl | H | H | CH₂ | H | bond | S | CF₃ | H |
| 45 | CH₃ | CH₃ | H | H | CH₂ | H | bond | S | CH(CH₃)₂ | H |
| 46 | CH₃ | CH₃ | H | H | CH₂ | H | bond | S | CH₃ | H |
| 47 | OCH₃ | CH₃ | H | H | CH₂ | H | bond | S | CF₃ | H |
| 48 | CH₃ | CH₃ | H | H | CH₂ | H | bond | S | C₂H₅ | H |
| 49 | CH₃ | CH₃ | H | H | CH₂ | H | bond | S | CF₃ | H |
| 50 | H | —CH₂CH₂CH₂— | | H | CH₂ | H | bond | S | H | 5-am-furan-2-yl |
| 51 | H | CH₃ | CH₃ | H | CH₂ | H | bond | S | H | 4-am-phenyl |
| 52 | CH₃ | CH₃ | H | H | CH₂ | H | bond | S | CF₃ | C(O)—NH—CH₃ |
| | | | | | | | | | |
|---|---|---|---|---|---|---|---|---|---|
| 53 | H | —CH₂CH₂CH₂— | H | CH₂ | H | bond | S | H | 5-ac-am-furan-2-yl |

where Et is CH₂CH₂; EtNH is CH₂CH₂NH; 4-SCHF₂—C₆H₄ is 4-difluoromethylsulfanylphenyl; 4-OMe-C₆H₄ is 4-methoxyphenyl; 5-am-furan-2-yl is 5-aminomethylfuran-2-yl; 4-amphenyl is 4-aminomethylphenyl; C(O)—NH—CH₃ is N-methyl-carboxamide; and 5-ac-am-furan-2-yl is 5-(N-acetylaminomethyl)-furan-2-yl;
or of formula I.b 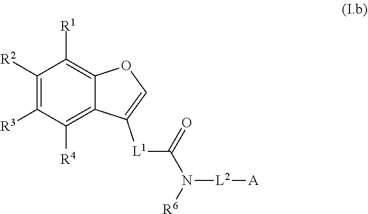
or a tautomer, or a pharmaceutically acceptable salt thereof, wherein the variables for a single compound have the meanings given in one line of the following table:

| | | | | | | | | |
|---|---|---|---|---|---|---|---|---|
| No. | R¹ | R² | R³ | R⁴ | L¹ | R⁶ | L² | A |
| | | | | | | | |
|---|---|---|---|---|---|---|---|
| 54 | H | —CH₂CH₂—CH₂— | H | CH₂ | H | bond | 1-methyl- |
| | | | | | | | pyrazol-3-yl |

or of formula I.c 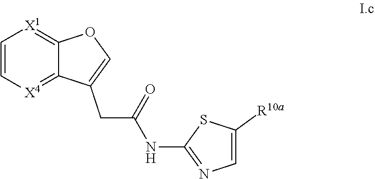
or a tautomer, or a pharmaceutically acceptable salt thereof, wherein the variables for a single compound have the meanings given in one line of the following table:

| No. | X¹ | X⁴ | R^{10a} |
|---|---|---|---|
| 55 | N | C | CH₃ |
| 56 | C | N | CH₃ |
| 57 | N | C | CF₃. |
